(19) United States
(12) Patent Application Publication
(10) Pub. No.: US 2024/0417436 A1
(43) Pub. Date: Dec. 19, 2024

(54) CONDITIONALLY ACTIVATED IMMUNOCYTOKINES AND METHODS OF USE

(71) Applicant: BRIGHT PEAK THERAPEUTICS AG, Basel (CH)

(72) Inventors: Vijaya Raghavan PATTABIRAMAN, Volketswil (CH); Bertolt KREFT, Kleinmachnow (DE); Grégory UPERT, Kembs (FR); Matilde ARÉVALO-RUIZ, Basel (CH); Nina RESCHKE, Birsfelden (CH); Davor BAJIC, Basel (CH); Olivier BORNERT, Baldenheim (FR); Arnaud GOEPFERT, Sierentz (FR)

(21) Appl. No.: 18/410,102

(22) Filed: Jan. 11, 2024

Related U.S. Application Data
(60) Provisional application No. 63/438,468, filed on Jan. 11, 2023.

(51) Int. Cl. C07K 14/55; C07K 16/28

(52) U.S. Cl. CPC C07K 14/55; C07K 16/2818; C07K 2317/92; C07K 2319/30; C07K 2319/50

(57) ABSTRACT

The present disclosure relates activatable immunocytokines targeted to immune checkpoint molecules such as PD-1, compositions comprising the activatable immunocytokines, and methods of use thereof. The present disclosure also relates activatable IL-2 polypeptides linked to anti-PD-1 polypeptides (e.g., anti-PD-1 antibodies).

### CROSS REFERENCE

This application claims the benefit of U.S. Provisional Application No. 63/438,468 filed Jan. 11, 2023, which application is incorporated herein by reference in its entirety.

### SEQUENCE LISTING

10001.11 The instant application contains a Sequence Listing which has been submitted electronically in XML file format and is hereby incorporated by reference in its entirety. Said XML copy, created on Jan. 31, 2024, is named 94917-0114_731201US_SL.xml and is 866,619 bytes in size.

### BACKGROUND

Each year, millions of new cases of cancer are diagnosed across the world, and over 600,000 people are estimated to die from the disease in the United States. Immunotherapies utilize the immune system of a subject to aid in the treatment of ailments. Immunotherapies can be designed to either activate or suppress the immune system depending on the nature of the disease being treated. A goal of various immunotherapies for the treatment of cancer is to stimulate the immune system so that it recognizes and destroys tumors or other cancerous tissue.

Programmed cell death protein 1 (PD-1) is a protein on the surface of cells that regulates the immune system's response to cells of the human body by downregulating the immune system and promoting self-tolerance by suppressing T cell inflammatory activity. Programmed cell death-ligand 1 (PD-L1) is a type 1 transmembrane protein that suppresses the adaptive arm of the immune system. The PD-1 and PD-L1 pathways represent adaptive immune system resistance mechanisms exerted by tumor cells in response to endogenous immune anti-tumor activity. PD-1 inhibitors, such as anti-PD-1 polypeptides and anti-PD-1 antigen binding fragments are checkpoint inhibitor anticancer agents that block the activity of PD-1 immune checkpoint proteins.

Single agent therapies alone, however, in many instances are insufficient in achieving durable responses in cancer patients. The cytokine IL-2 is a potent regulator of the adaptive immune system. When used in concert with a PD-1 inhibitor IL-2 increases the activity of tumor antigen specific effector T cells. However, IL-2 can cause off target effects in non-tumor tissues such as vascular leak syndrome. Thus, there is a need for improved therapies to specifically treat tumors.

### BRIEF SUMMARY

Described herein are activatable immunocytokines which contain polypeptides which bind to immune checkpoint molecules, such as anti-programmed cell death protein 1 (PD-1) antibodies, and interleukin 2 (IL-2) and uses thereof. In one aspect is a composition comprising: a polypeptide which selectively binds to programmed cell death protein 1 (PD-1); an IL-2 polypeptide; and a linker connecting the polypeptide which selectively binds to PD-1 to the IL-2 polypeptide; and a cleavable moiety attached to the IL-2 polypeptide. In some embodiments the cleavable moiety is attached to the IL-2 polypeptide such that the activity of the IL-2 immunocytokine is altered or reduced as compared to the corresponding polypeptide without the cleavable moiety. In some embodiments, cleavage of the cleavable moiety causes activation of the IL-2 in the immunocytokine.

In some embodiments, the cleavable moiety is selected so that it is selectively or preferentially cleaved in or near a target tissue (e.g., a tumor microenvironment). In some embodiments, the cleavable moiety is cleaved by a protease (e.g., a protease which is present at elevated levels at or near a tumor microenvironment). In some embodiments, immunocytokines containing an activatable IL-2 have the advantage of selectively treating the target tissue while sparing off target effects at non-target tissues.

In some embodiments, the cleavable moiety is attached to the IL-2 polypeptide of the immunocytokine at one or more internal residue side chains. In some embodiments, the attachment of a cleavable moiety sterically blocks or otherwise reduces the ability of the IL-2 of the immunocytokine to bind to an IL-2 receptor subunit or complex when the cleavable moiety is intact.

In some embodiments, the cleavable moiety is attached to the IL-2 polypeptide at multiple points of attachment. In some embodiments, the cleavable moiety constricts the conformation of the IL-2 polypeptide so that the binding of the IL-2 immunocytokine to the IL-2 receptor, or a subunit thereof, is reduced. In some embodiments, the cleavable moiety is attached to a side chain of an amino acid residue of the IL-2 polypeptide and the amino or carboxyl terminus or to an additional side chain of the IL-2 polypeptide (e.g., the cleavable moiety is attached to the side chain of two separate amino acid residues). In some embodiments, cleavage of the cleavable moiety allows for a natural conformation or surface of the IL-2, thereby increasing binding to the IL-2 receptor or a subunit thereof.

In some embodiments, upon activation of the IL-2 polypeptide of the activatable immunocytokine, the IL-2 polypeptide exhibits a binding profile which is biased in favor of the IL-2 receptor beta subunit over the alpha subunit. In some embodiments, this biasing of the IL-2 polypeptide to the beta subunit results in the IL-2s ability to preferentially stimulate effector T cells and/or natural killer (NK) cells while sparing the stimulation of regulatory T cells. In some embodiments, the activated IL-2 polypeptide exhibits substantially reduced binding to the IL-2 receptor alpha subunit (or IL-2 receptor αβγ complex) compared to wild type IL-2 but retains binding to the IL-2 receptor beta subunit (or the IL-2 receptor R complex), thereby allowing the activatable immunocytokine to stimulate desired T cells upon activation.

The activatable immunocytokines of the instant disclosure can be used in therapeutic applications. In some embodiments, an activatable immunocytokine of the instant disclosure is useful in the treatment of cancer.

In one aspect, provided herein, is an activatable immunocytokine, comprising: an antibody or antigen binding fragment thereof specific for programmed cell death protein 1 (PD-1); an interleukin-2 (IL-2) polypeptide; a linker connecting the antibody or antigen binding fragment thereof to the IL-2 polypeptide; and a cleavable moiety attached to the IL-2 polypeptide, wherein the IL-2 polypeptide exhibits an enhanced ability to bind to at least one IL-2 receptor subunit after cleavage of the cleavable moiety compared to the activity before cleavage of the cleavable moiety.

In some embodiments, the cleavable moiety comprises a cleavable peptide. In some embodiments, the cleavable peptide is a protease cleavable peptide. In some embodiments, wherein the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the cleavable peptide is cleavable by multiple proteases. In some embodiments, cleavage of the cleavable peptide leaves 1, 2, 3, 4, 5, 6, 7, 8, 9, or 10 amino acids attached to the side chain of the amino acid residue to which the cleavable moiety is attached. In some embodiments, the cleavable peptide comprises an amino acid sequence having at least about 80%, at least about 90%, or 100% identity to a sequence set forth in any one of Table 1B, Table 1C, or Table 1D. In some embodiments, the cleavable peptide comprises an amino acid sequence having at least about 80%, at least about 90%, or 100% identity to a sequence set forth in any one of Table 1B or Table 1C. In some embodiments, the cleavable peptide comprises an amino acid sequence having at least about 80%, at least about 90%, or 100% identity to a sequence set forth in Table 1D.

In some embodiments, the cleavable moiety is attached to a side chain of an amino acid residue of the IL-2 polypeptide. In some embodiments, the C-terminus of the cleavable peptide is attached to the side chain of the amino acid residue of the IL-2 polypeptide, optionally through a linking group. In some embodiments, the amino acid residue to which the cleavable moiety is attached is lysine, glutamate, glutamine, aspartate, asparagine, tyrosine, serine, threonine, cysteine, or an unnatural amino acid. In some embodiments, the amino acid residue to which the cleavable moiety is attached is a lysine or glutamate. In some embodiments, the amino acid residue to which the cleavable moiety is attached is substituted relative to the corresponding residue in SEQ ID NO: 1.

In some embodiments, the cleavable moiety is attached to a residue which contacts the IL-2 receptor beta subunit or the IL-2 receptor gamma subunit during binding to the IL-2 receptor. In some embodiments, the cleavable moiety is attach to a residue selected from residues 9, 11, 13, 15, 16, 19, 20, 22, 23, 29, 32, 84, 88, 91, 123, 126, and 129 of the IL-2 polypeptide, wherein residue position numbering is based on SEQ ID NO: 1 as a reference sequence. In some embodiments, the cleavable moiety is attached to residue 9, 11, 13, 15, 16 19, 22, 23, 29, or 32 of the IL-2 polypeptide, wherein residue position numbering is based on SEQ ID NO: 1 as a reference sequence.

In some embodiments, the cleavable moiety is attached to the IL-2 polypeptide at an additional point of attachment. In some embodiments, the additional point of attachment is to the N-terminus of the IL-2 polypeptide.

In some embodiments, the additional point of attachment is to a side chain of another amino acid residue of the IL-2 polypeptide. In some embodiments, the additional point of attachment is to residue 9 of the IL-2 polypeptide, wherein residue position numbering is based on SEQ ID NO: 1 as a reference sequence.

In some embodiments, the cleavable moiety is attached to residue 23 of the IL-2 polypeptide and the additional point of attachment is to the N-terminus of the IL-2 polypeptide. In some embodiments, the N-terminus of the IL-2 polypeptide is the amino acid residue at a position corresponding to the first residue of SEQ ID NO: 1. In some embodiments, the cleavable moiety comprises a cleavable peptide. In some embodiments, the C-terminus of the cleavable peptide is attached to the N-terminus of the IL-2 polypeptide and the N-terminus of the cleavable peptide is attached to residue 23 of the IL-2 polypeptide. In some embodiments, the cleavable peptide comprises the sequence set forth in SEQ ID NO: 617 or 633. In some embodiments, residue 23 of the IL-2 polypeptide comprises a carboxylic acid side chain. In some embodiments, residue 23 of the IL-2 polypeptide is glutamate.

In some embodiments, the cleavable moiety is attached to the IL-2 polypeptide through a linking group. In some embodiments, the cleavable moiety is attached to the side chain of the amino acid residue through a linking group.

In some embodiments, the IL-2 polypeptide exhibits reduced binding to the IL-2 receptor alpha subunit compared to wild type IL-2. In some embodiments, the IL-2 polypeptide comprises at least one modification which reduces the affinity of the IL-2 polypeptide to the IL-2 receptor alpha compared to wild type IL-2. In some embodiments, the IL-2 polypeptide comprises at least one polymer covalently attached to a residue selected from residues 35, 37, 38, 41, 42, 43, 44, 45, 60, 61, 62, 64, 65, 68, 69, 71, 72, 104, 105, and 107, wherein residue position numbering is based on SEQ ID NO: 1 as a reference sequence. In some embodiments, the IL-2 polypeptide comprises at least one polymer covalently attached to a residue selected from residue 42 and 45, wherein residue position numbering is based on SEQ ID NO: 1 as a reference sequence. In some embodiments, wherein the IL-2 polypeptide comprises polymers covalently attached at residues 42 and 45, wherein residue position numbering is based on SEQ ID NO: 1 as a reference sequence. In some embodiments, the IL-2 polypeptide is synthetic. In some embodiments, the IL-2 polypeptide comprises an amino acid sequence having at least about 80%, at least about 85%, at least about 90%, or at least about 95% sequence identity SEQ ID NO: 2 or SEQ ID NO: 3.

In some embodiments, the antibody or antigen binding fragment thereof is a monoclonal antibody, a humanized antibody, a grafted antibody, a chimeric antibody, a human antibody, a deimmunized antibody, or a bispecific antibody. In some embodiments, the antibody or antigen binding fragment thereof an antigen binding fragment, wherein the antigen binding fragment comprises a Fab, a Fab′, a F(ab′)2, a bispecific F(ab′)2, a trispecific F(ab′)2, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a VHH, a minibody, an intrabody, fusion proteins comprising an antibody portion (a domain antibody), a single chain binding polypeptide, a scFv-Fc, a Fab-Fc, a bispecific T cell engager (BiTE), a tetravalent tandem diabody (TandAb), a Dual-Affinity Re-targeting Antibody (DART), a bispecific antibody (bscAb), a single domain antibody (sdAb), a fusion protein, or a bispecific disulfide-stabilized Fv antibody fragment (dsFv-dsFv′). In some embodiments, the antibody or antigen binding fragment thereof comprises an IgG, an IgM, an IgE, an IgA, an IgD, or is derived therefrom. In some embodiments, the antibody or antigen binding fragment thereof comprises an IgG1 or an IgG4, or is derived therefrom.

In some embodiments, the antibody or antigen binding fragment thereof comprises tislelizumab, baizean, sintilimab, tamrelizumab, emiplimab, cemiplimab, lambrolizumab, pembrolizumab, nivolumab, prolgolimab, forteca, penpulimab, zimberelimab, balstilimab, genolimzumab, geptanolimab, dostarlimab, serplulimab, retifanlimab, sasanlimab, spartalizumab, cetrelimab, tebotelimab, cadonilimab, pidilizumab, budigalimab, LZM-009, or a modified version thereof. In some embodiments, the antibody or antigen binding fragment thereof comprises nivolumab, pembrolizumab, LZM-009, or cemiplimab, or a modified version thereof.

In some embodiments, the linker is a chemical linker. In some embodiments, the linker comprises a polymer. In some embodiments, wherein the polymer is a water soluble polymer. In some embodiments, the polymer comprises poly(ethylene glycol). In some embodiments, the linker comprises a structure

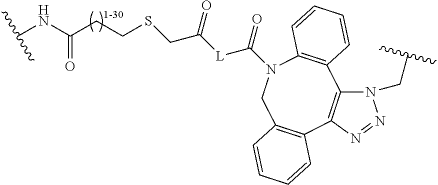

wherein

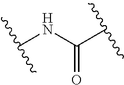 [SMILES: *NC(*)=O]

is the first point of attachment to a lysine residue of the antibody or antigen binding fragment;
L is a tether group; and

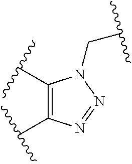 [SMILES: *Cn1nnc(*)c1*]

is a point of attachment to a tether group which connects to the IL-2 polypeptide.

In some embodiments, the linker comprises a point of attachment to the antibody or antigen binding fragment thereof at a non-terminal residue of the antibody or antigen binding fragment thereof. In some embodiments, the point of attachment to the antibody or antigen binding fragment thereof is to a residue in an Fc region of the antibody or antigen binding fragment thereof the point of attachment to the antibody or antigen binding fragment thereof is at a position of a K246 amino acid residue, a K248 amino acid residue, a K288 amino acid residue, a K290 amino acid residue, a K317 amino acid residue, or a combination thereof (Eu numbering). In some embodiments, the point of attachment to the antibody or antigen binding fragment thereof is at the K248 amino acid residue (Eu numbering).

In some embodiments, the linker comprises a point of attachment to the IL-2 polypeptide. In some embodiments, the point of attachment to the IL-2 polypeptide is at a residue in the region of amino acid residues 30-110 of the modified IL-2 polypeptide, wherein amino acid residue position numbering of the IL-2 polypeptide is based on SEQ ID NO: 1 as a reference sequence. In some embodiments, the point of attachment to the IL-2 polypeptide is at an amino acid residue selected from the group consisting of amino acid residue 35, 37, 38, 41, 42, 43, 44, 45, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the point of attachment to the IL-2 polypeptide is at amino acid residue 42 or 45, wherein amino acid residue position numbering of the modified IL-2 polypeptide is based on SEQ ID NO: 1 as a reference sequence. In some embodiments, the point of attachment to the IL-2 polypeptide is at amino acid residue F42Y or Y45. In some embodiments, the point of attachment to the IL-2 polypeptide is to the N-terminus of the IL-2 polypeptide.

In an aspect herein is a method of manufacturing an activatable immunocytokine provided herein. In some embodiments, the method comprises providing the antibody or antigen binding fragment which binds to PD-1, wherein the antibody or antigen binding fragment comprises a first conjugation handle. In some embodiments, the method comprises providing the IL-2 polypeptide, wherein the IL-2 polypeptide comprise a second conjugation handle complementary to the first conjugation handle. In some embodiments, the method comprises contacting the first conjugation handle with the second conjugation handle to form the activatable immunocytokine.

In one aspect herein is a pharmaceutical composition comprising an activatable immunocytokine provided herein and a pharmaceutically acceptable carrier.

In another aspect provided herein a method of treating cancer in a subject in need thereof, comprising: administering to the subject a therapeutically effective amount of an activatable immunocytokine provided herein or a pharmaceutical composition provided herein. In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer. In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIGS. 1A and 1B shows a schematic of an exemplary mechanism of an activatable immunocytokine composition provided herein comprising an activatable IL-2 polypeptide and an anti-PD-1 antibody.

FIG. 1A shows the activatable immunocytokine in the uncleaved state, representative of the state of the activatable IL-2 and PD-1 immunocytokine in non-disease tissue (left) and cleavage after entering the tumor microenvironment (TME), resulting in a more-active (“unmasked”) immunocytokine (right).

FIG. 1B shows the cleavage product of an activatable immunocytokine (“unmasked” activatable immunocytokine) in the tumor microenvironment engaging with an activated T cell.

FIG. 1C shows an illustration of a mechanism of action of an exemplary activatable immunocytokine provided herein.

FIG. 2A shows a plot comparing the concentration where the indicated IL-2 polypeptide variant shows 50% of maximal activation of IL-2 reporter (EC50) for the indicated activatable IL-2 polypeptides of or control IL-2 polypeptide CMP-003.

FIG. 2B shows a plot comparing the concentration where the indicated activatable and cleaved IL-2 polypeptide variants show 50% of maximal activation of the IL-2 reporter (EC50), as well as control IL-2 polypeptide CMP-003. The activatable IL-2 polypeptides described therein include attachment of a cleavable peptide to a single residue of the IL-2 polypeptide. Activatable IL-2 polypeptides were tested either intact (black) or MMP2 treated (grey).

FIG. 2C shows a plot comparing the concentration where the indicated activatable and cleaved IL-2 polypeptide variants show 50% of maximal activation of the IL-2 reporter (EC50), as well as control IL-2 polypeptide CMP-003. The activatable IL-2 polypeptides described therein include attachment of a cleavable peptide to the IL-2 polypeptide at 2 residues, thereby forming a macrocyclic structure. Activatable IL-2 polypeptides were tested either intact (black) or MMP2 treated (grey).

FIG. 3A shows bilayer interferometry traces for synthetic IL-2 cytokine CMP-003, and overlaid model traces. Binding data is shown for IL-2Rβ (CD122) (left) and IL-2Rβγ (CD122 CD132 dimer)(right).

FIG. 3D shows bilayer interferometry traces for intact activatable IL-2 cytokine CMP-136, and overlaid model traces (smoothed lines). Binding data is shown for IL-2Rβ (left) and IL-2Rβγ (right).

FIG. 3C shows bilayer interferometry traces for intact activatable IL-2 cytokine CMP-145, and overlaid model traces (smoothed lines). Binding data is shown for IL-2Rβ (left) and IL-2Rβγ (right).

FIG. 3D shows bilayer interferometry traces for intact activatable IL-2 cytokine CMP-138, and overlaid model traces (smoothed lines). Binding data is shown for IL-2Rβ (left) and IL-2Rβγ (right).

FIG. 3E shows bilayer interferometry traces for intact activatable IL-2 cytokine CMP-151, and overlaid model traces (smoothed lines). Binding data is shown for IL-2Rβ (left) and IL-2Rβγ (right).

FIG. 4A shows a plot of the average result of pSTAT5 assays for the indicated IL-2 polypeptides.

FIG. 4B shows a plot of the average EC50 result of pSTAT5 assays for the indicated IL-2 molecules with a comparison of intact (uncleaved) IL-2 molecules (black) vs MMP2 treated (cleaved) IL-2 molecules (gray).

FIG. 4C shows a plot of the average EC50 result of pSTAT5 assays for cyclic activatable IL-2 molecules with a comparison of intact (uncleaved) IL-2 molecules (black) vs MMP2 treated (cleaved) IL-2 molecules (gray).

FIG. 5A shows site-selective modification of anti-PD1 antibody by chemical modification technology to introduce one or two conjugation handles.

FIG. 5B shows Q-TOF mass spectra of unmodified Pembrolizumab and Pembrolizumab with a DBCO conjugation handle.

FIG. 5C shows site-selective conjugation of modified IL2 cytokine to generate a PD1-IL2 with DAR1, DAR 2 or mixed DAR between 1 and 2.

FIG. 6A shows the results of a HEK Blue IL-2 reporter assay for intact and MMP2 cleaved activatable IL-2/anti-PD-1 antibody immunocytokines.

FIG. 6B shows results of a HEK Blue IL2 receptor (CD122/CD132) reporter assay for intact and MMP2 treated activatable immunocytokines, with approximate fold-change compared to CMP-016 (non-activatable control) indicated.

FIG. 6C shows results of a HEK Blue IL2 receptor (CD122/CD132) reporter assay for intact and MMP9 treated activatable immunocytokines, with approximate fold-change compared to CMP-016 (non-activatable control) indicated.

FIG. 6D shows results of a HEK Blue IL2 receptor reporter assay for the indicated molecules in intact form and after treatment with the indicated proteases.

FIG. 6E shows results of a HEK Blue IL2 receptor reporter assay for the indicated molecules in intact form and after treatment with the indicated proteases.

FIG. 7A shows the KD (nM) values of the indicated activatable immunocytokines (and corresponding controls) with CD122 before and after treatment with MMP2 as measured by biolayer interferometry (BLI).

FIG. 7B shows the KD (nM) values of the indicated activatable immunocytokines (and corresponding controls) with CD122/CD132 before and after treatment with MMP2 as measured by BLI.

FIG. 8 shows pSTAT5 levels of human pan T cells after treatment with the indicated activatable immunocytokines before and after MMP treatment. Cells were gated on CD8 memory Teff cells.

FIG. 9A shows average EC50s for intact and cleaved (MMP2 treated) activatable immunocytokines for in a pSTAT5 assay on NK cells.

FIG. 9B shows average EC50s for intact and cleaved (protease treated) activatable immunocytokines for in a pSTAT5 assay on NK cells.

FIG. 9C shows average EC50s for intact and cleaved (protease treated) activatable immunocytokines for in a pSTAT5 assay on NK cells.

FIG. 9D shows average EC50s for control immunocytokine CMP-016 and CMP-421, CMP-419, and CMP-420, which simulate various cleavage products of CMP-416.

FIG. 9E shows dose response curves for a pSTAT5 assay performed on CD8 T-memory effector cells using various activatable immunocytokines (intact and protease treated).

FIG. 10 shows EC50s for intact and cleaved activatable immunocytokines in a pSTAT5 assay on CD8 cells derived from mouse splenocytes.

FIG. 11A shows HEK Blue IL2 reporter assay results for activatable immunocytokines incubated in human plasma for the indicated time periods.

FIG. 11B shows HEK Blue IL2 reporter assay results for activatable immunocytokines incubated in mouse (C57BL/6) plasma for the indicated time periods.

FIG. 12A shows weight loss in B16F10-Ova bearing C57BL/6 mice upon treatment with the indicated molecules at 1 mg/kg (single dose).

FIG. 12B shows weight loss in B16F10-Ova bearing C57BL/6 mice upon treatment with the indicated molecules at the indicated dose (single administration).

FIG. 12C shows tumor growth inhibition in B16F10-Ova bearing C57BL/6 mice on Day 8 of the study, with the indicated graphs marked with * showing significant tumor growth inhibition.

FIG. 12D shows Kaplan-Meier survival analysis of B16F10-Ova bearing C57BL/6 mice following intervention with the indicated molecule and dose (single administration). Groups marked with * showed significant survival compared to vehicle.

FIG. 13A shows average EC50s for intact and MMP treated activatable immunocytokines in a pSTAT5 assay on NK92 PDV cells.

FIG. 13B shows average EC50s for intact molecules and molecules pre-treated with protease or cancer cell homogenate in a pSTAT5 assay on NK92 PD1⁺ cells.

FIG. 14 shows representative SDS-PAGE gel analysis of the indicated immunocytokines.

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

### Activatable Immunocytokines

Disclosed herein are activatable immunocytokines. In some embodiments, the activatable immunocytokines comprise immune checkpoint inhibitor molecules, such as anti-PD-1 polypeptides (e.g., anti-PD-1 antibodies or antigen binding fragments thereof), attached (e.g., conjugated) to activatable IL-2 polypeptides. In some embodiments, the IL-2 polypeptides are linked to a cleavable moiety that alters or reduces its activity until cleaved. In some embodiments, cleavage of the cleavable moiety causes an increase the activity of the IL-2 polypeptide.

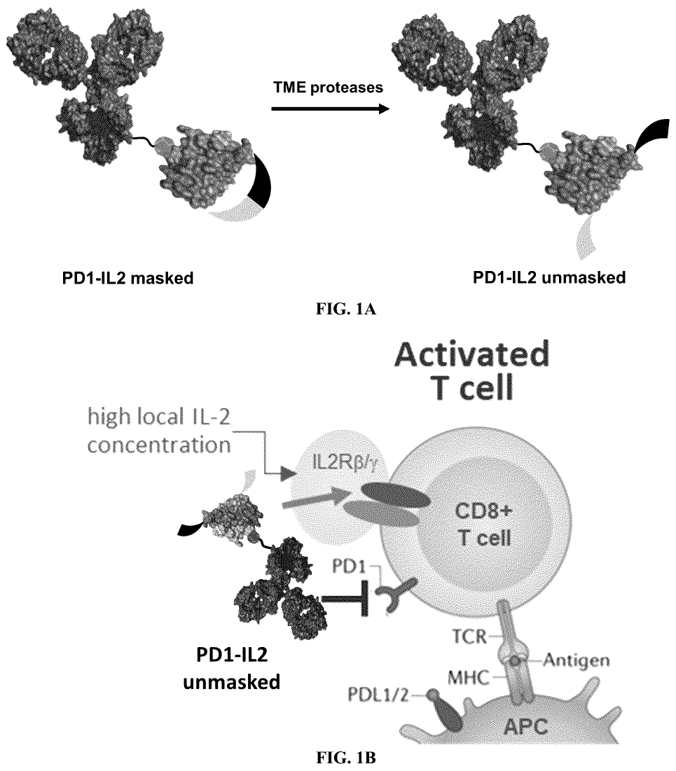

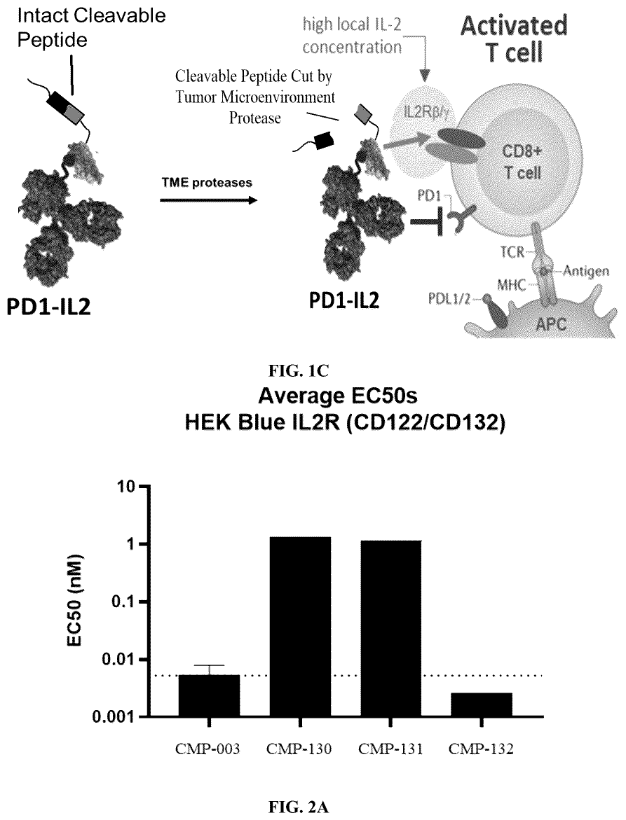

FIGS. 1A and 1B illustrate an exemplary activatable immunocytokine comprising an anti-PD-1 antibody conjugated to an activatable IL-2 polypeptide. The exemplary activatable immunocytokine pictured comprises one activatable IL-2 polypeptide conjugated to antibody. Alternative embodiments with two activatable IL-2 polypeptides (one attached to each Fc region) are also contemplated as within the scope of the instant disclosure. The activatable immunocytokine shown at left of FIG. 1A has the IL-2 polypeptide in the “inactive” state with its cleavable peptide still in intact form, thereby forming a cyclic structure (e.g., the cleavable peptide is attached to a side chain of a residue of the IL-2 polypeptide (optionally by a linking group), such as residue 23, and to the N-terminus of the IL-2 polypeptide (optionally by a linking group)). The IL-2 polypeptide at right of FIG. 1A has been “activated” by cleavage of the cleavable moiety by a tumor microenvironment protease (e.g., matriptase, MMP, etc.), thereby breaking the cyclic structure formed by the cleavable peptide. The activated IL-2 polypeptide is now able bind to the IL-2 receptor βγ complex on the indicated T cell, as depicted in FIG. 1B. The function of the PD-1 antibody in some instances (including that shown) is twofold. First, binding of the antibody to PD-1 on the surface of the T cell brings the IL-2 in close proximity to the T cell located in the tumor microenvironment, allowing for cleavage of the cleavable peptide and high local concentration of active IL-2 polypeptide in the environment. Second, binding of PD-1 by the antibody blocks interaction with PD-L1, which can tamp down the stimulation of the T cell by the IL-2 polypeptide.

FIG. 1C illustrates an alternative exemplary activatable immunocytokine comprising an anti-PD-1 antibody conjugated to an activatable IL-2 polypeptide compared to that described in FIGS. 1A and 1B. The exemplary activatable immunocytokine pictured in FIG. 1C comprises one activatable IL-2 polypeptide conjugated to antibody. The activatable immunocytokine shown at left of FIG. 1C has the IL-2 polypeptide in the “inactive” state with its cleavable peptide still in intact form. In the embodiment shown, a blocking group (e.g., a PEG group) is attached to the cleavable peptide, though in certain embodiments described herein such a blocking group is not required (e.g., the cleavable peptide itself is sufficient to inactivate the IL-2 polypeptide). The IL-2 polypeptide at right of FIG. 1C has been “activated” by cleavage of the cleavable moiety by a tumor microenvironment protease (e.g., matriptase, MMP, etc.). Residual portions of the cleavable peptide remain tethered to the IL-2 polypeptide (optionally by linking groups). However, the blocking group is released upon cleavage. Despite the presence of the linking group and the residual portion of the cleavable peptide, the IL-2 polypeptide is nevertheless more active and better able to bind its receptor than its counterpart with the intact cleavable peptide attached to the other Fc chain. The activated IL-2 polypeptide is now able bind to the IL-2 receptor βγ complex on the indicated T cell.

The immune checkpoint inhibitor (e.g., anti-PD-1 antibody)/activatable IL-2 polypeptide immunocytokines (referred to herein as activatable immunocytokines) of the disclosure can have superior efficacy and potentially improved tolerability by a subject with reduced off target effects. In particular, the “activatable” nature of the IL-2 polypeptide can prevent off target effects owing to binding of the IL-2 receptors in other tissues. In some embodiments, the activatable immunocytokines of the disclosure can directly target tumor-infiltrating lymphocytes (TILs). In some embodiments, the activatable immunocytokines can significantly reduce the therapeutic dose of the anti-PD-1 polypeptide or IL-2 polypeptide for a subject with a disease, such as cancer. In some embodiments, the activatable immunocytokines specifically target tumors while sparing non-tumor tissues from off target effects.

The activatable immunocytokines can act by one or more modes of action. In some embodiments, the activatable immunocytokines can be activated in the tumor microenvironment. In some embodiments, the activatable immunocytokine can inhibit PD-1 by targeting PD-1 and CD8+ T cells within tumors. In some embodiments, the activatable immunocytokines can activate T cells and NK cells via IL-2R βγ.

In one aspect, provided herein, is an activatable immunocytokine, comprising: an antibody or antigen binding fragment thereof specific for an immune checkpoint molecule (e.g., PD-1); an interleukin-2 (IL-2) polypeptide; a linker connecting the antibody or antigen binding fragment thereof to the IL-2 polypeptide; and a cleavable moiety attached to the IL-2 polypeptide, wherein the IL-2 polypeptide exhibits an enhanced ability to bind to at least one IL-2 receptor subunit (e.g., the IL-2 receptor beta subunit) after cleavage of the cleavable moiety compared to the ability before cleavage of the cleavable moiety.

In one aspect, provided herein, is an activatable immunocytokine, comprising: an antibody or antigen binding fragment thereof specific for an immune checkpoint molecule (e.g., PD-1); an interleukin-2 (IL-2) polypeptide; a linker connecting the antibody or antigen binding fragment thereof to the IL-2 polypeptide; and a cleavable moiety attached to the IL-2 polypeptide, wherein the IL-2 polypeptide exhibits an enhanced ability to signal through the IL-2 receptor βγ complex after cleavage of the cleavable moiety compared to the ability before cleavage of the cleavable moiety.

In one aspect, provided herein, is an activatable immunocytokine, comprising: an antibody or antigen binding fragment thereof specific for an immune checkpoint molecule (e.g., PD-1); an interleukin-2 (IL-2) polypeptide; a linker connecting the antibody or antigen binding fragment thereof to the IL-2 polypeptide; and a cleavable moiety attached to the IL-2 polypeptide, wherein the IL-2 polypeptide exhibits an enhanced ability to stimulate T_{eff} cells (e.g., induce STAT5 phosphorylation) after cleavage of the cleavable moiety compared to the ability before cleavage of the cleavable moiety.

In one aspect, provided herein, is an activatable immunocytokine, comprising: an antibody or antigen binding fragment thereof specific for an immune checkpoint molecule (e.g., PD-1); an interleukin-2 (IL-2) polypeptide; a linker connecting the antibody or antigen binding fragment thereof to the IL-2 polypeptide; and a cleavable moiety attached to a side chain of an amino acid residue the IL-2 polypeptide.

In one aspect, provided herein, is an activatable immunocytokine, comprising: an antibody or antigen binding fragment thereof specific for an immune checkpoint molecule (e.g., PD-1); an interleukin-2 (IL-2) polypeptide; a linker connecting the antibody or antigen binding fragment thereof to the IL-2 polypeptide; and a cleavable moiety attached to the IL-2 polypeptide, wherein the IL-2 polypeptide exhibits substantially no binding to the IL-2 receptor alpha subunit (or αβγ complex) before or after cleavage of the cleavable moiety but exhibits enhanced ability to bind to the IL-2 receptor beta subunit (or βγ complex) after cleavage of the cleavable moiety compared to the ability before cleavage of the cleavable moiety.

In some embodiments, the activatable immunocytokines comprise an antibody or antigen binding fragment specific for programed cell death protein 1 (PD-1), an IL-2 polypeptide, a linker connecting the antibody or antigen binding fragment to the IL-2 polypeptide and a cleavable moiety attached to the IL-2 polypeptide. In some embodiments, the cleavage of the cleavable moiety causes the IL-2 polypeptide to exhibit an enhanced ability to bind to at least one IL-2 receptor subunit after cleavage as compared to the binding ability of the IL-2 before cleavage of the cleavable moiety.

### IL-2 Polypeptides Comprising Cleavable Moieties of Activatable Immunocytokines

The activatable immunocytokines provided herein comprise IL-2 polypeptides attached to cleavable moieties (also referred to herein as IL-2 polypeptides comprising cleavable moieties or activatable IL-2 polypeptides). The IL-2 polypeptides comprise cleavable moieties provided herein in order to provide an IL-2 polypeptide which is deactivated with the cleavable moiety attached and which shows greater activity upon cleavage of the cleavable moiety.

In some embodiments, the IL-2 polypeptide comprises modification(s) which diminish the ability of the IL-2 polypeptide to bind to or signal through the IL-2 receptor alpha unit (or the IL-2 receptor αβγ complex) but spare the ability of the IL-2 polypeptide to bind to and signal through the IL-2 receptor beta subunit (or the IL-2 receptor βγ complex). Signaling through the IL-2 receptor alpha subunit (or the IL-2 receptor αβγ complex) is associated with activation of regulatory T cells (Tregs), whereas signaling through the IL-2 receptor beta subunit (or the IL-2 receptor Oy complex) is associated with activation of effector T cells (Teffs) (e.g., CD8+ effector T cells) and/or natural killer (NK) cells. Thus, in some embodiments, an IL-2 polypeptide of an activatable immunocytokine provided herein exhibits an ability to enhance Teff and/or NK cell proliferation while sparing Tregs after cleavage of the cleavable moiety. In some embodiments, the IL-2 polypeptide with the intact cleavable moiety attached exhibits a reduced ability enhance Teff and/or NK cell proliferation compared to the IL-2 polypeptide after cleavage of the cleavable moiety. In some embodiments, the differential activity of the IL-2 polypeptide before and after cleavage of the cleavable moiety is retained when the IL-2 polypeptide is incorporated into an activatable immunocytokine as provided herein. In some embodiments, the cleavable moiety is configured to be selectively or preferentially cleaved in or near a microenvironment of a tumor.

In some embodiments, the IL-2 polypeptide comprises a cleavable moiety attached to the IL-2 polypeptide (e.g., at a side chain of an amino acid residue) that reduces the activity of the IL-2 polypeptide. In some embodiments, the intact cleavable moiety reduces the ability of the IL-2 polypeptide to signal through the IL-2 receptor beta subunit (or the IL-2 receptor βγ complex). In some embodiments, cleavage of the cleavable moiety enhances the ability of the IL-2 polypeptide to signal through the IL-2 receptor beta subunit (or the IL-2 receptor βγ complex).

In one aspect, provided herein, is an activatable immunocytokine comprising an IL-2 polypeptide which displays substantially no binding to the IL-2 receptor alpha (or the IL-2 receptor αβγ complex) and a cleavable moiety which reduces the ability of the IL-2 polypeptide to bind to the IL-2 receptor beta (or the IL-2 receptor βγ complex), and wherein cleavage of the cleavable moiety enhances the ability of the IL-2 polypeptide to bind to the IL-2 receptor beta (or the IL-2 receptor βγ complex), and wherein the IL-2 polypeptide is linked to an immune checkpoint inhibitor molecule (e.g., an anti-PD-1 antibody or antigen binding fragment thereof).

In one aspect provided herein is an activatable immunocytokine, comprising: an IL-2 polypeptide comprising a cleavable moiety attached to a side chain of an amino acid residue of the IL-2 polypeptide, and wherein the IL-2 polypeptide is linked to an immune checkpoint binding molecule (e.g., an anti-PD-1 antibody or antigen binding fragment thereof).

In one aspect provided herein is an activatable immunocytokine, comprising: an IL-2 polypeptide comprising a cleavable moiety attached to a side chain of an amino acid residue of the IL-2 polypeptide, wherein the IL-2 polypeptide displays an altered activity after cleavage of the cleavable moiety compared to the activity of the activatable IL-2 polypeptide before cleavage of the cleavable moiety, and wherein the IL-2 polypeptide is linked to an immune checkpoint inhibitor molecule (e.g., an anti-PD-1 antibody or antigen binding fragment thereof).

In one aspect provided herein is an activatable immunocytokine, comprising: an IL-2 polypeptide comprising a cleavable moiety attached to a side chain of an amino acid residue of the IL-2 polypeptide, wherein the IL-2 polypeptide displays an enhanced ability to bind to at least one IL-2 receptor subunit after cleavage of the cleavable moiety compared to the activity of the activatable IL-2 polypeptide before cleavage of the cleavable moiety, and wherein the IL-2 polypeptide is linked to an immune checkpoint inhibitor molecule (e.g., an anti-PD-1 antibody or antigen binding fragment thereof).

In one aspect provided herein is an activatable immunocytokine comprising an IL-2 polypeptide comprising a cleavable moiety attached to a residue in the region of residues 1-35 of the IL-2 polypeptide, wherein residue position numbering is based on SEQ ID NO: 1 as a reference sequences, and wherein the IL-2 polypeptide exhibits a greater affinity for the IL-2 receptor beta subunit after cleavage of the cleavable moiety compared to the activatable IL-2 polypeptide before cleavage of the cleavable moiety, and wherein the IL-2 polypeptide is linked to an immune checkpoint binding molecule (e.g., an anti-PD-1 antibody or antigen binding fragment thereof).

In one aspect provided herein is an activatable immunocytokine comprising an IL-2 polypeptide comprising a cleavable moiety, wherein the IL-2 polypeptide exhibits a greater affinity for the IL-2 receptor beta subunit after cleavage of the cleavable moiety compared to the activatable IL-2 polypeptide before cleavage of the cleavable moiety, and wherein the IL-2 polypeptide is linked to an immune checkpoint binding molecule (e.g., an anti-PD-1 antibody or antigen binding fragment thereof).

### Cleavable Moieties Attached to IL-2 Polypeptides of Activatable Immunocytokines

In some embodiments, the IL-2 polypeptide of the activatable immunocytokine comprises a cleavable moiety attached to the IL-2 polypeptide (e.g., to a side chain of an amino acid residue). In some embodiments, the cleavable moiety is attached in a manner such that it alters an activity of the activatable IL-2 polypeptide relative to the same IL-2 polypeptide without the cleavable moiety. In some embodiments, cleavage of the cleavable moiety restores at least a portion of the activity of the IL-2 polypeptide compared to the activatable IL-2 polypeptide with the cleavable moiety intact (e.g., the relevant activity is enhanced by 2-fold, 3-fold, 5-fold, 10-fold, 20-fold, 50-fold, etc. after cleavage).

In some embodiments, cleavage of the cleavable moiety leaves a residual portion of the cleavable moiety attached to the IL-2 polypeptide of the activatable immunocytokine. In such cases, the residual portion of the cleavable moiety preferably has a smaller impact on the activity than the intact cleavable moiety. In some embodiments, both the residual portion of the cleavable moiety and the intact cleavable moiety may have an impact on the activity relative to a version of the IL-2 polypeptide to which no group is attached (e.g., a version of the IL-2 polypeptide with the substitution at the point of attachment which allows for the attachment of the cleavable moiety, or to a version of the IL-2 polypeptide which is wild type at the relevant residue). For example, an activatable IL-2 polypeptide (e.g., the IL-2 polypeptide with the intact cleavable moiety attached) may have an activity which is reduced by 100-fold compared to the IL-2 polypeptide with no group attached and the IL-2 polypeptide with the residual portion attached may have an activity which is reduced by only 5-fold compared to the IL-2 polypeptide with no group attached.

In some embodiments, the cleavable moiety is selected such that it is preferentially cleaved (e.g., cleaved at a faster rate or cleaved in more abundance) at a designated target tissue of a subject. In some embodiments, the cleavable moiety is preferentially at or near a target tissue of the subject such that the cleavable moiety is cleaved at a rate of least 2-fold, at least 4-fold, at least 8-fold, at least 10-fold, at least 15-fold, at least 20-fold, at least 50-fold, at least 100-fold, at least 200-fold, at least 500-fold, or at least 1000-fold greater than cleavage of the cleavable moiety at a different tissue. In some embodiments, the target tissue is diseased tissue of the subject. In some embodiments, the target tissue is cancer tissue of the subject. In some embodiments, the target tissue is a tumor microenvironment. In some embodiments, the target tissue is a tumor. In some embodiments, the target tissue is at or near a tumor.

In some embodiments, the cleavable moiety is attached directly to the IL-2 polypeptide of the activatable immunocytokine (e.g., by a bond between the cleavable moiety itself (e.g., a portion of the cleavable moiety necessary for its desired or optimal cleavage, such as an amino acid residue of a protease recognition sequence) and the IL-2 polypeptide). In some embodiments, the cleavable moiety is attached to the IL-2 polypeptide via a linking group. In some embodiments, the cleavable moiety is attached to a side chain of an amino acid residue of the IL-2 polypeptide through a linking group. The linking group can be any suitable structure that provides a connection via a chain of atoms between the point of attachment to the IL-2 polypeptide (e.g., the side chain of an amino acid residue) and the cleavable moiety. In some embodiments, the linking group is attached to the IL-2 polypeptide via a reaction with a suitable reactive group capable of reacting with a side chain of the IL-2 polypeptide to form a bond (e.g., an amide, an ester, a carbamate, a carbonate, a carbamide, a thioether, or a disulfide bond is formed). In some embodiments, the linking group is attached to the IL-2 polypeptide via an amide, an ester, a carbamate, a carbonate, a carbamide, a thioether, or a disulfide bond. The linking group can be selected to impart desired properties to the final IL-2 polypeptide of the activatable immunocytokine (e.g., with the intact cleavable moiety attached) or to IL-2 polypeptide with a residual portion of the cleavable moiety attached (e.g., after cleavage). In some embodiments, the linking group remains attached to the IL-2 polypeptide after cleavage of the cleavable moiety.

In some embodiments, the linking group is a chemical linking group. In some embodiments, the linking group comprises at least one portion which is not comprised of amino acid residues. In some embodiments, the linking group comprises a polymer. In some embodiments, the linking group comprises a non-polymer. In some embodiments, the linking group comprises a polymer and a non-polymer (e.g., a polymeric portion and a non-polymeric portion).

In some embodiments, the linking group comprises a polymer. In some embodiments, the linking group comprises a water soluble polymer. In some embodiments, the linking group comprises poly(alkylene oxide), polysaccharide, poly(vinyl pyrrolidone), poly(vinyl alcohol), polyoxazoline, poly(acryloylmorpholine), or a combination thereof. In some embodiments, the linking group comprises poly(alkylene oxide). In some embodiments, the poly(alkylene oxide) is polyethylene glycol or polypropylene glycol, or a combination thereof. In some embodiments, the poly(alkylene oxide) is polyethylene glycol. In some embodiments, the polymer is linear. In some embodiments, the polymer is branched.

In some embodiments, the linking group comprises polyethylene glycol. In some embodiments, the linking group comprises from 2-100 ethylene glycol units in a polyethylene glycol chain. In some embodiments, the linking group comprises from 2 to 50, 2 to 40, 2 to 30, 2 to 20, 2 to 10, 5 to 50, 5 to 40, 5 to 30, 5 to 20, 5 to 10, 10 to 50, 10 to 40, 10 to 30, or 10 to 20 ethylene glycol units in a polyethylene glycol chain. In some embodiments, the linking group comprises 2, 3, 4, 5, 6, 7, 8, 9, 10, 11, 12, 13, 14, 15, 16, 17, 18, 19, or 20 ethylene glycol units in a polyethylene glycol chain. In some embodiments, the linking group comprises 2 to 6 ethylene glycol units. In some embodiments, the linking group comprises 4 ethylene glycol units. In some embodiments, the linking group comprises 6 to 12 ethylene glycol units. In some embodiments, the linking group comprises 9 ethylene glycol units. In some embodiments, the linking group comprises 12 to 20 ethylene glycol units. In some embodiments, the linking group comprises 16 ethylene glycol units. In some embodiments, the linking group comprises 20 to 36 ethylene glycol units. In some embodiments, the linking group comprises 24 ethylene glycol units.

In some embodiments, the linking group comprises a non-polymer. In some embodiments, the non-polymer comprises a dicarboxylic acid group (e.g., a malonyl, a succinyl, a glutaryl, an adipiyl, a pimelyl, or a suberyl, group), an amino acid group (e.g., a glycyl, a 3-amino-propanyl, a 4-amino-butanyl, a 5-amino pentanyl, or a 6-amino hexyanyl group), or a diamino group (e.g., an ethylene diaminyl, a propylene diaminyl, a butylene diaminyl, a pentylene diaminyl, or hexylene diaminyl group). In some embodiments, the non-polymer comprises succinimidyl 4-(N-maleimidomethyl)cyclohexane-1-carboxylate; Maleimidocaproyl; Valine-citrulline; Allyl(4-methoxyphenyl)dimethylsilane; 6-(Allyloxycarbonylamino)-1-hexanol; 4-Aminobutyraldehyde diethyl acetal; or (E)-N-(2-Aminoethyl)-4-{2-[4-(3-azidopropoxy)phenyl]diazenyl}benzamide.

In some embodiments, the linking group comprises a structure

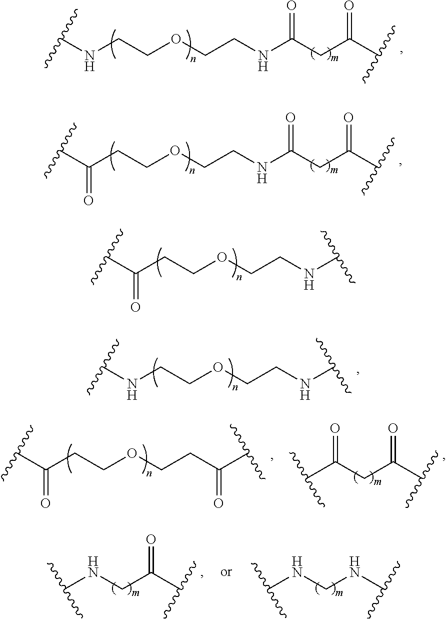

wherein m (if present) is an integer from 1 to 12 and n (if present) is an integer from 2 to 50. In some embodiments, m is 2, 3, or 4. In some embodiments, m is 3. In some embodiments, n is an integer from 2 to 30. In some embodiments, n is 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is an integer from 2 to 6, from 6 to 12, from 12 to 20, or from 20 to 30. In some embodiments, n is 4. In some embodiments, n is 9. In some embodiments, n is 16. In some embodiments, n is 27. In some embodiments, each

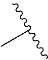

is a point of attachment to either the IL-2 polypeptide or the cleavable moiety (e.g., the cleavable peptide).

In some embodiments, the linking group is a linking peptide group. In some embodiments, the linking peptide group is attached the N-terminus or the C-terminus of the protein. In some embodiments, the linking peptide group is attached to the N-terminus or the C-terminus of a cleavable peptide.

In some embodiments, the linking group comprises a linear chain of from 1 to 100 atoms between the protein and the cleavable moiety. In some embodiments, the linking group comprises a linear chain of from 1 to 100, 1 to 50, 1 to 40, 1 to 30, 1 to 20, 5 to 100, 10 to 50, 10 to 20, 20 to 100, or 20 to 50 atoms between the protein and the cleavable moiety.

In some embodiments, the cleavable moiety attached to the IL-2 polypeptide of the activatable immunocytokine is a cleavable peptide. In some embodiments, the cleavable peptide is cleavable by one or more proteases. In some embodiments, the cleavable peptide is cleavable by one or more proteases associated with a tumor or tumor microenvironment. In some embodiments, the cleavable peptide is preferentially or selectively cleaved in or near a tumor microenvironment. In some embodiments, the cleavable peptide is preferentially or selectively cleaved by one or more proteases associated with a tumor or tumor microenvironment.

In some embodiments, the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the cleavable peptide is cleavable by an MMP. In some embodiments, the cleavable peptide is cleavable by a matriptase. In some embodiments, the cleavable peptide is cleavable by a plasminogen activator. In some embodiments, the cleavable peptide is cleavable by a legumain. In some embodiments, the cleavable peptide is cleavable by a protease set forth in Table 1A.

TABLE IA
Tissue/Tumor specific proteases

| | | |
|---|---|---|
| Protease | Specificity | Other Aspects |

Secreted by CD8+ Cytotoxic T cells

| | | |
|---|---|---|
| Granzyme B (grB) | Cleaves after Asp | Type of serine protease; Strongly |
| | | implicated in inducing perforin-dependent |
| | | target cell apoptosis |
| Granzyme A (grA) | trypsin-like, cleaves after basic | Type of serine protease. |
| | residues | |
| Granzyme H (grH) | Unknown substrate specificity | Type of serine protease; |
| | | Other granzymes are also secreted by killer |
| | | T cells, but not all are present in humans |
| Caspase-8 | Cleaves after Asp residue | Type of cysteine protease; plays and |
| | | essential role in TCR-induced cellular |
| | | expansion. |
| Mucosa-associated | Cleaves after Arg residues | Type of cysteine protease; likely acts both |
| lymphoid tissue | | as a scaffold and proteolytically active |
| (MALTI) | | enzyme in the CBM-dependent signaling |
| | | pathway. |
| Tryptase | Target: angiotensin I, | Type of mast cell-specific serine protease; |
| | fibrinogen, prourokinase, | trypsin-like; resistant to inhibition by |
| | TGFB; preferentially cleaves | macromolecular protease inhibitors |
| | proteins after lysine or | expressed in mammals due to their |
| | arginine residues. | tetrameric structure, with all sites facing |
| | | narrow central pore; also associated with |
| | | inflammation. |

Associated with Inflammation:

| | | |
|---|---|---|
| Thrombin | Targets: FGF-2 | Type of serine protease; modulates activity |
| | HB-EGF, Osteo-pontin, | of vascular growth factors, chemokines |
| | PDGF, VEGF | and extracellular proteins; strengthens |
| | | VEGF-induced proliferation; induces cell |
| | | migration; angiogenic factor; regulates |
| | | hemostasis |
| Chymase | Exhibit chymotrypsin-like | Type of mast cell-specific serine protease. |
| | specificity, cleaving proteins | |
| | after aromatic amino acid | |
| | residues. | |
| Carboxypeptidase A | Cleaves amino acid residues | Type of zinc-dependent metalloproteinase |
| (MC-CPA) | from C-terminal end of | |
| | peptides and proteins | |
| Kallikreins | Targets: high molecular | Type of serine protease; modulate |
| | weight kininogen, pro- | relaxation response; contribute to |
| | urokinase | inflammatory response; activates pro- |
| | | apoptotic signaling |
| Elastase | Targets: E-cadherin, GM-CSF, | Type of neutrophil serine protease; |
| | IL~1, IL-2, IL-6, IL-8, | degrades ECM components; regulates |
| | p38^{MAPK}, TNFα, VE-cadherin. | inflammatory response; activates pro- |
| | | apoptotic signaling. |
| Cathepsin G | Targets: ENA-78, IL-8, MCP- | Type of serine protease; degrades ECM |
| | 1, MMP-2, MTI-MMP, PAI- | components; chemo-attractant of |
| | 1, RANTES, TGFβ, TNFα | leukocytes; regulates inflammatory |
| | | response; promotes apoptosis. |
| PR-3 | Targets: ENA-78, IL-8, IL-18, | Type of serine protease; promotes |
| | JNK, p38^{MAPK}, TNFα | inflammatory response; activates pro- |
| | | apoptotic signaling. |
| Granzyme M (GrM) | Cleaves after Met and other | Type of serine protease; only expressed in |
| | long, unbranched hydrophobic | NK cells. |
| | residue. | |
| Calpains | Cleave between Arg and Gly | Family of cysteine proteases; calcium- |
| | | dependent; activation is involved in the |
| | | process of numerous inflammation- |
| | | associated diseases. |

In some embodiments, the cleavable peptide is cleavable by multiple proteases. In some embodiments, the cleavable peptide is cleavable by multiple classes of proteases. In some embodiments, the cleavable peptide is cleavable by 2, 3, or 4 different proteases. In some embodiments, the cleavable peptide comprises multiple cleavage sites. In some embodiments, the cleavable peptide comprises 2, 3, 4, or more cleavage sites. In some embodiments, the cleavable peptide comprises 2 cleavage sites. In some embodiments, the cleavable peptide comprises 3 cleavage sites. In some embodiments, the cleavable peptide comprises 4 cleavage sites. In some embodiments, each of the cleavage sites is cleavable by a different protease.

In some embodiments, the cleavable peptide is cleavable by a matrix metalloprotease and a legumain. In some embodiments, the cleavable peptide is cleavable by a matrix metalloprotease and a matriptase. In some embodiments, the cleavable peptide is cleavable by a matrix metalloprotease and a plasminogen activator. In some embodiments, the cleavable peptide is cleavable by a legumain and a matriptase. In some embodiments, the cleavable peptide is cleavable by a legumain and a plasminogen activator. In some embodiments, the cleavable peptide is cleavable by a matriptase and a plasminogen activator. In some embodiments, the cleavable peptide is cleavable by a matrix metalloprotease, a legumain, and a matriptase. In some embodiments, the cleavable peptide is cleavable by a matrix metalloprotease, a matriptase, and a plasminogen activator. In some embodiments, the cleavable peptide is cleavable by a matrix metalloprotease, a legumain, and a plasminogen activator.

In some embodiments, the cleavable peptide has a length of from 2 to 30 amino acids. In some embodiments, the cleavable peptide has a length of at most 30, 25, 20, 19, 18, 17, 16, 15, 14, 13, 12, 11, 10, 9, 8, 7, 6, or 5 amino acids. In some embodiments, the cleavable peptide has a length of at least 4, 5, 6, 7, 8, 9, or 10 amino acids. In some embodiments, the cleavable peptide has a length of from 4 to 30, 4 to 25, 4 to 20, 4 to 18, 4 to 16, 4 to 15, 4 to 12, 4 to 10, 5 to 30, 5 to 25, 5 to 20, 5 to 18, 5 to 16, 5 to 15, 5 to 12, or 5 to 10 amino acids.

In some embodiments, cleavage of the cleavable peptide leaves a residual portion of the cleavable peptide attached to the IL-2 polypeptide of the activatable immunocytokine. In some embodiments, cleavage of the cleavable peptide leaves a residual portion of the cleavable peptide attached to the side chain of the amino acid residue to which the cleavable peptide is attached. In some embodiments, cleavage of the cleavable peptide leaves at least one amino acid of the cleavable peptide still attached to the IL-2 polypeptide. In some embodiments, cleavage of the cleavable peptide leaves at least one amino acid of the cleavable peptide still attached to the side chain of the amino acid residue to which the cleavable peptide is attached. In some embodiments, cleavage of the cleavable peptide leaves 1, 2, 3, 4, 5, 6, 7, 8, 9, or 10 amino acids attached to the side chain of the amino acid residue to which the cleavable moiety is attached. In some embodiments, cleavage of the cleavable peptide leaves at most 10 amino acids of the cleavable peptide attached to the IL-2 polypeptide. In some embodiments, cleavage of the cleavable peptide leaves at most 5 amino acids of the cleavable peptide attached to the IL-2 polypeptide. In some embodiments, cleavage of the cleavable peptide leaves 1 amino acid of the cleavable peptide attached to the IL-2 polypeptide. In some embodiments, cleavage of the cleavable peptide leaves 2 amino acids of the cleavable peptide attached to the IL-2 polypeptide. In some embodiments, cleavage of the cleavable peptide leaves 3 amino acids of the cleavable peptide attached to the IL-2 polypeptide. In some embodiments, cleavage of the cleavable peptide leaves 4 amino acids of the cleavable peptide attached to the IL-2 polypeptide. In some embodiments, cleavage of the cleavable peptide leaves 5 amino acids of the cleavable peptide attached to the IL-2 polypeptide. In some embodiments, the reference to amino acids attached to the IL-2 polypeptide described in this paragraph refers only to those at a single point of attachment (e.g., in a case where the cleavable peptide is attached to the IL-2 polypeptide at two points of attachment, the number of amino acids attached in this paragraph refers to the number of amino acids attached to either or both of the first and second points of attachment).

Exemplary cleavable peptide sequences which can be incorporated into an activatable protein as provided herein can be found in any one of U.S. Patent Publication Nos: US2010/0189651, US2016/0289324, US2018/0125988, US2019/0153115, US2020/0385469, US2021/0260163, US2022/0048949, US2022/0267400, US2021/0115102, US2022/0002370, US2021/0163562, US20200392235, US2021/0139553, US2021/0317177, US2020/0283489, US2021/0002343, US2021/0292421, US2021/0284728, US2021/0269530, US2022/0054544, US2021/0355219, US2022/0073613, US2021/0047406, and/or Patent Cooperation Treaty (PCT) Publication Nos: WO2021/202675, WO2021/062406, WO2021/142471, WO2021/216468, WO2021/119516, WO2021/253360, WO2021/146455, WO2021/202678, WO2021/202673, WO2021/189139, WO2020/232303, WO2022/115865, WO2021/202678, and/or Chen et. al., J Bio Chem, 277, V6 P4485-4491. (2002), each of which is hereby incorporated by reference as if set forth herein in its entirety.

In some embodiments, the cleavable peptide comprises an amino acid sequence having at least about 80%, at least about 900%, or 100% identity to a sequence set forth in Table 1B, Table 1C, or Table 1D. In some embodiments, the cleavable peptide comprises an amino acid sequence having at least about 80%, at least about 90%, or 100% identity to a sequence set forth in Table 1B or Table 1C. In some embodiments, the cleavable peptide comprises an amino acid sequence set forth in Table 1B. In some embodiments, the cleavable peptide comprises an amino acid sequence set forth in Table 1C. In some embodiments, the cleavable peptide comprises an amino acid sequence set forth in Table 1D.

TABLE 1B
Exemplary Cleavage Sequences

| SEQ ID | Cleavable Peptide | |
|---|---|---|
| NO: | Sequence | Protease |
| 101 | SGGPGPAGMKGLPGS | MMP9 |
| 102 | EAGRSANHEPLGLVAT | MMP2-7-9-14 + |
| | | matriptase + uPA + |
| | | Legumain |
| 103 | PQASTGRSGG | MMP9 + matriptase + |
| | | uPA |
| 104 | PQGSTGRAAG | MMP9 + matriptase + |
| | | uPA |
| 105 | PPASSGRAGG | MMP9 + matriptase + |
| | | uPA |
| 106 | RSGVPLSLYSGSGGGK | MMP7-9-10 |
| 107 | RSGMPYDLYHPSGK | MMP7-9-10 |
| 108 | RGPDSGGFMLTSGK | MMP7-9-10 |
| 109 | RGSGHEQLTVSGGSK | MMP7-9-10 |
| 110 | RSGRAAAVKSPSGK | MMP7-9-10 |
| 111 | RGSGISSGLLSGRSDNHSGK | MMP7-9-10 |
| 112 | RGDLLAVVAASGGK | MMP7-9-10 |
| 113 | RGGISSGLLSGRSGK | MMP7-9-10 |
| 114 | SGGGKKLADEPEGGS | Meprin A/B |
| 115 | GGGKFLADEPEGG | Meprin A/B (High |
| | | Efficiency) |
| 116 | ARLQSAAP | Cathepsin S, K, L |
| 117 | ARLQSAAPAGLKGA | Cathepsin S + MMP9 + |
| | | Meprin A |
| 118 | GSGGPGPAGMHGLPGGS | MMP9 (High |
| | | Efficiency) |
| 119 | GGGSHTGRSAYFGGGS | uPA |
| 120 | SGGPGPAGLKGAPGS | MMP9-2 |
| 121 | VPLSLYSG | MMP2-7-9 |
| 122 | SGLLSGRSDNH | uPA + Matriptase + |
| | | legumain |
| 123 | SGRSDNIGGGS | uPA |
| 124 | LQESLRSKESGRSDI | MMP-14 + uPA |
| 125 | ISSGLLSGRSDNH | uPA |
| 126 | LSGRSDDH | uPA |
| 127 | ISSGLLSGRSDQH | uPA |
| 128 | ISSGLLSGRSDNI | uPA |
| 129 | PLGLAG | MMP2-7-9 |
| 130 | GPAGMKGL | MMP |
| 131 | LSGRSDQH | MMP |
| 132 | LSGRSDNI | MMP |
| 133 | ISSGLLSGRSDNH | MMP |
| 134 | GPLGVRG | MMP |
| 135 | GPLGLAR | MMP |
| 136 | GPAALVGA | MMP |
| 137 | GPAALIGG | MMP |
| 138 | GPLNLVGR | MMP |
| 139 | GPAGLVAD | MMP |
| 140 | GPANLVAP | MMP |
| 141 | VPLSLYSG | MMP |
| 142 | SGESPAYYTA | MMP |
| 143 | GGPRGLG | MMP |
| 144 | HSSKLQ | MMP |
| 145 | HSSKLQL | MMP |
| 146 | KRALGLPG | MMP7 |
| 147 | LEATA | MMP9 |
| 148 | GGAANLVRGG | MMP11 |
| 149 | SGRIGFLRTA | MMP14 |
| 150 | PLGLA | MMP |
| 151 | ESPAYYTA | MMP |
| 152 | RLQLKL | MMP |
| 153 | RLQLKAC | MMP |
| 154 | SGRSA | uPA |
| | (D-Ala)FK | uPA |
| 156 | GGGRR | uPA |
| 157 | GFLG | Lysosomal Enzyme |
| 158 | HSSKLQEDA | Prostate Specific |
| | | Antigen |
| 159 | LVLASSSFGY | HSV Protease |
| 160 | GVSQNYPIVG | HIV Protease |
| 161 | GVVQASCRLA | CMV Protease |
| 162 | DPRSFL | Thrombin |
| 163 | PPRSFL | Thrombin |
| 164 | DEVD | Caspase-3 |
| 165 | DEVDP | Caspase-3 |
| 166 | KGSGDVEG | Caspase-3 |
| 167 | GWEHDG | Interleukin 1β |
| | | converting enzyme |
| 168 | EDDDDKA | Enterokinase |
| 169 | KQEQNPGST | FAP |
| 170 | GKAFRR | Kallikrein 2 |
| 171 | DAFK | Plasmin |
| 172 | DVLK | Plasmin |
| 173 | DAFK | Plasmin |
| 174 | ALLLALL | TOP |
| 175 | SGAKPRALTA | MMP A3 |
| 176 | SGLRLAAITA | MMP B49 |
| 177 | SGESLAYYTA | MMP B74 |
| 178 | SGESPAYYTA | MMP B74P |
| 179 | SGESLRYYTA | MMP B74R |
| 180 | SGRSLSRLTA | MMP C9 |
| 181 | SGRSLRRLTA | MMP C9R |
| 182 | SGAVSWLLTA | MMP A13 |
| 183 | SGAPSWLLTA | MMP A13P |
| 184 | SGAVRWLLTA | MMP A13R |
| 185 | SGANISDLTA | MMP B37 |
| 186 | SGNRYSSLTA | MMP A34 |
| 187 | SGHMHKALTA | MMP A21 |
| 188 | SGHMHKALTA | MMP A21A |
| | EVCit(p-amino- | Cathepsin B |
| | benzyloxycarbonyl) | |
| 190 | GEEGEEPLGLAG | |
| 191 | GPLGLAG | |
| 192 | EAGRSANHTPAGLTGP | |
| 193 | GEAGRSANHTPAGLTGP | |

TABLE 1C
Additional Exemplary Cleavage Sequences

| SEQ ID | Cleavable Peptide |
|---|---|
| NO: | Sequence |
| 201 | AGDKSPLGLAG |
| 202 | AGDRSPLGLAG |
| 203 | AGDRSAPLGLAG |
| 204 | AWGRSPLGLAG |
| 205 | AWGRSAPLGLAG |
| 206 | AWGKSPLGLAG |
| 207 | GAFKSPLGLAG |
| 208 | SGRSAPLALAG |
| 209 | SGRSPLGLAG |
| 628 | RQRRSAAPLGLAG |
| 609 | RQRRSAPLGLAG |
| 610 | RQRRSPLGLAG |
| 625 | RQRRS-Nle-PLGLAG |
| 214 | RSGRSAPLGLAG |
| 215 | RSGKSAPLGLAG |
| 216 | FTARSAPLGLAG |
| 218 | FTAKSPLGLAG |
| 219 | RYGRSAPLGLAG |
| 220 | RYGRSPLGLAG |
| 627 | RYGKSAPLGLAG |
| 222 | KYGRSAPLGLAG |
| 223 | KWGRSAPLGLAG |
| 626 | KWGKSAPLGLAG |
| 225 | KWGRSPLGLAG |
| 611 | SGRVLTLRKAGPAGLVG |
| 612 | SGRVLTLRKAGPANLVG |
| 613 | SGRVLRKAGPAGLVG |
| 614 | SGRVLRKAGPANLVG |
| 605 | SGRVLGPAGLVG |
| 606 | SGRVLGPANLVG |
| 607 | SGRVLPAGLVG |
| 615 | SGRVLPANLVG |
| 616 | SGRVAGLVG |
| 617 | SGRVANLVG |
| 236 | SSRGRRGPLGLAG |
| 237 | SSRGPLGLAG |
| 238 | SSRGPRGLAG |
| 601 | SSRGPASNRRLPLGLAG |
| 618 | PASNRRLPLGLAG |
| 602 | SSRAVFRKNLGPLGLAG |
| 619 | SSRVERKPANLAG |
| 604 | SGRVLTLRKAPWGLLE |
| 620 | SGRVLTLRKAALPLAM |
| 603 | SGRVLRKAGPQPLVD |
| 246 | SGRVLPLNLSG |
| 608 | SGRVLGPLNLSG |
| 621 | SSRGRRGPYMLQG |
| 622 | SSRGPYMLQG |
| 250 | SGRVLPLGLAG |
| 251 | SGRVLPMSLRM |
| 623 | SGRVLPLGMRA |
| 253 | SGRVLPLGLRA |
| 624 | SGRVLPYAMTA |
| 255 | SGRVLPLGFMG |

TABLE 1D
Further Exemplary Cleavage Sequences and
portions thereof

| SEQ ID | |
|---|---|
| NO: | Sequence |
| 601 | SSRGPASNRRLPLGLAG |
| 602 | SSRAVFRKNLGPLGLAG |
| 603 | SGRVLRKAGPQPLVD |
| 604 | SGRVLTLRKAPWGLLE |
| 605 | SGRVLGPAGLVG |
| 606 | SGRVLGPANLVG |
| 607 | SGRVLPAGLVG |
| 608 | SGRVLGPLNLSG |
| 609 | RQRRSAPLGLAG |
| 610 | RQRRSPLGLAG |
| 611 | SGRVLTLRKAGPAGLVG |
| 612 | SGRVLTLRKAGPANLVG |
| 613 | SGRVLRKAGPAGLVG |
| 614 | SGRVLRKAGPANLVG |
| 615 | SGRVLPANLVG |
| 616 | SGRVAGLVG |
| 617 | SGRVANLVG |
| 618 | PASNRRLPLGLAG |
| 619 | SSRVFRKPANLAG |
| 620 | SGRVLTLRKAALPLAM |
| 621 | SSRGRRGPYMLQG |
| 622 | SSRGPYMLQG |
| 623 | SGRVLPLGMRA |
| 624 | SGRVLPYAMTA |
| 625 | RQRRS-Nle-PLGLAG |
| 626 | KWGKSAPLGLAG |
| 627 | RYGKSAPLGLAG |
| 628 | RQRRSAAPLGLAG |
| 629 | RQRRSVVGG |
| 630 | SPLGLAGS |
| 631 | RGRKVANLVG |
| 632 | RQRKVANLVG |
| 633 | RGRRVANLVG |
| 634 | RGRKSPANLVG |
| 635 | RGRKPYMLQG |
| 636 | RGRKPY-Nle-LQG |
| 637 | RGRKSPYMLQG |
| 638 | RGRKSPY-Nle-LQG |
| 639 | RGRKPQPLVD |
| 640 | RGRKSPQPLVD |
| 641 | RGRKSQPLVD |
| 642 | SGRVAPYMLQG |
| 643 | SGRVAPY-Nle-LQG |
| 644 | SGRVYMLQG |
| 645 | SGRVY-Nle-LQG |
| 646 | SGRVAPQPLVD |
| 647 | SGRVQPLVD |
| 648 | RGRRGP |
| 649 | RGRRPLGLAG |
| 650 | RGRRVANPLGLAGSG |
| 651 | RGRRPLGLAGGSG |
| 652 | RGRRHSSKLQ |
| 653 | SGRVANPLGGSG |
| 654 | SGRVANYFGKL |
| 655 | RGRRVANYFGKL |
| 656 | SGRPLGYFGKL |
| 657 | RGRRPLGYFGKL |
| 658 | RGRRVANPLGYFGKL |
| 659 | RGRRSGRAANLVRPLGYFGKL |
| 660 | RGRRAANLVRPLGYFGKL |
| 661 | HSSKLQYFGKL |
| 662 | RGRRHSSKLQPLGYFGKL |
| 663 | SGRHSSKLQPLGYFGKL |
| 664 | GSGSGSGS |
| 665 | SSLYSSPG |
| 666 | SSLQSSPG |
| 667 | SQYQSSPG |
| 668 | SQLYSSPG |
| 669 | SSQYSSPG |
| 670 | ISQYSSAT |
| 671 | KLYSSKQ |
| 672 | KLFSSKQ |
| 673 | RRLHYSL |
| 674 | RRLNYSL |
| 675 | RSSYRSL |
| 676 | RSSYYSL |
| 677 | KSKQHSL |
| 678 | HSSKLQL |
| 679 | GSSYYSGA |
| 680 | GSSVYSGR |
| 681 | SS-Nle-YSSAG |

The cleavable peptide can be attached to the IL-2 polypeptide (e.g., a side chain of an amino acid residue of the IL-2 polypeptide) in a variety of ways. In some embodiments, the cleavable peptide is covalently attached to the IL-2 polypeptide (e.g., a side chain of an amino acid residue of the IL-2 polypeptide). In some embodiments, the cleavable peptide is attached to the IL-2 polypeptide through its C-terminal carboxyl, its N-terminal amine, or through a side chain of the cleavable peptide. In some embodiments, the cleavable peptide is directly attached to the IL-2 polypeptide. In some embodiments, the cleavable peptide is directly attached to a side chain of an amino acid residue of the IL-2 polypeptide.

In some embodiments, the cleavable peptide is attached to the IL-2 polypeptide of the activatable immunocytokine through the C-terminus of the cleavable peptide. In some embodiments, the C-terminal carboxyl group of the cleavable peptide is directly attached to a side chain of an amino acid residue of the IL-2 polypeptide. In some embodiments, the C-terminal carboxyl group of the cleavable peptide is directly attached to a side chain amine of an amino acid residue of the IL-2 polypeptide (e.g., as an amide bond). In some embodiments, the side chain amine is of a lysine residue of the IL-2 polypeptide. In some embodiments, the side chain amine is of an unnatural amino acid residue of the IL-2 polypeptide (e.g., ornithine, homolysine, or another amine containing unnatural amino acid). In some embodiments, the C-terminal carboxyl group of the cleavable peptide is attached to a side chain of an amino acid residue of the IL-2 polypeptide through a linking group (e.g., any of the linking groups provided herein). In some embodiments, the C-terminal carboxyl group of the cleavable peptide attached to the linking group by an amide bond formed between the C-terminal carboxyl and an amine group of the linking group.

In some embodiments, the cleavable peptide is attached to the IL-2 polypeptide of the activatable immunocytokine through the N-terminus of the cleavable peptide. In some embodiments, the N-terminal amine group of the cleavable peptide is directly attached to a side chain of an amino acid residue of the IL-2 polypeptide. In some embodiments, the N-terminal amine group of the cleavable peptide is directly attached a side chain carboxyl of an amino acid residue of the IL-2 polypeptide (e.g., as an amide bond). In some embodiments, the side chain carboxyl is of a glutamate or aspartate residue of the IL-2 polypeptide. In some embodiments, the side chain carboxyl is of an unnatural amino acid residue of the IL-2 polypeptide (e.g., 2-amino adipic acid, 2-amino pimelic acid, or another carboxyl containing unnatural amino acid). In some embodiments, the N-terminal amine group of the cleavable peptide is attached to a side chain of an amino acid residue of the IL-2 polypeptide through a linking group (e.g., any of the linking groups provided herein). In some embodiments, the N-terminal carboxyl group of the cleavable peptide attached to the linking group by an amide bond formed between the N-terminal amine and a carboxyl group of the linking group.

In some embodiments, the cleavable peptide is attached to the IL-2 polypeptide of the activatable immunocytokine through a side chain of an amino acid residue of the cleavable peptide. In some embodiments, a side chain of an amino acid residue of the cleavable peptide is directly attached the IL-2 polypeptide (e.g., to a side chain of an amino acid residue of the protein). In some embodiments, a side chain of an amino acid residue of the cleavable peptide is attached to the IL-2 polypeptide through a linking group (e.g., any of the linking groups provided herein).

In some embodiments, the cleavable moiety can be cleaved by a reduction or oxidation reaction. In some instances, different tissues of a subject can exhibit different redox potentials depending on the activity of the tissue. For example, tumors and tumor microenvironments are associated with having substantially greater reduction potentials than other healthy tissues. Thus, in some embodiments, cleavable moieties provided herein utilize this property to allow preferential cleavage and activation of a protein at the tissue site. In some embodiments, the cleavable moiety contains a site of cleavage that can be cleaved specifically by a reduction or oxidation reaction. In some embodiments, the cleavable moiety contains a site of cleavage that can be cleaved at a site preferred by a reduction or oxidation reaction. In some embodiments, the specific cleavage site is a redox sensitive cleavage site.

In some embodiments, the redox sensitive cleavage site is preferentially cleaved at or near a target tissue of the subject such that the specific cleavage site is cleaved at a rate of least 2-fold, at least 4-fold, at least 8-fold, at least 10-fold, at least 15-fold, at least 20-fold, at least 50-fold, at least 100-fold, at least 200-fold, at least 500-fold, or at least 1000-fold greater than cleavage of the specific cleavage site at a different tissue. In some embodiments, the redox cleavage site is preferentially cleaved in a reducing environment. In some embodiments, the redox cleavage site is preferentially cleaved in a reducing environment relative to blood. In some embodiments, the redox cleavage site is preferentially cleaved in a reducing environment relative to interstitial fluids. In some embodiments, the redox cleavage site is preferentially cleaved in a reducing environment relative to lymphatic fluid. In some embodiments, the redox cleavage site is preferentially cleaved in a reducing environment of a tumor microenvironment.

In some embodiments, the redox sensitive cleavage site is a disulfide. In some embodiments, the cleavable moiety is not a disulfide bond between two cysteines in the native protein. In some embodiments, the cleavable moiety is not a disulfide bond between the side chains of two amino acids of the protein.

In some embodiments, the cleavable moiety comprises a pH sensitive cleavage site. The pH of circulating blood is generally buffered in a narrow range of between 7.31 to 7.45. Variances outside this range typically result in acidosis or alkalosis, which are serious medical conditions. The tumor microenvironment is characteristically more acidic than circulating blood pH due to a metabolic dysregulation in tumor cells known as the Warburg effect: Growing tumor cells demonstrate a high rate of glycolysis followed by fermentation of pyruvate to lactic acid in the cytoplasm rather than oxidation of pyruvate in the mitochondrial TCA cycle. To maintain the pH of their cytoplasm, tumor cells transport hydrogen ions to the extracellular environment, resulting in an acidic tumor microenvironment. In some embodiments, the pH sensitive cleavage site is selected to preferentially cleave at a target tissue. In some embodiments, the target tissue is associated with a certain pH or a difference in pH compared to other local tissues.

In some embodiments, the pH sensitive cleavage site is cleaved at a pH below physiological blood pH (e.g., below about 7.3). In some embodiments, the pH sensitive cleavage site is preferentially cleaved at a pH below 7.3, below 7.2, below 7.1, or below 7.0. In some embodiments, the pH sensitive cleavage site is preferentially cleaved at acidic pHs. In some embodiments, the pH sensitive cleavage site is preferentially cleaved at a pH of below 7, 6.5, 6, 5.5, or 5.

In some embodiments, the pH sensitive cleavage site is preferentially cleaved at or near a target tissue of the subject such that the specific cleavage site is cleaved at a rate of least 2-fold, at least 4-fold, at least 8-fold, at least 10-fold, at least 15-fold, at least 20-fold, at least 50-fold, at least 100-fold, at least 200-fold, at least 500-fold, or at least 1000-fold greater than cleavage of the specific cleavage site at a different tissue. In some embodiments, the tissue is a tumor microenvironment.

### Points of Attachment of Cleavable Moieties to IL-2 Polypeptides of Activatable Immunocytokines

In some embodiments, the cleavable moiety is attached to a side chain of an amino acid residue of the IL-2 polypeptide (either directly or through a suitable linking group) of the immunocytokine composition). The cleavable moiety can be attached to the side chain of an amino acid at any desired position, and can be attached to any suitable amino acid type.

The cleavable moiety can be attached to a side chain of a wide variety different amino acid residues. In some embodiments, the amino acid residue to which the cleavable moiety is attached is lysine, glutamate, glutamine, aspartate, asparagine, tyrosine, serine, threonine, cysteine, or an unnatural amino acid. In some embodiments, the amino acid residue to which the cleavable moiety is attached is lysine, glutamate, glutamine, aspartate, asparagine, tyrosine, serine, threonine, cysteine, or an unnatural amino acid. In some embodiments, the amino acid residue to which the cleavable moiety is attached is lysine, glutamate, aspartate, tyrosine, serine, threonine, cysteine, or an unnatural amino acid. In some embodiments, the amino acid residue to which the cleavable moiety is attached is lysine, glutamate, aspartate, tyrosine, cysteine, or an unnatural amino acid. In some embodiments, the amino acid residue to which the cleavable moiety is attached is lysine, glutamate, aspartate, cysteine, or an unnatural amino acid. In some embodiments, the amino acid residue to which the cleavable moiety is attached is lysine, glutamate, aspartate, or cysteine. In some embodiments, the amino acid residue to which the cleavable moiety is attached is a lysine. In some embodiments, the amino acid residue to which the cleavable moiety is attached is a lysine or glutamate. In some embodiments, the amino acid residue to which the cleavable moiety is attached is a glutamate. In some embodiments, the amino acid residue to which the cleavable moiety is attached is an aspartate. In some embodiments, the amino acid residue to which the cleavable moiety is attached is a cysteine.

In some embodiments, the amino acid residue to which the cleavable moiety is attached is a lysine. In some embodiments, the cleavable moiety is attached to the lysine via an amide bond between the side chain amine of the lysine and a carboxyl group of the cleavable moiety or a linking group.

In some embodiments, the amino acid residue to which the cleavable moiety is attached is a glutamate. In some embodiments, the cleavable moiety is attached to the glutamate via an amide bond between the side chain carboxyl of the glutamate and an amine group of the cleavable moiety or a linking group. In some embodiments, the cleavable moiety is attached to the glutamate via an ester bond between the side chain carboxyl of the glutamate and a hydroxyl group of the cleavable moiety or a linking group.

In some embodiments, the amino acid residue to which the cleavable moiety is attached is an aspartate. In some embodiments, the cleavable moiety is attached to the aspartate via an amide bond between the side chain carboxyl of the aspartate and an amine group of the cleavable moiety or a linking group. In some embodiments, the cleavable moiety is attached to the aspartate via an ester bond between the side chain carboxyl of the glutamate and a hydroxyl group of the cleavable moiety or a linking group.

In some embodiments, the amino acid residue to which the cleavable moiety is attached is a cysteine. In some embodiments, the cleavable moiety is attached to the cysteine via thioether bond between the side chain thiol of the cysteine and an alkyl or aryl group of the cleavable moiety or a linking group. In some embodiments, the bond between the cleavable moiety or linking group and the cysteine sulfhydryl is formed from a reaction of the sulfhydryl with a suitable reactive group (e.g., a maleimide, an a,b-unsaturated carbonyl, an a-halo carbonyl).

In some embodiments, the amino acid residue to which the cleavable moiety is attached is a tyrosine. In some embodiments, the cleavable moiety is attached to the tyrosine via an ether bond between the side chain phenol of the tyrosine and an alkyl or aryl group of the cleavable moiety or a linking group.

In some embodiments, the amino acid residue to which the cleavable moiety is attached is the amino acid present at that position in a wild type IL-2 polypeptide. In some embodiments, the amino acid residue to which the cleavable moiety is attached is substituted relative to the amino acid at the corresponding position in the wild type version of the IL-2 polypeptide. In some embodiments, the amino acid residue to which the cleavable moiety is attached is substituted to a lysine, glutamate, glutamine, aspartate, asparagine, tyrosine, serine, threonine, cysteine, or an unnatural amino acid.

In some embodiments, the amino acid residue to which the cleavable moiety is attached is an unnatural amino acid. In some embodiments, the unnatural amino acid is comprises a side chain having a functional group that allows for attachment of the cleavable moiety or linking group to the unnatural amino acid. In some embodiments, the unnatural amino acid comprises a side chain functional group which is the same as that of a natural amino acid (e.g., carboxyl, amine, phenol, thiol). In some embodiments, unnatural amino acid comprises a side chain amine. In some embodiments, the unnatural amino acid is a lysine analog (e.g., ornithine, homolysine, 2,4-diaminobutyric acid, etc.). In some embodiments, the unnatural amino acid comprises a side chain carboxyl. In some embodiments, the unnatural amino acid is a glutamate analog (e.g., 2-amino adipic acid, 2-amino pimelic acid). In some embodiments, the unnatural amino acid comprises a side chain thiol. In some embodiments, the unnatural amino acid is a cysteine analog (e.g., homocysteine, another amino acid containing an alkyl group side chain with a thiol, etc.).

In some embodiments, the unnatural amino acid residue to which the cleavable moiety is attached comprises a conjugation handle for the attachment of the cleavable moiety or a linking group. In some embodiments, the conjugation handle facilitates the attachment of the cleavable moiety to the IL-2 polypeptide through a reaction with a complementary conjugation handle on the cleavable moiety or linking group. Thus, in such cases, the activatable protein will comprise a reaction product of the conjugation handle and the complementary conjugation handle. The conjugation handle can be any suitable conjugation handle, including any of the conjugation handles provided herein. Non-limiting examples of unnatural amino acid residues comprising conjugation handles can be found, for example, in Patent Cooperation Treaty Pub. Nos. WO2015/054658, WO2014/036492, WO2021/133839, WO2006/069246, and WO2007/079130, each of which is incorporated by reference as if set forth in its entirety.

In some embodiments, the amino acid to which the cleavable moiety is attached (e.g., at the side chain) is an internal amino acid of the IL-2 polypeptide. In some embodiments, the amino acid to which the cleavable moiety is attached is a non-terminal residue. In some embodiments, the cleavable moiety is attached to the side chain of the N-terminal or C-terminal residue of the IL-2 polypeptide.

In some embodiments, the cleavable moiety is attached to the IL-2 polypeptide of the activatable immunocytokine at a residue of the IL-2 polypeptide such that binding of the IL-2 polypeptide to at least one IL-2 receptor subunit is reduced (e.g., compared to a corresponding IL-2 polypeptide without the cleavable moiety attached). In some embodiments, the cleavable moiety is attached to the IL-2 polypeptide at a residue of the IL-2 polypeptide such that binding of the IL-2 polypeptide to an IL-2 receptor beta and/or gamma subunit is reduced. In some embodiments, the cleavable moiety is attached to the IL-2 polypeptide at a residue of the IL-2 polypeptide such that binding of the IL-2 polypeptide to an IL-2 receptor beta subunit is reduced. In some embodiments, the cleavable moiety is attached to the IL-2 polypeptide at a residue of the IL-2 polypeptide such that binding of the IL-2 polypeptide to an IL-2 receptor gamma subunit is reduced.

In some embodiments, the cleavable moiety is attached to the IL-2 polypeptide of the activatable immunocytokine at a residue of the IL-2 polypeptide such that binding to an IL-2 receptor complex is reduced (e.g., compared to a corresponding IL-2 polypeptide without the cleavable moiety attached). In some embodiments, the cleavable moiety is attached to the IL-2 polypeptide at a residue of the IL-2 polypeptide such that binding to an IL-2 receptor Oy complex is reduced.

In some embodiments, the cleavable moiety is attached to the IL-2 polypeptide of the activatable immunocytokine at or near a residue of the IL-2 polypeptide which interacts with an IL-2 receptor beta or gamma subunit, or an IL-2 receptor βγ complex. Interactions of IL-2 with the IL-2 receptor and its associated subunits have been explored and analyzed by X-ray co-crystal structures, for example at least Stauber et al. in “Crystal structure of the IL-2 signaling complex: Paradigm for a heterotrimeric cytokine receptor” (Proc Natl Acad Sci USA. 2006 Feb. 21; 103(8): 2788-279), which is hereby incorporated by reference as if set forth herein in its entirety. In some embodiments, the cleavable moiety is attached to an amino acid residue which is within at most 20, 15, 10, 9, 8, 7, 6, 5, 4, 3, 2, or 1 amino acids of a residue which interacts with the IL-2 receptor beta or gamma subunit, or the IL-2 receptor βγ complex. In some embodiments, the cleavable moiety is attached to an amino acid residue which interacts with the IL-2 receptor beta or gamma subunit, or the IL-2 receptor βγ complex.

In some embodiments, the cleavable moiety is attached to a residue selected from residues 9, 11, 13, 15, 16, 19, 20, 22, 23, 26, 29, 32, 84, 88, 91,123,126, and 129 of the IL-2 polypeptide of the activatable immunocytokine, wherein residue position numbering is based on SEQ ID NO: 1 as a reference sequence. In some embodiments, the cleavable moiety is attached to residue 9, 11, 13, 15, 16, 19, 22, 23, 29, or 32 of the IL-2 polypeptide. In some embodiments, the cleavable moiety is attached to residue 9 of the IL-2 polypeptide. In some embodiments, the cleavable moiety is attached to residue 11 of the IL-2 polypeptide. In some embodiments, the cleavable moiety is attached to residue 13 of the IL-2 polypeptide. In some embodiments, the cleavable moiety is attached to residue 15 of the IL-2 polypeptide. In some embodiments, the cleavable moiety is attached to residue 16 of the IL-2 polypeptide. In some embodiments, the cleavable moiety is attached to residue 20 of the IL-2 polypeptide. In some embodiments, the cleavable moiety is attached to residue 22 of the IL-2 polypeptide. In some embodiments, the cleavable moiety is attached to residue 23 of the IL-2 polypeptide. In some embodiments, the cleavable moiety is attached to residue 26 of the IL-2 polypeptide. In some embodiments, the cleavable moiety is attached to residue 29 of the IL-2 polypeptide. In some embodiments, the cleavable moiety is attached to residue 32 of the IL-2 polypeptide. In some embodiments, the cleavable moiety is attached to residue 84 of the IL-2 polypeptide. In some embodiments, the cleavable moiety is attached to residue 88 of the IL-2 polypeptide. In some embodiments, the cleavable moiety is attached to residue 91 of the IL-2 polypeptide. In some embodiments, the cleavable moiety is attached to residue 123 of the IL-2 polypeptide. In some embodiments, the cleavable moiety is attached to residue 126 of the IL-2 polypeptide. In some embodiments, the cleavable moiety is attached to residue 129 of the IL-2 polypeptide.

In some embodiments, the cleavable moiety is attached to a residue in the region of residues 1-35 of the IL-2 polypeptide of the activatable immunocytokine, wherein residue position numbering is based on SEQ ID NO: 1 as a reference sequence. In some embodiments, the cleavable moiety is attached to any one of residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35. In some embodiments, the cleavable moiety is attached to a residue in the region of residues 5-35, 5-32, 5-30, 5-25, 5-23, 9-35, 9-32, 9-30, 9-25, 9-23, 15-35, 15-32, 15-30, 15-25, or 15-23 of the IL-2 polypeptide.

In some embodiments, the amino acid residue to which the cleavable moiety is attached is substituted relative to the corresponding amino acid residue of SEQ ID NO: 1. The substitution can be any suitable amino acid, including any of those provided supra (e.g., lysine, glutamate, glutamine, aspartate, asparagine, tyrosine, serine, threonine, cysteine, an unnatural amino acid, etc.).

### Additional Points of Attachment of Cleavable Moieties to IL-2 Polypeptides of Activatable Immunocytokines

In some embodiments, the cleavable moiety is further attached to the IL-2 polypeptide of the activatable immunocytokine at an additional point of attachment to the IL-2 polypeptide (e.g., a second point of attachment distinct from the side chain of the amino acid residue to which the cleavable moiety is attached discussed above). In some embodiments, cleavage of the cleavable moiety breaks the connection between the two points of attachment through the cleavable moiety.

In some embodiments, attachment of the cleavable moiety to the additional point of attachment results in a macrocyclic structure being formed. In some embodiments, the macrocyclic structure is formed between the cleavable moiety, any intervening linking groups (if present) between the cleavable moiety and either or both points of attachment to the IL-2 polypeptide, and any intervening amino acids of the IL-2 polypeptide. In some embodiments, cleavage of the cleavable moiety breaks the macrocyclic structure.

In some embodiments, attachment of the cleavable moiety to two points of the IL-2 polypeptide of the activatable immunocytokine (e.g., the side chain of an amino acid residue of the IL-2 polypeptide and the additional point of attachment) can act to lock the activatable IL-2 polypeptide in a less active conformation than wild type IL-2. In some embodiments, cleavage of the cleavable moiety structure frees the “activated” protein to adopt a new conformation.

The additional point of attachment can be to any amino acid residue, in particular those described above for points of attachment of cleavable moieties (e.g., to any of the types of residues described above), and can optionally be attached through an additional linking group as described above. The additional linking group can be attached to the cleavable moiety at an additional point in the same manner as described above, and the additional linking group can have any of the structures or compositions described herein (e.g., any of the structures or compositions for the first linking group). Where the cleavable moiety is attached to the IL-2 polypeptide at two sites by two linking groups, the two linking groups can be the same or different.

In some embodiments, the additional point of attachment is to a side chain of another amino acid residue of the IL-2 polypeptide of the activatable immunocytokine (e.g., a different amino acid residue from the first point at which the cleavable moiety is attached). In some embodiments, the cleavable moiety is attached directly to a side chain of a first amino acid residue and is further attached directly to a side chain of a second amino acid residue. In some embodiments, the cleavable moiety is attached directly to a side chain of a first amino acid residue and is further attached to a side chain of a second amino acid residue through a linking group. In some embodiments, the cleavable moiety is attached to a side chain of a first amino acid residue through a linking group and is further directly attached to a side chain of a second amino acid residue. In some embodiments, the cleavable moiety is attached to a side chain of a first amino acid residue through a first linking group and is further attached to a side chain of a second amino acid residue through a second linking group.

In some embodiments, the additional point of attachment is to a terminal reside of the IL-2 polypeptide of the activatable immunocytokine. In some embodiments, the additional point of attachment is to the N-terminus or the C-terminus of the IL-2 polypeptide. In some embodiments, the additional point of attachment is to the N-terminus or the C-terminus of the IL-2 polypeptide through a linking group. In some embodiments, the additional point of attachment is to the N-terminus of the IL-2 polypeptide. In some embodiments, the additional point of attachment is to the N-terminus of the IL-2 polypeptide through a linking group. In some embodiments, the additional point of attachment is to the C-terminus of the IL-2 polypeptide. In some embodiments, the additional point of attachment is to the C-terminus of the IL-2 polypeptide through a linking group.

In embodiments where a cleavable peptide is attached to the N-terminus or the C-terminus of the protein, the cleavable peptide can be linked to the protein via a peptide linking group. Non-limiting examples of peptide linking groups include, but are not limited to (GS)ₙ (SEQ ID NO: 275), (GGS)ₙ (SEQ ID NO: 276), (GGGS)ₙ (SEQ ID NO: 277), (GGSG)ₙ (SEQ ID NO: 278), or (GGSGG)ₙ (SEQ ID NO: 279), (GGGGS)ₙ (SEQ ID NO: 280), wherein n is 1, 2, 3, 4, 5, 6, 7, 8, 9, or 10. For example, a peptide linking group can be (GGGGS)₃ (SEQ ID NO: 281) or (GGGGS)₄ (SEQ ID NO: 282).

In some embodiments, the additional point of attachment of the cleavable moiety is to a side chain of another amino acid residue of the IL-2 polypeptide of the activatable immunocytokine. In some embodiments, the additional point of attachment is to a side chain of another amino acid residue which is less than 30, 25, 20, or 15 amino acids away from the first amino acid connected to the cleavable moiety.

In some embodiments, the additional point of attachment is to a residue in the region of residues 1-40 of the IL-2 polypeptide of the activatable immunocytokine. In some embodiments, the additional point of attachment is to a residue in the region of residues 5-40, 5-35, 5-30, 5-25, 5-20, 5-15, or 5-10. In some embodiments, the additional point of attachment is to any one of residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 of the IL-2 polypeptide. In some embodiments, the additional point of attachment is to residue 9 of the IL-2 polypeptide.

In some embodiments, the cleavable moiety is attached to residue 23 of the IL-2 polypeptide of the activatable immunocytokine and comprises an additional point of attachment to residue 9 of the IL-2 polypeptide. In some embodiments, the cleavable moiety is attached to residue 23 of the IL-2 polypeptide and comprises an additional point of attachment to the N-terminus of the IL-2 polypeptide.

In some embodiments, the cleavable moiety is attached to residue 11 of the IL-2 polypeptide of the activatable immunocytokine and comprises an additional point of attachment to residue 9 of the IL-2 polypeptide. In some embodiments, the cleavable moiety is attached to residue 11 of the IL-2 polypeptide and comprises an additional point of attachment to the N-terminus of the IL-2 polypeptide.

In some embodiments, the cleavable moiety is attached to residue 13 of the IL-2 polypeptide of the activatable immunocytokine and comprises an additional point of attachment to residue 9 of the IL-2 polypeptide. In some embodiments, the cleavable moiety is attached to residue 13 of the IL-2 polypeptide and comprises an additional point of attachment to the N-terminus of the IL-2 polypeptide.

In some embodiments, the cleavable moiety is attached to residue 15 of the IL-2 polypeptide of the activatable immunocytokine and comprises an additional point of attachment to residue 9 of the IL-2 polypeptide. In some embodiments, the cleavable moiety is attached to residue 15 of the IL-2 polypeptide and comprises an additional point of attachment to the N-terminus of the IL-2 polypeptide.

In some embodiments, the cleavable moiety is attached to residue 19 of the IL-2 polypeptide of the activatable immunocytokine and comprises an additional point of attachment to residue 9 of the IL-2 polypeptide. In some embodiments, the cleavable moiety is attached to residue 19 of the IL-2 polypeptide and comprises an additional point of attachment to the N-terminus of the IL-2 polypeptide.

In some embodiments, the cleavable moiety is attached to residue 22 of the IL-2 polypeptide of the activatable immunocytokine and comprises an additional point of attachment to residue 9 of the IL-2 polypeptide. In some embodiments, the cleavable moiety is attached to residue 22 of the IL-2 polypeptide and comprises an additional point of attachment to the N-terminus of the IL-2 polypeptide.

In some embodiments, the cleavable moiety is attached to residue 26 of the IL-2 polypeptide of the activatable immunocytokine and comprises an additional point of attachment to residue 9 of the IL-2 polypeptide. In some embodiments, the cleavable moiety is attached to residue 26 of the IL-2 polypeptide and comprises an additional point of attachment to the N-terminus of the IL-2 polypeptide.

In some embodiments, the cleavable moiety is attached to residue 29 of the IL-2 polypeptide of the activatable immunocytokine and comprises an additional point of attachment to residue 9 of the IL-2 polypeptide. In some embodiments, the cleavable moiety is attached to residue 29 of the IL-2 polypeptide and comprises an additional point of attachment to the N-terminus of the IL-2 polypeptide.

In some embodiments, the cleavable moiety is attached to residue 32 of the IL-2 polypeptide of the activatable immunocytokine and comprises an additional point of attachment to residue 9 of the IL-2 polypeptide. In some embodiments, the cleavable moiety is attached to residue 32 of the IL-2 polypeptide and comprises an additional point of attachment to the N-terminus of the IL-2 polypeptide.

Exemplary points of attachment, additional points of attachment, and orientations of cleavable peptides to IL-2 polypeptides of activatable immunocytokines as provided herein are provided in the table below.

| | | | Linking Group | | | Linking Group |
|---|---|---|---|---|---|---|
| | First Residue | Position of | Between First | Second Residue | Position of | Between Second |
| | Position of | Cleavable Peptide | Residue Position | Position of IL-2 | Cleavable Peptide | Residue Position |
| Exemplary | IL-2 Attached | Attached to First | of IL-2 and | Attached to | Attached to Second | of IL-2 and |
| Orientation | to Cleavable | Residue Position | Cleavable | Cleavable | Residue Position | Cleavable |
| Number | Peptide | of IL-2 | Peptide | Peptide | of IL-2 | Peptide |
| 1 | 32 | N- | PEG9-Glutaryl | 1 (N- | C-terminus | PEG9 |
| | | terminus | | terminus | | |
| | | | | of IL-2) | | |
| 2 | 32 | N- | PEG4-Glutaryl | 1 (N- | C-terminus | PEG4 |
| | | terminus | | terminus | | |
| | | | | of IL-2) | | |
| 3 | 32 | N- | PEG16- | 1 (N- | C-terminus | PEG16 |
| | | terminus | Glutaryl | terminus | | |
| | | | | of IL-2) | | |
| 4 | 32 | N- | PEG9-Glutaryl | 1 (N- | C-terminus | PEG24 |
| | | terminus | | terminus | | |
| | | | | of IL-2) | | |
| 5 | 32 | N- | PEG9-Glutaryl | 1 (N- | C-terminus | None |
| | | terminus | | terminus | | |
| | | | | of IL-2) | | |
| 6 | 32 | N- | Glutaryl | 1 (N- | C-terminus | PEG9 |
| | | terminus | | terminus | | |
| | | | | of IL-2) | | |
| 7 | 29 | N- | PEG9 | 1 (N- | C-terminus | PEG9 |
| | | terminus | | terminus | | |
| | | | | of IL-2) | | |
| 8 | 29 | N- | PEG24 | 1 (N- | C-terminus | PEG24 |
| | | terminus | | terminus | | |
| | | | | of IL-2) | | |
| 9 | 29 | N- | PEG4 | 1 (N- | C-terminus | PEG4 |
| | | terminus | | terminus | | |
| | | | | of IL-2) | | |
| 10 | 29 | N- | PEG16 | 1 (N- | C-terminus | PEG16 |
| | | terminus | | terminus | | |
| | | | | of IL-2) | | |
| 11 | 29 | N- | PEG9 | 1 (N- | C-terminus | None |
| | | terminus | | terminus | | |
| | | | | of IL-2) | | |
| 12 | 29 | N- | None | 1 (N- | C-terminus | PEG9 |
| | | terminus | | terminus | | |
| | | | | of IL-2) | | |
| 13 | 19 | N- | None | 1 (N- | C-terminus | PEG4 |
| | | terminus | | terminus | | |
| | | | | of IL-2) | | |
| 14 | 19 | N- | PEG4 | 1 (N- | C-terminus | None |
| | | terminus | | terminus | | |
| | | | | of IL-2) | | |
| 15 | 22 | N- | None | 1 (N- | C-terminus | PEG4 |
| | | terminus | | terminus | | |
| | | | | of IL-2) | | |
| 16 | 22 | N- | PEG4 | 1 (N- | C-terminus | None |
| | | terminus | | terminus | | |
| | | | | of IL-2) | | |
| 17 | 26 | N- | None | 1 (N- | C-terminus | PEG9 |
| | | terminus | | terminus | | |
| | | | | of IL-2) | | |
| 18 | 26 | N- | PEG9 | 1 (N- | C-terminus | None |
| | | terminus | | terminus | | |
| | | | | of IL-2) | | |
| 19 | 23 | C- | None | 1 (N- | N-terminus | PEG9- |
| | | terminus | | terminus | | Glutaryl |
| | | | | of IL-2) | | |
| 20 | 23 | C- | None | 1 (N- | N-terminus | PEG4- |
| | | terminus | | terminus | | Glutaryl |
| | | | | of IL-2) | | |
| 21 | 23 | C- | None | 1 (N- | N-terminus | Glutaryl |
| | | terminus | | terminus | | |
| | | | | of IL-2) | | |
| 22 | 23 | C- | PEG4 | 9 | N-terminus | None |
| | | terminus | | | | |
| 23 | 23 | C- | None | 9 | N-terminus | PEG4 |
| | | terminus | | | | |
| 24 | 23 | C- | None | 9 | N-terminus | None |
| | | terminus | | | | |
| 25 | 23 | N- | None | 1 (N- | C-terminus | PEG4 |
| | | terminus | | terminus | | |
| | | | | of IL-2) | | |
| 26 | 23 | N- | PEG4 | 1 (N- | C-terminus | None |
| | | terminus | | terminus | | |
| | | | | of IL-2) | | |
| 27 | 23 | N- | None | 1 (N- | C-terminus | PEG9 |
| | | terminus | | terminus | | |
| | | | | of IL-2) | | |
| 28 | 23 | N- | PEG9 | 1 (N- | C-terminus | None |
| | | terminus | | terminus | | |
| | | | | of IL-2) | | |
| 29 | 23 | N- | None | 1 (N- | C-terminus | None |
| | | terminus | | terminus | | |
| | | | | of IL-2) | | |

In some embodiments, the additional point of attachment of the cleavable moiety is to a residue which is substituted relative to the corresponding amino acid residue of SEQ ID NO: 1. The substitution can be any suitable amino acid, including any of those provided supra (e.g., lysine, glutamate, glutamine, aspartate, asparagine, tyrosine, serine, threonine, cysteine, an unnatural amino acid, etc.). In some embodiments, the additional point of attachment of the cleavable moiety is to a residue which is the natural residue at the corresponding position in SEQ ID NO: 1.

In embodiments wherein the cleavable moiety comprises multiple (e.g., 2) points of attachment to the IL-2 polypeptide of the activatable immunocytokine, cleavage of the cleavable linker can leave a portion of the cleavable linker attached to each point of attachment (or to one point of attachment). The portion attached to each point of attachment can be any of the portions remaining attached provided herein. For example, where a cleavable peptide is the cleavable moiety and comprises two points of attachment to the IL-2 polypeptide, cleavage of the cleavable peptide can leave a number of amino acids attached to the first point of attachment (e.g., 1, 2, 3, 4, 5, 6, 7, 8, 9, 10, or more amino acids) and can leave a number of amino acids attached to the second point of attachment (e.g., 1, 2, 3, 4, 5, 6, 7, 8, 9, 10, or more amino acids).

In some embodiments, an activatable IL-2 polypeptide herein comprises a cleavable moiety (e.g., a cleavable peptide) attached to residue 23 and the N-terminus (e.g., at a position corresponding to residue 1 of SEQ ID NO: 1, or another residue of SEQ ID NO: 1 wherein the sequence of SEQ ID NO: 1 has been truncated). In some embodiments, the N-terminus of the IL-2 polypeptide is the amino acid residue at a position corresponding to the first residue of SEQ ID NO: 1. In some embodiments, the cleavable moiety comprises a cleavable peptide. In some embodiments, the cleavable peptide comprises any one of the sequences described herein (e.g., in Table 1C or Table 1D, such as SEQ ID NO. 617 or 633). In some embodiments, the cleavable peptide is one cleavable by matriptase and/or an MMP. In some embodiments, the cleavable peptide is directly attached to the N-terminus of the IL-2 polypeptide (e.g., the C-terminus of the cleavable peptide forms an amide bond with the N-terminus of the IL-2 polypeptide). In some embodiments, the cleavable peptide is directly attached to residue 23 of the IL-2 polypeptide (e.g., the N-terminus of the cleavable peptide forms an amide bond with a carboxylic acid group of the side chain of residue 23). In some embodiments, residue 23 comprises a side chain with a carboxylic acid. In some embodiments, residue 23 of the IL-2 polypeptide is glutamate.

### Additional Moieties Attached to the Cleavable Moieties

In some embodiments, the cleavable moiety attached to the IL-2 polypeptide of the activatable immunocytokine is further attached to an additional moiety. The additional moiety to which the cleavable moiety is attached can be any desired additional moiety discussed (e.g., antibodies or antigen binding fragments thereof, targeting peptides, Fc domains, etc.), polymers (e.g., water soluble polymers, such as poly(ethylene glycol)), lipids, nanoparticles, nucleic acids (e.g., aptamers), small molecules, or other functionalities). In some embodiments, the additional moiety is positioned on the cleavable moiety such that cleavage of the cleavable moiety causes the additional moiety to no longer be attached to the IL-2 polypeptide. The additional moiety can be attached to the cleavable peptide either directly or through a suitable linking group.

In some embodiments, the cleavable moiety is further attached to a polymer. In some embodiments, the polymer is a chemical polymer. In some embodiments, the polymer comprises a water soluble polymer. In some embodiments, the polymer comprises poly(alkylene oxide), polysaccharide, poly(vinyl pyrrolidone), poly(vinyl alcohol), polyoxazoline, poly(acryloylmorpholine), or a combination thereof. In some embodiments, the polymer comprises poly(alkylene oxide). In some embodiments, the polymer is polyethylene glycol or polypropylene glycol, or a combination thereof. In some embodiments, the polymer is polyethylene glycol.

In some embodiments, the polymer attached to the cleavable moiety is linear. In some embodiments, the polymer is branched. In some embodiments, a branched polymer comprises a plurality of polymeric chains attached to a support structure with multiple points of attachment. In some embodiments, the branched polymer comprises a lysine with two polymers covalently attached. In some embodiments, the support structure comprises one or more lysine residues. In some embodiments, the support structure comprises a plurality of lysines coupled together. In some embodiments, the support structure comprises 1, 2, 3, 4, or more lysines coupled together, either through the alpha amine or the side chain amine to the carboxyl of another lysine. In some embodiments, the branched polymer comprises 2, 3, 4, or more polymeric chains. In some embodiments, the branched polymer comprises a structure of

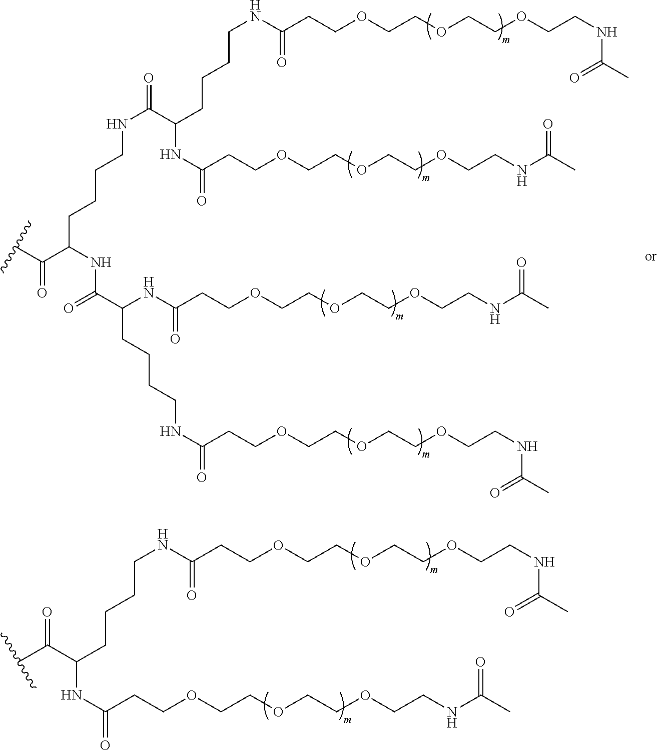

wherein each m is independently an integer from 2-50.

In some embodiments, the polymer attached to the cleavable moiety has a molecular weight of from about 0.1 kDa to about 50 kDa. In some embodiments, the polymer has a molecular weight of at least 0.1 kDa, at least 0.5 kDa, at least 1 kDa, at least 2 kDa, at least 3 kDa, at least 5 kDa, at least 10 kDa, at least 20 kDa, or at least 30 kDa. In some embodiments, the polymer has a molecular weight of at most about 50 kDa, 40 kDa, 30 kDa, 20 kDa, 15 kDa, 10 kDa, 5 kDa, 3 kDa, 2 kDa, or 1 kDa. In some embodiments, the polymer has a molecular weight of from about 0.1 kDa to about 50 kDa, about 0.1 kDa to about 40 kDa, about 0.1 kDa to about 30 kDa, about 0.1 kDa to about 20 kDa, about 0.1 kDa to about 15 kDa, about 0.1 kDa to about 10 kDa, about 0.1 kDa to about 5 kDa, about 0.1 kDa to about 3 kDa, about 0.1 kDa to about 2 kDa, about 0.1 kDa to about 1 kDa, 0.5 kDa to about 50 kDa, about 0.5 kDa to about 40 kDa, about 0.5 kDa to about 30 kDa, about 0.5 kDa to about 20 kDa, about 0.5 kDa to about 15 kDa, about 0.5 kDa to about 10 kDa, about 0.5 kDa to about 5 kDa, about 0.5 kDa to about 3 kDa, about 0.5 kDa to about 2 kDa, about 0.5 kDa to about 1 kDa, about 1 kDa to about 50 kDa, about 1 kDa to about 30 kDa, about 1 kDa to about 15 kDa, about 1 kDa to about 10 kDa, about 1 kDa to about 5 kDa about 1 kDa to about 3 kDa, about 1 kDa to about 2 kDa, about 3 kDa to about 50 kDa, about 3 kDa to about 30 kDa, about 3 kDa to about 15 kDa, about 3 kDa to about 10 kDa, about 5 kDa to about 50 kDa, about 5 kDa to about 30 kDa, about 5 kDa to about 20 kDa, about 10 kDa to about 50 kDa, or about 10 kDa to about 30 kDa.

### Additional Modifications to IL-2 Polypeptides of Activatable Immunocytokines

In some embodiments, the IL-2 polypeptide comprising a cleavable moiety of an activatable immunocytokine comprises additional modifications in addition to the attachment of the cleavable moiety (e.g., the one or two points of attachment of the cleavable moiety and corresponding amino acid substitutions to effectuate the attachment of the cleavable moiety). Additional modifications to the IL-2 polypeptide can included without limitation amino acid substitutions, deletions, additions, attachment of polymer moieties, conjugation to other moieties (e.g., additional polypeptides), and/or fusions to other polypeptides. Unless otherwise specified, modifications to indicated amino acid residue numbers of an IL-2 polypeptide described herein utilize SEQ ID NO: 1 as a reference sequence (wild type IL-2).

In some embodiments, the IL-2 polypeptide of the activatable immunocytokine comprising a cleavable moiety comprises modifications which bias the IL-2 polypeptide in favor of signaling through the IL-2 receptor beta subunit (or the IL-2 receptor βγ complex) over the IL-2 receptor alpha subunit (or the IL-2 receptor αβγ complex). Wild type IL-2 (SEQ ID NO: 1) displays a greater ability to bind and signal through the IL-2 receptor alpha subunit (or the IL-2 receptor αβγ complex) than the IL-2 receptor beta subunit (or the IL-2 receptor βγ complex), thereby generally biasing IL-2 in favor of stimulating Treg cells. Conversely, in some embodiments, the IL-2 polypeptides of the activatable IL-2 polypeptides provided herein are biased in favor binding to the IL-2 receptor beta subunit (or the IL-2 receptor βγ complex). In some embodiments, this is accomplished through modification(s) which enhance the binding of the IL-2 polypeptide to the IL-2 receptor beta subunit (or the IL-2 receptor βγ complex), through modification(s) which reduce the binding of the IL-2 polypeptide to the IL-2 receptor alpha subunit (or the IL-2 receptor αβγ complex), or a combination of these modifications. Non-limiting examples of modifications which bias IL-2 polypeptides towards IL-2 receptor beta signaling are described in, for example, Patent Cooperation Treaty Publication Nos. WO2021/140416, WO2012/065086, WO2019/028419, WO2012/107417, WO2018/119114, WO2012/062228, WO2019/104092, WO2012/088446, and WO2015/164815, each of which is hereby incorporated by reference as if set forth herein in its entirety. It is contemplated any suitable modifications provided therein are compatible with embodiments of the instant disclosure.

In addition to modifications of IL-2 which may affect binding to one or more IL-2 receptor subunits (such as the alpha subunit), the IL-2 polypeptide comprising a cleavable moiety of the activatable immunocytokine provided herein may also comprise one or more modifications which improve the stability or pharmacokinetic properties of the IL-2 polypeptide. For example, the IL-2 polypeptide provided herein can comprise the modifications relative to SEQ NO: 1 which are contained in aldesluekin (Proleukin®), namely a deletion of the N-terminal A residue and a C125S substitution relative to SEQ ID NO: 1 (Wild type IL-2).

In some embodiments, the IL-2 polypeptide comprising a cleavable moiety of the activatable immunocytokine described herein contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more modified amino acid residues compared to SEQ ID NO: 1. In some embodiments, the IL-2 polypeptide of the activatable IL-2 polypeptide composition comprises an amino acid sequence having at least 80%, 85%, 900%, 91%, 92%, 93%, 94%, 95%, 96%, 97%, 98%, or 99% sequence identity to the sequence set forth in SEQ ID NO: 1. In some embodiments, the IL-2 polypeptide of the activatable IL-2 polypeptide comprises an amino acid sequence having at least 80%, 85%, 90%, 91%, 92%, 93%, 94%, 95%, 96%, 97%, 98%, or 99% sequence identity to the sequence set forth in SEQ ID NO: 2. In some embodiments, the IL-2 polypeptide of the activatable IL-2 polypeptide comprises an amino acid sequence having at least 80%, 85%, 90%, 91%, 92%, 93%, 94%, 95%, 96%, 97%, 98%, or 99% sequence identity to the sequence set forth in SEQ ID NO: 3.

In some embodiments, the IL-2 polypeptide comprising a cleavable moiety of the activatable immunocytokine described herein comprises at least 1, at least 2, at least 3, at least 4, at least 5, at least 6, at least 7, or at least 9 amino acid substitutions, wherein the amino acid substitutions are relative to SEQ ID NO: 1. In some embodiments, the IL-2 polypeptide comprises 1 to 9 amino acid substitutions. In some embodiments, the IL-2 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-2 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-2 polypeptide comprises at most 4 amino acid substitutions, 5 amino acid substitutions, 6 amino acid substitutions, 7 amino acid substitutions, or 9 amino acid substitutions.

In some embodiments, the IL-2 polypeptide comprising the cleavable moiety of the activatable immunocytokine described herein comprises at least 1, at least 2, at least 3, at least 4, at least 5, at least 6, at least 7, or at least 9 amino acid substitutions, wherein the amino acid substitutions are relative to SEQ ID NO: 2. In some embodiments, the IL-2 polypeptide comprises 1 to 9 amino acid substitutions. In some embodiments, the IL-2 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-2 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-2 polypeptide comprises at most 4 amino acid substitutions, 5 amino acid substitutions, 6 amino acid substitutions, 7 amino acid substitutions, or 9 amino acid substitutions.

Non-limiting examples of modifications to IL-2 polypeptides comprising a cleavable moiety of activatable immunocytokines include amino acid substitutions shown in Table 2A below. In some embodiments, the IL-2 polypeptide comprises 1, 2, 3, 4, 5, or more of the amino acid substitutions set forth in Table 2A.

TABLE 2A

| WT IL-2 Residue | WT IL-2 | |
|---|---|---|
| Number* | Residue | Mutations |
| 35 | K | D, I, L, M, N, P, Q, T, Y |
| 36 | L | A, D, E, F, G, H, I, K, M, N, P, R, S, |
| | | W, Y |
| 38 | R | A, D, G, K, N, P, S, Y |
| 40 | L | D, G, N, S, Y |
| 41 | T | E, G, Y |
| 42 | F | A, D, E, G, I, K, L, N, Q, R, S, T, V, |
| | | Y |
| 43 | K | H, Y |
| 44 | F | K, Y |
| 45 | Y | A, D, E, G, K, L, N, Q, R, S, T, V |
| 46 | M | I, Y |
| 61 | E | K, M, R, Y |
| 62 | E | D, L, T, Y |
| 64 | K | D, E, G, L, Q, R, Y |
| 65 | P | D, E, F, G, H, I, K, L, N, Q, R, S, T, V, |
| | | W, Y |
| 66 | L | A, F, Y |
| 67 | E | A, Y |
| 68 | E | V, Y |
| 72 | L | A, D, E, G, K, N, Q, R, S, T, Y |
| 125 | C | S |

*Residue position numbering based on SEQ ID NO: 1 as a reference sequence

In some embodiments, the IL-2 polypeptide comprising the cleavable moiety of the activatable immunocytokine comprises 1, 2, 3, 4, 5, or more of the amino acid substitutions set forth in Table 2B.

TABLE 2B

| WT IL-2 | | |
|---|---|---|
| Residue | WT IL-2 | |
| Number* | Residue | Mutations |
| 20 | D | T, Y |
| 35 | K | D, I, L, M, N, P, Q, TY |
| 38 | R | A, D, G, K, N, P, S, Y |
| 42 | F | A, D, E, G, I, K, L, N, Q, R, S, T, V, Y |
| 43 | K | H, Y |
| 45 | Y | A, D, E, G, K, L, N, Q, R, S, T, V, Y |
| 62 | E | D, L, T, Y |
| 65 | P | D, E, F, G, H, I, K, L, N, Q, R, S, T, V, |
| | | W, Y |
| 68 | E | V, Y |
| 72 | L | A, D, E, G, K, N, Q, R, S, T, Y |
| 125 | C | S |

*Residue position numbering based on SEQ ID NO: 1 as a reference sequence

In some embodiments, the IL-2 polypeptide comprising the cleavable moiety of the activatable immunocytokine comprises at least one modification is in the range of amino acid residues 30-75 of SEQ ID NO: 1. In some embodiments, the IL-2 polypeptide comprises at least one polymer attachment to the residue at position 42 and/or 45 and/or an amino acid substitution at residue position 42 and/or 45. In some embodiments, one modification is at amino acid residue 42. In some embodiments, one modification is a F42Y substitution. In some embodiments, one modification is a polymer attached to residue F42Y. In some embodiments, one modification is at residue 45. In some embodiments, the modification at residue 45 is a polymer attached to residue 45. In some embodiments, the modification at residue 45 is a polymer attached to residue Y45. In some embodiments, the IL-2 polypeptide comprises a first polymer attached at residue F42Y and a second polymer attached at residue Y45. In some embodiments, at least one of the first polymer or the second polymer comprises a conjugation handle covalently attached thereto. In some embodiments, the conjugation handle is attached to the polymer attached at residue F42Y. In some embodiments, the conjugation handle comprises an azide. In some embodiments, the IL-2 polypeptide comprises a deletion of residue 1 from SEQ ID NO: 1. In some embodiments, the IL-2 polypeptide comprises a C125S substitution. In some embodiments, the IL-2 polypeptide further comprises one or more substitutions of a synthetic IL-2 polypeptide as provided herein (e.g., Hse or Nle substitutions).

In one aspect, provided herein, is an IL-2 polypeptide of an activatable immunocytokine as provided herein (e.g., with a cleavable moiety attached to at least one residue of the IL-2 polypeptides provided herein), comprising a first polymer covalently attached at residue 42 and a second polymer covalently attached at residue 45, wherein residue position numbering of the IL-2 polypeptide is based on SEQ ID NO: 1 as a reference sequence. In some embodiments, the first polymer and the second polymer are the same. In some embodiments, the first polymer and the second polymer are different. In some embodiments, at least one of the first polymer or the second polymers comprises a conjugation handle (e.g., an azide). In some embodiments, the conjugation handle forms a reaction product with a complementary conjugation handle to form a part of the linker to the immune checkpoint inhibitor molecule (e.g., the anti-PD-1 antibody or antigen binding fragment thereof). In some embodiments, at least one of the first polymer or the second polymer is comprised in the linker to the immune checkpoint inhibitor molecule (e.g., the anti-PD-1 antibody or antigen binding fragment thereof). In some embodiments, each polymer is attached through a tyrosine residue. In some embodiments, the IL-2 polypeptide comprises an amino acid sequence at least 80%, at least 85%, at least 90%, at least 95%, at least 98%, or at least 99% identical to the amino acid sequence of SEQ ID NO: 1. In some embodiments, the IL-2 polypeptide comprises an amino acid sequence at least 80%, at least 85%, at least 90%, at least 95%, at least 98%, or at least 99% identical to the amino acid sequence of SEQ ID NO: 2. In some embodiments, the IL-2 polypeptide comprises a C125S or C125A substitution relative to SEQ ID NO: 1. In some embodiments, the IL-2 polypeptide comprises a deletion of A1 from the sequence of SEQ ID NO: 1. In some embodiments, the IL-2 polypeptide comprises amino acid substitutions at 1, 2, 3, or 4 methionine residues from SEQ ID NO: 1. In some embodiments, the IL-2 polypeptide further comprises unnatural amino acid substitutions at residues M23, M39, and/or M46. In some embodiments, the unnatural amino acid residues substituted for the methionines are each independently norleucine or O-methyl-homoserine. In some embodiments, the IL-2 polypeptide further comprises homoserine Hse 41, Hse 71, and Hse 104. In some embodiments, the IL-2 polypeptide.

In some embodiments, the IL-2 polypeptide (e.g., the IL-2 polypeptide comprising the cleavable moiety) of the activatable immunocytokine comprises a polymer attached to a residue of the IL-2 polypeptide (e.g., a polymer in addition one which may attach the cleavable moiety as part of a linking group and/or which may attach to the immune checkpoint inhibitor molecule (e.g., the anti-PD-1 antibody or antigen binding fragment thereof) as part of the linker). In some embodiments, the polymer is attached to a different residue than the residue to which the cleavable moiety is attached. In some embodiments, the polymer is attached to a different residue than the residue to which the linker is attached.

In some embodiments, the polymer (e.g., a polymer which is not attached to the cleavable moiety and/or which is not comprised in the linker) is attached to an amino acid residue of the IL-2 polypeptide of the activatable immunocytokine. In some embodiments, the polymer is attached to any amino acid residue of the IL-2 polypeptide (e.g., at a position corresponding to any one of positions 1-133 of SEQ ID NO: 1). In some embodiments, the polymer is attached at a non-terminal residue (e.g., a residue other than the C-terminal residue or N-terminal residue) of the IL-2 polypeptide (e.g., a residue at position corresponding to any one of positions 2-132 of SEQ ID NO. 1). In some embodiments, the polymer is attached at a terminal residue of the IL-2 polypeptide, wherein the IL-2 polypeptide has been extended or truncated by one or more amino acids relative to SEQ ID NO: 1 (e.g., the polymer is attached to a residue corresponding to residue 2 of SEQ ID NO: 1 and residue 1 of SEQ ID NO: 1 has been deleted). In some embodiments, the polymer is attached to the N-terminal residue of the IL-2 polypeptide. In some embodiments, the polymer is attached to the N-terminal amine of the IL-2 polypeptide. In some embodiments, the polymer is attached to the C-terminal residue of the IL-2 polypeptide. In some embodiments, the polymer is attached to the C-terminal carboxyl group of the IL-2 polypeptide.

In some embodiments, the polymer (e.g., a polymer which is not attached to the cleavable moiety and/or which is not comprised in the linker) is attached to the IL-2 polypeptide of the activatable immunocytokine at a residue in a region comprising residues 2-132, wherein residue position numbering is based on SEQ ID NO: 1 as a reference sequence. In some embodiments, the polymer is attached to the IL-2 polypeptide at a residue in a region comprising residues 30-75. In some embodiments, the polymer is attached to the IL-2 polypeptide at a residue in a region comprising residues 35-55, residues 35-50, residues 35-45, residues 30-50, residues 4045, residues 60-75, residues 60-70, residues 65-70, or residues 2-5. In some embodiments, the polymer is attached to the IL-2 polypeptide at a residue selected from residue 65, 66, 67, 68, 69, and 70. In some embodiments, the polymer is attached to the IL-2 polypeptide at a residue selected from residue 40, 41, 42, 43, 44, and 45. In some embodiments, the polymer is attached to the IL-2 polypeptide at residue 42 or 45. In some embodiments, the polymer is attached to the IL-2 polypeptide at residue 42. In some embodiments, the polymer is attached to the IL-2 polypeptide at residue 45.

In some embodiments, the polymer (e.g., a polymer which is not attached to the cleavable moiety and/or which is not comprised in the linker) is attached to the IL-2 polypeptide of the activatable immunocytokine at a residue which disrupts binding of the IL-2 polypeptide with the IL-2 receptor alpha subunit (IL-2Rα). Examples of these residues include residues 3, 5, 34, 35, 36, 37, 38, 40, 41, 42, 43, 44, 45, 60, 61, 62, 63, 64, 65, 67, 68, 69, 71, 72,103,104, 105, and 107, as described in, for example, PCT Pub. Nos. WO2019/028419, WO2020/056066, WO2021/140416, and WO2021/216478 each of which is hereby incorporated by reference as if set forth in its entirety. In some embodiments, the polymer is covalently attached at a residue selected from residues corresponding to residues 3, 5, 34, 35, 36, 37, 38, 40, 41, 42, 43, 44, 45, 60, 61, 62, 63, 64, 65, 67, 68, 69, 71, 72,103,104, 105, and 107 of SEQ ID NO: 1. In some embodiments, the polymer is covalently attached at residue 1, 35, 37, 38, 41, 42, 43, 44, 45, 60, 61, 62, 64, 65, 68, 69, 71, 72, 104, 105, or 107 of the IL-2 polypeptide. In some embodiments, the polymer is covalently attached at residue 5. In some embodiments, the polymer is covalently attached at residue 38. In some embodiments, the polymer is covalently attached at residue 42. In some embodiments, the polymer is covalently attached at residue 45. In some embodiments, the polymer is covalently attached at residue 61. In some embodiments, the polymer is covalently attached at residue 65. In some embodiments, the polymer is covalently attached at residue 68.

In some embodiments, the residue to which the polymer (e.g., a polymer which is not attached to the cleavable moiety and/or which is not comprised in the linker) is attached is a natural amino acid residue. In some embodiments, the residue to which the polymer is covalently attached is selected from cysteine, aspartate, asparagine, glutamate, glutamine, serine, threonine, lysine, and tyrosine. In some embodiments, the residue to which the polymer is covalently attached is selected from asparagine, aspartic acid, cysteine, glutamic acid, glutamine, lysine, and tyrosine. In some embodiments, the polymer is covalently attached to a cysteine. In some embodiments, the polymer is covalently attached to a lysine. In some embodiments, the polymer is covalently attached to a glutamine. In some embodiments, the polymer is covalently attached to an asparagine. In some embodiments, the residue to which the polymer is attached is a tyrosine. In some embodiments, the residue to which the polymer is attached is the natural amino acid in that position in SEQ ID NO: 1 (e.g., Y45 or A1).

In some embodiments, the polymer (e.g., a polymer which is not attached to the cleavable moiety and/or which is not comprised in the linker) is attached to a different natural amino acid which is substituted at the relevant position. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired reactive group, such as a CLICK chemistry reagent such as an azide, alkyne, etc.). In some embodiments, the polymer is covalently attached site-specifically to a natural amino acid.

In some embodiments, the polymer (e.g., a polymer which is not attached to the cleavable moiety and/or which is not comprised in the linker) is attached to a tyrosine residue. In some embodiments, the polymer attached to the tyrosine residue has a structure

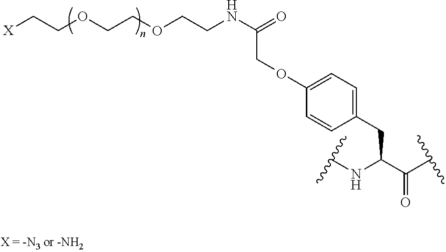

wherein n is an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 9. In some embodiments, n is 8. In some embodiments, n is 6. In some embodiments, n is 12. In some embodiments, the polymer attached to the tyrosine residue is at residue F42Y. In some embodiments, the polymer attached to the tyrosine residue is at Y45. In some embodiments, the IL-2 polypeptide comprises two polymers attached to tyrosine residues at F42Y and Y45. In some embodiments, the two polymers are the same size. In some embodiments, one of the two polymers comprises the azide and the other polymer comprises the amine. In some embodiments, the polymer at F42Y comprises the azide and the polymer at Y45 comprises the amine. In some embodiments, the polymer comprising the azide is used to form a part of the linker to the immune checkpoint inhibitor molecule (e.g., the anti-PD-1 antibody or antigen binding fragment thereof).

In some embodiments, the polymer (e.g., a polymer which is not attached to the cleavable moiety and/or which is not comprised in the linker) is attached at an unnatural amino acid residue. In some embodiments, the unnatural amino acid residue comprises a conjugation handle. In some embodiments, the conjugation handle facilitates the addition of the polymer to the modified IL-2 polypeptide. The conjugation handle can be any of the conjugation handles provided herein, and is preferably a different conjugation handle which is non-reactive with a conjugation handle used to attach or form part of the linker (where a conjugation handle is used to form the linker). In some embodiments, the polymer is covalently attached site-specifically to the unnatural amino acid. Non-limiting examples of amino acid residues comprising conjugation handles can be found, for example, in PCT Pub. Nos. WO2015/054658, WO2014/036492, and WO2021/133839, WO2006/069246, and WO2007/079130, each of which is incorporated by reference as if set forth in its entirety. In some embodiments, the polymer is attached to an unnatural amino acid residue without use of a conjugation handle.

In some embodiments, the polymer (e.g., a polymer which is not attached to the cleavable moiety and/or which is not comprised in the linker) is covalently attached at residue 42. In some embodiments, the polymer is covalently attached at residue F42E, F42D, F42Q, F42K, F42N, or F42Y. In some embodiments, the polymer is covalently attached at residue F42Y. In some embodiments, the polymer is covalently attached to an unnatural amino acid at residue 42.

In some embodiments, the polymer (e.g., a polymer which is not attached to the cleavable moiety and/or which is not comprised in the linker) is covalently attached at residue 45. In some embodiments, the polymer is covalently attached at residue Y45, Y45E, Y45C, Y45D, Y45Q, Y45K, or Y45N. In some embodiments, the polymer is covalently attached at residue Y45. In some embodiments, the polymer is covalently attached to an unnatural amino acid at residue 45.

In some embodiments, the polymer (e.g., a polymer which is not attached to the cleavable moiety and/or which is not comprised in the linker) is covalently attached at residue 65. In some embodiments, the polymer is covalently attached at residue P65C, P65D, P65Q, P65E, P65N, P65K, or P65Y. In some embodiments, the polymer is covalently attached to an unnatural amino acid at residue 65.

In some embodiments, the polymer (e.g., a polymer which is not attached to the cleavable moiety and/or which is not comprised in the linker) is covalently attached at residue 5. In some embodiments, the polymer is covalently attached at residue S5C, S5D, S5Q, S5K, S5N, S5K, or S5Y. In some embodiments, the polymer is covalently attached to an unnatural amino acid at residue 5.

In some embodiments, the polymer (e.g., a polymer which is not attached to the cleavable moiety and/or which is not comprised in the linker) is covalently attached at residue 1. In some embodiments, the polymer is covalently attached at residue A1. In some embodiments, the polymer is covalently attached to the N-terminal amine of the IL-2 polypeptide.

In some embodiments, the polymer (e.g., a polymer which is not attached to the cleavable moiety and/or which is not comprised in the linker) comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is polysaccharide. In some embodiments, the water-soluble polymer is poly(ethylene oxide) (PEG).

In some embodiments, the polymer (e.g., a polymer which is not attached to the cleavable moiety and/or which is not comprised in the linker) has a molecular weight of from about 0.1 kDa to about 50 kDa. In some embodiments, the polymer has a molecular weight of from about 0.1 kDa to about 0.5 kDa from about 0.1 kDa to about 1 kDa, from about 0.1 kDa to about 2 kDa, from about 0.1 kDa to about 5 kDa from about 0.2 kDa to about 1 kDa, from about 0.2 kDa to about 2 kDa, from about 0.2 kDa to about 5 kDa, from about 0.2 kDa to about 10 kDa, from about 0.2 kDa to about 30 kDa, from about 0.5 kDa to about 2 kDa, from about 0.5 kDa to about 5 kDa, from about 0.5 kDa to about 10 kDa, from about 0.5 kDa to about 30 kDa, from about 1 kDa to about 5 kDa, from about 1 kDa to about 10 kDa, from about 1 kDa to about 30 kDa, from about 1 kDa to about 50 kDa, from about 2 kDa to about 10 kDa, from about 2 kDa to about 30 kDa, from about 2 kDa to about 50 kDa, from about 5 kDa to about 30 kDa, or from about 5 kDa to about 50 kDa. In some embodiments, the polymer has a molecular weight of at least about 0.2 kDa, at least about 0.5 kDa, at least about 1 kDa, at least about 2 kDa, at least about 5 kDa, at least about 10 kDa, or at least about 30 kDa. In some embodiments, the polymer has a molecular weight of at most about 30 kDa, at most about 10 kDa, at most about 5 kDa, at most about 2 kDa, at most about 1 kDa, at most about 0.5 kDa or at most about 0.2 kDa. In some embodiments, the polymer has a molecular weight of about 0.5 kDa, about 1 kDa, about 2 kDa, about 3 kDa, about 4 kDa, about 5 kDa, about 7.5 kDa, about 10 kDa, about 12.5 kDa, about 15 kDa, about 20 kDa, about 25 kDa, about 30 kDa, about 35 KDa, about 40 kDa, about 45 kDa, or about 50 kDa. In some embodiments, the polymer is a PEG polymer.

In some embodiments, the polymer (e.g., a polymer which is not attached to the cleavable moiety and/or which is not comprised in the linker) is an end-capped polymer. In some embodiments, the polymer is an end-capped polyethylene glycol. In some embodiments, the polymer is end-capped with a functional group selected from amine, alkoxy (e.g., methoxy, ethoxy, propoxy, etc.), hydroxyl, amide (e.g., —NH(C═O)(C₁-C₄ alkyl), carboxylate, and ester (e.g., methyl ester, ethyl ester, etc.). In some embodiments, the polymer as an amine end-capped PEG.

In some embodiments, the IL-2 polypeptide comprises two polymers (e.g., polymers not attached to the cleavable moiety) covalently attached to two separate residues of the IL-2 polypeptide. In some embodiments, the two polymers are a first polymer and a second polymer. Each of the first polymer and the second polymer can be attached to the IL-2 polypeptide at any of the residues as provided herein and can be any of the polymers provided herein (e.g., having any combination of sizes as provided herein). In some embodiments, both of the first polymer and the second polymer are the same size or about the same size. In some embodiments, both polymers are at most about 1 kDa. In some embodiments, one polymer is substantially larger than the other. In some embodiments, one polymer is at most about 1 kDa and the other polymer is at least about 5 kDa. In some embodiments, one polymer comprises a conjugation handle and the second polymer does not. In some embodiments, the conjugation handle is used to form the linker with the immune checkpoint inhibitor molecule (e.g., an anti-PD-1 antibody or antigen binding fragment thereof). In some embodiments, one of the polymers comprises a portion of the linker between IL-2 polypeptide and the immune checkpoint inhibitor molecule (e.g., an anti-PD-1 antibody or antigen binding fragment thereof).

In some embodiments, the IL-2 polypeptide comprising a cleavable moiety of the activatable immunocytokine is a synthetic IL-2 polypeptide. In some embodiments, the synthetic IL-2 polypeptide is prepared from one or more chemically synthesized fragments. Synthetic IL-2 polypeptides have been previously described, at least in PCT Publication No. WO2021/140416, US Patent Application Publication No. US2021/0155665, and Asahina et al., Angew. Chem. Int. Ed. 2015, 54, 8226-8230, each of which is incorporated by reference as if set forth herein in its entirety.

Any IL-2 polypeptide of an activatable immunocytokine provided herein may be prepared as a synthetic IL-2 polypeptide (e.g., having any of the amino acid substitutions (e.g., F42Y, C125S, etc.) or other modifications (e.g., polymer attachment) provided herein in conjunction with the substitutions provided herein for synthetic IL-2 polypeptides, such as homoserine or norleucine residues). In some embodiments, the IL-2 polypeptide comprises any of the amino acid substitutions present in a synthetic IL-2 polypeptide as provided herein (e.g., one or more homoserine or norleucine residues as provided herein). In some embodiments, a synthetic IL-2 polypeptide exhibits a similar or substantially identical activity to a corresponding recombinant IL-2 (e.g., a synthetic IL-2 polypeptide having the same functional modifications to the structure or sequence of the IL-2 polypeptide).

In some embodiments, the synthetic IL-2 polypeptide of the activatable immunocytokine comprises a homoserine (Hse) residue located in any one of residues 35-45. In some embodiments, the synthetic IL-2 polypeptide comprises a Hse residue located in any one of residues 61-81. In some embodiments, the synthetic IL-2 polypeptide comprises a Hse residue located in any one of residues 94-114. In some embodiments, the synthetic IL-2 polypeptide comprises 1, 2, 3, or more Hse residues. In some embodiments, the synthetic IL-2 polypeptide comprises Hse41, Hse71, Hse104, or a combination thereof. In some embodiments, the synthetic IL-2 polypeptide comprises Hse41, Hse71, and Hse104. In some embodiments, the synthetic IL-2 polypeptide comprises at least two amino acid substitutions, wherein the at least two amino acid substitutions are selected from (a) a homoserine (Hse) residue located in any one of residues 35-45; (b) a homoserine residue located in any one of residues 61-81; and (c) a homoserine residue located in any one of residues 94-114. In some embodiments, the synthetic IL-2 polypeptide comprises Hse41 and Hse71. In some embodiments, the synthetic IL-2 polypeptide comprises Hse41 and Hse104. In some embodiments, the synthetic IL-2 polypeptide comprises Hse71 and Hse104. In some embodiments, the synthetic IL-2 polypeptide comprises Hse41. In some embodiments, the synthetic IL-2 polypeptide comprises Hse71. In some embodiments, the synthetic IL-2 polypeptide comprises Hse104. In some embodiments, the synthetic IL-2 polypeptide comprises 1, 2, 3, or more norleucine (Nle) residues. In some embodiments, the synthetic IL-2 polypeptide comprises a Nle residue located in any one of residues 18-28. In some embodiments, the synthetic IL-2 polypeptide comprises one or more Nle residues located in any one of residues 34-50. In some embodiments, the synthetic IL-2 polypeptide comprises a Nle residue located in any one of residues 20-60. In some embodiments, the synthetic IL-2 polypeptide comprises three Nle substitutions. In some embodiments, the synthetic IL-2 polypeptide comprises Nle23, Nle39, and Nle46.

In some embodiments, the IL-2 polypeptide comprising a cleavable moiety of the activatable immunocytokine comprises an amino acid sequence having at least about 80%, at least about 85%, at least about 90%, at least about 95%, at least about 96%, at least about 97%, at least about 98%, or at least about 99% sequence identity to the sequence set forth in SEQ ID NO: 1 (wild type IL-2). In some embodiments, the IL-2 polypeptide comprises an amino acid sequence having at least about 80% sequence identity to the sequence set forth in SEQ ID NO: 1. In some embodiments, the IL-2 polypeptide comprises an amino acid sequence having at least about 85% sequence identity to the sequence set forth in SEQ ID NO: 1. In some embodiments, the IL-2 polypeptide comprises an amino acid sequence having at least about 90% sequence identity to the sequence set forth in SEQ ID NO: 1. In some embodiments, the IL-2 polypeptide comprises an amino acid sequence having at least about 95% sequence identity to the sequence set forth in SEQ ID NO: 1.

In some embodiments, the IL-2 polypeptide comprising a cleavable moiety of the activatable immunocytokine comprises an amino acid sequence having at least about 80%, at least about 85%, at least about 90%, at least about 95%, at least about 96%, at least about 97%, at least about 98%, at least about 99%, or 100% sequence identity to the sequence set forth in SEQ ID NO: 2. In some embodiments, the IL-2 polypeptide comprises an amino acid sequence having at least about 80% sequence identity to the sequence set forth in SEQ ID NO: 2. In some embodiments, the IL-2 polypeptide comprises an amino acid sequence having at least about 85% sequence identity to the sequence set forth in SEQ ID NO: 2. In some embodiments, the IL-2 polypeptide comprises an amino acid sequence having at least about 90% sequence identity to the sequence set forth in SEQ ID NO: 2. In some embodiments, the IL-2 polypeptide comprises an amino acid sequence having at least about 95% sequence identity to the sequence set forth in SEQ ID NO: 2. In some embodiments, the IL-2 polypeptide comprises an amino acid sequence having at least about 96% sequence identity to the sequence set forth in SEQ ID NO: 2. In some embodiments, the IL-2 polypeptide comprises an amino acid sequence having at least about 97% sequence identity to the sequence set forth in SEQ ID NO: 2. In some embodiments, the IL-2 polypeptide comprises an amino acid sequence having at least about 98% sequence identity to the sequence set forth in SEQ ID NO: 2. In some embodiments, the IL-2 polypeptide comprises an amino acid sequence having at least about 99% sequence identity to the sequence set forth in SEQ ID NO: 2. In some embodiments, the IL-2 polypeptide comprises the sequence of SEQ ID NO: 2. In some embodiments, the IL-2 polypeptide comprising the above indicated sequence identity to SEQ ID NO: 2 comprises one or more polymers attached to the IL-2 polypeptide as provided herein (e.g., at residues F42Y and Y45), and/or a linker attached as provided herein (e.g., at reside F42Y).

In some embodiments, the IL-2 comprising a cleavable moiety of the activatable immunocytokine comprises amino acid sequence having at least about 80%, at least about 85%, at least about 90%, at least about 95%, at least about 96%, at least about 97%, at least about 98%, at least about 99%, or 100% sequence identity to the sequence set forth in SEQ ID NO: 3. In some embodiments, the IL-2 polypeptide comprises an amino acid sequence having at least about 80% sequence identity to the sequence set forth in SEQ ID NO: 3. In some embodiments, the IL-2 polypeptide comprises an amino acid sequence having at least about 85% sequence identity to the sequence set forth in SEQ ID NO: 3. In some embodiments, the IL-2 polypeptide comprises an amino acid sequence having at least about 90% sequence identity to the sequence set forth in SEQ ID NO: 3. In some embodiments, the IL-2 polypeptide comprises an amino acid sequence having at least about 95% sequence identity to the sequence set forth in SEQ ID NO: 3. In some embodiments, the IL-2 polypeptide comprises an amino acid sequence having at least about 96% sequence identity to the sequence set forth in SEQ ID NO: 3. In some embodiments, the IL-2 polypeptide o comprises an amino acid sequence having at least about 97% sequence identity to the sequence set forth in SEQ ID NO: 3. In some embodiments, the IL-2 polypeptide comprises an amino acid sequence having at least about 98% sequence identity to the sequence set forth in SEQ ID NO: 3. In some embodiments, the IL-2 polypeptide comprises an amino acid sequence having at least about 99% sequence identity to the sequence set forth in SEQ ID NO: 3. In some embodiments, the IL-2 polypeptide comprises the sequence of SEQ ID NO: 3.

### Activity of Activatable IL-2 Polypeptides and “Activated” IL-2 Polypeptides of Activatable Immunocytokines

In some instances, at least one activity of the IL-2 polypeptides of the activatable immunocytokines provided herein is altered upon cleavage of the cleavable moiety. In some embodiments, an ability of the IL-2 polypeptide to bind to at least one IL-2 receptor subunit (or a complex thereof) (e.g., the beta or gamma subunits) is enhanced upon cleavage of the cleavable moiety. In some embodiments, an ability of the IL-2 polypeptide to bind to a different IL-2 receptor subunit (e.g., the alpha subunit) is substantially unaffected (e.g., when both the activatable IL-2 polypeptide and the IL-2 polypeptide after cleavage of the cleavable moiety show insubstantial binding to the IL-2 receptor alpha subunit). The activities and altered activities for IL-2 polypeptides of activatable immunocytokines described herein can refer to the activity of the IL-2 polypeptide itself (e.g., without being attached to the immune checkpoint inhibitor molecule, such as the anti-PD-1 antibody or antigen binding fragment thereof) or can refer to the activity of the IL-2 polypeptide as part of the activatable immunocytokine (e.g., the activities herein describe the activity of the IL-2 polypeptide when attached to the immune checkpoint inhibitor molecule, such as the anti-PD-1 antibody or antigen binding fragment thereof).

In some embodiments, the activatable IL-2 polypeptide of the activatable immunocytokine exhibits reduced binding to the IL-2 receptor alpha subunit compared to wild type IL-2. In some embodiments, the IL-2 polypeptide after cleavage of the cleavable moiety exhibits reduced binding to the IL-2 receptor alpha subunit compared to wild type IL-2. In some embodiments, the activatable IL-2 polypeptide and the IL-2 polypeptide after cleavage of the cleavable moiety exhibit reduced binding to the IL-2 receptor alpha subunit compared to wild type IL-2. In some embodiments, one or both of the activatable IL-2 polypeptide and/or the IL-2 polypeptide after cleavage of the cleavable moiety exhibit binding to the IL-2 receptor alpha subunit (or the IL-2 receptor αβγ complex) which is reduced by at least 5-fold, 10-fold, 20-fold, 50-fold, 100-fold, 200-fold, 500-fold, or 1000-fold compared to wild type IL-2. In some embodiments, one or both of the activatable IL-2 polypeptide and/or the IL-2 polypeptide after cleavage of the cleavable moiety exhibit binding to the IL-2 receptor alpha subunit (or the IL-2 receptor αβγ complex) with a K_{D} of at least 100 nM, at least 500 nM, at least 1 micromolar, at least 10 micromolar, or at least 100 micromolar. In some embodiments, the IL-2 polypeptide after cleavage of the cleavable moiety exhibit binding to the IL-2 receptor alpha subunit (or the IL-2 receptor αβγ complex) with a K_{D} of at least 1 micromolar. In some embodiments, the activatable IL-2 polypeptide exhibits binding to the IL-2 receptor alpha subunit (or the IL-2 receptor βγ complex) with a K_{D} of at least 1 micromolar. In some embodiments, one or both of the activatable IL-2 polypeptide and/or the IL-2 polypeptide after cleavage of the cleavable moiety exhibit substantially no binding to the IL-2 receptor alpha subunit (or the IL-2 receptor αβγ complex). In some embodiments, one or both of the activatable IL-2 polypeptide and the IL-2 polypeptide after cleavage of the cleavable moiety exhibit reduced ability to signal (e.g., activate the JAK-STAT pathway) through the IL-2 receptor alpha subunit or the IL-2 receptor αβγ complex (e.g., as measured by a reporter assay). In some embodiments, the ability of one or both of the IL-2 polypeptide and the IL-2 polypeptide after cleavage of the cleavable moiety to signal (e.g., activate the JAK-STAT pathway) through the IL-2 receptor alpha subunit or the IL-2 receptor αβγ complex (e.g., as measured by a reporter assay) is reduced by at least 5-fold, 10-fold, 50-fold, 100-fold, 500-fold, or 1000-fold (e.g., as determined by comparing EC50 values).

In some embodiments, the activatable IL-2 polypeptide of the activatable immunocytokine exhibits reduced binding to the IL-2 receptor beta subunit compared to the IL-2 polypeptide after cleavage of the cleavable moiety. In some embodiments, cleavage of the cleavable moiety enhances binding of the IL-2 polypeptide to the IL-2 receptor beta subunit by a factor of at least 2-fold, at least 3-fold, at least 4-fold, at least 5-fold, at least 6-fold, at least 7-fold, at least 8-fold, at least 9-fold, or at least 10-fold compared to the activatable IL-2 polypeptide. In some embodiments, cleavage of the cleavable moiety enhances binding of the IL-2 polypeptide by a factor of at least 2-fold compared to the activatable IL-2 polypeptide. In some embodiments, cleavage of the cleavable moiety enhances binding of the IL-2 polypeptide by a factor of at least 5-fold compared to the activatable IL-2 polypeptide. In some embodiments, cleavage of the cleavable moiety enhances binding of the IL-2 polypeptide by a factor of at least 10-fold compared to the activatable IL-2 polypeptide. In some embodiments, binding ability of the IL-2 polypeptide and the activatable IL-2 polypeptide with the IL-2 receptor beta subunit is determined by measuring and/or comparing the K_{D} values. In some embodiments, the IL-2 polypeptide after cleavage of the cleavable moiety exhibits a K_{D} value for binding to the IL-2 receptor beta subunit which is at most 10 nM, at most 20 nM, at most 50 nM, at most 100 nM, at most 200 nM, or at most 500 nM. In some embodiments, the IL-2 polypeptide after cleavage of the cleavable moiety exhibits a K_{D} value for binding to the IL-2 receptor beta subunit which is at most 50 nM. In some embodiments, the IL-2 polypeptide after cleavage of the cleavable moiety exhibits a K_{D} value for binding to the IL-2 receptor beta subunit which is at most 200 nM.

In some embodiments, the activatable IL-2 polypeptide of the activatable immunocytokine exhibits reduced binding to the IL-2 receptor βγ complex compared to the IL-2 polypeptide after cleavage of the cleavable moiety. In some embodiments, cleavage of the cleavable moiety enhances binding of the IL-2 polypeptide to the IL-2 receptor βγ complex by a factor of at least 2-fold, at least 3-fold, at least 4-fold, at least 5-fold, at least 6-fold, at least 7-fold, at least 8-fold, at least 9-fold, or at least 10-fold compared to the activatable IL-2 polypeptide. In some embodiments, cleavage of the cleavable moiety enhances binding of the IL-2 polypeptide by a factor of at least 2-fold compared to the activatable IL-2 polypeptide. In some embodiments, cleavage of the cleavable moiety enhances binding of the IL-2 polypeptide by a factor of at least 5-fold compared to the activatable IL-2 polypeptide. In some embodiments, cleavage of the cleavable moiety enhances binding of the IL-2 polypeptide by a factor of at least 10-fold compared to the activatable IL-2 polypeptide. In some embodiments, binding ability of the IL-2 polypeptide and the activatable IL-2 polypeptide with the IL-2 receptor βγ complex is determined by measuring and/or comparing the K_{D} values. In some embodiments, the IL-2 polypeptide after cleavage of the cleavable moiety exhibits a K_{D} value for binding to the IL-2 receptor βγ complex which is at most 10 nM, at most 20 nM, at most 50 nM, at most 100 nM, at most 200 nM, or at most 500 nM. In some embodiments, the IL-2 polypeptide after cleavage of the cleavable moiety exhibits a K_{D} value for binding to the IL-2 receptor βγ complex which is at most 20 nM. In some embodiments, the IL-2 polypeptide after cleavage of the cleavable moiety exhibits a K_{D} value for binding to the IL-2 receptor βγ complex which is at most 50 nM. In some embodiments, the IL-2 polypeptide after cleavage of the cleavable moiety exhibits a K_{D} value for binding to the IL-2 receptor βγ complex which is at most 200 nM.

In some embodiments, the activatable IL-2 polypeptide the activatable immunocytokine exhibits reduced ability to signal through the IL-2 receptor βγ complex (e.g., activate the JAK-STAT pathway) compared to the IL-2 polypeptide after cleavage of the cleavable moiety. In some embodiments, cleavage of the cleavable moiety enhances the ability of the IL-2 polypeptide to signal through the IL-2 receptor βγ complex by a factor of at least 2-fold, at least 3-fold, at least 4-fold, at least 5-fold, at least 6-fold, at least 7-fold, at least 8-fold, at least 9-fold, or at least 10-fold compared to the activatable IL-2 polypeptide. In some embodiments, cleavage of the cleavable moiety enhances the ability of the IL-2 polypeptide to signal through the IL-2 receptor βγ complex by a factor of at least 2-fold compared to the activatable IL-2 polypeptide. In some embodiments, cleavage of the cleavable moiety enhances the ability of the IL-2 polypeptide to signal through the IL-2 receptor βγ complex by a factor of at least 5-fold compared to the activatable IL-2 polypeptide. In some embodiments, cleavage of the cleavable moiety enhances the ability of the IL-2 polypeptide to signal through the IL-2 receptor βγ complex by a factor of at least 10-fold compared to the activatable IL-2 polypeptide. In some embodiments, the ability of the IL-2 polypeptide and the activatable IL-2 polypeptide to signal through the IL-2 receptor βγ complex is determined by measuring and/or comparing half-maximal effective concentrations (EC₅₀s).

In some embodiments, EC₅₀s are determined using a reporter assay (e.g., HEK-Blue™ IL-2 Cell assay from Invitrogen). In some embodiments, the IL-2 polypeptide of the activatable immunocytokine after cleavage of the cleavable moiety exhibits an EC₅₀ value for signaling through the IL-2 receptor f complex which is at most 0.01 nM, at most 0.02 nM, at most 0.05 nM, at most 0.1 nM, at most 0.2 nM, at most 0.5 nM, at most 1 nM, at most 2 nM, or at most 5 nM. In some embodiments, the IL-2 polypeptide after cleavage of the cleavable moiety exhibits an EC₅₀ value for signaling through the IL-2 receptor βγ complex which is at most 0.01 nM. In some embodiments, the IL-2 polypeptide after cleavage of the cleavable moiety exhibits an EC₅₀ value for signaling through the IL-2 receptor βγ complex which is at most 0.05 nM. In some embodiments, the IL-2 polypeptide after cleavage of the cleavable moiety exhibits an EC₅₀ value for signaling through the IL-2 receptor βγ complex which is at most 0.1 nM.

In some embodiments, EC₅₀s are determined using a STAT5 activation assay in T cells. In some embodiments, the T cells are T_{eff} cells. In some embodiments, the IL-2 polypeptide of the activatable immunocytokine after cleavage of the cleavable moiety exhibits an EC₅₀ value for STAT5 activation which is at most 1 nM, at most 2 nM, at most 5 nM, at most 10 nM, at most 20 nM, or at most 50 nM. In some embodiments, the IL-2 polypeptide after cleavage of the cleavable moiety exhibits an EC₅₀ value for STAT5 activation which is at most 1 nM. In some embodiments, the IL-2 polypeptide after cleavage of the cleavable moiety exhibits an EC₅₀ value for STAT5 activation which is at most 5 nM. In some embodiments, the IL-2 polypeptide after cleavage of the cleavable moiety exhibits an EC₅₀ value for STAT5 activation which is at most 10 nM.

### Exemplary Activatable IL-2 Polypeptides

The instant disclosure contains numerous examples of IL-2 polypeptides which can be part of activatable immunocytokines, including those shown in the table below. In some embodiments, the IL-2 polypeptide of the activatable immunocytokine is an activatable IL-2 polypeptides shown in Table 3 below (e.g., any one of SEQ ID NOs: 4-52, 54, or 55), or any analogous activatable IL-2 polypeptide (e.g., an IL-2 polypeptide having a cleavable peptide attached at the indicated point of attachments, by the indicated linking groups, etc.).

TABLE 3
IL-2 Polypeptides, Including Activatable IL-2 Polypeptides

| SEQ ID | Example | Substitute | |
|---|---|---|---|
| NO(S): | # | Ref | Sequence |
| 1 | | | APTSSSTKKTQLQLEHLLLDLQMILNGINNYKNPKLTRMLTFKFYMP |
| (WT-IL-2) | | | KKATELKHLQCLEEELKPLEEVLNLAQSKNFHLRPRDLISNINVIVLE |
| | | | LKGSETTFMCEYADETATIVEFLNRWITFCQSIISTLT |
| 2 | | | APTSSSTKKTQLQLEHLLLDLQ(Nle)ILNGINNYKNPKLTR(Nle)L(Hse) |
| | | | YKFY(Ne)PKKATELKHLQCLEEELKPLEEVL(Hse)LAQSKNFHLRPR |
| | | | DLISNINVIVLELKGSETTF(Hse)CEYADETATIVEFLNRWITFSQSIIST |
| | | | LT |
| 3 | Ref | CMP- | APTSSSTKKTQLQLEHLLLDLQ-Nle-ILNGINNYKNPKLTR-Nle-L-Hse-Yn3-K |
| | | 003 | F-Ygp-NlePKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| | | | Ac-PLGLAG NH |
| 4 | 3 | CMP- 118 | 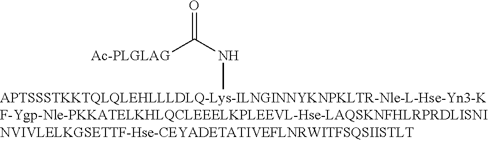 |
| 5 | 4 | CMP- 119 | 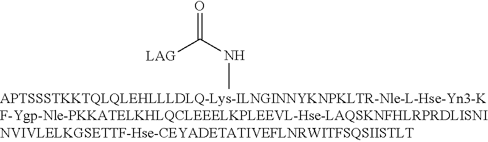 |
| 6 | 5 | CMP- 120 | 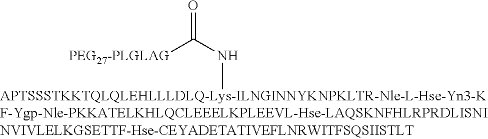 |
| 7 | 6 | CMP- 121 | 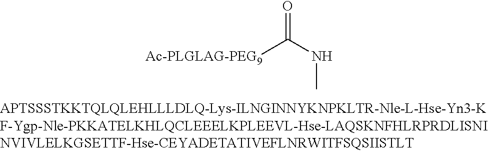 |
| 8 | 7 | CMP- 122 | 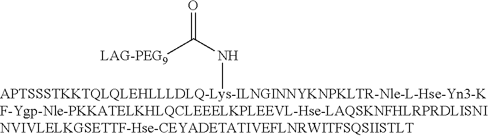 |
| 9 | 8 | CMP- 123 | 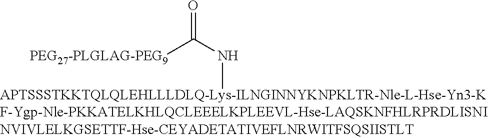 |
| 10 | 9 | CMP- 124 | 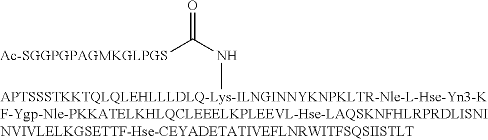 |
| 11 | 10 | CMP- 125 | 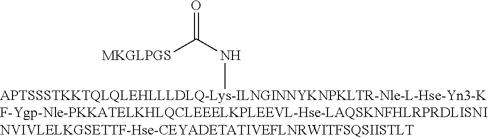 |
| 12 | 11 | CMP- 126 | 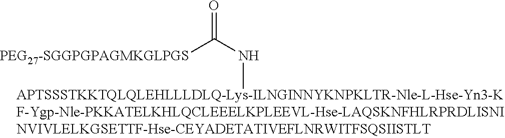 |
| 13 | 12 | CMP- 127 | 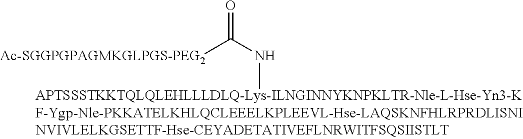 |
| 14 | 13 | CMP- 128 | 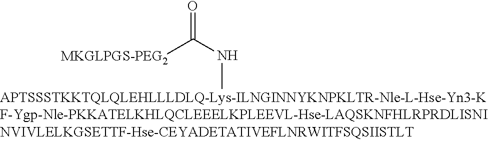 |
| 15 | 14 | CMP- 129 | 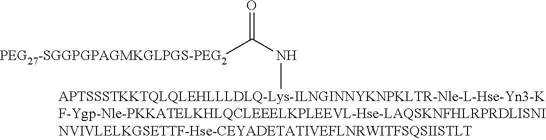 |
| 16 | 15 | CMP- 141 | 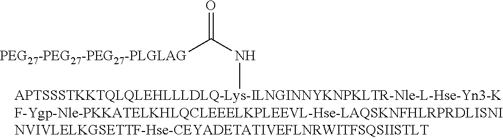 |
| 17 | 16 | CMP- 142 | 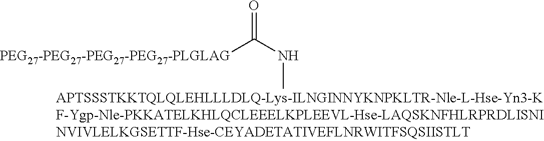 |
| 18 | 17 | CMP- 143 | 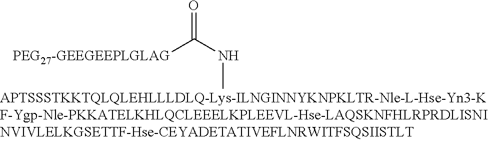 |
| 19 | 18 | CMP- 144 | 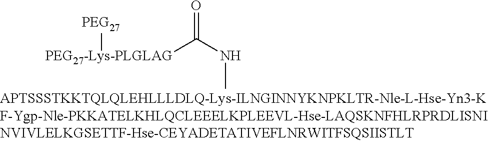 |
| 20 | 19 | CMP- 145 | 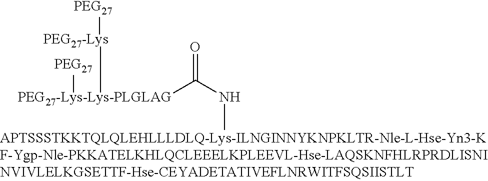 |
| 21 | 20 | CMP- 146 | 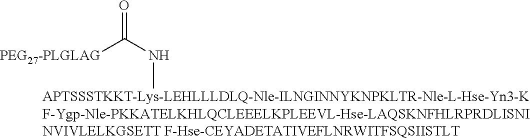 |
| 22 | 21 | CMP- 147 | 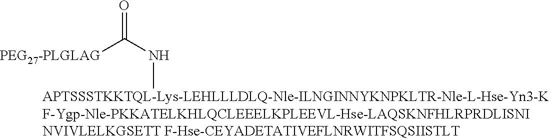 |
| 23 | 22 | CMP- 148 | 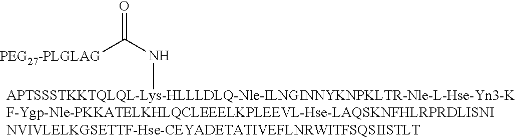 |
| 24 | 23 | CMP- 149 | 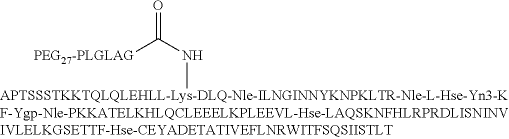 |
| 25 | 24 | CMP- 150 | 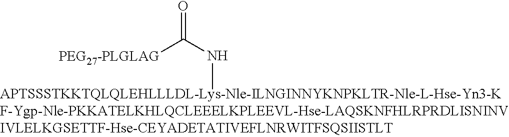 |
| 26 | 25 | CMP- 162 | 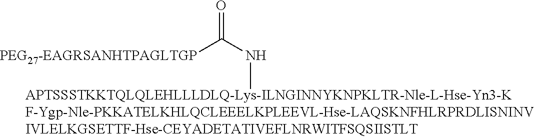 |
| 27 | 26 | CMP- 133 | 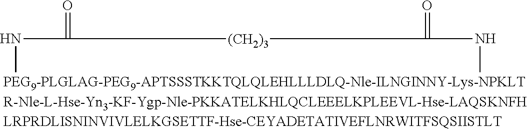 |
| 28 | 27 | CMP- 134 | 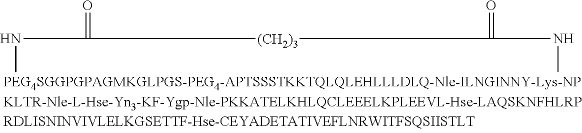 |
| 29 | 28 | CMP- 135 | 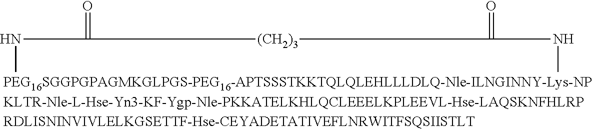 |
| 30 | 29 | CMP- 140 | 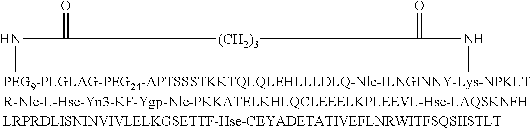 |
| 31 | 30 | CMP- 152 | 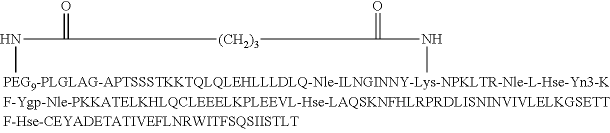 |
| 32 | 31 | CMP- 153 | 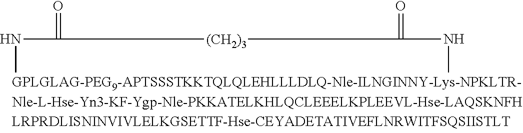 |
| 33 | 32 | CMP- 136 | 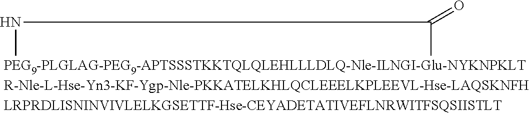 |
| 34 | 33 | CMP- 137 | 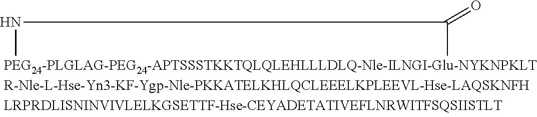 |
| 35 | 34 | CMP- 138 | 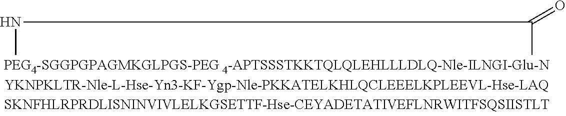 |
| 36 | 35 | CMP- 139 | 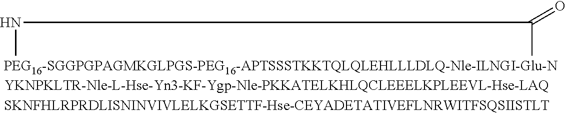 |
| 37 | 36 | CMP- 154 | 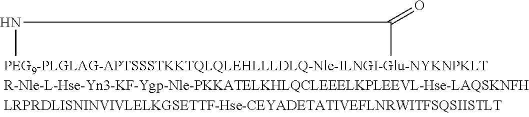 |
| 38 | 37 | CMP- 155 | 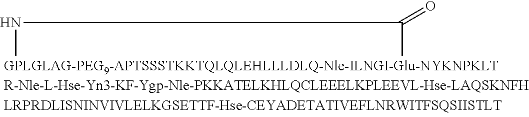 |
| 39 | 38 | CMP- 156 | 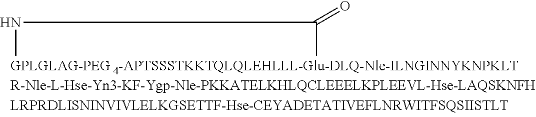 |
| 40 | 39 | CMP- 157 | 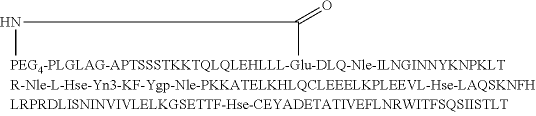 |
| 41 | 40 | CMP- 158 | 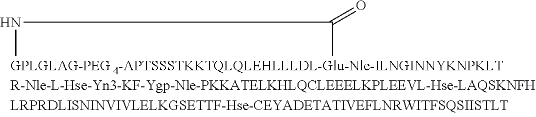 |
| 42 | 41 | CMP- 159 | 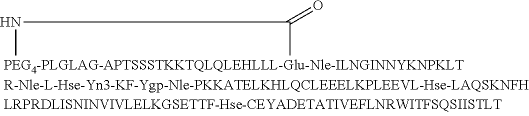 |
| 43 | 42 | CMP- 160 | 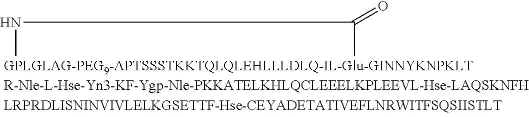 |
| 44 | 43 | CMP- 161 | 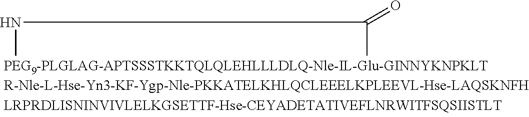 |
| 45 | 44 | CMP- 162 | 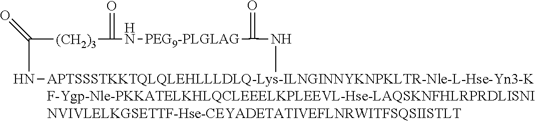 |
| 46 | 45 | CMP- 163 | 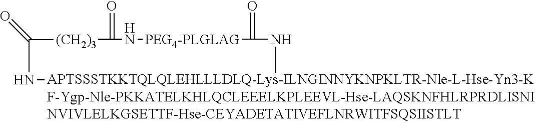 |
| 47 | 46 | CMP- 164 | 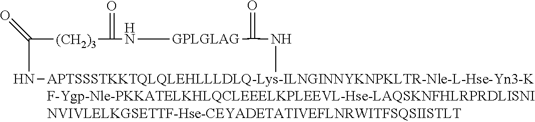 |
| 48 | 47 | CMP- 165 | 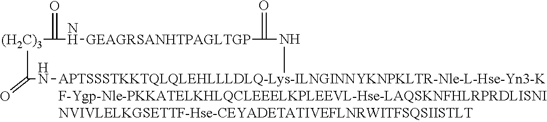 |
| 49 | 48 | CMP- 166 | 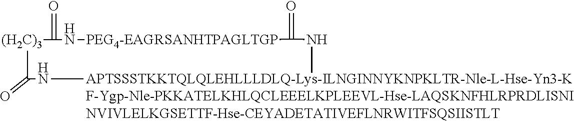 |
| 50 | 49 | CMP- 167 | 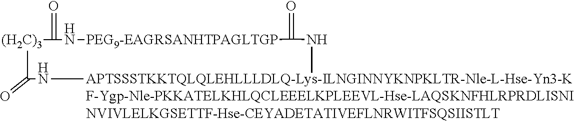 |
| 51 | 50 | CMP- 168 | 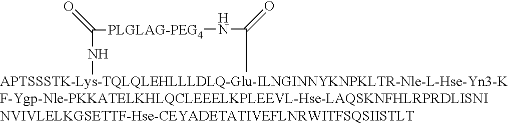 |
| 52 | 51 | CMP- 169 | 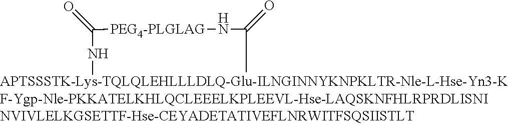 |
| 53 | 52 | CMP- | APTSSSTKKTQLQLE-Dab-LLLDLQ-Nle-ILNGINNYKNPKLTR-Nle-L-Hse-Yn3-KF-Ygp-Nle- |
| | | 130 | PKKATELKHLQCLEEELKPLEEVL-Hse-LAQSKNFHLRPRDLISNINVIVLELKGSETT |
| | | | F-Hse-CEYADETATIVEFLNRWITFSQSISTLT |
| 54 | 53 | CMP- 131 | 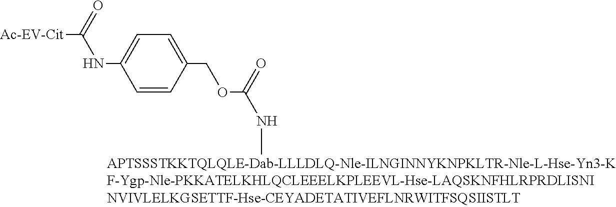 |
| 55 | 54A | CMP- 132 | 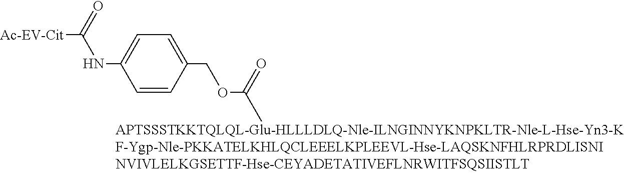 |
| 56 | 54I | CMP- 300 | 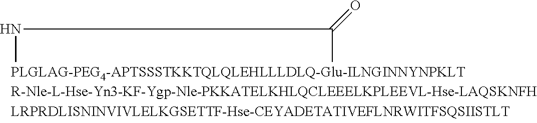 |
| 57 | | CMP- 301 | 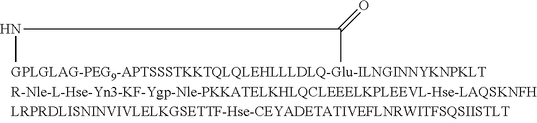 |
| 58 | | CMP- 302 | 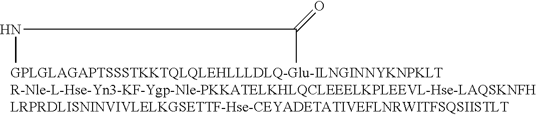 |
| 59 | | CMP- 303 | 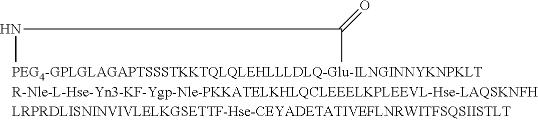 |
| 60 | | CMP- 304 | 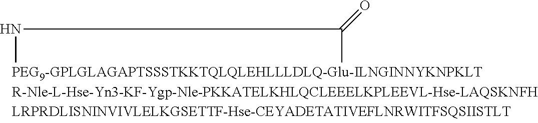 |
| 61 | | CMP- 305 | 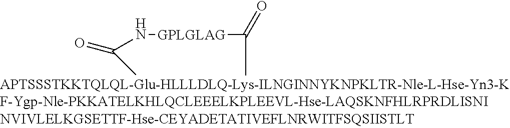 |
| 62 | 54D | CMP- 310 | 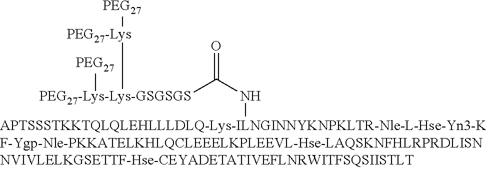 |
| 63 | 54E | CMP- 311 | 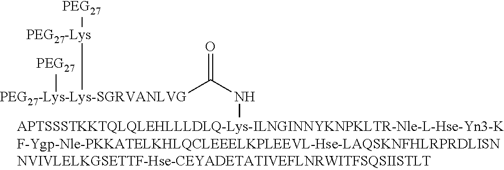 |
| 64 | | CMP- 312 | 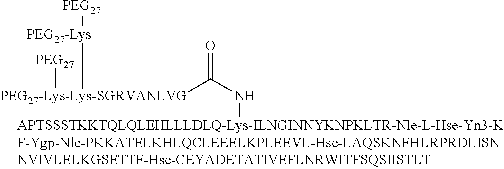 |
| 65 | 54B | CMP- 313 | 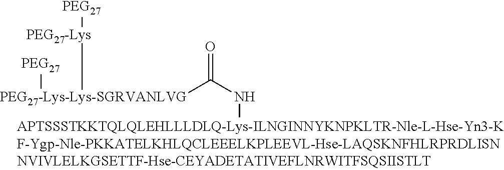 |
| 66 | 54C | CMP- 314 | 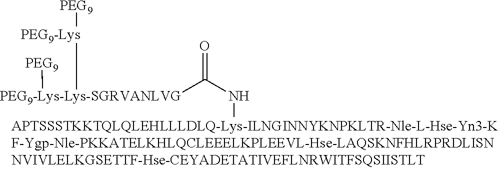 |
| 67 | | CMP- 315 | 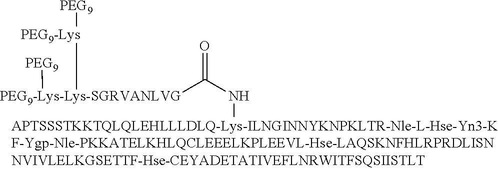 |
| 68 | | CMP- 316 | 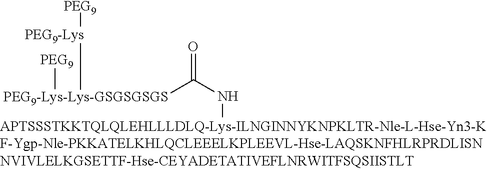 |
| 69 | | CMP- 317 | 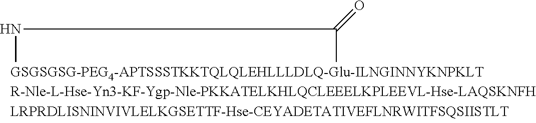 |
| 70 | 54J | CMP- 318 | 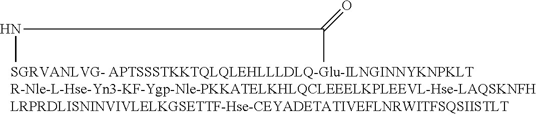 |
| 71 | 54K | CMP- 319 | 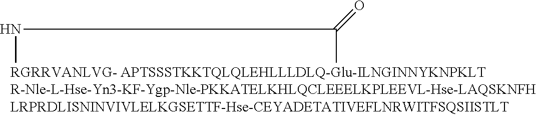 |
| 72 | 54L | CMP- 320 | 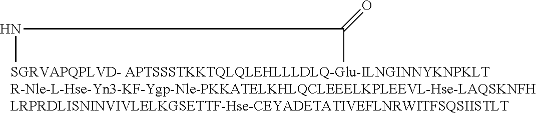 |
| 73 | 54M | CMP- 321 | 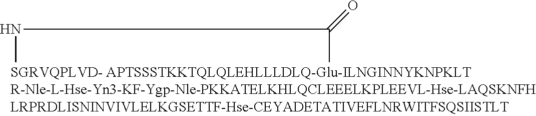 |
| 74 | | CMP- 322 | 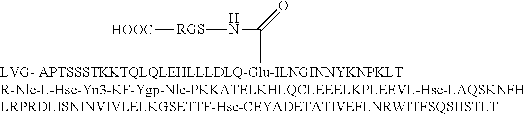 |
| 75 | | CMP- 323 | 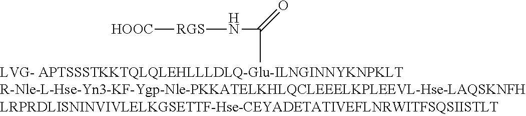 |
| 76 | 54N | CMP- 324 | 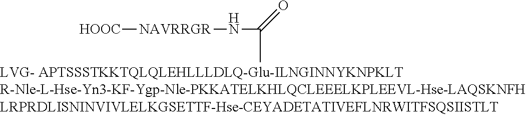 |
| 77 | 54O | CMP- 325 | 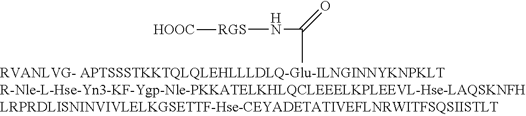 |
| 78 | 54G | CMP- 326 | 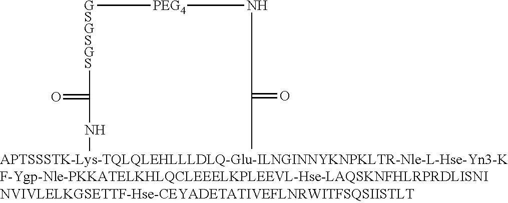 |
| 79 | 54H | CMP- 327 | 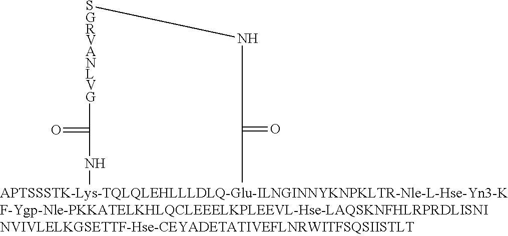 |
| 80 | | CMP- 328 | 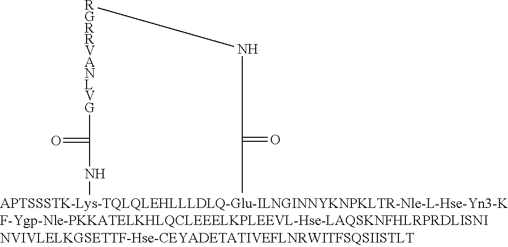 |
| 81 | | CMP- 329 | 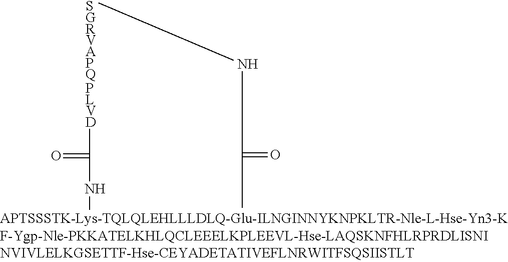 |
| 82 | | CMP- | [symbol] |
| | | 330 | |
| 83 | 54F | CMP-331 | |

[symbol]

indicates data missing or illegible when filed

In Table 3 above, Nle is a norleucine residue, H-se is a homoserine residue, Dab is 2,4-diamino butyric acid, Cit is a citrulline residue, Yn3 is a tyrosine residue modified with an azide-capped PEG9 group (see below), and Ygp is a tyrosine residue modified with an amino-capped PEG8 group (see below).

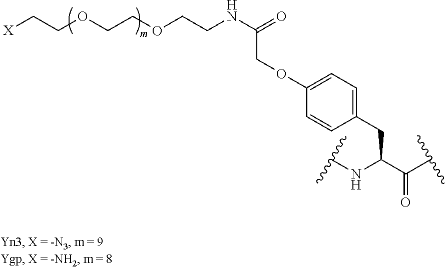

### Points of Attachment of Linkers to IL-2 Polypeptides

The IL-2 polypeptides (e.g., an activatable IL-2 polypeptide) provided herein are connected to the immune checkpoint inhibitor molecule (e.g., an anti-PD-1 antibody or antigen binding fragment thereof) through a linker. As discussed below, the linker can be attached to the immune checkpoint inhibitor molecule (e.g., an antibody or antigen binding fragment thereof, such as an anti-PD-1 antibody or antigen binding fragment thereof) at any of the positions provide herein, and may be of any suitable structure, including those provided herein below. The linker is also attached to an IL-2 polypeptide at a point of attachment as provided herein.

In some embodiments, the linker is attached to the IL-2 polypeptide at an amino acid residue. In some embodiments, the linker is attached at an amino acid residue corresponding to any one of amino acid residues 1-133 of SEQ ID NO: 1. In some embodiments, the linker is attached at a non-terminal amino acid residue (e.g., any one of amino acid residues 2-132 of SEQ ID NO: 1, or any one of amino acid residues 1-133 of SEQ ID NO: 1, wherein either the N-terminus or C-terminus has been extended by one or more amino acid residues). In some embodiments, the linker is attached at a non-terminal amino acid residue of the IL-2 polypeptide, wherein the IL-2 polypeptide comprises either an N-terminal truncation or a C-terminal truncation relative to SEQ ID NO: 1.

In some embodiments, the linker is attached to the IL-2 polypeptide at an amino acid residue which interacts with an IL-2 receptor (IL-2R) protein or subunit. In some embodiments, the linker is attached at an amino acid residue which interacts with the IL-2R alpha subunit (IL-2Ra), the IL-2R beta subunit (IL-2Rβ), or the IL-2R gamma subunit (IL-2Rγ). In some embodiments, the linker is attached at an amino acid residue which interacts with the IL-2R alpha subunit (IL-2Ra). In some embodiments, the linker is attached at an amino acid residue which interacts with the IL-2R beta subunit (IL-2Rβ). In some embodiments, the linker is attached at an amino acid residue which interacts with the IL-2R gamma subunit (IL-2Rγ).

In some embodiments, the point of attachment to the IL-2 polypeptide is selected such that the interaction of the IL-2 polypeptide with at least one IL-2 receptor subunit is decreased or blocked. In some embodiments, the point of attachment is selected such that interaction of the IL-2 polypeptide with the IL-2Rα is reduced or blocked. In some embodiments, the point of attachment is selected such that interaction of the IL-2 polypeptide with the IL-2Rβ is retained.

In some embodiments, the linker is attached to the IL-2 polypeptide at a residue which disrupts binding of the IL-2 polypeptide with the IL-2 receptor alpha subunit (IL-2Ra). Examples of these residues include residues 3, 5, 34, 35, 36, 37, 38, 40, 41, 42, 43, 44, 45, 60, 61, 62, 63, 64, 65, 67, 68, 69, 71, 72,103,104, 105, and 107, as described in, for example, PCT Pub. Nos. WO2019/028419, WO2020/056066, WO2021/140416, and WO2021/216478 each of which is hereby incorporated by reference as if set forth in its entirety.

In some embodiments, the linker is attached to the IL-2 polypeptide at an amino acid residue at any one of positions 1-110, wherein residue position numbering of the modified IL-2 polypeptide is based on SEQ ID NO: 1 as a reference sequence. In some embodiments, the linker is attached to the IL-2 polypeptide at an amino acid residue at any one of positions 1-10, 1-20, 1-30, 30-50, 30-70, 30-100, 40-50, 40-70, 40-100, or 40-110. In some embodiments, the linker is attached to the IL-2 polypeptide at an amino acid residue at any one of positions 1, 35, 37, 38, 41, 42, 43, 44, 45, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the linker is attached to the IL-2 polypeptide at an amino acid residue at any one of positions 1, 35, 37, 38, 41, 42, 43, 44, 45, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the linker is attached to the IL-2 polypeptide at an amino acid residue at any one of positions 1, 35, 37, 38, 41, 42, 43, 44, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the linker is attached to the IL-2 polypeptide at an amino acid residue at any one of positions 1, 35, 37, 38, 41, 43, 44, 45, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the linker is attached to the IL-2 polypeptide at an amino acid residue at any one of positions 1, 35, 37, 38, 39, 40, 41, 42, 43, 44, 45, or 46. In some embodiments, the linker is attached to the IL-2 polypeptide at an amino acid residue at any one of positions 1, 41, 42, 43, 44, and 45, wherein amino acid residue position numbering of the modified IL-2 polypeptide is based on SEQ ID NO. 1 as a reference sequence. In some embodiments, the linker is attached at amino acid residue 1, 42 or 45. In some embodiments, the linker is attached at amino acid residue 1. In some embodiments, the linker is attached at amino acid residue 42. In some embodiments, the linker is attached at amino acid residue 45. In some embodiments, the linker is attached at amino acid residue 35. In some embodiments, the linker is attached at amino acid residue 37. In some embodiments, the linker is attached at amino acid residue 38. In some embodiments, the linker is attached at amino acid residue 41. In some embodiments, the linker is attached at amino acid residue 43. In some embodiments, the linker is attached at amino acid residue 44. In some embodiments, the linker is attached at amino acid residue 60. In some embodiments, the linker is attached at amino acid residue 61. In some embodiments, the linker is attached at amino acid residue 62. In some embodiments, the linker is attached at amino acid residue 64. In some embodiments, the linker is attached at amino acid residue 65. In some embodiments, the linker is attached at amino acid residue 68. In some embodiments, the linker is attached at amino acid residue 69. In some embodiments, the linker is attached at amino acid residue 71. In some embodiments, the linker is attached at amino acid residue 72. In some embodiments, the linker is attached at amino acid residue 104. In some embodiments, the linker is attached at amino acid residue 105. In some embodiments, the linker is attached at amino acid residue 107.

In some embodiments, the linker is attached to a residue which is a natural amino acid residue of an IL-2 polypeptide as set forth in SEQ ID NO: 1. In some embodiments, the linker is attached to an amino acid residue which is a modified version of the natural amino acid residue of an IL-2 polypeptide as set forth in SEQ ID NO: 1. Non-limiting examples of such modification include incorporation or attachment of a conjugation handle to the natural amino acid residue (including through a linker), or attachment of the linker to the natural amino acid using any compatible method. In some embodiments, the linker is attached to an amino acid residue which is a substituted amino acid residue compared to the IL-2 polypeptide of SEQ ID NO: 1. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired reactive group or conjugation handle, such as a CLICK chemistry reagent such as an azide, alkyne, etc.).

In some embodiments, the linker is attached at an unnatural amino acid residue. In some embodiments, the unnatural amino acid residue comprises a conjugation handle. In some embodiments, the conjugation handle facilitates the addition of the linker to the IL-2 polypeptide. The conjugation handle can be any of the conjugation handles provided herein. In some embodiments, the linker is covalently attached site-specifically to the unnatural amino acid. Non-limiting examples of amino acid residues comprising conjugation handles can be found, for example, in PCT Pub. Nos. WO2015054658A1, WO2014036492A1, and WO2021133839A1 WO2006069246A2, and WO2007079130A2, each of which is incorporated by reference as if set forth in its entirety.

In some embodiments, the linker is attached to an amino acid residue which has been substituted with a natural amino acid. In some embodiments, the linker is attached to an amino acid residue which has been substituted with a cysteine, lysine, or tyrosine residue. In some embodiments, the linker is attached to a residue which has been substituted with a cysteine residue. In some embodiments, the linker is attached to an amino acid residue which has been substituted with a lysine residue. In some embodiments, the linker is attached to an amino acid residue which has been substituted with a tyrosine residue.

In some embodiments, the linker is attached to the amino acid residue at the N-terminal A1, K35, F42Y, K43, F44Y, or Y45. In some embodiments, the linker is attached to the amino acid residue at the N-terminal, A1, F42Y or Y45. In some embodiments, the linker is attached to the amino terminal residue. In some embodiments, the linker is attached to amino acid residue A1. In some embodiments, the linker is attached to amino acid residue F42Y. In some embodiments, the linker is attached to amino acid residue Y45.

### Immune Checkpoint Inhibitor Molecules

The activatable immunocytokine compositions provided herein comprise an immune checkpoint inhibitor molecule. In some embodiments, the immune checkpoint inhibitor molecule specifically binds to at least one immune checkpoint molecule. In some embodiments, the immune checkpoint molecule is an inhibitory immune checkpoint molecule. In some embodiments, an inhibitory immune checkpoint molecule is an immune system regulator implicated in the deactivation or lowering of an immune response. In some embodiments, an inhibitory immune checkpoint molecule has an effect on an immune response when it binds to its complementary checkpoint molecule.

In some embodiments, the immune checkpoint molecule which is bound by the immune checkpoint inhibitor molecule of the activatable immunocytokine is adenosine A2A receptor (A2AR), adenosine A2B receptor (A2BR), B7-H3, B7-H4, B and T lymphocyte Attenuater (BTLA), Cytotoxic T-Lymphocyte-Associated protein 4 (CTLA-4), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform2 (NOX2), Programmed cell death protein 1 (PD-1), Programmed death ligand 1 (PD-L1), Programmed death ligand 2 (PD-L2), T-cell immunoreceptor with Ig and ITIM domains (TIGIT), T-cell immunoglobulin domain and Mucin domain 3 (TIM-3), V-domain Ig suppressor of T cell activation (VISTA), Sialic acid-binding immunoglobulin-type lectin 7 (SIGLEC7), Sialic acid-binding immunoglobulin-type lectin 9 (SIGLEC9), or any combination thereof. In some embodiments, the immune checkpoint molecule which is bound the immune checkpoint inhibitor molecule is PD-1, PD-L1, PD-L2, or any combination thereof. In some embodiments, the immune checkpoint molecule which is bound the immune checkpoint inhibitor molecule is PD-1, PD-L1, or both. In some embodiments, the immune checkpoint molecule which is bound the immune checkpoint inhibitor molecule is PD-1.

In some embodiments, the immune checkpoint inhibitor molecule which is part of an activatable immunocytokine composition is a polypeptide which binds specifically to the immune checkpoint molecule (e.g., PD-1). In some embodiments, the polypeptide is an antibody or antigen binding fragment thereof.

The activatable immunocytokines provided herein utilize linkers to attach the immune checkpoint inhibitor molecules (e.g., anti-PD-1 antibodies or antigen binding fragments thereof) to the IL-2 polypeptides and derivatives thereof. In some embodiments, the linkers are attached to both the immune checkpoint inhibitor molecule and the IL-2 polypeptide at specific residues or a specific subset of residues. In some embodiments, the linkers are attached to each moiety in a site-selective manner, such that a population of the activatable immunocytokine is substantially uniform. This can be accomplished in a variety of ways as provided herein, including by site-selectively adding reagents for a conjugation reaction to a group to be conjugated, synthesizing, or otherwise preparing a moiety to be conjugated with a desired reagent for a conjugation reaction, or a combination of these two approaches. Using these approaches, the sites of attachment (such as specific amino acid residues) of the linker to each moiety can be selected with precision. Additionally, these approaches allow a variety of linkers to be employed for the composition which are not limited to amino acid residues as is required for fusion proteins. This combination of linker choice and precision attachment to the moieties allows the linker to also, in some embodiments, perform the function of modulating the activity of one of the groups (e.g., the IL-2 polypeptide), for example if the linker is attached to the IL-2 polypeptide at a position that interacts with a receptor of the IL-2 polypeptide.

In some embodiments, an immune checkpoint inhibitor molecule of the disclosure (e.g., an anti-PD-1 antibody or antigen binding fragment thereof) specifically binds to the immune checkpoint molecule (e.g., PD-1). An immune checkpoint inhibitor molecule selectively binds or preferentially binds to a target if it binds with greater affinity, avidity, more readily, and/or with greater duration than it binds to other substances. As such, “specific binding” or “preferential binding” does not necessarily require (although it can include) exclusive binding. Generally, but not necessarily, reference to specific binding means preferential binding where the affinity of the antibody, or antigen binding fragment thereof, is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for unrelated amino acid sequences. Binding of the immune checkpoint inhibitor molecule to the immune checkpoint molecule can block interaction of the immune checkpoint molecule with its ligand. For example, an anti-PD-1 antibody or an anti-PD-1 antigen binding fragment of the disclosure can block interaction of PD-1 with its ligand (e.g., PD-L1).

As used herein, the term “antibody” refers to an immunoglobulin (Ig), polypeptide, or a protein having a binding domain which is, or is homologous to, an antigen binding domain. The term further includes “antigen binding fragments” and other interchangeable terms for similar binding fragments as described below. Native antibodies and native immunoglobulins (Igs) are generally heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“Vii”) followed by a number of constant domains (“C_{H}”). Each light chain has a variable domain at one end (“W”) and a constant domain (“C.”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

In some instances, an antibody or an antigen binding fragment comprises an isolated antibody or antigen binding fragment, a purified antibody or antigen binding fragment, a recombinant antibody or antigen binding fragment, a modified antibody or antigen binding fragment, or a synthetic antibody or antigen binding fragment.

Antibodies and antigen binding fragments herein can be partly or wholly synthetically produced. An antibody or antigen binding fragment can be a polypeptide or protein having a binding domain which can be, or can be homologous to, an antigen binding domain. In one instance, an antibody or an antigen binding fragment can be produced in an appropriate in vivo animal model and then isolated and/or purified.

Depending on the amino acid sequence of the constant domain of its heavy chains, immunoglobulins (Igs) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. An Ig or portion thereof can, in some cases, be a human Ig. In some instances, a C3 domain can be from an immunoglobulin. In some cases, a chain or a part of an antibody or antigen binding fragment, a modified antibody or antigen binding fragment, or a binding agent can be from an Ig. In such cases, an Ig can be IgG, an IgA, an IgD, an IgE, or an IgM, or is derived therefrom. In cases where the Ig is an IgG, it can be a subtype of IgG, wherein subtypes of IgG can include IgG1, an IgG2a, an IgG2b, an IgG3, or an IgG4. In some cases, a C_{H}3 domain can be from an immunoglobulin selected from the group consisting of an IgG, an IgA, an IgD, an IgE, and an IgM, or derived therefrom. In some embodiments, an antibody or antigen binding fragment described herein comprises an IgG or is derived therefrom. In some instances, an antibody or antigen binding fragment comprises an IgG1 or is derived therefrom. In some instances, an antibody or antigen binding fragment comprises an IgG4 or is derived therefrom. In some embodiments, an antibody or antigen binding fragment described herein comprises an IgM, is derived therefrom, or is a monomeric form of IgM. In some embodiments, an antibody or antigen binding fragment described herein comprises an IgE or is derived therefrom. In some embodiments, an antibody or antigen binding fragment described herein comprises an IgD or is derived therefrom. In some embodiments, an antibody or antigen binding fragment described herein comprises an IgA or is derived therefrom.

The “light chains” of antibodies (immunoglobulins) from any vertebrate species can be assigned to one of two clearly distinct types, called kappa (“W” or “K”) or lambda (“X”), based on the amino acid sequences of their constant domains.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (e.g., Kabat et al., Sequences of Proteins of Immunological Interest, (5th Ed., 1991, National Institutes of Health, Bethesda Md. (1991), pages 647-669; hereafter “Kabat”); and (2) an approach based on crystallographic studies of antigen-antibody complexes (Al-lazikani et al. (1997) J. Molec. Biol. 273:927-948)). As used herein, a CDR may refer to CDRs defined by either approach or by a combination of both approaches.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3, and FR4), largely adopting a β-sheet configuration interspersed with three CDRs which form loops connecting and, in some cases, part of the β-sheet structure. The CDRs in each chain are held together in close proximity by the FRs and, with the CDRs from the other chain, contribute to the formation of the antigen binding site of antibodies (see, Kabat).

The terms “hypervariable region” and “CDR” when used herein, refer to the amino acid residues of an antibody which are responsible for antigen binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2), and 89-97 (CDRL3), and in the heavy chain variable domain the CDRs typically correspond to approximately residues 31-35 (CDRH1), 50-65 (CDRH2), and 95-102 (CDRH3) according to Kabat. It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2), and 91-96 (CDRL3), and in the heavy chain variable domain, the CDRs typically correspond to approximately residues 26-32 (CDRH1), 53-55 (CDRH2), and 96-101 (CDRH3) according to Chothia and Lesk (J. Mol. Biol., 196: 901-917 (1987)).

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat. As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, Id. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

In the present disclosure, the following abbreviations (in the parentheses) are used in accordance with the customs, as necessary: heavy chain (H chain), light chain (L chain), heavy chain variable region (VH), light chain variable region (VL), complementarity determining region (CDR), first complementarity determining region (CDR1), second complementarity determining region (CDR2), third complementarity determining region (CDR3), heavy chain first complementarity determining region (VH CDR1), heavy chain second complementarity determining region (VH CDR2), heavy chain third complementarity determining region (VH CDR3), light chain first complementarity determining region (VL CDR1), light chain second complementarity determining region (VL CDR2), and light chain third complementarity determining region (VL CDR3).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is generally defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, C_{H}2 and C_{H}3.

“Antibodies” useful in the present disclosure encompass, but are not limited to, monoclonal antibodies, polyclonal antibodies, chimeric antibodies, bispecific antibodies, multispecific antibodies, heteroconjugate antibodies, humanized antibodies, human antibodies, grafted antibodies, deimmunized antibodies, mutants thereof, fusions thereof, immunoconjugates thereof, antigen binding fragments thereof, and/or any other modified configuration of the immunoglobulin molecule that comprises an antigen recognition site of the required specificity, including glycosylation variants of antibodies, amino acid sequence variants of antibodies, and covalently modified antibodies. In certain embodiments of the methods and conjugates provided herein, the antibody requires an Fc region to enable attachment of a linker between the antibody and the protein (e.g., attachment of the linker using an affinity peptide, such as in AJICAP™ technology).

In some instances, an antibody is a monoclonal antibody. As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies and is not to be construed as requiring production of the antibody by any particular method.

In some instances, an antibody is a humanized antibody. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody.

If needed, an antibody or an antigen binding fragment described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen binding fragments described herein can be carried out via the use of software and specific databases. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7, and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

An antibody can be a human antibody. As used herein, a “human antibody” means an antibody having an amino acid sequence corresponding to that of an antibody produced by a human and/or that has been made using any suitable technique for making human antibodies. This definition of a human antibody includes antibodies comprising at least one human heavy chain polypeptide or at least one human light chain polypeptide. One such example is an antibody comprising murine light chain and human heavy chain polypeptides. In one embodiment, the human antibody is selected from a phage library, where that phage library expresses human antibodies. Human antibodies can also be made by introducing human immunoglobulin loci into transgenic animals, e.g., mice in which the endogenous immunoglobulin genes have been partially or completely inactivated. Alternatively, the human antibody may be prepared by immortalizing human B lymphocytes that produce an antibody directed against a target antigen (such B lymphocytes may be recovered from an individual or may have been immunized in vitro).

Any of the antibodies herein can be bispecific. Bispecific antibodies are antibodies that have binding specificities for at least two different antigens and can be prepared using the antibodies disclosed herein. Traditionally, the recombinant production of bispecific antibodies was based on the coexpression of two immunoglobulin heavy chain-light chain pairs, with the two heavy chains having different specificities. Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations.

According to one approach to making bispecific antibodies, antibody variable domains with the desired binding specificities (antibody-antigen combining sites) are fused to immunoglobulin constant domain sequences. The fusion can be with an immunoglobulin heavy chain constant domain, comprising at least part of the hinge, CH2 and CH3 regions. The first heavy chain constant region (CH1), containing the site necessary for light chain binding, can be present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some instances, an antibody herein is a chimeric antibody. “Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin, is inserted in place of the murine Fc. Chimeric or hybrid antibodies also may be prepared in vitro using suitable methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Provided herein are antibodies and antigen binding fragments thereof, modified antibodies and antigen binding fragments thereof, and binding agents that specifically bind to one or more epitopes on one or more target antigens. In one instance, a binding agent selectively binds to an epitope on a single antigen. In another instance, a binding agent is bivalent and either selectively binds to two distinct epitopes on a single antigen or binds to two distinct epitopes on two distinct antigens. In another instance, a binding agent is multivalent (i.e., trivalent, quatravalent, etc.) and the binding agent binds to three or more distinct epitopes on a single antigen or binds to three or more distinct epitopes on two or more (multiple) antigens.

Antigen binding fragments of any of the antibodies herein are also contemplated. The terms “antigen binding portion of an antibody,” “antigen binding fragment,” “antigen binding domain,” “antibody fragment,” or a “functional fragment of an antibody” are used interchangeably herein to refer to one or more fragments of an antibody that retain the ability to specifically bind to an antigen. Representative antigen binding fragments include, but are not limited to, a Fab, a Fab′, a F(ab′)₂, a bispecific F(ab′)₂, a trispecific F(ab′)₂, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a VHH, a minibody, an intrabody, fusion proteins comprising an antibody portion (e.g., a domain antibody), a single chain binding polypeptide, a scFv-Fc, a Fab-Fc, a bispecific T cell engager (BiTE; two scFvs produced as a single polypeptide chain, where each scFv comprises an amino acid sequences a combination of CDRs or a combination of VL/VL described herein), a tetravalent tandem diabody (TandAb; an antibody fragment that is produced as a non-covalent homodimer folder in a head-to-tail arrangement, e.g., a TandAb comprising an scFv, where the scFv comprises an amino acid sequences a combination of CDRs or a combination of VL/VL described herein), a Dual-Affinity Re-targeting Antibody (DART; different scFvs joined by a stabilizing interchain disulphide bond), a bispecific antibody (bscAb; two single-chain Fv fragments joined via a glycine-serine linker), a single domain antibody (sdAb), a fusion protein, a bispecific disulfide-stabilized Fv antibody fragment (dsFv-dsFv′; two different disulfide-stabilized Fv antibody fragments connected by flexible linker peptides). In certain embodiments of the invention, a full length antibody (e.g., an antigen binding fragment and an Fc region) are preferred.

Heteroconjugate polypeptides comprising two covalently joined antibodies or antigen binding fragments of antibodies are also within the scope of the disclosure. Suitable linkers may be used to multimerize binding agents. Non-limiting examples of linking peptides include, but are not limited to, (GS)ₙ (SEQ ID NO: 275), (GGS)ₙ (SEQ ID NO: 276), (GGGS)ₙ (SEQ ID NO: 277), (GGSG)ₙ (SEQ ID NO: 278), or (GGSGG)ₙ (SEQ ID NO: 279), (GGGGS)ₙ (SEQ ID NO: 280), wherein n is 1, 2, 3, 4, 5, 6, 7, 8, 9, or 10. For example, a linking peptide can be (GGGGS)₃ (SEQ ID NO: 281) or (GGGGS)₄ (SEQ ID NO: 282). In some embodiments, a linking peptide bridges approximately 3.5 nm between the carboxyl terminus of one variable region and the amino terminus of the other variable region. Linkers of other sequences have been designed and used. Linkers can in turn be modified for additional functions, such as attachment of drugs or attachment to solid supports.

As used herein, the term “avidity” refers to the resistance of a complex of two or more agents to dissociation after dilution. Apparent affinities can be determined by methods such as an enzyme-linked immunosorbent assay (ELISA) or any other suitable technique. Avidities can be determined by methods such as a Scatchard analysis or any other suitable technique.

As used herein, the term “affinity” refers to the equilibrium constant for the reversible binding of two agents and is expressed as K_{D}. The binding affinity (K_{D}) of an antibody or antigen binding fragment herein can be less than 500 nM, 475 nM, 450 nM, 425 nM, 400 nM, 375 nM, 350 nM, 325 nM, 300 nM, 275 nM, 250 nM, 225 nM, 200 nM, 175 nM, 150 nM, 125 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween. Binding affinity may be determined using surface plasmon resonance (SPR), KINEXA® Biosensor, scintillation proximity assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay.

As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

Also provided herein are affinity matured antibodies. The following methods may be used for adjusting the affinity of an antibody and for characterizing a CDR. One way of characterizing a CDR of an antibody and/or altering (such as improving) the binding affinity of a polypeptide, such as an antibody, is termed “library scanning mutagenesis.” Generally, library scanning mutagenesis works as follows. One or more amino acid position in the CDR is replaced with two or more (such as 3, 4, 5, 6, 7, 8, 9, 10, 11, 12, 13, 14, 15, 16, 17, 18, 19, or 20) amino acids. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, for example, about 20-80 clones (depending on the complexity of the library), from each library can be screened for binding specificity or affinity to the target polypeptide (or other binding target), and candidates with increased, the same, decreased, or no binding are identified. Binding affinity may be determined using Biacore surface plasmon resonance analysis, which detects differences in binding affinity of about 2-fold or greater.

In some instances, an antibody or antigen binding fragment is bispecific or multispecific and can specifically bind to more than one antigen. In some cases, such a bispecific or multispecific antibody or antigen binding fragment can specifically bind to 2 or more different antigens. In some cases, a bispecific antibody or antigen binding fragment can be a bivalent antibody or antigen binding fragment. In some cases, a multi specific antibody or antigen binding fragment can be a bivalent antibody or antigen binding fragment, a trivalent antibody or antigen binding fragment, or a quadravalent antibody or antigen binding fragment.

An antibody or antigen binding fragment described herein can be isolated, purified, recombinant, or synthetic.

The antibodies described herein may be made by any suitable method. Antibodies can often be produced in large quantities, particularly when utilizing high level expression vectors.

### Anti-PD-1 Immune Checkpoint Inhibitor Molecules

In some preferred embodiments, the immune checkpoint inhibitor molecule of an activatable immunocytokine is one which binds to PD-1. Programmed cell death protein 1 (also known as PD-1 and CD279), is a cell surface receptor that plays an role in down-regulating the immune system and promoting self-tolerance by suppressing T cell inflammatory activity. PD-1 is an immune cell inhibitory molecule that is expressed on activated B cells, T cells, and myeloid cells. PD-1 represents an immune checkpoint and guards against autoimmunity via a dual mechanism of promoting apoptosis (programmed cell death) in antigen-specific T-cells in lymph nodes while reducing apoptosis in regulatory T cells. PD-1 is a member of the CD28/CTLA-4/ICOS costimulatory receptor family that delivers negative signals that affect T and B cell immunity. PD-1 is monomeric both in solution as well as on cell surface, in contrast to CTLA-4 and other family members that are all disulfide-linked homodimers. Signaling through the PD-1 inhibitory receptor upon binding its ligand, PD-L1, suppresses immune responses against autoantigens and tumors and plays a role in the maintenance of peripheral immune tolerance. The interaction between PD-1 and PD-L1 results in a decrease in tumor infiltrating lymphocytes, a decrease in T cell receptor mediated proliferation, and immune evasion by the cancerous cells. A non-limiting, exemplary, human PD-1 amino acid sequence is

(SEQ ID NO: 98)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

In some embodiments, an activatable immunocytokine of the instant disclosure comprises a polypeptide which is specific for PD-1. In some embodiments, the activatable immunocytokine comprises an anti-PD-1 antibody or antigen binding fragment thereof. Such activatable immunocytokines as provided herein are in some embodiments effective for simultaneously delivering the IL-2 polypeptide and the polypeptide which selectively binds to PD-1 to a target cell, such as a CD8+T effector (T_{eff}) cell. In some embodiments, the IL-2 polypeptide of the activatable immunocytokine delivered to the target cell is selectively activated only when the activatable immunocytokine is delivered to a target tissue (e.g., a tumor microenvironment). In some embodiments, simultaneous delivery of both agents to the same cell and selective activation of the IL-2 polypeptide has numerous potential benefits, including potentially improved IL-2 polypeptide selectivity for cells in a target vicinity, potentially enhanced therapeutic potential of the IL-2 polypeptide owing to higher local concentration due to targeting of the IL-2 polypeptide to target cells by the anti-PD-1 polypeptide, and potentially reduced risk of side effects from administering IL-2 therapies.

In some embodiments, an anti-PD1 antibody or an anti-PD1 antigen binding fragment incorporated into an activatable immunocytokine of the disclosure comprises a combination of a heavy chain variable region (VH) and a light chain variable region (VL) described herein. In another embodiment, an anti-PD1 antibody or an anti-PD1 antigen binding fragment of the disclosure comprises a combination of complementarity determining regions (VH CDR1, VH CDR2, VH CDR3, VL CDR1, VL CDR2, and VL CDR3) described herein. In some embodiments, an anti-PD-1 antibody or an anti-PD-1 antigen binding fragment of the disclosure comprises a modified Tislelizumab, Baizean, 0KVO4111B3N, BGB-A317, hu317-1/IgG4mt2, Sintilimab, Tyvyt, IBI-308, Toripalimab, TeRuiPuLi, Terepril, Tuoyi, JS-001, TAB-001, Camrelizumab, HR-301210, INCSHR-01210, SHR-1210, Cemiplimab, Cemiplimab-rwlc, LIBTAYO®, 6QVLO57INT, H4H7798N, REGN-2810, SAR-439684, Lambrolizumab, Pembrolizumab, KEYTRUDA®, MK-3475, SCH-900475, h409A11, Nivolumab, Nivolumab BMS, OPDIVO®, BMS-936558, MDX-1106, ONO-4538, Prolgolimab, Forteca, BCD-100, Penpulimab, AK-105, Zimberelimab, AB-122, GLS-010, WBP-3055, Balstilimab, 1Q2QT5M7EO, AGEN-2034, AGEN-2034w, Genolimzumab, Geptanolimab, APL-501, CBT-501, GB-226, Dostarlimab, ANB-011, GSK-4057190A, POGVQ9A4S5, TSR-042, WBP-285, Serplulimab, HLX-10, CS-1003, Retifanlimab, 2Y3T51FO1Z, INCMGA-00012, INCMGA-0012, MGA-012, Sasanlimab, LZZOIC2EWP, PF-06801591, RN-888, Spartalizumab, NVP-LZV-184, PDR-001, QOG25L6Z8Z, Relatlimab/nivolumab, BMS-986213, Cetrelimab, JNJ-3283, JNJ-63723283, LYK98WP91F, Tebotelimab, MGD-013, BCD-217, BAT-1306, HX-008, MEDI-5752, JTX-4014, Cadonilimab, AK-104, BI-754091, Pidilizumab, CT-011, MDV-9300, YBL-006, AMG-256, RG-6279, RO-7284755, BH-2950, IBI-315, RG-6139, RO-7247669, ONO-4685, AK-112, 609-A, LY-3434172, T-3011, MAX-10181, AMG-404, IBI-318, MGD-019, INCB-086550, ONCR-177, LY-3462817, RG-7769, RO-7121661, F-520, XmAb-23104, Pd-1-pik, SG-001, S-95016, Sym-021, LZM-009, Budigalimab, 6VDO4TY3OO, ABBV-181, PR-1648817, CC-90006, XmAb-20717, 2661380, AMP-224, B7-DCIg, EMB-02, ANB-030, PRS-332, [89Zr]Deferoxamide-Pembrolizumab, 89Zr-Df-Pembrolizumab, [89Zr]Df-Pembrolizumab, STI-1110, STI-A1110, CX-188, mPD-1 Pb-Tx, MCLA-134, 244C8, ENUM 224C8, ENUM C8, 388D4, ENUM 388D4, ENUM D4, MEDI0680, or AMP-514, or comprises the CDRs of any of these.

In some embodiments, an anti-PD-1 antibody or an anti-PD-1 antigen binding fragment of the disclosure comprises a modified Tislelizumab, Sintilimab, Toripalimab, Terepril, Camrelizumab, Cemiplimab, Pembrolizumab Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotelimab, Cadonilimab, Pidilizumab, LZM-009, or Budigalimab.

In some embodiments, the anti-PD-1 polypeptide is Nivolumab, Pembrolizumab, LZM-009, Dostarlimab, Sintilimab, Spartalizumab, Tislelizumab, or Cemiplimab. In some embodiment, the anti-PD-1 polypeptide is Dostarlimab, Sintilimab, Spartalizumab, or Tislelizumab. In some embodiments, the anti-PD-1 polypeptide is Nivolumab, Pembrolizumab, LZM-009, or Cemiplimab. In some embodiments, the anti-PD-1 polypeptide is modified Pembrolizumab.

It is contemplated that generic or biosimilar versions of the named antibodies herein which share the same amino acid sequence as the indicated antibodies are also encompassed when the name of the antibody is used. In some embodiments, the anti-PD-1 antibody is a biosimilar of Tislelizumab, Sintilimab, Toripalimab, Terepril, Camrelizumab, Cemiplimab, Pembrolizumab Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotelimab, Cadonilimab, A Pidilizumab, LZM-009, or Budigalimab. In some embodiments, the anti-PD-1 antibody is a biosimilar of any one of the antibodies provided herein.

TABLE 4 provides the sequences of exemplary anti-PD-1 polypeptides (e.g., anti-PD-1 antibodies) and anti-PD-1 antigen binding fragments that can be modified to prepare activatable immunocytokines as provided herein. TABLE 4 also shows provides combinations of CDRs that can be utilized in a modified anti-PD-1 activatable immunocytokines. Reference to an anti-PD-1 polypeptide herein may alternatively refer to an anti-PD-1 antigen binding fragment.

In some instances, the SEQ ID NOs listed in Table 4 contain full-length heavy or light chains of the indicated antibodies with the VH or VL respectively indicated in bold. Where there is a reference herein to a VH or VL of a SEQ ID NO in Table 4 which contains a full-length heavy or light chain, it is intended to reference the bolded portion of the sequence. For example, reference to “a VH having an amino acid sequence shown in SEQ ID NO: 332” refers to the bolded portion of SEQ ID NO: 332 in Table 4.

TABLE 4

| Antibody or | | |
|---|---|---|
| Ag-binding | | SEQ ID |
| fragment | Sequence | NO: |
| Tislelizumab, | QVQLQESGPGLVKPSETLSLTCTVSGFSLTSYGVHWIRQPPG | 332 |
| Baizean, | KGLEWIGVIYADGSTNYNPSLKSRVTISKDTSKNQVSLKLSS | |
| 0KVO411B3N, | VTAADTAVYYCARAYGNYWYIDVWGQGTTVTVSSASTKGPSV | |
| BGB-A317, | FPLAPCSRSTSESTAALGCLVKDYFPEPVTVSWNSGALTSGV | |
| hu317-1/ | HTFPAVLQSSGLYSLSSVVTVPSSSLGTKTYTCNVDHKPSNT | |
| IgG4mt2 | KVDKRVESKYGPPCPPCPAPPVAGGPSVFLFPPKPKDTLMIS | |
| Heavy Chain | RTPEVTCVVVAVSQEDPEVQFNWYVDGVEVHNAKTKPREEQF | |
| (VH in Bold) | NSTYRVVSVLTVVHQDWLNGKEYKCKVSNKGLPSSIEKTISK | |
| | AKGQPREPQVYTLPPSQEEMTKNQVSLTCLVKGFYPSDIAVE | |
| | WESNGQPENNYKTTPPVLDSDGSFFLYSKLTVDKSRWQEGNV | |
| | FSCSVMHEALHNHYTQKSLSLSLGK | |
| Tislelizumab, | DIVMTQSPDSLAVSLGERATINCKSSESVSNDVAWYQQKPGQ | 333 |
| Baizean, | PPKLLINYAFHRFTGVPDRFSGSGYGTDFTLTISSLQAEDVA | |
| 0KVO411B3N, | VYYCHQAYSSPYTFGQGTKLEIKRTVAAPSVFIFPPSDEQLK | |
| BGB-A317, | SGTASVVCLLNNFYPREAKVQWKVDNALQSGNSQESVTEQDS | |
| hu317-1/ | KDSTYSLSSTLTLSKADYEKHKVYACEVTHQGLSSPVTKSFN | |
| IgG4mt2 | RGEC | |
| Light Chain | | |
| (VL in Bold) | | |
| Sintilimab, | QVQLVQSGAEVKKPGSSVKVSCKASGGTFSSYAISWVRQAPG | 334 |
| Tyvyt, IBI-308 | QGLEWMGLIIPMEDTAGYAQKFQGRVAITVDESTSTAYMELS | |
| Heavy Chain | SLRSEDTAVYYCARAEHSSTGTFDYWGQGTLVTVSSASTKGP | |
| | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLM | |
| | ISRTPEVTCVVVDVSQEDPEVQFNWYVDGVEVHNAKTKPREE | |
| | QFNSTYRVVSVLTVLHQDWLNGKEYKCKVSNKGLPSSIEKTI | |
| | SKAKGQPREPQVYTLPPSQEEMTKNQVSLTCLVKGFYPSDIA | |
| | VEWESNGQPENNYKTTPPVLDSDGSFFLYSRLTVDKSRWQEG | |
| | NVFSCSVMHEALHNHYTQKSLSLSLGK | |
| Sintilimab, | DIQMTQSPSSVSASVGDRVTITCRASQGISSWLAWYQQKPGK | 335 |
| Tyvyt, IBI-308 | APKLLISAASSLQSGVPSRFSGSGSGTDFTLTISSLQPEDFA | |
| Light Chain | TYYCQQANHLPFTFGGGTKVEIKRTVAAPSVFIFPPSDEQLK | |
| (VL in Bold) | SGTASVVCLLNNFYPREAKVQWKVDNALQSGNSQESVTEQDS | |
| | KDSTYSLSSTLTLSKADYEKHKVYACEVTHQGLSSPVTKSFN | |
| | RGEC | |
| Toripalimab, | QGQLVQSGAEVKKPGASVKVSCKASGYTFTDYEMHWVRQAPI | 336 |
| TeRuiPuLi, | HGLEWIGVIESETGGTAYNQKFKGRVTITADKSTSTAYMELS | |
| Terepril, Tuoyi, | SLRSEDTAVYYCAREGITTVATTYYWYFDVWGQGTTVTVSSA | |
| JS-001, TAB-001 | STKGPSVFPLAPCSRSTSESTAALGCLVKDYFPEPVTVSWNS | |
| Heavy Chain | GALTSGVHTFPAVLQSSGLYSLSSVVTVPSSSLGTKTYTCNV | |
| (VH in Bold) | DHKPSNTKVDKRVESKYGPPCPPCPAPEFLGGPSVFLFPPKP | |
| | KDTLMISRTPEVTCVVVDVSQEDPEVQFNWYVDGVEVHNAKT | |
| | KPREEQFNSTYRVVSVLTVLHQDWLNGKEYKCKVSNKGLPSS | |
| | IEKTISKAKGQPREPQVYTLPPSQEEMTKNQVSLTCLVKGFY | |
| | PSDIAVEWESNGQPENNYKTTPPVLDSDGSFFLYSRLTVDKS | |
| | RWQEGNVFSCSVMHEALHNHYTQKSLSLSLGK | |
| Toripalimab, | DVVMTQSPLSLPVTLGQPASISCRSSQSIVHSNGNTYLEWYL | 337 |
| TeRuiPuLi, | QKPGQSPQLLIYKVSNRFSGVPDRFSGSGSGTDFTLKISRVE | |
| Terepril, Tuoyi, | AEDVGVYYCFQGSHVPLTFGQGTKLEIKRTVAAPSVFIFPPS | |
| JS-001, TAB-001 | DEQLKSGTASVVCLLNNFYPREAKVQWKVDNALQSGNSQESV | |
| Light Chain | TEQDSKDSTYSLSSTLTLSKADYEKHKVYACEVTHQGLSSPV | |
| (VL in Bold) | TKSFNRGEC | |
| Camrelizumab, | EVQLVESGGGLVQPGGSLRLSCAASGFTFSSYMMSWVRQAPG | 338 |
| HR-301210, | KGLEWVATISGGGANTYYPDSVKGRFTISRDNAKNSLYLQMN | |
| INCSHR-01210, | SLRAEDTAVYYCARQLYYFDYWGQGTTVTVSSASTKGPSVFP | |
| SHR-1210 | LAPCSRSTSESTAALGCLVKDYFPEPVTVSWNSGALTSGVHT | |
| Heavy Chain | FPAVLQSSGLYSLSSVVTVPSSSLGTKTYTCNVDHKPSNTKV | |
| (VH in Bold) | DKRVESKYGPPCPPCPAPEFLGGPSVFLFPPKPKDTLMISRT | |
| | PEVTCVVVDVSQEDPEVQFNWYVDGVEVHNAKTKPREEQFNS | |
| | TYRVVSVLTVLHQDWLNGKEYKCKVSNKGLPSSIEKTISKAK | |
| | GQPREPQVYTLPPSQEEMTKNQVSLTCLVKGFYPSDIAVEWE | |
| | SNGQPENNYKTTPPVLDSDGSFFLYSRLTVDKSRWQEGNVFS | |
| | CSVMHEALHNHYTQKSLSLSLGK | |
| Camrelizumab, | DIQMTQSPSSLSASVGDRVTITCLASQTIGTWLTWYQQKPGK | 339 |
| HR-301210, | APKLLIYTATSLADGVPSRFSGSGSGTDFTLTISSLQPEDFA | |
| INCSHR-01210, | TYYCQQVYSIPWTFGGGTKVEIKRTVAAPSVFIFPPSDEQLK | |
| SHR-1210 | SGTASVVCLLNNFYPREAKVQWKVDNALQSGNSQESVTEQDS | |
| Light Chain | KDSTYSLSSTLTLSKADYEKHKVYACEVTHQGLSSPVTKSFN | |
| (VL in Bold) | RGEC | |
| Cemiplimab, | EVQLLESGGVLVQPGGSLRLSCAASGFTFSNFGMTWVRQAPG | 340 |
| Cemiplimab- | KGLEWVSGISGGGRDTYFADSVKGRFTISRDNSKNTLYLQMN | |
| rwlc, | SLKGEDTAVYYCVKWGNIYFDYWGQGTLVTVSSASTKGPSVF | |
| LIBTAYO ®, | PLAPCSRSTSESTAALGCLVKDYFPEPVTVSWNSGALTSGVH | |
| 6QVL057INT, | TFPAVLQSSGLYSLSSVVTVPSSSLGTKTYTCNVDHKPSNTK | |
| H4H7798N, | VDKRVESKYGPPCPPCPAPEFLGGPSVFLFPPKPKDTLMISR | |
| REGN-2810, | TPEVTCVVVDVSQEDPEVQFNWYVDGVEVHNAKTKPREEQFN | |
| SAR-439684 | STYRVVSVLTVLHQDWLNGKEYKCKVSNKGLPSSIEKTISKA | |
| Heavy Chain | KGQPREPQVYTLPPSQEEMTKNQVSLTCLVKGFYPSDIAVEW | |
| (VH in Bold) | ESNGQPENNYKTTPPVLDSDGSFFLYSRLTVDKSRWQEGNVF | |
| | SCSVMHEALHNHYTQKSLSLSLGK | |
| Cemiplimab, | DIQMTQSPSSLSASVGDSITITCRASLSINTFLNWYQQKPGK | 341 |
| Cemiplimab- | APNLLIYAASSLHGGVPSRFSGSGSGTDFTLTIRTLQPEDFA | |
| rwlc, | TYYCQQSSNTPFTFGPGTVVDFRRTVAAPSVFIFPPSDEQLK | |
| LIBTAYO ®, | SGTASVVCLLNNFYPREAKVQWKVDNALQSGNSQESVTEQDS | |
| 6QVL057INT, | KDSTYSLSSTLTLSKADYEKHKVYACEVTHQGLSSPVTKSEN | |
| H4H7798N, | RGEC | |
| REGN-2810, | | |
| SAR-439684 | | |
| Light Chain | | |
| (VL in Bold) | | |
| Lambrolizumab, | QVQLVQSGVEVKKPGASVKVSCKASGYTFTNYYMYWVRQAPG | 342 |
| Pembrolizumab, | QGLEWMGGINPSNGGTNFNEKFKNRVTLTTDSSTTTAYMELK | |
| KEYTRUDA ®, | SLQFDDTAVYYCARRDYRFDMGFDYWGQGTTVTVSSASTKGP | |
| MK-3475, | SVFPLAPCSRSTSESTAALGCLVKDYFPEPVTVSWNSGALTS | |
| SCH-900475, | GVHTFPAVLQSSGLYSLSSVVTVPSSSLGTKTYTCNVDHKPS | |
| h409A11 | NTKVDKRVESKYGPPCPPCPAPEFLGGPSVFLFPPKPKDTLM | |
| Heavy Chain | ISRTPEVTCVVVDVSQEDPEVQFNWYVDGVEVHNAKTKPREE | |
| (VH in Bold) | QFNSTYRVVSVLTVLHQDWLNGKEYKCKVSNKGLPSSIEKTI | |
| | SKAKGQPREPQVYTLPPSQEEMTKNQVSLTCLVKGFYPSDIA | |
| | VEWESNGQPENNYKTTPPVLDSDGSFFLYSRLTVDKSRWQEG | |
| | NVFSCSVMHEALHNHYTQKSLSLSLGK | |
| Lambrolizumab, | EIVLTQSPATLSLSPGERATLSCRASKGVSTSGYSYLHWYQQ | 343 |
| Pembrolizumab, | KPGQAPRLLIYLASYLESGVPARFSGSGSGTDFTLTISSLEP | |
| KEYTRUDA ®, | EDFAVYYCQHSRDLPLTFGGGTKVEIKRTVAAPSVFIFPPSD | |
| MK-3475, | EQLKSGTASVVCLLNNFYPREAKVQWKVDNALQSGNSQESVT | |
| SCH-900475, | EQDSKDSTYSLSSTLTLSKADYEKHKVYACEVTHQGLSSPVT | |
| h409A11 | KSFNRGEC | |
| Light Chain | | |
| (VL in Bold) | | |
| Lambrolizumab, | QVQLVQSGVEVKKPGASVKVSCKASGYTFTNYYMYWVRQAPG | 344 |
| Pembrolizumab | QGLEWMGGFPSNGGTNFNEKFKNRVTLTTDSSTTTAYMELKS | |
| KEYTRUDA ®, | LQFDDTAVYYCARRDYRFDMGFDYWGQGTTVTVSS | |
| MK-3475, | | |
| SCH-900475, | | |
| h409A11 | | |
| VH | | |
| Lambrolizumab, | EIVLTQSPATLSLSPGERATLSCRASKGVSTSGYSYLHWYQQ | 345 |
| Pembrolizumab, | KPGQAPRLLIYLASYLESGVPARFSGSGSGTDFTLTISSLEP | |
| KEYTRUDA ®, | EDFAVYYCQHSRDLPLTFGGGTKVEIK | |
| MK-3475, | | |
| SCH-900475, | | |
| b409A11 | | |
| VL | | |
| Nivolumab, | QVQLVESGGGVVQPGRSLRLDCKASGITFSNSGMHWVRQAPG | 346 |
| Nivolumab | KGLEWVAVIWYDGSKRYYADSVKGRFTISRDNSKNTLFLQMN | |
| BMS, | SLRAEDTAVYYCATNDDYWGQGTLVTVSS | |
| OPDIVO ®, | | |
| BMS-936558, | | |
| MDX-1106, | | |
| ONO-4538 | | |
| VH | | |
| Nivolumab, | EIVLTQSPATLSLSPGERATLSCRASQSVSSYLAWYQQKPGQ | 347 |
| Nivolumab | APRLLIYDASNRATGIPARFSGSGSGTDFTLTISSLEPEDFA | |
| BMS, | VYYCQQSSNWPRTFGQGTKVEIK | |
| OPDIVO ®, | | |
| BMS-936558, | | |
| MDX-1106, | | |
| ONO-4538 | | |
| VL | | |
| Prolgolimab, | QVQLVQSGGGLVQPGGSLRLSCAASGFTFSSYWMYWVRQVPG | 348 |
| Forteca, | KGLEWVSAIDTGGGRTYYADSVKGRFAISRVNAKNTMYLQMN | |
| BCD-100 | SLRAEDTAVYYCARDEGGGTGWGVLKDWPYGLDAWGQGTLVT | |
| Heavy Chain | VSSASTKGPSVFPLAPSSKSTSGGTAALGCLVKDYFPEPVTV | |
| (VH in Bold) | SWNSGALTSGVHTFPAVLQSSGLYSLSSVVTVPSSSLGTQTY | |
| | ICNVNHKPSNTKVDKRVEPKSCDKTHTCPPCPAPEAAGGPSV | |
| | FLFPPKPKDTLMISRTPEVTCVVVDVSHEDPEVKFNWYVDGV | |
| | EVHNAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPGK | |
| Prolgolimab, | QPVLTQPLSVSVALGQTARITCGGNNIGSKNVHWYQQKPGQA | 349 |
| Forteca, | PVLVIYRDSNRPSGIPERFSGSNSGNTATLTISRAQAGDEAD | |
| BCD-100 | YYCQVWDSSTAVFGTGTKLTVLQRTVAAPSVFIFPPSDEQLK | |
| Light Chain | SGTASVVCLLNNFYPREAKVQWKVDNALQSGNSQESVTEQDS | |
| (VL in Bold) | KDSTYSLSSTLTLSKADYEKHKVYACEVTHQGLSSPVTKSFN | |
| | RGEC | |
| Balstilimab, | QVQLVESGGGVVQPGRSLRLSCAASGFTFSSYGMHWVRQAPG | 350 |
| IQ2QT5M7EO, | KGLEWVAVIWYDGSNKYYADSVKGRFTISRDNSKNTLYLQMN | |
| AGEN-2034, | SLRAEDTAVYYCASNGDHWGQGTLVTVSSASTKGPSVFPLAP | |
| AGEN-2034w | CSRSTSESTAALGCLVKDYFPEPVTVSWNSGALTSGVHTFPA | |
| Heavy Chain | VLQSSGLYSLSSVVTVPSSSLGTKTYTCNVDHKPSNTKVDKR | |
| (VH in Bold) | VESKYGPPCPPCPAPEFLGGPSVFLFPPKPKDTLMISRTPEV | |
| | TCVVVDVSQEDPEVQFNWYVDGVEVHNAKTKPREEQFNSTYR | |
| | VVSVLTVLHQDWLNGKEYKCKVSNKGLPSSIEKTISKAKGQP | |
| | REPQVYTLPPSQEEMTKNQVSLTCLVKGFYPSDIAVEWESNG | |
| | QPENNYKTTPPVLDSDGSFFLYSRLTVDKSRWQEGNVFSCSV | |
| | MHEALHNHYTQKSLSLSLG | |
| Balstilimab, | EIVMTQSPATLSVSPGERATLSCRASQSVSSNLAWYQQKPGQ | 351 |
| IQ2QT5M7EO, | APRLLIYGASTRATGIPARFSGSGSGTEFTLTISSLQSEDFA | |
| AGEN-2034, | VYYCQQYNNWPRTFGQGTKVEIKRTVAAPSVFIFPPSDEQLK | |
| AGEN-2034w | SGTASVVCLLNNFYPREAKVQWKVDNALQSGNSQESVTEQDS | |
| Light Chain | KDSTYSLSSTLTLSKADYEKHKVYACEVTHQGLSSPVTKSFN | |
| (VL in Bold) | RGEC | |
| Dostarlimab, | EVQLLESGGGLVQPGGSLRLSCAASGFTFSSYDMSWVRQAPG | 352 |
| ANB-011, | KGLEWVSTISGGGSYTYYQDSVKGRFTISRDNSKNTLYLQMN | |
| GSK-4057190A, | SLRAEDTAVYYCASPYYAMDYWGQGTTVTVSSASTKGPSVFP | |
| P0GVQ9A4S5, | LAPCSRSTSESTAALGCLVKDYFPEPVTVSWNSGALTSGVHT | |
| TSR-042, | FPAVLQSSGLYSLSSVVTVPSSSLGTKTYTCNVDHKPSNTKV | |
| WBP-285 | DKRVESKYGPPCPPCPAPEFLGGPSVFLFPPKPKDTLMISRT | |
| Heavy Chain | PEVTCVVVDVSQEDPEVQFNWYVDGVEVHNAKTKPREEQFNS | |
| (VH in Bold) | TYRVVSVLTVLHQDWLNGKEYKCKVSNKGLPSSIEKTISKAK | |
| | GQPREPQVYTLPPSQEEMTKNQVSLTCLVKGFYPSDIAVEWE | |
| | SNGQPENNYKTTPPVLDSDGSFFLYSRLTVDKSRWQEGNVFS | |
| | CSVMHEALHNHYTQKSLSLSLGK | |
| Dostarlimab, | DIQLTQSPSFLSAYVGDRVTITCKASQDVGTAVAWYQQKPGK | 353 |
| ANB-011, | APKLLIYWASTLHTGVPSRFSGSGSGTEFTLTISSLQPEDFA | |
| GSK-4057190A, | TYYCQHYSSYPWTFGQGTKLEIKRTVAAPSVFIFPPSDEQLK | |
| P0GVQ9A4S5, | SGTASVVCLLNNFYPREAKVQWKVDNALQSGNSQESVTEQDS | |
| TSR-042, | KDSTYSLSSTLTLSKADYEKHKVYACEVTHQGLSSPVTKSFN | |
| WBP-285 | RGEC | |
| Light Chain | | |
| (VL in Bold) | | |
| Serplulimab, | QVQLVESGGGLVKPGGSLRLSCAASGFTFSNYGMSWIRQAPG | 354 |
| HLX-10 | KGLEWSTISGGGSNIYYADSVKGRFTISRDNAKNSLYLQMNS | |
| Heavy Chain | LRAEDTAVYYCVSYYYGIDFWGQGTSVTVSSASKYGPSVFPL | |
| (VH in Bold) | APCSRSTSESTAALGCLVKDYFPEPVTVSWNSGALTSGVHTF | |
| | PAVLQSSGLYSLSSVVTVPSSSLGTKTYTCNVDHKPSNTKVD | |
| | KRVESKYGPPCPPCPAPEFLGGPSVFLFPPKPKDTLMISRTP | |
| | EVTCVVVDVSQEDPEVQFNWYVDGVEVHNAKTKPREEQFNST | |
| | YRVVSVVLTVLHQDWLNGKEYKCKVSNKGLPSSIEKTISKAK | |
| | GQPREPQVYTLPPSQEEMTKNQVSLTCLVKGFYPSDIAVEWE | |
| | SNGQPENNYKTTPPVLDSDGSFFLYSRLTVDKSRWQEGNVFS | |
| | CSVMHEALHNHYTQKSLSLSLGK | |
| Serplulimab, | DIQMTQSPSSLSASVGDRVTITCKASQDVTTAVAWYQQKPGK | 355 |
| HLX-10 | APKLLIYWASTRHTGVPSRFSGSGSGTDFTLTISSLQPEDFA | |
| Light Chain | TYYCQQHYTIPWTFGGGTKLEIKRTVAAPSVFIFPPSDEQLK | |
| (VL in Bold) | SGTASVVCLLNNFYPREAKVQWKVDNALQSGNSQESVTEQDS | |
| | KDSTYSLSSTLTLSKADYEKHKVYACEVTHQGLSSPVTKSFN | |
| | RGEC | |
| Retifanlimab, | QVQLVQSGAEVKKPGASVKVSCKASGYSFTSYWMNWVRQAPG | 356 |
| 2Y3T5IF01Z, | QGLEWIGVIHPSDSETWLDQKFKDRVTITVDKSTSTAYMELS | |
| INCMGA-00012, | SLRSEDTAVYYCAREHYGTSPFAYWGQGTLVTVSSASTKGPS | |
| INCMGA-0012, | VFPLAPCSRSTSESTAALGCLVKDYFPEPVTVSWNSGALTSG | |
| MGA-012 | VHTFPAVLQSSGLYSLSSVVTVPSSSLGTKTYTCNVDHKPSN | |
| Heavy Chain | TKVDKRVESKYGPPCPPCPAPEFLGGPSVFLFPPKPKDTLMI | |
| (VH in Bold) | SRTPEVTCVVVDVSQEDPEVQFNWYVDGVEVHNAKTKPREEQ | |
| | FNSTYRVVSVLTVLHQDWLNGKEYKCKVSNKGLPSSIEKTIS | |
| | KAKGQPREPQVYTLPPSQEEMTKNQVSLTCLVKGFYPSDIAV | |
| | EWESNGQPENNYKTTPPVLDSDGSFFLYSRLTVDKSRWQEGN | |
| | VFSCSVMHEALHNHYTQKSLSLSLG | |
| Retifanlimab, | EIVLTQSPATLSLSPGERATLSCRASESVDNYGMSFMNWFQQ | 357 |
| 2Y3T5IF01Z, | KPGQPPKLLIHAASNQGSGVPSRFSGSGSGTDFTLTISSLEP | |
| INCMGA-00012, | EDFAVYFCQQSKEVPYTFGGGTKVEIKRTVAAPSVFIFPPSD | |
| INCMGA-0012, | EQLKSGTASVVCLLNNFYPREAKVQWKVDNALQSGNSQESVT | |
| MGA-012 | EQDSKDSTYSLSSTLTLSKADYEKHKVYACEVTHQGLSSPVT | |
| Light Chain | KSFNRGEC | |
| (VL in Bold) | | |
| Sasanlimab, | QVQLVQSGAEVKKPGASVKVSCKASGYTFTSYWINWVRQAPG | 358 |
| LZZ0IC2EWP, | QGLEWMGNIYPGSSLTNYNEKFKNRVTMTRDTSTSTVYMELS | |
| PF-06801591, | SLRSEDTAVYYCARLSTGTFAYWGQGTLVTVSSASTKGPSVF | |
| RN-888 | PLAPCSRSTSESTAALGCLVKDYFPEPVTVSWNSGALTSGVH | |
| Heavy Chain | TFPAVLQSSGLYSLSSVVTVPSSSLGTKTYTCNVDHKPSNTK | |
| (VH in Bold) | VDKRVESKYGPPCPPCPAPEFLGGPSVFLFPPKPKDTLMISR | |
| | TPEVTCVVVDVSQEDPEVQFNWYVDGVEVHNAKTKPREEQFN | |
| | STYRVVSVLTVLHQDWLNGKEYKCKVSNKGLPSSIEKTISKA | |
| | KGQPREPQVYTLPPSQEEMTKNQVSLTCLVKGFYPSDIAVEW | |
| | ESNGQPENNYKTTPPVLDSDGSFFLYSRLTVDKSRWQEGNVF | |
| | SCSVMHEALHNHYTQKSLSLSLGK | |
| Sasanlimab, | DIVMTQSPDSLAVSLGERATINCKSSQSLWDSGNQKNFLTWY | 359 |
| LZZ0IC2EWP, | QQKPGQPPKLLIYWTSYRESGVPDRESGSGSGTDFTLTISSL | |
| PF-06801591, | QAEDVAVYYCQNDYFYPHTFGGGTKVEIKRTVAAPSVFIFPP | |
| RN-888 | SDEQLKSGTASVVCLLNNFYPREAKVQWKVDNALQSGNSQES | |
| Light Chain | VTEQDSKDSTYSLSSTLTLSKADYEKHKVYACEVTHQGLSSP | |
| (VL in Bold) | VTKSFNRGEC | |
| Spartalizumab, | EVQLVQSGAEVKKPGESLRISCKGSGYTFTTYWMHWVRQATG | 360 |
| NVP-LZV-184, | QGLEWMGNIYPGTGGSNFDEKFKNRVTITADKSTSTAYMELS | |
| PDR-001, | SLRSEDTAVYYCTRWTTGTGAYWGQGTTVTVSSASTKGPSVF | |
| QOG25L6Z8Z | PLAPCSRSTSESTAALGCLVKDYFPEPVTVSWNSGALTSGVH | |
| Heavy Chain | TFPAVLQSSGLYSLSSVVTVPSSSLGTKTYTCNVDHKPSNTK | |
| (VH in Bold) | VDKRVESKYGPPCPPCPAPEFLGGPSVFLFPPKPKDTLMISR | |
| | TPEVTCVVVDVSQEDPEVQFNWYVDGVEVHNAKTKPREEQFN | |
| | STYRVVSVLTVLHQDWLNGKEYKCKVSNKGLPSSIEKTISKA | |
| | KGQPREPQVYTLPPSQEEMTKNQVSLTCLVKGFYPSDIAVEW | |
| | ESNGQPENNYKTTPPVLDSDGSFFLYSRLTVDKSRWQEGNVF | |
| | SCSVMHEALHNHYTQKSLSLSLG | |
| Spartalizumab, | EIVLTQSPATLSLSPGERATLSCKSSQSLLDSGNQKNFLTWY | 361 |
| NVP-LZV-184, | QQKPGQAPRLLIYWASTRESGVPSRFSGSGSGTDFTFTISSL | |
| PDR-001, | EAEDAATYYCQNDYSYPYTFGQGTKVEIKRTVAAPSVFIFPP | |
| QOG25L6Z8Z | SDEQLKSGTASVVCLLNNFYPREAKVQWKVDNALQSGNSQES | |
| Light Chain | VTEQDSKDSTYSLSSTLTLSKADYEKHKVYACEVTHQGLSSP | |
| (VL in Bold) | VTKSFNRGEC | |
| Cetrelimab, | QVQLVQSGAEVKKPGSSVKVSCKASGGTFSSYAISWVRQAPG | 362 |
| JNJ-3283, | QGLEWMGGIIPIFDTANYAQKFQGRVTITADESTSTAYMELS | |
| JNJ-63723283, | SLRSEDTAVYYCARPGLAAAYDTGSLDYWGQGTLVTVSSAST | |
| LYK98WP91F | KGPSVFPLAPCSRSTSESTAALGCLVKDYFPEPVTVSWNSGA | |
| Heavy Chain | LTSGVHTFPAVLQSSGLYSLSSVVTVPSSSLGTKTYTCNVDH | |
| (VH in Bold) | KPSNTKVDKRVESKYGPPCPPCPAPEFLGGPSVFLFPPKPKD | |
| | TLMISRTPEVTCVVVDVSQEDPEVQFNWYVDGVEVHNAKTKP | |
| | REEQFNSTYRVVSVLTVLHQDWLNGKEYKCKVSNKGLPSSIE | |
| | KTISKAKGQPREPQVYTLPPSQEEMTKNQVSLTCLVKGFYPS | |
| | DIAVEWESNGQPENNYKTTPPVLDSDGSFFLYSRLTVDKSRW | |
| | QEGNVFSCSVMHEALHNHYTQKSLSLSLGK | |
| Cetrelimab, | EIVLTQSPATLSLSPGERATLSCRASQSVRSYLAWYQQKPGQ | 363 |
| JNJ-3283, | APRLLIYDASNRATGIPARFSGSGSGTDFTLTISSLEPEDFA | |
| JNJ-63723283, | VYYCQQRNYWPLTFGQGTKVEIKRTVAAPSVFIFPPSDEQLK | |
| LYK98WP91F | SGTASVVCLLNNFYPREAKVQWKVDNALQSGNSQESVTEQDS | |
| Light Chain | KDSTYSLSSTLTLSKADYEKHKVYACEVTHQGLSSPVTKSFN | |
| (VL in Bold) | RGEC | |
| Tebotelimab, | DIQMTQSPSSLSASVGDRVTITCRASQDVSSVVAWYQQKPGK | 364 |
| MGD-013 | APKLLIYSASYRYTGVPSRFSGSGSGTDFTLTISSLQPEDFA | |
| Heavy Chain | TYYCQQHYSTPWTFGGGTKLEIKGGGSGGGGQVQLVQSGAEV | |
| (VH in Bold) | KKPGASVKVSCKASGYSFTSYWMNWVRQAPGQGLEWIGVIHP | |
| | SDSETWLDQKFKDRVTITVDKSTSTAYMELSSLRSEDTAVYY | |
| | CAREHYGTSPFAYWGQGTLVTVSSGGCGGGEVAACEKEVAAL | |
| | EKEVAALEKEVAALEKESKYGPPCPPCPAPEFLGGPSVFLFP | |
| | PKPKDTLYITREPEVTCVVVDVSQEDPEVQFNWYVDGVEVHN | |
| | AKTKPREEQFNSTYRVVSVLTVLHQDWLNGKEYKCKVSNKGL | |
| | PSSIEKTISKAKGQPREPQVYTLPPSQEEMTKNQVSLTCLVK | |
| | GFYPSDIAVEWESNGQPENNYKTTPPVLDSDGSFFLYSRLTV | |
| | DKSRWQEGNVFSCSVMHEALHNHYTQKSLSLSLG | |
| Tebotelimab, | EIVLTQSPATLSLSPGERATLSCRASESVDNYGMSFMNWFQQ | 365 |
| MGD-013 | KPGQPPKLLIHAASNQGSGVPSRFSGSGSGTDFTLTISSLEP | |
| Light Chain | EDFAVYFCQQSKEVPYTFGGGTKVEIKGGGSGGGGQVQLVQS | |
| (VL in Bold) | GAEVKKPGASVKVSCKASGYTFTDYNMDWVRQAPGQGLEWMG | |
| | DINPDNGVTIYNQKFEGRVTMTTDTSTSTAYMELRSLRSDDT | |
| | AVYYCAREADYFYFDYWGQGTTLTVSSGGCGGGKVAACKEKV | |
| | AALKEKVAALKEKVAALKE | |
| Pidilizumab, | QVQLVQSGSELKKPGASVKISCKASGYTFTNYGMNWVRQAPG | 366 |
| CT-011, | QGLQWMGWINTDSGESTYAEEFKGRFVFSLDTSVNTAYLQIT | |
| MDV-9300 | SLTAEDTGMYFCVRVGYDALDYWGQGTLVTVSSASTKGPSVF | |
| Heavy Chain | PLAPSSKSTSGGTAALGCLVKDYFPEPVTVSWNSGALTSGVH | |
| (VH in Bold) | TFPAVLQSSGLYSLSSVVTVPSSSLGTQTYICNVNHKPSNTK | |
| | VDKRVEPKSCDKTHTCPPCPAPELLGGPSVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPIEKTI | |
| | SKAKGQPREPQVYTLPPSREEMTKNQVSLTCLVKGFYPSDIA | |
| | VEWESNGQPENNYKTTPPVLDSDGSFFLYSKLTVDKSRWQQG | |
| | NVFSCSVMHEALHNHYTQKSLSLSPGK | |
| Pidilizumab, | EIVLTQSPSSLSASVGDRVTITCSARSSVSYMHWFQQKPGKA | 367 |
| CT-011, | PKLWIYRTSNLASGVPSRFSGSGSGTSYCLTINSLQPEDFAT | |
| MDV-9300 | YYCQQRSSFPLTFGGGTKLEIKRTVAAPSVFIFPPSDEQLKS | |
| Light Chain | GTASVVCLLNNFYPREAKVQWKVDNALQSGNSQESVTEQDSK | |
| (VL in Bold) | DSTYSLSSTLTLSKADYEKHKVYACEVTHQGLSSPVTKSFNR | |
| | GEC | |
| SG-001 VH | QVQLVESGGGVVQPGRSLRLTCKASGLTFSSSGMHWVRQAPG | 368 |
| | KGLEWVAVIWYDGSKRYYADSVKGRFTISRDNSKNTLFLQMN | |
| | SLRAEDTAVYYCATNNDYWGQGTLVTVSS | |
| SG-001 VL | EIVLTQSPATLSLSPGERATLSCRASQSVSSYLAWYQQKPGQ | 369 |
| | APRLLIYTASNRATGIPARFSGSGSGTDFTLTISSLEPEDFA | |
| | VYYCQQYSNWPRTFGQGTKVEIK | |
| mpLZM-009 | EVQLQQSGPVLVKPGASVKMSCKASGYTFTSYYMYWVKQSHG | 370 |
| VH (Murine | KSLEWIGGVNPSNGGTNFNEKFKSKATLTVDKSSSTAYMELN | |
| Precursor of | SLTSEDSAVYYCARRDYRYDMGFDYWGQGTTLTVSS | |
| LZM-009) | | |
| mpLZM-009 | QIVLTQSPAIMSASPGEKVTMTCRASKGVSTSGYSYLHWYQQ | 371 |
| VL (Murine | KPGSSPRLLIYLASYLESGVPVRFSGSGSGTSYSLTISRMEA | |
| Precursor of | EDAATYYCQHSRELPLTFGTGTRLEIK | |
| LZM-009) | | |
| LZM-009 VH | QVQLVQSGAEVKKPGASVKVSCKASGYTFTSYYMYWVRQAPG | 372 |
| | QGLEWMGGVNPSNGGTNFNEKFKSRVTITADKSTSTAYMELS | |
| | SLRSEDTAVYYCARRDYRYDMGFDYWGQGTTVTVSS | |
| LZM-009 VL | EIVLTQSPATLSLSPGERATISCRASKGVSTSGYSYLHWYQQ | 373 |
| | KPGQAPRLLIYLASYLESGVPARFSGSGSGTDFTLTISSLEP | |
| | EDFATYYCQHSRELPLTFGTGTKVEIK | |
| Budigalimab, | EIQLVQSGAEVKKPGSSVKVSCKASGYTFTHYGMNWVRQAPG | 374 |
| 6VDO4TY3OO, | QGLEWVGWVNTYTGEPTYADDFKGRLTFTLDTSTSTAYMELS | |
| ABBV-181, | SLRSEDTAVYYCTREGEGLGFGDWGQGTTVTVSSASTKGPSV | |
| PR-1648817 | FPLAPSSKSTSGGTAALGCLVKDYFPEPVTVSWNSGALTSGV | |
| Heavy Chain | HTFPAVLQSSGLYSLSSVVTVPSSSLGTQTYICNVNHKPSNT | |
| (VH in Bold) | KVDKKVEPKSCDKTHTCPPCPAPEAAGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKT | |
| | ISKAKGQPREPQVYTLPPSREEMTKNQVSLTCLVKGFYPSDI | |
| | AVEWESNGQPENNYKTTPPVLDSDGSFFLYSKLTVDKSRWQQ | |
| | GNVFSCSVMHEALHNHYTQKSLSLSPGK | |
| Budigalimab, | DVVMTQSPLSLPVTPGEPASISCRSSQSIVHSHGDTYLEWYL | 375 |
| 6VDO4TY3OO, | QKPGQSPQLLIYKVSNRFSGVPDRFSGSGSGTDFTLKISRVE | |
| ABBV-181, | AEDVGVYYCFQGSHIPVTFGQGTKLEIKRTVAAPSVFIFPPS | |
| PR-1648817 | DEQLKSGTASVVCLLNNFYPREAKVQWKVDNALQSGNSQESV | |
| Light Chain | TEQDSKDSTYSLSSTLTLSKADYEKHKVYACEVTHQGLSSPV | |
| (VL in Bold) | TKSFNRGEC | |
| Lambrolizumab, | NYYMY | 376 |
| Pembrolizumab, | | |
| KEYTRUDA ®, | | |
| MK-3475, | | |
| SCH-900475, | | |
| h409A11 | | |
| VH CDR1 | | |
| Lambrolizumab, | GINPSNGGTNFNEKFKN | 377 |
| Pembrolizumab, | | |
| KEYTRUDA ®, | | |
| MK-3475, | | |
| SCH-900475, | | |
| h409A11 | | |
| VH CDR2 | | |
| Lambrolizumab, | RDYRFDMGFDY | 378 |
| Pembrolizumab, | | |
| KEYTRUDA ®, | | |
| MK-3475, | | |
| SCH-900475, | | |
| h409A11 | | |
| VH CDR3 | | |
| Lambrolizumab, | RASKGVSTSGYSYLH | 379 |
| Pembrolizumab, | | |
| KEYTRUDA ®, | | |
| MK-3475, | | |
| SCH-900475, | | |
| h409A11 | | |
| VL CDR1 | | |
| Lambrolizumab, | LASYLES | 380 |
| Pembrolizumab, | | |
| KEYTRUDA ®, | | |
| MK-3475, | | |
| SCH-900475, | | |
| h409A11 | | |
| VL CDR2 | | |
| Lambrolizumab, | QHSRDLPLT | 381 |
| Pembrolizumab, | | |
| KEYTRUDA ®, | | |
| MK-3475, | | |
| SCH-900475, | | |
| h409All | | |
| VL CDR3 | | |
| Nivolumab, | NSGMH | 382 |
| Nivolumab | | |
| BMS, | | |
| OPDIVO ®, | | |
| BMS-936558, | | |
| MDX-1106, | | |
| ONO-4538 | | |
| VH CDR1 | | |
| Nivolumab, | VIWYDGSKRYYADSVKG | 383 |
| Nivolumab | | |
| BMS. | | |
| OPDIVO ®, | | |
| BMS-936558, | | |
| MDX-1106, | | |
| ONO-4538 | | |
| VH CDR2 | | |
| Nivolumab, | NDDY | 384 |
| Nivolumab | | |
| BMS, | | |
| OPDIVO ®, | | |
| BMS-936558, | | |
| MDX-1106, | | |
| ONO-4538 | | |
| VH CDR3 | | |
| Nivolumab, | RASQSVSSYLA | 385 |
| Nivolumab | | |
| BMS, | | |
| OPDIVO ®, | | |
| BMS-936558, | | |
| MDX-1106, | | |
| ONO-4538 | | |
| VL CDR1 | | |
| Nivolumab, | DASNRAT | 386 |
| Nivolumab | | |
| BMS, | | |
| OPDIVO ®, | | |
| BMS-936558, | | |
| MDX-1106, | | |
| ONO-4538 | | |
| VL CDR2 | | |
| Nivolumab, | QQSSNWPRT | 387 |
| Nivolumab | | |
| BMS, | | |
| OPDIVO ®, | | |
| BMS-936558, | | |
| MDX-1106, | | |
| ONO-4538 | | |
| VL CDR3 | | |
| Serplulimab, | FTFSNYGMS | 388 |
| HLX-10 | | |
| VH CDR1 | | |
| Serplulimab, | TISGGGSNIY | 389 |
| HLX-10 | | |
| VH CDR2 | | |
| Serplulimab, | VSYYYGIDF | 390 |
| HLX-10 | | |
| VH CDR3 | | |
| Serplulimab, | KASQDVTTAVA | 391 |
| HLX-10 | | |
| VL CDR1 | | |
| Serplulimab, | WASTRHT | 392 |
| HLX-10 | | |
| VL CDR2 | | |
| Serplulimab, | QQHYTIPWT | 393 |
| HLX-10 | | |
| VL CDR3 | | |
| SG-001 | GLTFSSSG | 394 |
| VH CDR1 | | |
| SG-001 | IWYDGSKR | 395 |
| VH CDR2 | | |
| SG-001 | ATNNDY | 396 |
| VH CDR3 | | |
| SG-001 | RASQSVSSYLA | 397 |
| VL CDR1 | | |
| SG-001 | TASNRAT | 398 |
| VL CDR2 | | |
| SG-001 | QQYSNWPRT | 399 |
| VL CDR3 | | |
| PD-1-Fc- | MQIPQAPWPWWAVLQLGWRPGWFLDSPDRPWNPPTFSPALLV | 400 |
| OX40L (Code), | VTEGDNATFTCSFSNTSESFVLNWYRMSPSNQTDKLAAFPED | |
| SL-279252 | RSQPGQDCRFRVTQLPNGRDFHMSVVRARRNDSGTYLCGAIS | |
| (Code), | LAPKAQIKESLRAELRVTERRAEVPTAHPSPSPRPAGQFQSK | |
| TAK-252 (Code) | YGPPCPSCPAPEFLGGPSVFLFPPKPKDTLMISRTPEVTCWV | |
| | DVSQEDPEVQFNWYVDGVEVHNAKTKPREEQFNSTYRVVSVL | |
| | TVLHQDWLSGKEYKCKVSSKGLPSSIEKTISNATGQPREPQV | |
| | YTLPPSQEEMTKNQVSLTCLVKGFYPSDIAVEWESNGQPENN | |
| | YKTTPPVLDSDGSFFLYSRLTVDKSSWQEGNVFSCSVMHEAL | |
| | HNHYTQKSLSLSLGKIEGRMDQVSHRYPRIQSIKVQFTEYKK | |
| | EKGFILTSQKEDEIMKVQNNSVIINCDGFYLISLKGYFSQEV | |
| | NISLHYQKDEEPLFQLKKVRSVNSLMVASLTYKDKVYLNVTT | |
| | DNTSLDDFHVNGGELILIHQNPGEFCVLMQIPQAPWPWWAVL | |
| | QLGWRPGWFLDSPDRPWNPPTFSPALLVVTEGDNATFTCSFS | |
| | NTSESFVLNWYRMSPSNQTDKLAAFPEDRSQPGQDCRFRVTQ | |
| | LPNGRDFHMSVVRARRNDSGTYLCGAISLAPKAQIKESLRAE | |
| | LRVTERRAEVPTAHPSPSPRPAGQFQQVSHRYPRIQSIKVQF | |
| | TEYKKEKGFILTSQKEDEIMKVQNNSVIINCDGFYLISLKGY | |
| | FSQEVNISLHYQKDEEPLFQLKKVRSVNSLMVASLTYKDKVY | |
| | LNVTTDNTSLDDFHVNGGELILIHQNPGEFCVL | |
| PD1-0103-0314, | EVQLLESGGGLVQPGGSLRLSCAASGFSFSSYTMSWVRQAPG | 401 |
| PD1-0103-0313, | KGLEWVATISGGGRDIYYPDSVKGRFTISRDNSKNTLYLQMN | |
| and PD1-0103- | SLRAEDTAVYYCVLLTGRVYFALDSWGQGTLVTVSS | |
| 0312 | | |
| VH | | |
| PD1-0103-0312 | DIVMTQSPDSLAVSLGERATINCKASESVDTSDNSFIHWYQQ | 402 |
| VL | KPGQSPKLLIYRSSTLESGVPDRFSGSGSGTDFTLTISSLQA | |
| | EDVAVYYCQQNYDVPWTFGQGTKVEIK | |
| PD1-0103-0313 | DVVMTQSPLSLPVTLGQPASISCRASESVDTSDNSFIHWYQQ | 403 |
| VL | RPGQSPRLLIYRSSTLESGVPDRFSGSGSGTDFTLKISRVEA | |
| | EDVGVYYCQQNYDVPWTFGQGTKVEIK | |
| PD1-0103-0314 | EIVLTQSPATLSLSPGERATLSCRASESVDTSDNSFIHWYQQ | 404 |
| VL | KPGQSPRLLIYRSSTLESGIPARFSGSGSGTDFTLTISSLEP | |
| | EDFAVYYCQQNYDVPWTFGQGTKVEIK | |
| PD1-0050 | DVQLQESGPGLVKPSQSLSLTCTVTGYSITSDYAWNWIRQFP | 405 |
| VH | GNKLEWMGYITYTGRTSYNPSLKSRISITRDTSKNQFFLQLN | |
| | SVTTEDTATYYCAREMDYYGSTLDYWGQGTTLTVSS | |
| PD1-0050 | KIVLTQSPASLAVSLRQRATISCRASESVDRYGNSFIHWYQQ | 406 |
| VL | KPGQPPKVLIYRASNLESGFPARFSGSGSRTDFTLTIDPVEA | |
| | DDAATYYCQQNNEDPYTFGSGTKLEIK | |
| PD1-0069 | QVQLQQSGPELVRPGVSVKISCKGSGYTFTDYAMHWVKQSHA | 407 |
| VH | RTLEWIGVISTYSGDTNYNQKFKDKATMTVDKSSSTAYLELA | |
| | RMTSEDSAIYYCARLGITTGFAYWGQGTLVTVSA | |
| PD1-0069 | DIVLTQSPASLAVSLGQRATISCRASKGVSTSSYSFMHWYQQ | 408 |
| VL | KPRQPPKLLIKYASYLESGVPARFSGSGSGTDFTLNIHPVEE | |
| | EDAATYYCHHSREFPWTFGGGTKLEIK | |
| PD1-0073 | EVKLVESGGGLVKPGGSLKLSCAASGFTFSNYGMSWIRQTPE | 409 |
| VH | KGLEWVATISGGGRDTYYPDSVKGRFTISRDNVKNNLYLQMS | |
| | SLRSEDTAFYYCASYYYGIDYWGQGTSVTVSS | |
| PD1-0073 | DIVMTQPHKEMSTSVGDRVRITCKASQDVTTAVAWYQQKPGQ | 410 |
| VL | SPKLLIYWASTRHTGVPDRFTGSGSGTEFTLTISSVQAEDLA | |
| | LYYCQQHYSIPWTFGGGTKLEIK | |
| PD1-0078 | QVQLQQPGAELVKPGASVKMSCKASGYTFTSTWMHWVKQRPG | 411 |
| VH | QGLEWIGAIDPSDSYTTYNQKFKGKATLTVDTSSTTAYMQLS | |
| | SLTSEDSAVYYCTRSPFDYWGQGTTLTVSS | |
| PD1-0078 | DIVMTQSHKEMSTSVGDRVSITCKASQDVSTAVAWYQQKPGQ | 412 |
| VL | SPKLLIYSASYRYTGVPDRFTGSGSGTDFTFAISSVQAEDLA | |
| | VYYCQQHYSHPFTFGSGTKLEIK | |
| PD1-0098 | DVQLQESGPGLVKPSQSLSLTCTVTGYSITSDYAWNWIRQFP | 413 |
| VH | GDKLEWLGYITYSGFTNYNPSLKSRISISRDTSKNQFFLQLN | |
| | SVATEDTATYYCARWHGSAPWYFDYWGRGTTLTVSS | |
| PD1-0098 | DVLMTQTPLSLPVSLGDQASISCRSSQNIVHSDGNTYLEWYL | 414 |
| VL | QKPGQSPNLLIYKVSRRFSGVPDRFSGSGSGTDFTLKISRVE | |
| | AEDLGVYYCFQGSHFPLTFGAGTKLELK | |
| PD1-0102 | DVQLQESGPDLVKPSQSLSLTCTVTGYSITSGYSWHWIRQFP | 415 |
| VH | GNKLEWMGFIHSSGDTNYNPSLKSRISFTRDTSKNQFFLQLS | |
| | SLTDEDTATYYCATYRNWYFDVWGAGTTVTVSS | |
| PD1-0102 | DIVMTQSPSSLTVTAGEKVTMRCKSSQSLLNSGTQKNYLTWY | 416 |
| VL | QQKPGQPPKLLIYWASTRESGVPNRFTGSGSGTDFTLTISSV | |
| | QAEDLSVYYCQSDYTFPLTFGGGTKLELK | |
| PD1-112 | QVQLVQSGVEVKKPGASVKVSCKASGYTFTNYYMYWVRQAPG | 417 |
| VH | QGLEWMGGINPSNGGTNFNEKFKNRVTLTTDSSTTTAYMELK | |
| US20200325238 | SLQFDDTAVYYCARRDYRFDMGFDYWGQGTTVTVSS | |
| PD1-112 | EIVLTQSPATLSLSPGERATLSCRASKGVSTSGYSYLHWYQQ | 418 |
| VL | KPGQAPRLLIYLASYLESGVPARFSGSGSGTDFTLTISSLEP | |
| US2020032523 | EDFAVYYCQHSRDLPLTFGGGTKVEIK | |
| PD1-114 | QVQLVESGGGVVQPGRSLRLDCKASGITFSNSGMHWVRQAPG | 419 |
| VH 114 | KGLEWVAVIWYDGSKRYYADSVKGRFTISRDNSKNTLFLQMN | |
| US2020032523 | SLRAEDTAVYYCATNDDYWGQGTLVTVSS | |
| PD1-114 | EIVLTQSPATLSLSPGERATLSCRASQSVSSYLAWYQQKPGQ | 420 |
| VL | APRLLIYDASNRATGIPARFSGSGSGTDFTLTISSLEPEDFA | |
| US20200325238 | VYYCQQSSNWPRTFGQGTKVEIK | |
| PD1-0103 | EVILVESGGGLVKPGGSLKLSCAASGFSFSSYTMSWVRQTPE | 421 |
| (Murine) | KRLDWVATISGGGRDIYYPDSVKGRFTISRDNAKNTLYLEMS | |
| VH | SLMSEDTALYYCVLLTGRVYFALDSWGQGTSVTVSS | |
| PD1-0103 | KIVLTQSPASLPVSLGQRATISCRASESVDTSDNSFIHWYQQ | 422 |
| (Murine) | RPGQSPKLLIYRSSTLESGVPARFSGSGSRTDFTLTIDPVEA | |
| VL | DDVATYYCQQNYDVPWTFGGGTKLEIK | |
| PD1-0103-0314, | GFSFSSYT | 423 |
| PD1-0103-0313, | | |
| PD1-0103-0312, | | |
| and PD1-0103 | | |
| (Murine) | | |
| VH CDR1 | | |
| PD1-0103-0314, | ISGGGRDI | 424 |
| PD1-0103-0313, | | |
| PD1-0103-0312, | | |
| and PD1-0103 | | |
| (Murine) | | |
| VH CDR2 | | |
| PD1-0103-0314, | VLLTGRVYFALDS | 425 |
| PD1-0103-0313, | | |
| PD1-0103-0312, | | |
| and PD1-0103 | | |
| (Murine) | | |
| VH CDR3 | | |
| PD1-0103-0314, | ESVDTSDNSF | 426 |
| PD1-0103-0313, | | |
| PD1-0103-0312, | | |
| and PD1-0103 | | |
| (Murine) | | |
| VL CDR1 | | |
| PD1-0103-0314, | RSS | 427 |
| PD1-0103-0313, | | |
| PD1-0103-0312, | | |
| and PD1-0103 | | |
| (Murine) | | |
| VL CDR2 | | |
| PD1-0103-0314, | NYDVPW | 428 |
| PD1-0103-0313, | | |
| PD1-0103-0312, | | |
| and PD1-0103 | | |
| (Murine) | | |
| VL CDR3 | | |
| PD1-0050 VH | GYSITSDY | 429 |
| CDR1 | | |
| PD1-0050 VH | YTG | 430 |
| CDR2 | | |
| PD1-0050 VH | MDYYGSTLD | 431 |
| CDR3 | | |
| PD1-0050 VL | SESVDRYGNSF | 432 |
| CDR1 | | |
| PD1-0050 VL | RAN | 433 |
| CDR2 | | |
| PD1-0050 VL | NNEDPY | 434 |
| CDR3 | | |
| PD1-0069 VH | GYTFTDY | 435 |
| CDR1 | | |
| PD1-0069 VH | YSG | 436 |
| CDR2 | | |
| PD1-0069 VH | GITTGFA | 437 |
| CDR3 | | |
| PD1-0069 VL | SKGVSTSSYSF | 438 |
| CDR1 | | |
| PD1-0069 VL | YAS | 439 |
| CDR2 | | |
| PD1-0069 VL | SREFPW | 440 |
| CDR3 | | |
| PD1-0073 VH | GFTFSNY | 441 |
| CDR1 | | |
| PD1-0073 VH | GGR | 442 |
| CDR2 | | |
| PD1-0073 VH | YYGID | 443 |
| CDR3 | | |
| PD1-0073 VL | SQDVTTA | 444 |
| CDR1 | | |
| PD1-0073 VL | WAS | 445 |
| CDR2 | | |
| PD1-0073 VL | HYSIPW | 446 |
| CDR3 | | |
| PD1-0078 VH | GYTFTST | 447 |
| CDR1 | | |
| PD1-0078 VH | SDS | 448 |
| CDR2 | | |
| PD1-0078 VH | PFD | 449 |
| CDR3 | | |
| PD1-0078 VL | SQDVSTA | 450 |
| CDR1 | | |
| PD1-0078 VL | SAS | 451 |
| CDR2 | | |
| PD1-0078 VL | HYSHPF | 452 |
| CDR3 | | |
| PD1-0098 VH | GYSITSDY | 453 |
| CDR1 | | |
| PD1-0098 VH | YSG | 454 |
| CDR2 | | |
| PD1-0098 VH | GSAPWYFD | 455 |
| CDR3 | | |
| PD1-0098 VL | SQNIVHSDGNTY | 456 |
| CDR1 | | |
| PD1-0098 VL | KVS | 457 |
| CDR2 | | |
| PD1-0098 VL | SHFPL | 458 |
| CDR3 | | |
| PD1-0102 VH | GYSITSGY | 459 |
| CDR1 | | |
| PD1-0102 VH | SSG | 460 |
| CDR2 | | |
| PD1-0102 VH | RNWYFD | 461 |
| CDR3 | | |
| PD1-0102 VL | SQSLLNSGTQKNY | 462 |
| CDR1 | | |
| PD1-0102 VL | WAS | 463 |
| CDR2 | | |
| PD1-0102 VL | DYTFPL | 464 |
| CDR3 | | |
| PD1-112VH | GYFTNYY | 465 |
| CDR1 | | |
| PD1-112VH | INPSNGGT | 466 |
| CDR2 | | |
| PD1-112VH | ARRDYRFDMGFDY | 467 |
| CDR3 | | |
| PD1-113VL | KGVSTSGYSY | 468 |
| CDR1 | | |
| PD1-113VL | LAS | 469 |
| CDR2 | | |
| PD1-113VL | QHSRDLPLT | 470 |
| CDR3 | | |
| PD1-114VH | GITFSNSG | 471 |
| CDR1 | | |
| PD1-114VH | IWYDGSKR | 472 |
| CDR2 | | |
| PD1-114VH | ATNDDY | 473 |
| CDR3 | | |
| PD1-115VL | QSVSSY | 474 |
| CDR1 | | |
| PD1-115VL | DAS | 475 |
| CDR2 | | |
| PD1-115VL | QQSSNWRRT | 476 |
| CDR3 | | |
| VH_17D8 VH | QVQLVESGGDVVQPGGSLRLSCAASGVAFSNYGMHWVRQAPG | 477 |
| | KGLEWVAVIWYDGSNKYYADSVKGRFTISRDNSKNMLYLQMN | |
| | SLRAEDTAMYYCARNDDYWGQGTLVTVSS | |
| VL_17D8 VL | EIVLTQSPATLSLSPGERATLSCRASQSVSSYLAWYQQKPGQ | 478 |
| | APRLIIYDASNRATGIPARFSGSGSGTDFTLTISSLEPEDFA | |
| | VYYCQQRSNWPLTFGGGTKVEIK | |
| VH_2D3 VH | QVQLVESGGDVVQPGRSLRLSCAASGLTFTNYGFHWVRQAPG | 479 |
| | KGLEWVAVIWYDGSKKYYADSVKGRFTISRDNSKNTLYLQMN | |
| | NLRAEDTAVYYCATGDDYWGQGTLVTVSS | |
| VL_2D3 VL | EIVLTQSPATLSLSPGERATLSCRASQSVSSYLAWYQQKPGQ | 480 |
| | APRLLIYDTSNRATGIPARFSGSGSGTDFTLTISSLEPEDFA | |
| | VYYCQQRSNWPLTFGGGTKVEIK | |
| VH_4H1 VH | QVYLVESGGGVVQPGRSLRLSCAASGFTFSNYGMHWVRQAPG | 481 |
| | KGLEWVALIWYDGSNKYYADSVKGRFTISRDNSKNTLYLQMT | |
| | SLRVEDTAVYYCASNVDHWGQGTLVTVSS | |
| VH_4A11 VH | QLQLQESGPGLVKPSETLSLTCTVSGGSLSRSSFFWGWIRQP | 482 |
| | PGKGLEWIGSIYYSGSTYYNPSLKSRVTISVDTSKNQFSLKL | |
| | SSVTAADTAVYYCVRDYDILIGDEDYWGQGTLVTVSS | |
| VH_7D3 VH | QVQLVESGGGVVQPGRSLRLSCTTSGITFSSYGFHWVRQAPG | 483 |
| | KGLEWVAVIWYDGSKKYYADSVKGRFTLSRDDSKNTLYLQMN | |
| | SLRAEDTAVYYCVTGDDYWGQGTLVTVSS | |
| VL_7D3 VL | EIVLTQSPATLSLSPGERATLSCRASQSVSSYLAWYQQKPGQ | 484 |
| | APRLLIYDASNRATGIPARFSGSGSGTDFTLTISSLEPEDFA | |
| | VYYCQQRSNWPLTFGGGTKVEIK | |
| VH_5F4 VH | QLQLQESGPGLVKPSETLSLTCSVSGGSLSRSSYFWGWIRQP | 485 |
| | PGKGLEWIASIFYSGETYENPSLKSRVTISVDTSRNQFSLKL | |
| | SSVTAADTAVYYCARDYDILTGDEDYWGQGTLVTVSS | |
| VL_5F4 VL | DIQMTQSPSSLSASVGDRVTITCRASQGISSWLAWYQQKPEK | 486 |
| | APKSLIYAASSLQSGVPSRFSGSGSGTDFTLTISSLQPEDFA | |
| | TYYCQQYYSYPRTFGQGTKVEIK | |
| 17D8_VH_CDR1 | NYGMH | 487 |
| 17D8_VH_CDR2 | VIWYDGSNKYYADSVKG | 488 |
| 17D8_VH_CDR3 | NDDY | 489 |
| 17D8_VL_CDR1 | RASQSVSSYLA | 490 |
| 17D8_VL_CDR2 | DASNRAT | 491 |
| 17D8_VL_CDR3 | QQRSNWPLT | 492 |
| 2D3_VH_CDR1 | NYGFH | 493 |
| 2D3_VH_CDR2 | VIWYDGSKKYYADSVKG | 494 |
| 2D3_VH_CDR3 | GDDY | 495 |
| 2D3_VL_CDR1 | RASQSVSSYLA | 496 |
| 2D3_VL_CDR2 | DTSNRAT | 497 |
| 2D3_VL_CDR3 | QQRSNWPLT | 498 |
| 4H1_VH_CDR1 | NYGMH | 499 |
| 4H1_VH_CDR2 | LIWYDGSNKYYADSVKG | 500 |
| 4H1_VH_CDR3 | NVDH | 501 |
| 4A11_VH_CDR1 | RSSFFWG | 502 |
| 4A11_VH_CDR2 | SIYYSGSTYYNPSLKS | 503 |
| 4A11_VH_CDR3 | DYDILTGDEDY | 504 |
| 7D3_VH_CDR1 | SYGFH | 505 |
| 7D3_VH_CDR2 | VIWYDGSKKYYADSVKG | 506 |
| 7D3_VH_CDR3 | GDDY | 507 |
| 7D3_VL_CDR1 | RASQSVSSYLA | 508 |
| 7D3_VL_CDR2 | DASNRAT | 509 |
| 7D3_VL_CDR3 | QQRSNWPLT | 510 |
| 5F4_VH_CDR1 | RSSYFWG | 511 |
| SF4_VH_CDR2 | SIFYSGETYFNPSLKS | 512 |
| 5F4_VH_CDR3 | DYDILTGDEDY | 513 |
| 5F4_VL_CDR1 | RASQGISSWLA | 514 |
| 5F4_VL_CDR2 | AASSLQS | 515 |
| 5F4_VL_CDR3 | QQYYSYPRT | 516 |

An anti-PD-1 polypeptide or an anti-PD-1 antigen binding fragment can comprise a heavy chain or VH having an amino acid sequence of any one of SEQ ID NOS: 332, 334, 336, 338, 340, 342, 344, 346, 348, 350, 352, 354, 356, 358, 360, 362, 364, 366, 368, 370, 372, 374, 401, 405, 407, 409, 411, 413, 415, 417, 419, 421, 477, 479, 481, 482, 483, and 485, or a portion corresponding to a VH thereof. An anti-PD-1 polypeptide or an anti-PD-1 antigen binding fragment can comprise a light chain or VL having an amino acid sequence of any one of SEQ ID NOS: 333, 335, 337, 339, 341, 343, 345, 347, 349, 351, 353, 355, 357, 359, 361, 363, 365, 367, 369, 371, 373, 375, 402, 403, 404, 406, 408, 410, 412, 414, 416, 418,420, 422, 478, 480, 484, and 486, or a portion corresponding to a VL thereof.

In one instance, an anti-PD-1 polypeptide or an anti-PD-1 antigen binding fragment comprises a VH having an amino acid sequence shown in SEQ ID NO: 332, and a VL having an amino acid sequence shown in SEQ ID NO: 333. In another instance, an anti-PD-1 polypeptide or an anti-PD-1 antigen binding fragment comprises a VH having an amino acid sequence shown in SEQ ID NO: 334, and a VL having an amino acid sequence shown in SEQ ID NO: 335. In another instance, an anti-PD-1 polypeptide or an anti-PD-1 antigen binding fragment comprises a VH having an amino acid sequence shown in SEQ ID NO: 336, and a VL having an amino acid sequence shown in SEQ ID NO: 337. In another instance, an anti-PD-1 polypeptide or an anti-PD-1 antigen binding fragment comprises a VH having an amino acid sequence shown in SEQ ID NO: 338, and a VL having an amino acid sequence shown in SEQ ID NO: 339. In another instance, an anti-PD-1 polypeptide or an anti-PD-1 antigen binding fragment comprises a VH having an amino acid sequence shown in SEQ ID NO: 340, and a VL having an amino acid sequence shown in SEQ ID NO: 341. In another instance, an anti-PD-1 polypeptide or an anti-PD-1 antigen binding fragment comprises a VH having an amino acid sequence shown in SEQ ID NO: 342, and a VL having an amino acid sequence shown in SEQ ID NO: 343. In another instance, an anti-PD-1 polypeptide or an anti-PD-1 antigen binding fragment comprises a VH having an amino acid sequence shown in SEQ ID NO: 344, and a VL having an amino acid sequence shown in SEQ ID NO: 345. In another instance, an anti-PD-1 polypeptide or an anti-PD-1 antigen binding fragment comprises a VH having an amino acid sequence shown in SEQ ID NO: 346, and a VL having an amino acid sequence shown in SEQ ID NO: 347. In another instance, an anti-PD-1 polypeptide or an anti-PD-1 antigen binding fragment comprises a VH having an amino acid sequence shown in SEQ ID NO: 48, and a VL having an amino acid sequence shown in SEQ ID NO: 349. In another instance, an anti-PD-1 polypeptide or an anti-PD-1 antigen binding fragment comprises a VH having an amino acid sequence shown in SEQ ID NO: 350, and a VL having an amino acid sequence shown in SEQ ID NO: 351. In another instance, an anti-PD-1 polypeptide or an anti-PD-1 antigen binding fragment comprises a VH having an amino acid sequence shown in SEQ ID NO: 352, and a VL having an amino acid sequence shown in SEQ ID NO: 353. In another instance, an anti-PD-1 polypeptide or an anti-PD-1 antigen binding fragment comprises a VH having an amino acid sequence shown in SEQ ID NO: 354, and a VL having an amino acid sequence shown in SEQ ID NO: 355. In another instance, an anti-PD-1 polypeptide or an anti-PD-1 antigen binding fragment comprises a VH having an amino acid sequence shown in SEQ ID NO: 356, and a VL having an amino acid sequence shown in SEQ ID NO: 357. In another instance, an anti-PD-1 polypeptide or an anti-PD-1 antigen binding fragment comprises a VH having an amino acid sequence shown in SEQ ID NO: 358, and a VL having an amino acid sequence shown in SEQ ID NO: 359. In another instance, an anti-PD-1 polypeptide or an anti-PD-1 antigen binding fragment comprises a VH having an amino acid sequence shown in SEQ ID NO: 360, and a VL having an amino acid sequence shown in SEQ ID NO: 361. In another instance, an anti-PD-1 polypeptide or an anti-PD-1 antigen binding fragment comprises a VH having an amino acid sequence shown in SEQ ID NO: 362, and a VL having an amino acid sequence shown in SEQ ID NO: 363. In another instance, an anti-PD-1 polypeptide or an anti-PD-1 antigen binding fragment comprises a VH having an amino acid sequence shown in SEQ ID NO: 364, and a VL having an amino acid sequence shown in SEQ ID NO: 365. In another instance, an anti-PD-1 polypeptide or an anti-PD-1 antigen binding fragment comprises a VH having an amino acid sequence shown in SEQ ID NO. 366, and a VL having an amino acid sequence shown in SEQ ID NO: 367. In another instance, an anti-PD-1 polypeptide or an anti-PD-1 antigen binding fragment comprises a VH having an amino acid sequence shown in SEQ ID NO: 368, and a VL having an amino acid sequence shown in SEQ ID NO: 369. In another instance, an anti-PD-1 polypeptide or an anti-PD-1 antigen binding fragment comprises a VH having an amino acid sequence shown in SEQ ID NO: 370, and a VL having an amino acid sequence shown in SEQ ID NO: 371. In another instance, an anti-PD-1 polypeptide or an anti-PD-1 antigen binding fragment comprises a VH having an amino acid sequence shown in SEQ ID NO: 372, and a VL having an amino acid sequence shown in SEQ ID NO: 373. In another instance, an anti-PD-1 polypeptide or an anti-PD-1 antigen binding fragment comprises a VH having an amino acid sequence shown in SEQ ID NO: 374, and a VL having an amino acid sequence shown in SEQ ID NO: 375. In another instance, an anti-PD-1 polypeptide or an anti-PD-1 antigen binding fragment comprises a VH having an amino acid sequence shown in SEQ ID NO: 401, and a VL having an amino acid sequence shown in SEQ ID NO: 402. In another instance, an anti-PD-1 polypeptide or an anti-PD-1 antigen binding fragment comprises a VH having an amino acid sequence shown in SEQ ID NO: 401, and a VL having an amino acid sequence shown in SEQ ID NO: 403. In another instance, an anti-PD-1 polypeptide or an anti-PD-1 antigen binding fragment comprises a VH having an amino acid sequence shown in SEQ ID NO: 401, and a VL having an amino acid sequence shown in SEQ ID NO: 404. In another instance, an anti-PD-1 polypeptide or an anti-PD-1 antigen binding fragment comprises a VH having an amino acid sequence shown in SEQ ID NO: 405, and a VL having an amino acid sequence shown in SEQ ID NO: 406. In another instance, an anti-PD-1 polypeptide or an anti-PD-1 antigen binding fragment comprises a VH having an amino acid sequence shown in SEQ ID NO: 407, and a VL having an amino acid sequence shown in SEQ ID NO: 408. In another instance, an anti-PD-1 polypeptide or an anti-PD-1 antigen binding fragment comprises a VH having an amino acid sequence shown in SEQ ID NO: 409, and a VL having an amino acid sequence shown in SEQ ID NO: 410. In another instance, an anti-PD-1 polypeptide or an anti-PD-1 antigen binding fragment comprises a VH having an amino acid sequence shown in SEQ ID NO: 411, and a VL having an amino acid sequence shown in SEQ ID NO: 412. In another instance, an anti-PD-1 polypeptide or an anti-PD-1 antigen binding fragment comprises a VH having an amino acid sequence shown in SEQ ID NO: 413, and a VL having an amino acid sequence shown in SEQ ID NO: 414. In another instance, an anti-PD-1 polypeptide or an anti-PD-1 antigen binding fragment comprises a VH having an amino acid sequence shown in SEQ ID NO: 415, and a VL having an amino acid sequence shown in SEQ ID NO: 416. In another instance, an anti-PD-1 polypeptide or an anti-PD-1 antigen binding fragment comprises a VH having an amino acid sequence shown in SEQ ID NO: 417, and a VL having an amino acid sequence shown in SEQ ID NO: 418. In another instance, an anti-PD-1 polypeptide or an anti-PD-1 antigen binding fragment comprises a VH having an amino acid sequence shown in SEQ ID NO: 419, and a VL having an amino acid sequence shown in SEQ ID NO: 420. In another instance, an anti-PD-1 polypeptide or an anti-PD-1 antigen binding fragment comprises a VH having an amino acid sequence shown in SEQ ID NO: 421, and a VL having an amino acid sequence shown in SEQ ID NO: 422. In another instance, an anti-PD-1 polypeptide or an anti-PD-1 antigen binding fragment comprises a VH having an amino acid sequence shown in SEQ ID NO: 477, and a VL having an amino acid sequence shown in SEQ ID NO: 478. In another instance, an anti-PD-1 polypeptide or an anti-PD-1 antigen binding fragment comprises a VH having an amino acid sequence shown in SEQ ID NO: 479, and a VL having an amino acid sequence shown in SEQ ID NO: 480. In another instance, an anti-PD-1 polypeptide or an anti-PD-1 antigen binding fragment comprises a VH having an amino acid sequence shown in SEQ ID NO: 481, and a VL having an amino acid sequence shown in SEQ ID NO: 347. In another instance, an anti-PD-1 polypeptide or an anti-PD-1 antigen binding fragment comprises a VH having an amino acid sequence shown in SEQ ID NO: 482, and a VL having an amino acid sequence shown in SEQ ID NO: 347. In another instance, an anti-PD-1 polypeptide or an anti-PD-1 antigen binding fragment comprises a VH having an amino acid sequence shown in SEQ ID NO: 483, and a VL having an amino acid sequence shown in SEQ ID NO: 484. In another instance, an anti-PD-1 polypeptide or an anti-PD-1 antigen binding fragment comprises a VH having an amino acid sequence shown in SEQ ID NO: 485, and a VL having an amino acid sequence shown in SEQ ID NO: 486.

In one instance, an anti-PD-1 polypeptide or an anti-PD-1 antigen binding fragment comprises a VH CDR1 having an amino acid sequence of SEQ ID NO: 376, a VH CDR2 having an amino acid sequence of SEQ ID NO: 377, a VH CDR3 having an amino acid sequence of SEQ ID NO: 378, VL CDR1 having an amino acid sequence of SEQ ID NO: 379, a VL CDR2 having an amino acid sequence of SEQ ID NO: 380, and a VL CDR3 having an amino acid sequence of SEQ ID NO: 381. In one instance, an anti-PD-1 polypeptide or an anti-PD-1 antigen binding fragment comprises a VH CDR1 having an amino acid sequence of SEQ ID NO: 382, a VH CDR2 having an amino acid sequence of SEQ ID NO: 383, a VH CDR3 having an amino acid sequence of SEQ ID NO: 384, VL CDR1 having an amino acid sequence of SEQ ID NO: 385, a VL CDR2 having an amino acid sequence of SEQ ID NO: 386, and a VL CDR3 having an amino acid sequence of SEQ ID NO: 387. In one instance, an anti-PD-1 polypeptide or an anti-PD-1 antigen binding fragment comprises a VH CDR1 having an amino acid sequence of SEQ ID NO: 388, a VH CDR2 having an amino acid sequence of SEQ ID NO: 389, a VH CDR3 having an amino acid sequence of SEQ ID NO: 390, VL CDR1 having an amino acid sequence of SEQ ID NO: 391, a VL CDR2 having an amino acid sequence of SEQ ID NO: 392, and a VL CDR3 having an amino acid sequence of SEQ ID NO: 393. In one instance, an anti-PD-1 polypeptide or an anti-PD-1 antigen binding fragment comprises a VH CDR1 having an amino acid sequence of SEQ ID NO: 394, a VH CDR2 having an amino acid sequence of SEQ ID NO: 395, a VH CDR3 having an amino acid sequence of SEQ ID NO: 396, VL CDR1 having an amino acid sequence of SEQ ID NO: 397, a VL CDR2 having an amino acid sequence of SEQ ID NO: 398, and a VL CDR3 having an amino acid sequence of SEQ ID NO: 399. In one instance, an anti-PD-1 polypeptide or an anti-PD-1 antigen binding fragment comprises a VH CDR1 having an amino acid sequence of SEQ ID NO: 423, a VH CDR2 having an amino acid sequence of SEQ ID NO: 424, a VH CDR3 having an amino acid sequence of SEQ ID NO: 425, VL CDR1 having an amino acid sequence of SEQ ID NO: 426, a VL CDR2 having an amino acid sequence of SEQ ID NO: 427, and a VL CDR3 having an amino acid sequence of SEQ ID NO: 428. In one instance, an anti-PD-1 polypeptide or an anti-PD-1 antigen binding fragment comprises a VH CDR1 having an amino acid sequence of SEQ ID NO: 429, a VH CDR2 having an amino acid sequence of SEQ ID NO: 430, a VH CDR3 having an amino acid sequence of SEQ ID NO: 431, VL CDR1 having an amino acid sequence of SEQ ID NO: 432, a VL CDR2 having an amino acid sequence of SEQ ID NO: 433, and a VL CDR3 having an amino acid sequence of SEQ ID NO: 434. In one instance, an anti-PD-1 polypeptide or an anti-PD-1 antigen binding fragment comprises a VH CDR1 having an amino acid sequence of SEQ ID NO: 435, a VH CDR2 having an amino acid sequence of SEQ ID NO. 436, a VH CDR3 having an amino acid sequence of SEQ ID NO: 437, VL CDR1 having an amino acid sequence of SEQ ID NO: 438, a VL CDR2 having an amino acid sequence of SEQ ID NO: 439, and a VL CDR3 having an amino acid sequence of SEQ ID NO: 440. In one instance, an anti-PD-1 polypeptide or an anti-PD-1 antigen binding fragment comprises a VH CDR1 having an amino acid sequence of SEQ ID NO: 441, a VH CDR2 having an amino acid sequence of SEQ ID NO: 442, a VH CDR3 having an amino acid sequence of SEQ ID NO: 443, VL CDR1 having an amino acid sequence of SEQ ID NO: 444, a VL CDR2 having an amino acid sequence of SEQ ID NO: 445, and a VL CDR3 having an amino acid sequence of SEQ ID NO: 446. In one instance, an anti-PD-1 polypeptide or an anti-PD-1 antigen binding fragment comprises a VH CDR1 having an amino acid sequence of SEQ ID NO: 447, a VH CDR2 having an amino acid sequence of SEQ ID NO: 448, a VH CDR3 having an amino acid sequence of SEQ ID NO: 449, VL CDR1 having an amino acid sequence of SEQ ID NO: 450, a VL CDR2 having an amino acid sequence of SEQ ID NO: 451, and a VL CDR3 having an amino acid sequence of SEQ ID NO: 452. In one instance, an anti-PD-1 polypeptide or an anti-PD-1 antigen binding fragment comprises a VH CDR1 having an amino acid sequence of SEQ ID NO: 453, a VH CDR2 having an amino acid sequence of SEQ ID NO: 454, a VH CDR3 having an amino acid sequence of SEQ ID NO: 455, VL CDR1 having an amino acid sequence of SEQ ID NO: 456, a VL CDR2 having an amino acid sequence of SEQ ID NO: 457, and a VL CDR3 having an amino acid sequence of SEQ ID NO: 458. In one instance, an anti-PD-1 polypeptide or an anti-PD-1 antigen binding fragment comprises a VH CDR1 having an amino acid sequence of SEQ ID NO: 459, a VH CDR2 having an amino acid sequence of SEQ ID NO: 460, a VH CDR3 having an amino acid sequence of SEQ ID NO: 461, VL CDR1 having an amino acid sequence of SEQ ID NO: 462, a VL CDR2 having an amino acid sequence of SEQ ID NO: 463, and a VL CDR3 having an amino acid sequence of SEQ ID NO: 464. In one instance, an anti-PD-1 polypeptide or an anti-PD-1 antigen binding fragment comprises a VH CDR1 having an amino acid sequence of SEQ ID NO: 465, a VH CDR2 having an amino acid sequence of SEQ ID NO: 466, a VH CDR3 having an amino acid sequence of SEQ ID NO: 467, VL CDR1 having an amino acid sequence of SEQ ID NO: 468, a VL CDR2 having an amino acid sequence of SEQ ID NO: 469, and a VL CDR3 having an amino acid sequence of SEQ ID NO: 470. In one instance, an anti-PD-1 polypeptide or an anti-PD-1 antigen binding fragment comprises a VH CDR1 having an amino acid sequence of SEQ ID NO: 471, a VH CDR2 having an amino acid sequence of SEQ ID NO: 472, a VH CDR3 having an amino acid sequence of SEQ ID NO: 473, VL CDR1 having an amino acid sequence of SEQ ID NO: 474, a VL CDR2 having an amino acid sequence of SEQ ID NO: 475, and a VL CDR3 having an amino acid sequence of SEQ ID NO: 476. In one instance, an anti-PD-1 polypeptide or an anti-PD-1 antigen binding fragment comprises a VH CDR1 having an amino acid sequence of SEQ ID NO: 487, a VH CDR2 having an amino acid sequence of SEQ ID NO: 488, a VH CDR3 having an amino acid sequence of SEQ ID NO: 489, VL CDR1 having an amino acid sequence of SEQ ID NO: 490, a VL CDR2 having an amino acid sequence of SEQ ID NO: 491, and a VL CDR3 having an amino acid sequence of SEQ ID NO: 492. In one instance, an anti-PD-1 polypeptide or an anti-PD-1 antigen binding fragment comprises a VH CDR1 having an amino acid sequence of SEQ ID NO: 493, a VH CDR2 having an amino acid sequence of SEQ ID NO: 494, a VH CDR3 having an amino acid sequence of SEQ ID NO: 495, VL CDR1 having an amino acid sequence of SEQ ID NO: 496, a VL CDR2 having an amino acid sequence of SEQ ID NO: 497, and a VL CDR3 having an amino acid sequence of SEQ ID NO: 498. In one instance, an anti-PD-1 polypeptide or an anti-PD-1 antigen binding fragment comprises a VH CDR1 having an amino acid sequence of SEQ ID NO: 499, a VH CDR2 having an amino acid sequence of SEQ ID NO: 500, a VH CDR3 having an amino acid sequence of SEQ ID NO: 501, VL CDR1 having an amino acid sequence of SEQ ID NO: 385, a VL CDR2 having an amino acid sequence of SEQ ID NO: 386, and a VL CDR3 having an amino acid sequence of SEQ ID NO: 387. In one instance, an anti-PD-1 polypeptide or an anti-PD-1 antigen binding fragment comprises a VH CDR1 having an amino acid sequence of SEQ ID NO: 502, a VH CDR2 having an amino acid sequence of SEQ ID NO: 503, a VH CDR3 having an amino acid sequence of SEQ ID NO: 504, VL CDR1 having an amino acid sequence of SEQ ID NO: 385, a VL CDR2 having an amino acid sequence of SEQ ID NO: 386, and a VL CDR3 having an amino acid sequence of SEQ ID NO: 387. In one instance, an anti-PD-1 polypeptide or an anti-PD-1 antigen binding fragment comprises a VH CDR1 having an amino acid sequence of SEQ ID NO: 505, a VH CDR2 having an amino acid sequence of SEQ ID NO: 506, a VH CDR3 having an amino acid sequence of SEQ ID NO: 507, VL CDR1 having an amino acid sequence of SEQ ID NO: 508, a VL CDR2 having an amino acid sequence of SEQ ID NO: 509, and a VL CDR3 having an amino acid sequence of SEQ ID NO: 510. In one instance, an anti-PD-1 polypeptide or an anti-PD-1 antigen binding fragment comprises a VH CDR1 having an amino acid sequence of SEQ ID NO: 511, a VH CDR2 having an amino acid sequence of SEQ ID NO: 512, a VH CDR3 having an amino acid sequence of SEQ ID NO: 513, VL CDR1 having an amino acid sequence of SEQ ID NO: 514, a VL CDR2 having an amino acid sequence of SEQ ID NO: 515, and a VL CDR3 having an amino acid sequence of SEQ ID NO: 516.

In one instance, an anti-PD-1 polypeptide comprises a fusion protein. Such fusion protein can be, for example, a two-sided Fc fusion protein comprising the extracellular domain (ECD) of programmed cell death 1 (PD-1) and the ECD of tumor necrosis factor (ligand) superfamily member 4 (TNFSF4 or OX40L) fused via hinge-CH2-CH3 Fc domain of human IgG4, expressed in CHO-K1 cells, where the fusion protein has an exemplary amino acid sequence of SEQ ID NO: 400.

In some embodiments, an activatable immunocytokine as described herein uses LZM-009 as the anti PD-1 antibody. LZM-009 is an IgG4 heavy chain antibody having a heavy chain sequence of QVQLVQSGAEVKKPGASVKVSCKASGYTFTSYYMYWVRQAPGQGLEWMGGVNPSNG GTNFNEKFKSRVTITADKSTSTAYMELSSLRSEDTAVYYCARRDYRYDMGFDYWGQGT TVTVSSASTKGPSVFPLAPCSRSTSESTAALGCLVKDYFPEPVTVSWNSGALTSGVHTFP AVLQSSGLYSLSSVVTVPSSSLGTKTYTCNVDHKPSNTKVDKRVESKYGPPCPPCPAPEF LGGPSVFLFPPKPKDTLMISRTPEVTCVVVDVSQEDPEVQFNWYVDGVEVHNAKTKPRE EQFNSTYRVVSVLTVLHQDWLNGKEYKCKVSNKGLPSSIEKTISKAKGQPREPQVYTLP PSQEEMTKNQVSLTCLVKGFYPSDIAVEWESNGQPENNYKTTPPVLDSDGSFFLYSRLT VDKSRWQEGNVFSCSVMHEALHNHYTQKSLSLSLGK (SEQ ID NO: 517) and a light chain sequence of EIVLTQSPATLSLSPGERATISCRASKGVSTSGYSYLHWYQQKPGQAPRLLIYLASYLESG VPARFSGSGSGTDFTLTISSLEPEDFATYYCQHSRELPLTFGTGTKVEIKRTVAAPSVFIFP PSDEQLKSGTASVVCLLNNFYPREAKVQWKVDNALQSGNSQESVTEQDSKDSTYSLSST LTLSKADYEKHKVYACEVTHQGLSSPVTKSFNRGEC (SEQ ID NO: 518).

### Modification to Fc Region of an Immune Checkpoint Inhibitor Molecule (e.g., Anti-PD-1 Antibody) in Activatable Immunocytokines

In some embodiments, an immune checkpoint inhibitor molecule of an activatable cytokine provided herein is an immune checkpoint inhibitor polypeptide, such as an antibody or antigen binding fragment (e.g., an anti-PD-1 antibody or antigen binding fragment thereof). In some embodiment, the immune checkpoint inhibitor polypeptides (e.g., anti-PD-1 antibody or antigen binding fragment thereof) comprise an Fc region, and the Fc region comprises at least one covalently linked linker. In some embodiments, the linker is covalently attached to an aspartate, asparagine, glutamate, glutamine, cysteine, or lysine residue. In some embodiments, the linker is covalently attached to a lysine or cysteine residue. In some embodiments, the linker is covalently attached to a lysine residue.

In some embodiments, the immune checkpoint inhibitor molecule (e.g., the anti-PD-1 antibody or antigen binding fragment thereof) comprises an Fc region. In some embodiments, the Fc region is an IgG Fc region, an IgA Fc region, an IgD Fc region, an IgM Fc region, or an IgE Fc region. In some embodiments, the Fc region is an IgG Fc region, an IgA Fc region, or an IgD Fc region. In some embodiments, the Fc region is a human Fc region. In some embodiments, the Fc region is a humanized. Fc region. In some embodiments, the Fc region is an IgG Fc region. In some instances, an IgG Fc region is an IgG1 Fc region, an IgG2a Fc region, or an IgG4 Fc region. In some instances, an IgG Fc region is an IgG1 Fc region, an IgG2a Fc region, or an IgG4 Fc region.

One or more mutations may be introduced in an Fc region to reduce Fc-mediated effector functions of an antibody or antigen-binding fragment such as, for example, antibody-dependent cellular cytotoxicity (ADCC) and/or complement function. In some instances, a modified Fc comprises a humanized IgG4 kappa isotype that contains a S228P Fc mutation. In some instances, a modified Fc comprises a human IgG1 kappa where the heavy chain CH2 domain is engineered with a triple mutation such as, for example: (a) L238P, L239E, and P335S; or (2) K248; K288; and K317.

In some embodiments, the Fc region comprises an amino acid sequence at least 80%, at least 85%, at least 90%, at least 91%, at least 92%, at least 93%, at least 94%, at least 95%, at least 96%, at least 97%, at least 98%, or at least 99% identical to a sequence as set forth in SEQ ID NO: 84 (Pro Lys Ser Cys Asp Lys Thr His Thr Cys Pro Pro Cys Pro Ala Pro Glu Xaa Xaa Gly Xaa Pro Ser Val Phe Leu Phe Pro Pro Lys Pro Lys Asp Thr Leu Met lie Ser Arg Thr Pro Glu Val Thr Cys Val Val Val Asp Val Ser His Glu Asp Pro Glu Val Lys Phe Asn Trp Tyr Val Asp Gly Val Glu Val His Asn Ala Lys Thr Lys Pro Arg Glu Glu Gin Tyr Asp Ser Thr Tyr Arg Val Val Ser Val Leu Thr Val Leu His Gln Asp Trp Leu Asn Gly Lys Glu Tyr Lys Cys Lys Val Ser Asn Lys Ala Leu Pro Ala Pro Ile Glu Lys Thr Ile Ser Lys Ala Lys Gly Gln Pro Arg Glu Pro Gln Val Tyr Thr Leu Pro Pro Ser Arg Xaa Glu Xaa Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Xaa Thr Pro Pro Val Leu Asp Ser Asp Gly Ser Phe Phe Leu Tyr Ser Lys Leu Thr Val Asp Lys Ser Arg Trp Gln Gin Gly Asn Val Phe Ser Cys Ser Val Met His Glu Ala Leu His Asn His Tyr Thr Gln Lys Ser Leu Ser Leu Ser Pro Gly, where Xaa can be any naturally occurring amino acid).

In some embodiments, the Fc region comprises one or more mutations which make the Fc region susceptible to modification or conjugation at a particular residue, such as by incorporation of a cysteine residue. In some embodiments, the cysteine residue is incorporated at a position which does not contain a cysteine in SEQ ID NO: 84. Alternatively, the Fc region could be modified to incorporate a modified natural amino acid or an unnatural amino acid which comprises a conjugation handle, such as one connected to the modified natural amino acid or unnatural amino acid through a tether group. In some embodiments, the Fc region does not comprise any mutations which facilitate the attachment of a linker to the IL-2 polypeptide of the activatable immunocytokine. In some embodiments, the linker is attached to a residue as set forth in SEQ ID NO: 84. In some embodiments, the linker is attached to a lysine residue of SEQ ID NO: 84.

In some embodiments, the linker can be covalently attached to one amino acid residue of an Fc region of the immune checkpoint inhibitor molecule (e.g., the anti-PD-1 antibody or antigen binding fragment thereof). In some embodiments, the linker is covalently attached to a non-terminal residue of the Fc region. In some embodiments, the non-terminal residue is in the CH1, CH2, or CH3 region of the anti-PD-1 polypeptide. In some embodiments, the non-terminal residue is in the CH2 region of the anti-PD-1 polypeptide.

In some embodiments, the linker is attached to the Fc region at an amino acid residue at any one of positions 10-90 of SEQ ID NO: 84. In some embodiments, the linker is attached to the Fc region at an amino acid residue at any one of positions 10-20, 10-30, 10-40, 10-50, 10-60, 10-70, 1-80, 10-90, 10-100, 10-110, 10-120, 10-130, 10-140, 10-150, 10-160, 10-170, 10-180, 10-190, or 10-200 of SEQ ID NO: 84. In some embodiments, the linker is attached to the Fc region at an amino acid residue at one of positions 10-30, 50-70, or 80-100 of SEQ ID NO: 84. In some embodiments, the linker is attached to the Fc region at an amino acid residue at any one of positions 20-40, 65-85, or 90-110 of SEQ ID NO: 84. In some embodiments, the linker is attached to the Fc region at an amino acid residue at one of positions 15-26, 55-65, or 85-90 of SEQ ID NO: 84. In some embodiments, the linker is attached to the Fc region at an amino acid residue at any one of positions 25-35, 70-80, or 95-105 of SEQ ID NO: 84. In some embodiments, the linker is attached to the Fc region at an amino acid residue at any one of positions 30, 32, 72, 74, 79 or 101 of SEQ ID NO: 84. In some embodiments, the linker is attached to the Fc region at an amino acid residue at any one of positions 30, 32, 72, 74, or 101 of SEQ ID NO: 84. In some embodiments, the linker is attached to the Fc region at an amino acid residue at any one of positions K30, K32, K72, K74, Q79, or K101 of SEQ ID NO: 84. In some embodiments, the linker is attached to the Fc region at an amino acid residue at any one of positions K30, K32, K72, K74, or K101 of SEQ ID NO: 84. In some embodiments, the linker is attached to the Fc region at amino acid residue 30 of SEQ ID NO: 84. In some embodiments, the linker is attached to the Fc region at amino acid residue 32 of SEQ ID NO: 84. In some embodiments, the linker is attached to the Fc region at amino acid residue 72 of SEQ ID NO: 84. In some embodiments, the linker is attached to the Fc region at amino acid residue 74 of SEQ ID NO: 84. In some embodiments, the linker is attached to the Fc region at amino acid residue 79 of SEQ ID NO: 84. In some embodiments, the linker is attached to the Fc region at amino acid residue 101 of SEQ ID NO: 84.

In some embodiments, the linker is covalently attached at an amino acid residue of the immune checkpoint inhibitor molecule (e.g., the anti-PD-1 antibody or antigen binding fragment thereof) such that the function of the immune checkpoint inhibitor molecule is maintained (e.g., without denaturing the polypeptide). For example, when the immune checkpoint inhibitor molecule (e.g., the anti-PD-1 antibody or antigen binding fragment thereof) is an antibody such as a human IgG (e.g., human IgG1), exposed lysine residues exposed glutamine residues and exposed tyrosine residues are present at the following positions (refer to web site imgt.org/IMGTScientificChart/Numbering/Hu_IGHGnber.html by EU numbering). Exemplary exposed Lysine Residues: CH2 domain (position 246, position 248, position 274, position 288, position 290, position 317, position 320, position 322, and position 338) CH3 domain (position 360, position 414, and position 439). Exemplary exposed Glutamine Residues: CH2 domain (position 295). Exemplary exposed Tyrosine Residues: CH2 domain (position 278, position 296, and position 300) CH3 domain (position 436).

The human IgG, such as human IgG1, may also be modified with a lysine, glutamine, or tyrosine residue at any one of the positions listed above in order provide a residue which is ideally surface exposed for subsequent modification.

In some embodiments, the linker is covalently attached at an amino acid residue in the constant region of the immune checkpoint inhibitor molecule, wherein the immune checkpoint inhibitor molecule is an antibody (e.g., an anti-PD-1 antibody). In some embodiments, the linker is covalently attached at an amino acid residue in the CH1, CH2, or CH3 region. In some embodiments, the linker is covalently attached at an amino acid residue in the CH2 region. In some embodiments, the linker may be covalently attached to one residue selected from the following groups of residues following EU numbering in human IgG Fc: amino acid residues 1-478, amino acid residues 2478, amino acid residues 1-477, amino acid residues 2477, amino acid residues 10467, amino acid residues 30447, amino acid residues 50-427, amino acid residues 100-377, amino acid residues 150-327, amino acid residues 200-327, amino acid residues 240-327, and amino acid residues 240-320.

In some embodiments, the linker is covalently attached to one lysine or glutamine residue of a human IgG Fc region (e.g., a human IgG Fc region of an anti-PD-1 antibody). In some embodiments, the linker is covalently attached at Lys 246 of an Fc region of the antibody (e.g., the anti-PD-1 antibody), wherein amino acid residue position number is based on Eu numbering. In some embodiments, the linker is covalently attached at Lys 248 of an Fc region of the antibody (e.g., the anti-PD-1 antibody), wherein amino acid residue position number is based on Eu numbering. In some embodiments, the linker is covalently attached at Lys 288 of an Fc region of the antibody (e.g., the anti-PD-1 antibody), wherein amino acid residue position number is based on Eu numbering. In some embodiments, the linker is covalently attached at Lys 290 of an Fc region of the antibody (e.g., the anti-PD-1 antibody), wherein amino acid residue position number is based on Eu numbering. In some embodiments, the linker is covalently attached at Gln 295 of an Fc region of the antibody (e.g., the anti-PD-1 antibody), wherein amino acid residue position number is based on Eu numbering. In some embodiments, the linker is covalently attached at Lys 317 of the antibody (e.g., the anti-PD-1 antibody), wherein amino acid residue position number is based on Eu numbering.

In some embodiments, the linker can be covalently attached to an amino acid residue selected from a subset of amino acid residues. In some embodiments, the subset comprises two three, four, five, six, seven, eight, nine, or ten amino acid residues of an Fc region of the antibody (e.g., the anti-PD-1 antibody). In some embodiments, the linker can be covalently attached to one of two lysine residues of an Fc region of the antibody (e.g., the anti-PD-1 antibody).

In some embodiments, the immune checkpoint inhibitor molecule (e.g., the anti-PD-1 antibody) will comprise two linkers covalently attached to the Fc region of the molecule. In some embodiments, each of the two linkers will be covalently attached to a different heavy chain of the anti-PD1 polypeptide. In some embodiments, each of the two linkers will be covalently attached to a different heavy chain of the immune checkpoint inhibitor molecule (e.g., the anti-PD-1 antibody) at a residue position which is the same. In some embodiments, each of the two linkers will be covalently attached to a different heavy chain of immune checkpoint inhibit molecule (e.g., the anti-PD-1 antibody) at a residue position which is different. When the two linkers are covalently attached to residue positions which differ, any combination of the residue positions provided herein may be used in combination.

In some embodiments, a first linker is covalently attached at Lys 248 of a first Fc region of the immune checkpoint inhibitor molecule (e.g., the anti-PD-1 antibody), and a second linker is covalently attached at Lys 288 of a second Fc region of the immune checkpoint inhibitor molecule (e.g., the anti-PD-1 antibody), wherein residue position number is based on Eu numbering. In some embodiments, a first linker is covalently attached at Lys 246 of a first Fc region of the immune checkpoint inhibitor molecule (e.g., the anti-PD-1 antibody), and a second linker is covalently attached at Lys 288 of a second Fc region of the immune checkpoint inhibitor molecule (e.g., the anti-PD-1 antibody), wherein residue position number is based on Eu numbering. In some embodiments, a first linker is covalently attached at Lys 248 of a first Fc region of the immune checkpoint inhibitor molecule (e.g., the anti-PD-1 antibody), and a second linker is covalently attached at Lys 317 of a second Fc region of the immune checkpoint inhibitor molecule (e.g., the anti-PD-1 antibody), wherein residue position number is based on Eu numbering. In some embodiments, a first linker is covalently attached at Lys 246 of a first Fc region of the immune checkpoint inhibitor molecule (e.g., the anti-PD-1 antibody), and a second linker is covalently attached at Lys 317 of a second Fc region of the immune checkpoint inhibitor molecule (e.g., the anti-PD-1 antibody), wherein residue position number is based on Eu numbering. In some embodiments, a first linker is covalently attached at Lys 288 of a first Fc region of the immune checkpoint inhibitor molecule (e.g., the anti-PD-1 antibody), and a second linker is covalently attached at Lys 317 of a second Fc region of the immune checkpoint inhibitor molecule (e.g., the anti-PD-1 antibody), wherein residue position number is based on Eu numbering.

In some embodiments, an immunocytokine composition provided herein (e.g., an anti-PD-1 antibody or antigen binding fragment attached to an IL-2 polypeptide through a linker) maintains binding affinity associated with the antibody or antigen binding fragments after formation of the linkage between the two groups (or a corresponding antibody or antigen binding fragment which is not attached to an IL-2 polypeptide). For example, in an immunocytokine composition comprising an antibody or antigen binding fragment linked to an IL-2 polypeptide, in some embodiments antibody or antigen binding fragment thereof retains binding to one or more Fc receptors. In some embodiments, the immunocytokine composition displays binding to one or more Fc receptors which is reduced by no more than about 5-fold, no more than about 10-fold, no more than about 15-fold, or no more than about 20-fold compared to the unattached antibody or antigen binding fragment. In some embodiments, the one or more Fc receptors is the FcRn receptor, the FcγRI receptor (CD64), the FcγRIIa receptor (CD32α), the FcγRIIβ receptor (CD32β), the FcγRIII receptor (CD16α), or any combination thereof. In some embodiments, binding of the immunocytokine composition to each of the FcRn receptor, the FcγRI receptor (CD64), the FcγRIIa receptor (CD32α), and the FcγRIIβ receptor (CD320), the FcγRIII receptor (CD16α), is reduced by no more than about 10-fold compared to the unattached antibody or antigen binding fragment.

In some embodiments, binding of the antibody or antigen binding fragment (e.g., the anti-PD-1 antibody or antigen binding fragment) is substantially unaffected by the conjugation with the IL-2 polypeptide. In some embodiments, the binding of antibody or antigen binding fragment to its target (e.g., PD-1) is reduced by no more than about 5% compared to the unconjugated antibody.

### Method of Modifying an Fc Region

Also provided herein are method of preparing a modified Fc region of an immune checkpoint inhibitor molecule (e.g., an anti-PD-1 antibody), such as for the attachment of a linker, a conjugation handle, or the IL-2 polypeptide to the immune checkpoint inhibitor molecule to form the activatable immunocytokine. A variety of methods for site-specific modification of Fc regions of antibodies or other polypeptides are known in the art. Modification with an affinity peptide configured to site-specifically attach linker to an Fc region (e.g., of an antibody such as an anti-PD-1 antibody)

In some embodiments, an Fc region of an immune checkpoint inhibitor molecule (e.g., an antibody or antigen binding fragment such as an anti-PD-1 antibody or antigen binding fragment thereof) is modified to incorporate a linker, a conjugation handle, or a combination thereof, which forms or is used to form the activatable immunocytokine. In some embodiments, the modification is performed by contacting the Fc region with an affinity peptide bearing a payload configured to attach a linker or other group to the Fc region, such as at a specific residue of the Fc region. In some embodiments, the linker (or portion thereof) is attached using a reactive group (e.g., a N-hydroxysuccinimide ester) which forms a bond with a residue of the Fc region. In some embodiments, the affinity peptide comprises a cleavable linkage. The cleavable linkage is configured on the affinity peptide such that after the linker or a portion thereof or other group is attached to the Fc region, the affinity peptide can be removed, leaving behind only the desired linker or portion thereof or other group attached to the Fc region. The linker or portion thereof or other group can then be used further to add attach the IL-2 polypeptide to the Fc region.

Non-limiting examples of such affinity peptides can be found at least in PCT Publication No. WO2018199337A1, PCT Publication No. WO2019240288A1, PCT Publication No. WO2019240287A1, and PCT Publication No. WO2020090979A1, each of which is incorporated by reference as if set forth herein in its entirety. In some embodiments, the affinity peptide is a peptide which has been modified to deliver the linker (or portion thereof)/conjugation handle payload to one or more specific residues of the Fc region of the antibody. In some embodiments, the affinity peptide has at least 80%, 85%, 90%, 91%, 92%, 93%, 94%, 95%, 96%, 97%, 98%, 99%, or 100% sequence identify to a peptide selected from among (1) QETNPTENLYFQQKNMQCQRRFYEALHDPNLNEEQRNARIRSIRDDDC (SEQ ID NO. 85); (2) QTADNQKNMQCQRRFYEALHDPNLNEEQRNARIRSIRDDCSQSANLLAEAQQLNDAQA PQA (SEQ ID NO: 86); (3) QETKNMQCQRRFYEALHDPNLNEEQRNARIRSIRDDDC (SEQ ID NO: 87); (4) QETFNKQCQRRFYEALHDPNLNEEQRNARIRSIRDDDC (SEQ ID NO: 88); (5) QETFNMQCQRRFYEALHDPNLNKEQRNARIRSIRDDDC (SEQ ID NO: 89); (6) QETFNMQCQRRFYEALHDPNLNEEQRNARIRSIKDDC (SEQ ID NO: 90); (7) QETMQCQRRFYEALHDPNLNEEQRNARIRSIKDDC (SEQ ID NO: 91); (8) QETQCQRRFYEALHDPNLNEEQRNARIRSIKDDC (SEQ ID NO. 92); (9) QETCQRRFYEALHDPNLNEEQRNARIRSIKDDC (SEQ ID NO: 93); (10) QETRGNCAYHKGQLVWCTYH (SEQ ID NO: 94); and (11) QETRGNCAYHKGQIIWCTYH (SEQ ID NO: 95), or a corresponding peptide which has been truncated at the N-terminus by one, two, three, four, or five residues. An exemplary affinity peptide with cleavable linkage and conjugation handle payload capable of attaching the payload to residue K248 of an antibody as provided herein is shown below (SEQ ID NO: 682) (as reported in Matsuda et al., “Chemical Site-Specific Conjugation Platform to Improve the Pharmacokinetics and Therapeutic Index of Antibody-Drug Conjugates,” Mol. Pharmaceutics 2021, 18, 11, 4058-4066).

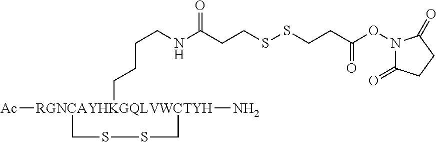

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide a sulfhydryl protecting group as a cleavable portion of the affinity peptide (e.g., the relevant portion of the affinity peptide would have a structure of

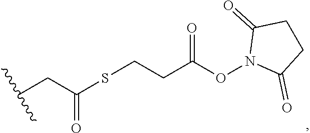 [SMILES: *CC(=O)SCCC(=O)ON1C(=O)CCC1=O]

or another of the cleavable linkers discussed below).

The affinity peptide of the disclosure can comprise a cleavable linkage. In some embodiments, the cleavable linkage of the affinity peptide connects the affinity peptide to the group which is to be attached to the Fc region and is configured such that the peptide can be cleaved after the group comprising the linker (or portion thereof) or conjugation handle has been attached. In some embodiments, the cleavable linkage is a divalent group. In some embodiments, the cleavable linkage can comprise a thioester group, an ester group, a sulfane group; a methanimine group; an oxyvinyl group; a thiopropanoate group; an ethane-1,2-diol group; an (imidazole-1-yl)methan-1-one group; a seleno ether group; a silylether group; a di-oxysilane group; an ether group; a di-oxymethane group; a tetraoxospiro[5.5]undecane group; an acetamidoethyl phosphoramidite group; a bis(methylthio)-pyrazolopyrazole-dione group; a 2-oxo-2-phenylethyl formate group; a 4-oxybenzylcarbamate group; a 2-(4-hydroxy-oxyphenyl)diazinyl)benzoic acid group; a 4-amino-2-(2-amino-2-oxoethyl)-4-oxobut-2-enoic acid group; a 2-(2-methylenehydrazineyl)pyridine group; an N′-methyleneformohydrazide group; or an isopropylcarbamate group, any of which is unsubstituted or substituted. Composition and points of attachment of the cleavable linker to the affinity peptide, as well as related methods of use, are described in, at least, PCT Publication No. WO2018199337A1, PCT Publication No. WO2019240288A1, PCT Publication No. WO2019240287A1, and PCT Publication No. WO2020090979A1.

In some embodiments, the cleavable linkage is:

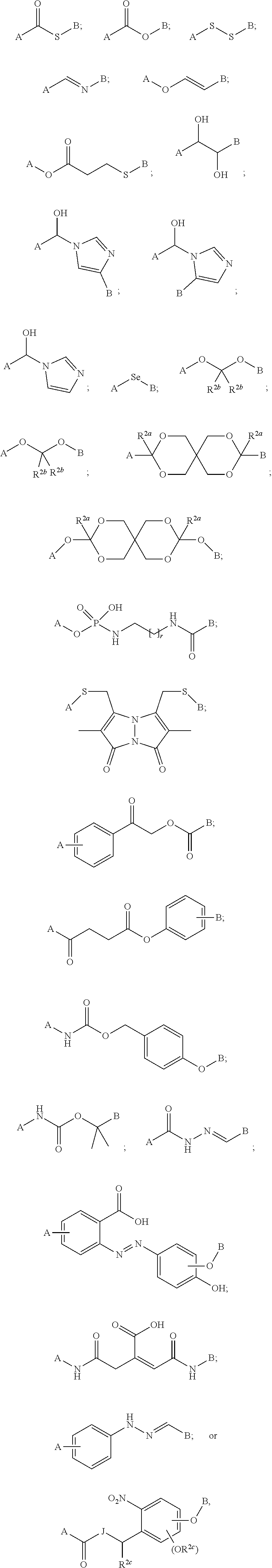

wherein:
one of A or B is a point of attachment the linker and the other of A or B is a point of attachment to the affinity peptide;
each R^{2a} is independently H or optionally substituted alkyl;
each R^{2b} is independently H or optionally substituted alkyl;
R^{2c} is a H or optionally substituted alkyl;
J is a methylene, a N, a S, a Si, or an O atom; and
r is 1, 2, 3, 4, 5, 6, 7, 8, 9, or 10.

The affinity peptide comprises a reactive group which is configured to enable the covalent attachment of the linker (or portion thereof)/conjugation handle to the Fc region. In some embodiments, the reactive group is selective for a functional group of a specific amino acid residue, such as a lysine residue, tyrosine residue, serine residue, cysteine residue, or an unnatural amino acid residue of the Fc region incorporated to facilitate the attachment of the linker. The reactive group may be any suitable functional group, such as an activated ester for reaction with a lysine (e.g., N-hydroxysuccinimide ester or a derivate thereof, a pentafluorophenyl ester, etc.) or a sulfhydryl reactive group for reaction with a cysteine (e.g., a Michael acceptor, such as an alpha-beta unsaturated carbonyl, a maleimide, an alpha-halo carbonyl, etc.). In some embodiments, the reactive group is:

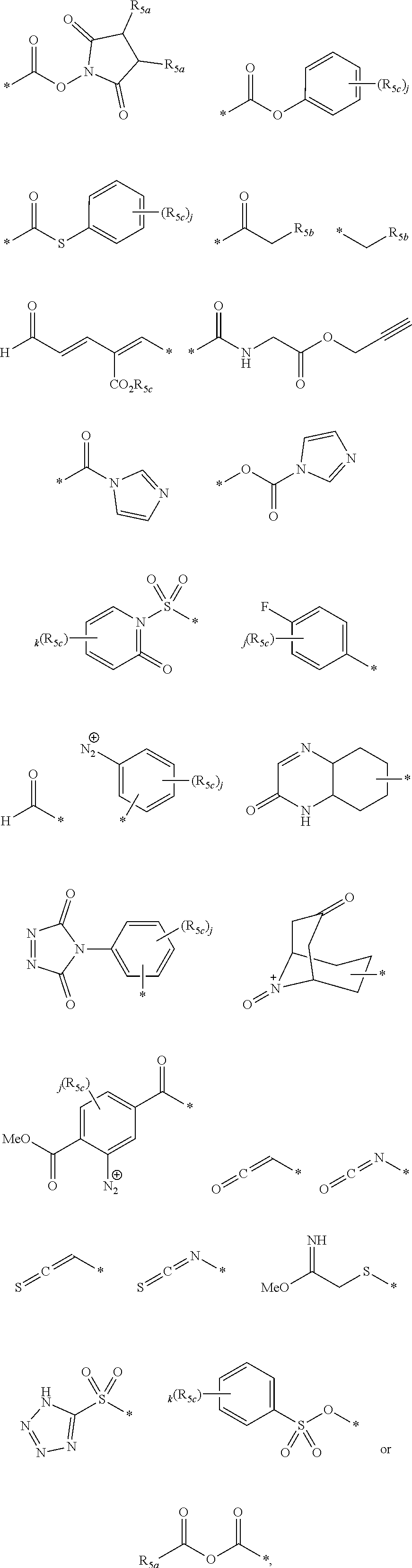

wherein:
each R₅ₐ, R_{5b}, and R_{5c} is independently H, halogen, or optionally substituted alkyl;
each j is 1, 2, 3, 4, or 5; and
each k is 1, 2, 3, 4, or 5.

In some embodiments, the affinity peptide is used to deliver a reactive moiety to the desired amino acid residue such that the reactive moiety is exposed upon cleavage of the cleavable linker. By way of non-limiting example, the reactive group forms a covalent bond with a desired residue of the Fc region of the polypeptide which selectively binds to anti-PD-1 due to an interaction between the affinity peptide and the Fc region. Following this covalent bond formation, the cleavable linker is cleaved under appropriate conditions to reveal a reactive moiety (e.g., if the cleavable linker comprises a thioester, a free sulfhydryl group is attached to the Fc region following cleavage of the cleavable linkage). This new reactive moiety can then be used to subsequently add an additional moiety, such as a conjugation handle, by way of reagent comprising the conjugation handle tethered to a sulfhydryl reactive group (e.g., alpha-halogenated carbonyl group, alpha-beta unsaturated carbonyl group, maleimide group, etc.).

In some embodiments, an affinity peptide is used to deliver a free sulfhydryl group to a lysine of the Fc region. In some embodiments, the free sulfhydryl group is then reacted with a bifunctional linking reagent to attach a new conjugation handle to the Fc region. In some embodiments, the new conjugation handle is then used to form the linker to the attached cytokine. In some embodiments, the new conjugation handle is an alkyne functional group. In some embodiments, the new conjugation handle is a DBCO functional group.

Exemplary bifunctional linking reagents useful for this purpose are of a formula A-B-C, wherein A is the sulfhydryl reactive conjugation handle (e.g., maleimide, α,β-unsaturated carbonyl, α-halogenated carbonyl), B is a linkage group, and C is the new conjugation handle (e.g., an alkyne such as DBCO). Specific non-limiting examples of bifunctional linking reagents include

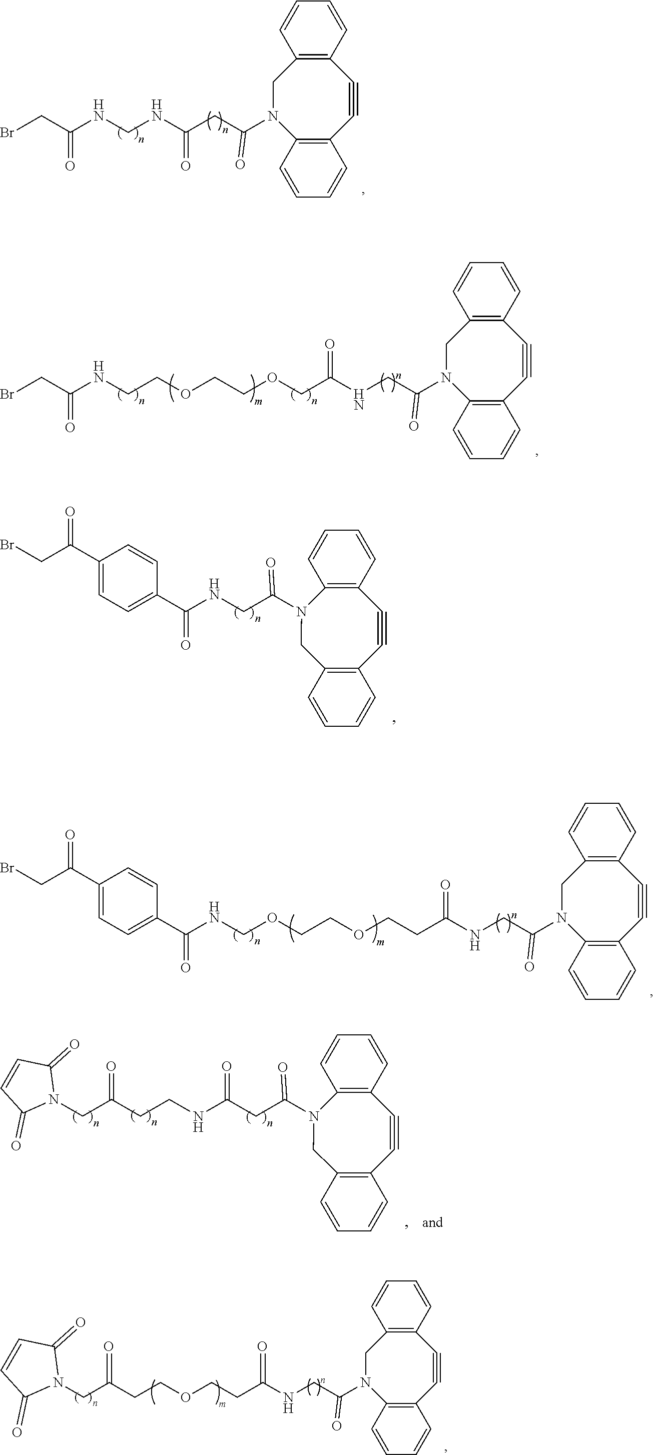

wherein each n is independently an integer from 1-6 and each m is independently an integer from 1-30, and related molecules (e.g., isomers).

Alternatively, the affinity peptide can be configured such that a conjugation handle is added to the Fc region (such as by a linker group) immediately after covalent bond formation between the reactive group and a residue of the Fc region. In such cases, the affinity peptide is cleaved and the conjugation handle is immediately ready for subsequent conjugation to the IL-2 polypeptide to form an activatable immunocytokine.

### Alternative Methods of Attachment of Linker (or Portion Thereof) to Immune Checkpoint Inhibitor Molecules—Enzyme Mediated

While the affinity peptide mediated modification of an immune checkpoint inhibitor molecule (e.g., an Fc region of an antibody such as an anti-PD-1 antibody) provided supra possesses many advantages over other methods which can be used to site-specifically modify the immune checkpoint inhibitor molecule (e.g., ease of use, ability to rapidly generate many different antibody conjugates, ability to use many “off-the-shelf” commercial antibodies without the need to do time consuming protein engineering, etc.), other methods of performing the modification are also contemplated as being within the scope of the present disclosure.

In some embodiments, the present disclosure relates generally to transglutaminase-mediated site-specific antibody-drug conjugates (ADCs) comprising: 1) glutamine-containing tags, endogenous glutamines (e.g., native glutamines without engineering, such as glutamines in variable domains, CDRs, etc.), and/or endogenous glutamines made reactive by antibody engineering or an engineered transglutaminase; and 2) amine donor agents comprising amine donor units, linkers, and agent moieties. Non-limiting examples of such transglutaminase mediated site-specific modifications can be found at least in publications WO2020188061, US2022133904, US2019194641, US2021128743, U.S. Pat. No. 9,764,038, U.S. Ser. No. 10/675,359, U.S. Pat. No. 9,717,803, U.S. Ser. No. 10/434,180, U.S. Pat. No. 9,427,478, which are incorporated by reference as if set forth herein in their entirety.

In another aspect, the disclosure provides an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or different amino acid), and wherein the engineered Fc-containing polypeptide conjugate comprises an amino acid substitution from glutamine to asparagine at position 295 (Q295N; EU numbering scheme).

In some embodiments, the acyl donor glutamine-containing tag is not spatially adjacent to a reactive Lys (e.g., the ability to form a covalent bond as an amine donor in the presence of an acyl donor and a transglutaminase) in the polypeptide or the Fc-containing polypeptide. In some embodiments, the polypeptide or the Fc-containing polypeptide comprises an amino acid modification at the last amino acid position in the carboxyl terminus relative to a wild-type polypeptide at the same position. The amino acid modification can be an amino acid deletion, insertion, substitution, mutation, or any combination thereof.

In some embodiments, the polypeptide conjugate comprises a full length antibody heavy chain and an antibody light chain, wherein the acyl donor glutamine-containing tag is located at the carboxyl terminus of a heavy chain, a light chain, or both the heavy chain and the light chain.

In some embodiments, the polypeptide conjugate comprises an antibody, wherein the antibody is a monoclonal antibody, a polyclonal antibody, a human antibody, a humanized antibody, a chimeric antibody, a bispecific antibody, a minibody, a diabody, or an antibody fragment. In some embodiments, the antibody is an IgG.

In another aspect, described herein is a method for preparing an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or a different amino acid), and wherein the engineered Fc-containing polypeptide conjugate comprises an amino acid substitution from glutamine to asparagine at position 295 (Q295N; EU numbering scheme), comprising the steps of: a) providing an engineered (Fc-containing polypeptide)-T molecule comprising the Fc-containing polypeptide and the acyl donor glutamine-containing tag; b) contacting the amine donor agent with the engineered (Fc-containing polypeptide)-T molecule in the presence of a transglutaminase; and c) allowing the engineered (Fc-containing polypeptide)-T to covalently link to the amine donor agent to form the engineered Fc-containing polypeptide conjugate.

In another aspect, described herein is a method for preparing an engineered polypeptide conjugate comprising the formula: polypeptide-T-A, wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the polypeptide, and wherein the acyl donor glutamine-containing tag comprises an amino acid sequence LLQGPX (SEQ ID NO: 96), wherein X is A or P, or GGLLQGPP (SEQ ID NO: 97), comprising the steps of: a) providing an engineered polypeptide-T molecule comprising the polypeptide and the acyl donor glutamine-containing tag; b) contacting the amine donor agent with the engineered polypeptide-T molecule in the presence of a transglutaminase; and c) allowing the engineered polypeptide-T to covalently link to the amine donor agent to form the engineered Fc-containing polypeptide conjugate.

In some embodiments, the engineered polypeptide conjugate (e.g., the engineered Fc-containing polypeptide conjugate, the engineered Fab-containing polypeptide conjugate, or the engineered antibody conjugate) as described herein has conjugation efficiency of at least about 51%. In another aspect, the invention provides a pharmaceutical composition comprising the engineered polypeptide conjugate as described herein (e.g., the engineered Fc-containing polypeptide conjugate, the engineered Fab-containing polypeptide conjugate, or the engineered antibody conjugate) and a pharmaceutically acceptable excipient.

In some embodiments, described herein is a method for conjugating a linker or portion thereof to an immune checkpoint inhibitor molecule to form an activatable immunocytokine, comprising the steps of: (a) providing an antibody (e.g., an anti-PD-1 antibody) having (e.g., within the primary sequence of a constant region) at least one acceptor amino acid residue (e.g., a naturally occurring amino acid) that is reactive with a linking reagent (linker) in the presence of a coupling enzyme, e.g., a transamidase; and (b) reacting said antibody with a linking reagent (e.g., a linker comprising a primary amine) comprising a reactive group (R), optionally a protected reactive group or optionally an unprotected reactive group, in the presence of an enzyme capable of causing the formation of a covalent bond between the acceptor amino acid residue and the linking reagent (other than at the R moiety), under conditions sufficient to obtain an antibody comprising an acceptor amino acid residue linked (covalently) to a reactive group (R) via the linking reagent. Optionally, said acceptor residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH2 domain Optionally, the antibody is free of heavy chain N297-linked glycosylation. Optionally, the acceptor glutamine is at position 295 and the residue at the +2 position is the residue at position 297 (EU index numbering) of an antibody heavy chain.

In one aspect, described herein is a method for conjugating a moiety of interest (Z) to immune checkpoint inhibitor molecule to form an activatable immunocytokine, comprising the steps of: (a) providing an antibody (e.g., an anti-PD-1 antibody) having at least one acceptor glutamine residue; and (b) reacting said antibody with a linker comprising a primary amine (a lysine-based linker) comprising a reactive group (R), preferably a protected reactive group, in the presence of a transglutaminase (TGase), under conditions sufficient to obtain an antibody comprising an acceptor glutamine linked (covalently) to a reactive group (R) via said linker. Optionally, said acceptor glutamine residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH2 domain Optionally, the antibody is free of heavy chain N297-linked glycosylation. Optionally, the acceptor glutamine is at position 295 and the residue at the +2 position is the residue at position 297 (EU index numbering) of an antibody heavy chain.

The antibody (e.g., the anti-PD-1 antibody) comprising an acceptor residue or acceptor glutamine residue linked to a reactive group (R) via a linker comprising a primary amine (a lysine-based linker) can thereafter be reacted with a reaction partner comprising a moiety of interest (Z) to generate an antibody comprising an acceptor residue or acceptor glutamine residue linked to a moiety of interest (Z) via the linker. Thus, in one embodiment, the method further comprises a step (c): reacting (i) an antibody of step b) comprising an acceptor glutamine linked to a reactive group (R) via a linker comprising a primary amine (a lysine-based linker), optionally immobilized on a solid support, with (ii) a compound comprising a moiety of interest (Z) and a reactive group (R′) capable of reacting with reactive group R, under conditions sufficient to obtain an antibody comprising an acceptor glutamine linked to a moiety of interest (Z) via a linker comprising a primary amine (a lysine-based linker). Preferably, said compound comprising a moiety of interest (Z) and a reactive group (R′) capable of reacting with reactive group R is provided at a less than 80 times, 40 times, 20 times, 10 times, 5 times or 4 molar equivalents to the antibody. In one embodiment, the antibody comprises two acceptor glutamines and the compound comprising a moiety of interest (Z) and a reactive group (R′) is provided at 10 or less molar equivalents to the antibody. In one embodiment, the antibody comprises two acceptor glutamines and the compound comprising a moiety of interest (Z) and a reactive group (R′) is provided at 5 or less molar equivalents to the antibody. In one embodiment, the antibody comprises four acceptor glutamines and the compound comprising a moiety of interest (Z) and a reactive group (R′) is provided at 20 or less molar equivalents to the antibody. In one embodiment, the antibody comprises four acceptor glutamines and the compound comprising a moiety of interest (Z) and a reactive group (R′) is provided at 10 or less molar equivalents to the antibody. In one embodiment, steps (b) and/or (c) are carried out in aqueous conditions. Optionally, step (c) comprises: immobilizing a sample of an antibody comprising a functionalized acceptor glutamine residue on a solid support to provide a sample comprising immobilized antibodies, reacting the sample comprising immobilized antibodies with a compound, optionally recovering any unreacted compound and re-introducing such recovered compound to the solid support for reaction with immobilized antibodies, and eluting the antibody conjugates to provide a composition comprising a Z moiety.

### Conjugation Handle Chemistry

In some embodiments, the appropriately modified Fc region of the immune checkpoint inhibitor molecule (e.g., an antibody or antigen binding fragment, such as an anti-PD-1 antibody or antigen binding fragment thereof) will comprise a conjugation handle which is used to conjugate the immune checkpoint inhibitor molecule to an IL-2 polypeptide as provided herein to form an activatable immunocytokine.

Any suitable reactive group capable of reacting with a complementary reactive group attached to the IL-2 polypeptide can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cycloctenes, or a metal-mediated process such as olefin metathesis and Suzuki-Miyaura or Sonogashira cross-coupling.

In some embodiments, the conjugation handle comprises a reagent for a “copper-free” alkyne-azide triazole-forming reaction. Non-limiting examples of alkynes for said alkyne-azide triazole-forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof). In some embodiments, the alkyne functional group is attached to the Fc region. In some embodiments, the azide functional group is attached to the Fc region.

In some embodiments, the conjugation handle comprises a reactive group selected from azide, alkyne, tetrazine, halide, sulfhydryl, disulfide, maleimide, activated ester, alkene, aldehyde, ketone, imine, hydrazine, and hydrazide. In some embodiments, the IL-2 polypeptide comprises a reactive group complementary to the conjugation handle of the Fc region. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

### Linker Structure

In some embodiments, the linker used to attach the immune checkpoint inhibitor molecule (e.g., the anti-PD-1 antibody or antigen binding fragment thereof) and the IL-2 polypeptide comprises points of attachment at both groups. The points of attachment can be any of the residues for facilitating the attachment as provided herein. The linker structure can be any suitable structure for creating the spatial attachment between the two moieties. In some embodiments, the linker provides covalent attachment of both moieties (e.g., the IL-2 polypeptide and the immune checkpoint inhibitor molecule, such as the anti-PD-1 antibody or antigen binding fragment thereof). In some embodiments, the linker is a chemical linker (e.g., not an expressed polypeptide as in a fusion protein).

In some embodiments, the linker is a chemical linker group. In some embodiments, the linker comprises at least one portion which is not comprised of amino acid residues. In some embodiments, the linker comprises a polymer. In some embodiments, the linker comprises a non-polymer. In some embodiments, the linker comprises a polymer and a non-polymer (e.g., a polymeric portion and a non-polymeric portion).

In some embodiments, the linker comprises a polymer. In some embodiments, the linker comprises a water soluble polymer. In some embodiments, the linker comprises poly(alkylene oxide), polysaccharide, poly(vinyl pyrrolidone), poly(vinyl alcohol), polyoxazoline, poly(acryloylmorpholine), or a combination thereof. In some embodiments, the linker comprises poly(alkylene oxide). In some embodiments, the poly(alkylene oxide) is polyethylene glycol or polypropylene glycol, or a combination thereof. In some embodiments, the poly(alkylene oxide) is polyethylene glycol.

In some embodiments, the linker is a bifunctional linker. In some embodiments, the bifunctional linker comprises an amide group, an ester group, an ether group, a thioether group, or a carbonyl group. In some embodiments, the linker comprises a non-polymer linker. In some embodiments, the linker comprises a non-polymer, bifunctional linker. In some embodiments, the non-polymer, bifunctional linker comprises succinimidyl 4-(N-maleimidomethyl)cyclohexane-1-carboxylate; Maleimidocaproyl; Valine-citrulline; Allyl(4-methoxyphenyl)dimethylsilane; 6-(Allyloxycarbonylamino)-1-hexanol; 4-Aminobutyraldehyde diethyl acetal; or (E)-N-(2-Aminoethyl)-4-{2-[4-(3-azidopropoxy)phenyl]diazenyl}benzamide hydrochloride.

The linker can be branched or linear. In some embodiments, the linker is linear. In some embodiments, the linker is branched. In some embodiments, the linker comprises a linear portion (e.g., between the first point of attachment and the second point of attachment) of a chain of at least 10, 20, 50, 100, 500, 1000, 2000, 3000, or 5000 atoms. In some embodiments, the linker comprises a linear portion of a chain of at least 10, 20, 30, 40, or 50 atoms. In some embodiments, the linker comprises a linear portion of at least 10 atoms. In some embodiments, the linker is branched and comprises a linear portion of a chain of at least 10, 20, 50, 100, 500, 1000, 2000, 3000, or 5000 atoms. In some embodiments, the linker comprises a linear chain of at most 5000, 3000, 2000, 1000, 500, 400, or 300 atoms.

In some embodiments, the linker has a molecular weight of about 50 Daltons to about 2000 Daltons. In some embodiments, the linker has a molecular weight of about 50 Daltons to about 5000 Daltons. In some embodiments, the linker has a molecular weight of 200 Daltons to 100,000 Daltons. In a preferred embodiments, the linker has a molecular weight of less than 5000 Daltons, less than 4000 Daltons, less than 3000 Daltons, or less than 2000 Daltons, and the linker is monodisperse (e.g., for a population of conjugate compositions herein, there is a high degree of uniformity of the linker between the immune checkpoint inhibitor molecule (e.g., the anti-PD-1 antibody or antigen binding fragment thereof) and the IL-2 polypeptide.

In some embodiments, the linker comprises a reaction product one or more pairs of conjugation handles and a complementary conjugation handle thereof. In some embodiments, the reaction product comprises a triazole, a hydrazone, pyridazine, a sulfide, a disulfide, an amide, an ester, an ether, an oxime, an alkene, or any combination thereof. In some embodiments, the reaction product comprises a triazole. The reaction product can be separated from the first point of attachment and the second point of attachment by any portion of the linker. In some embodiments, the reaction product is substantially in the center of the linker. In some embodiments, the reaction product is substantially closer to one point of attachment than the other.

In some embodiments, the linker comprises a structure of Formula (X)

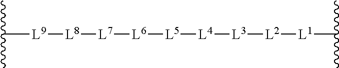

wherein each of L¹, L², L³, L⁴, L⁵, L⁶, L⁷, L⁸, and L⁹ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —C(═O)(C₁-C₆ alkylene)S—, —S(C₁-C₆ alkylene)C(═O)—, —S(═O)—, —S(═O)₂—, —C(═O)—, —(C₁-C₆ alkylene)C(═O)—, —C(═O) (C₁-C₆ alkylene)-, —C(═O)(C₁-C₆ alkylene)C(═O)—, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}—C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}—C(═O)NR^{L}—, —NR^{L}C(═S)NR^{L}—, —CR^{L}=N—, —N═CR^{L}, —NR—S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}, —(C₁-C₆ alkyl)(CH₂—CH₂—O)_{qa}—, (C₁-C₆ alkyl)(O—CH₂—CH₂)_{qb}—, (CH₂—CH(CH₃)—O)_{qc}—, —(O—CH(CH)—CH₂)_{qd}—, a reaction product of a conjugation handle and a complementary conjugation handle, or absent; (C₁-C₆ alkylene)
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₅ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

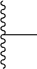

is a point of attachment to the immune checkpoint inhibitor molecule (e.g., the anti-PD-1 antibody or antigen binding fragment thereof) or the IL-2 polypeptide.

In some embodiments, the linker comprises a structure of Formula (X′)

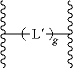

wherein each L′ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}=N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, (O—CH₂—CH₂)_{qb}—, —(CH₂—CH(CH₃)—O)_{qc}—, —(O—CH(CH₃)—CH₂)_{qa}—, a reaction product of a conjugation handle and a complementary conjugation handle, or absent;
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₅ cycloalkyl, substituted or unsubstituted C2-C7 heterocycloalkyl, substituted or unsubstituted aryl, or substituted or unsubstituted heteroaryl;
each of qa, qb, qc and qd is independently an integer from 1-100, and,
g is an integer from 1-100,
wherein each

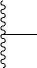

is a point of attachment to the modified IL-2 polypeptide or immune checkpoint inhibitor molecule (e.g., the anti-PD-1 antibody or antigen binding fragment thereof).

In some embodiments, the linker of Formula (X) or of Formula (X′) comprises the structure:

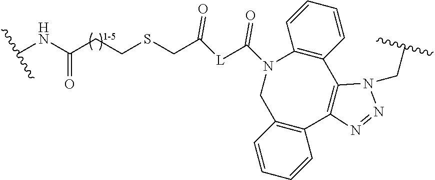

wherein

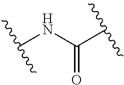 [SMILES: *NC(*)=O]

is the point of attachment to a lysine residue of the immune checkpoint inhibitor molecule (e.g., the anti-PD-1 antibody or antigen binding fragment thereof);
L is a tether group; and

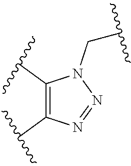 [SMILES: *Cn1nnc(*)c1*]

is a point of attachment to a tether group which connects to the IL-2 polypeptide, or a regioisomer thereof.

In some embodiments, L has a structure

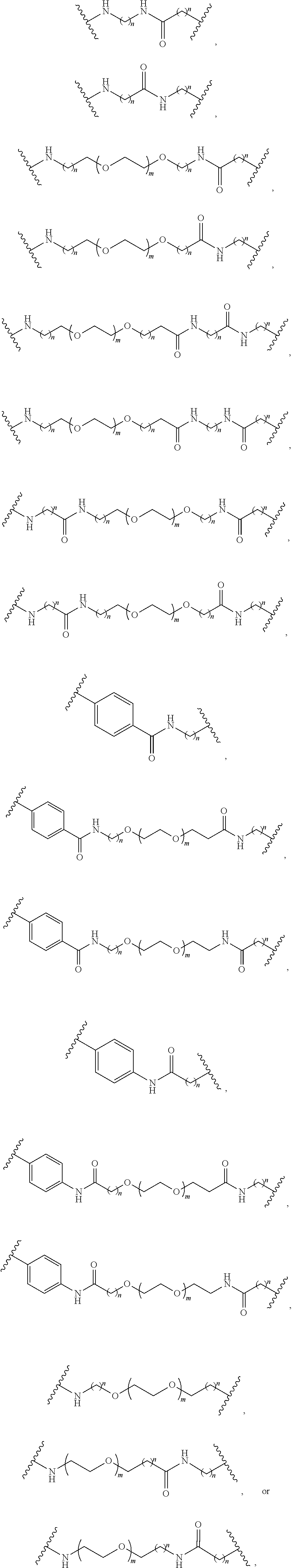

wherein each n is independently an integer from 1-6 and each m is an integer from 1-30. In some embodiments, each m is independently 2 or 3. In some embodiments, each n is an integer from 1-24, from 1-18, from 1-12, or from 1-6.

In some embodiments, the linker of Formula (X) or of Formula (X′) comprises the structure:

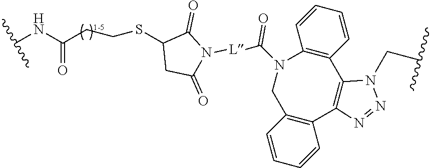

wherein

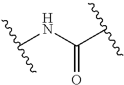 [SMILES: *NC(*)=O]

is the first point of attachment to a lysine residue of the immune checkpoint inhibitor molecule (e.g., the anti-PD-1 antibody or antigen binding fragment thereof);
L″ is a tether group; and

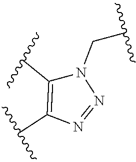 [SMILES: *Cn1nnc(*)c1*]

is a point of attachment to a tether group which connects to the IL-2 polypeptide, or a regioisomer thereof. In the structure above, in some embodiments, the succinimide is hydrolyzed at one of the N—C(═O) bonds.

In some embodiments, L″ has a structure

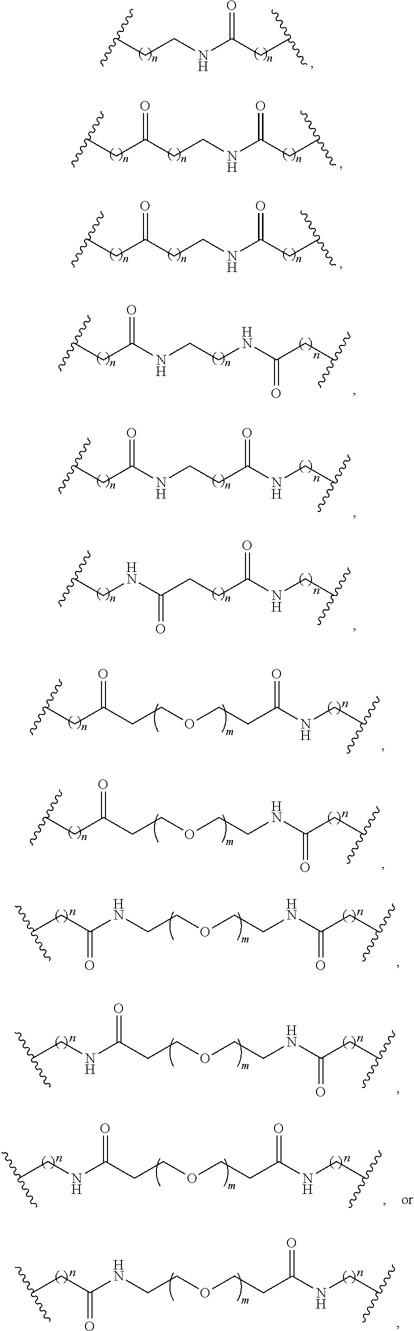

wherein each n is independently an integer from 1-6 and each m is independently an integer from 1-30. In some embodiments, each m is independently 2 or 3. In some embodiments, each m is an integer from 1-24, from 1-18, from 1-12, or from 1-6.

In some embodiments, L or L″ comprises 1,2,3, 4, 5, 6, 7, 8, 9, 10, 11, 12, 13, 14, 15, 16, 17, 18, 19, 20, 21, 22, 23, 24, 25, 26, 27, 28, 29, 30 or more subunits each independently selected from

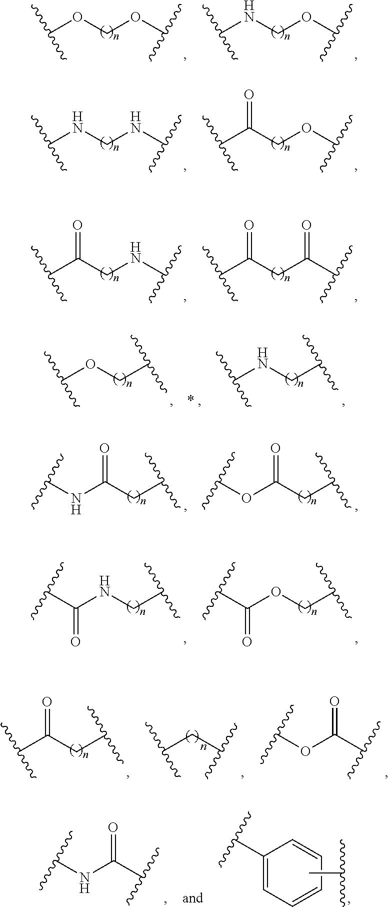

wherein each n is independently an integer from 1-30. In some embodiments, each n is independently an integer from 1-6. In some embodiments, L or L″ comprise 1, 2, 3, 4, 5, 6, 7, 8, 9, or 10 of the subunits.

In some embodiments, L or L″ is a structure of Formula (X″)

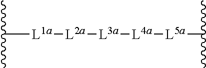

wherein each of L^{1a}, L^{2a}, L^{3a}, L^{4a}, L^{5a}, is independently —O—, —NR^{La}—, —(C₁-C₆ alkylene)NR^{La}—, —NR^{La}(C₁-C₆ alkylene)-, —N(R^{L})₂⁺—, —(C₁-C₆ alkylene)N(R^{L})₂⁺(C₁-C₆ alkylene)-, —N(R^{L})₂⁺—, —OP(═O)(OR^{La})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O)(C₁-C₆ alkylene)-, —C(═O)O—, —OC(═O)—, —OC(═O)O—, —C(═O)NR^{La}—, —C(═O)NR^{La}(C₁-C₆ alkylene)-, —(C₁-C₆ alkylene)C(═O)NR^{La}—, —NR^{La}—C(═O)—, —(C₁-C₆ alkylene)NR^{La}C(═O)—, —NR^{La}C(═O)(C₁-C₆ alkylene)-, —OC(═O)NR^{La}—, —NR^{La}C(═O)O—, —NR^{La}C(═O)NR^{La}—, —NR^{La}C(═S)NR^{La}—, —CR^{La}=N—, —N═CR^{La}—NR^{La}S(═O)₂—, —S(═O)₂NR^{La}—, —C(═O)NR^{La}S(═O)₂—, —S(═O)₂NR^{La}—C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qe}—, —(O—CH₂—CH₂)_{qf}—, —(CH₂—CH(CH₃)—O)_{qg}—, —(O—CH(CR-+CH₂)_{qh}—, a reaction product of a conjugation handle and a complementary conjugation handle, or absent;
each R^{La} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₅ cycloalkyl, substituted or unsubstituted C₂-C₇ heterocycloalkyl, substituted or unsubstituted aryl, or substituted or unsubstituted heteroaryl; and
each of qe, qf, qg and qh is independently an integer from 1-100.

In some embodiments, L or L″ comprises a linear chain of 2 to 10, 2 to 15, 2 to 20, 2 to 25, or 2 to 30 atoms. In some embodiments, the linear chain comprises one or more alkyl groups (e.g., lower alkyl (C₁-C₄)), one or more aromatic groups (e.g., phenyl), one or more amide groups, one or more ether groups, one or more ester groups, or any combination thereof.

In some embodiments, the tether group which connects to the first point of attachment (e.g., the point of attachment to the IL-2 polypeptide) comprises poly(ethylene glycol). In some embodiments, the tether group comprises about 2 to about 30 poly(ethylene glycol) units. In some embodiments, the tether group which connects to the first point of attachment (e.g., the point of attachment to the IL-2 polypeptide) is a functionality attached to an IL-2 polypeptide provided herein which comprises an azide (e.g., the triazole is the reaction product of the azide).

In some embodiments, each reaction product of a conjugation handle and a complementary conjugation handle independently comprises a triazole, a hydrazone, pyridazine, a sulfide, a disulfide, an amide, an ester, an ether, an oxime, or an alkene. In some embodiments, each reaction product of a conjugation handle and a complementary conjugation handle comprises a triazole. In some embodiments, each reaction product of a conjugation handle and a complementary conjugation handle independently comprises a structure of

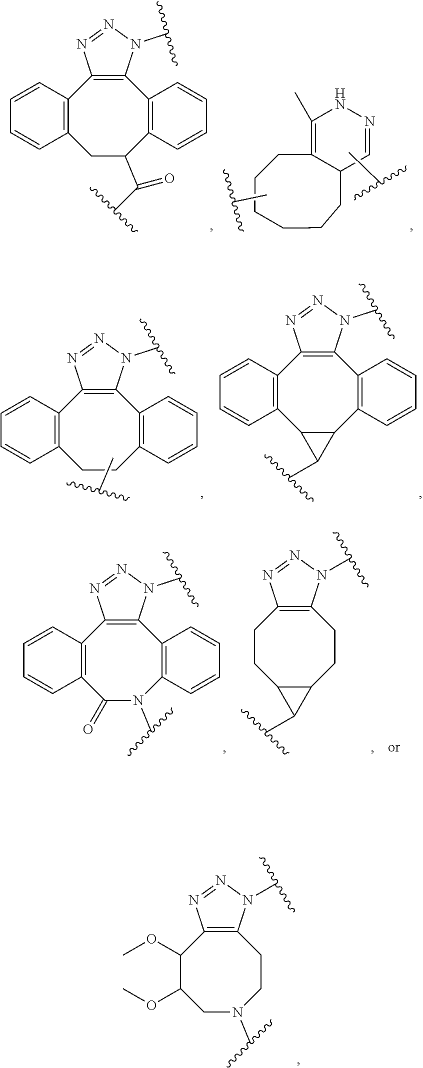

or a regioisomer or derivative thereof.

In some embodiments, the linker is a cleavable linker. In some embodiments, the cleavable linker is cleaved at, near, or in a tumor microenvironment. In some embodiments, the cleavable linker is mechanically or physically cleaved at, near, or in the tumor microenvironment. In some embodiments, the cleavable linker is chemically cleaved at, near, or in a tumor microenvironment. In some embodiments, the cleavable linker is a reduction sensitive linker. In some embodiments, the cleavable linker is an oxidation sensitive linker. In some embodiments, the cleavable linker is cleaved as a result of pH at, near, or in the tumor microenvironment. In some embodiments, the cleavable linker is cleaved by a tumor metabolite at, near, or in the tumor microenvironment. In some embodiments, the cleavable linker is cleaved by a protease at, near, or in the tumor microenvironment.

### Stoichiometry of Activatable Immunocytokines

In some embodiments, an activatable immunocytokine as provided herein can have a specified ratio of the number of immune checkpoint inhibitor molecules (e.g., anti-PD-1 antibodies or antigen binding fragments thereof) and IL-2 polypeptides. In some embodiments, the ratio of immune checkpoint inhibitor molecules to IL-2 polypeptides is 1 to 1 or 1 to 2. In some embodiments, a population of activatable immunocytokines provided herein has a ratio of immune checkpoint inhibitor molecules to IL-2 polypeptides of from about 1 to 1 to about 1 to 2 (e.g., can include individual activatable immunocytokines which comprise 1 IL-2 polypeptides and individual activatable immunocytokines which comprises 2 IL-2 polypeptides).

In some embodiments, wherein the immune checkpoint inhibitor molecule is an antibody or an antigen binding fragment thereof (e.g., an anti-PD-1 antibody or antigen binding fragment thereof), the ratio of immune checkpoint inhibitor molecule to IL-2 polypeptide in the activatable immunocytokine can be referred as a drug-antibody ration (DAR). In some embodiments, an activatable immunocytokine has a DAR of 1 or 2. In some embodiments, a population of activatable immunocytokines has a DAR of from 1 to 2.

### Pharmaceutical Compositions

In one aspect, described herein is a pharmaceutical composition comprising: an activatable immunocytokine described herein; and a pharmaceutically acceptable carrier or excipient. In some embodiments, the pharmaceutical composition further comprises one or more excipients, wherein the one or more excipients include, but are not limited to, a carbohydrate, an inorganic salt, an antioxidant, a surfactant, a buffer, or any combination thereof. In some embodiments the pharmaceutical composition further comprises one, two, three, four, five, six, seven, eight, nine, ten, or more excipients, wherein the one or more excipients include, but are not limited to, a carbohydrate, an inorganic salt, an antioxidant, a surfactant, a buffer, or any combination thereof.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

Alternately, or in addition, the pharmaceutical composition further comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

Alternately, or in addition, the pharmaceutical composition comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

Alternately, or in addition, the pharmaceutical composition further comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

Alternately, or in addition, the pharmaceutical composition further comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, or combinations thereof.

In some embodiments, the pharmaceutical composition is formulated for parenteral or enteral administration. In some embodiments, the pharmaceutical composition is formulated for intravenous (IV) or subcutaneous (SQ) administration. In some embodiments, the pharmaceutical composition is in a lyophilized form.

In one aspect, described herein is a liquid or lyophilized composition that comprises a described an activatable immunocytokine. In some embodiments, the activatable immunocytokine is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising about 10 mM Na₂HPO₄ buffer, about 0.022% SDS, and about 50 mg/mL mannitol, and having a pH of about 7.5.

### Dosage Forms

The activatable immunocytokines described herein can be in a variety of dosage forms. In some embodiments, the activatable immunocytokine is dosed as a reconstituted lyophilized powder. In some embodiments, the activatable immunocytokine is dosed as a suspension. In some embodiments, the activatable immunocytokine is dosed as a solution. In some embodiments, the activatable immunocytokine is dosed as an injectable solution. In some embodiments, the activatable immunocytokine is dosed as an IV solution. In some embodiments, the activatable immunocytokine is administered by subcutaneous or intramuscular administration.

### Methods of Treatment

In one aspect, described herein, is a method of treating cancer in a subject in need thereof, comprising: administering to the subject an effective amount of a activatable immunocytokine or a pharmaceutical composition as described herein. In some embodiments, the cancer is a solid cancer. A cancer or tumor can be, for example, a primary cancer or tumor or a metastatic cancer or tumor. Cancers and tumors to be treated include, but are not limited to, a melanoma, a lung cancer (e.g., a non-small cell lung cancer (NSCLC), a small cell lung cancer (SCLC), etc.), a carcinoma (e.g., a cutaneous squamous cell carcinoma (CSCC), a urothelial carcinoma (UC), a renal cell carcinoma (RCC), a hepatocellular carcinoma (HCC), a head and neck squamous cell carcinoma (HNSCC), an esophageal squamous cell carcinoma (ESCC), a gastroesophageal junction (GEJ) carcinoma, an endometrial carcinoma (EC), a Merkel cell carcinoma (MCC), etc.), a bladder cancer (BC), a microsatellite instability high (MSI-H)/mismatch repair-deficient (dMMR) solid tumor (e.g., a colorectal cancer (CRC)), a tumor mutation burden high (TMB-H) solid tumor, a triple-negative breast cancer (TNBC), a gastric cancer (GC), a cervical cancer (CC), a pleural mesothelioma (PM), classical Hodgkin's lymphoma (cHL), or a primary mediastinal large B cell lymphoma (PMBCL).

In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

An effective response is achieved when the subject experiences partial or total alleviation or reduction of signs or symptoms of illness, reduction of tumor burden, prolonging of time to increased tumor burden (progression of tumor), and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms or cancer burden remain static and do not worsen. Further indications of treatment of indications are described in more detail below. In some instances, a cancer or tumor is reduced by at least 2%, 5%, 10%, 15%, 20%, 25%, 30%, 35%, 40%, 45%, 50%, 55%, 60%, 65%, 70%, 75%, 80%, 85%, 90%, 95%, or 100%.

### Methods of Manufacturing

In one aspect, described herein, is a method of making an activatable immunocytokine comprising providing an immune checkpoint inhibitor molecule (e.g., an anti-PD-1 antibody or antigen binding fragment thereof) comprising a reactive group (e.g., a conjugation handle), contacting the reactive group with a complementary reactive group attached to an IL-2 polypeptide provided herein, and forming the composition. The resulting composition is any of the compositions provided herein.

In some embodiments, the immune checkpoint inhibitor molecule is an antibody or an antigen binding fragment thereof. In some embodiments, the immune checkpoint inhibitor molecule is an anti-PD-1 antibody or antigen binding fragment thereof. In some embodiments, the immune checkpoint inhibitor molecule is an antibody. In some embodiments, the immune checkpoint inhibitor molecule is an anti-PD-1 antibody. In some embodiments, providing the antibody comprising the reactive group comprises attaching the reactive group to the antibody. In some embodiments, the reactive group is added site-specifically. In some embodiments, attaching the reactive group to the antibody comprises contacting the antibody with an affinity group comprising a reactive functionality which forms a bond with a specific residue of the antibody. In some embodiments, attaching the reactive group to the antibody comprises contacting the antibody with an enzyme. In some embodiments, the enzyme is configured to site-specifically attach the reactive group to a specific residue of the antibody. In some embodiments, the enzyme is glycosylation enzyme or a transglutaminase enzyme.

In some embodiments, the method further comprises attaching the complementary reactive group to the IL-2 polypeptide. In some embodiments, attaching the complementary reactive group to the IL-2 polypeptide comprises chemically synthesizing the IL-2 polypeptide

In some embodiments, the method comprises making the IL-2 polypeptide. In some embodiments, the method of making the IL-2 polypeptide comprises synthesizing two or more fragments of the IL-2 polypeptide and ligating the fragments. In some embodiments, the method of making the IL-2 polypeptide comprises a. synthesizing two or more fragments of the IL-2 polypeptide, b. ligating the fragments; and c. folding the ligated fragments.

In some embodiments, the two or more fragments of the IL-2 polypeptide are synthesized chemically. In some embodiments, the two or more fragments of the IL-2 polypeptide are synthesized by solid phase peptide synthesis. In some embodiments, the two or more fragments of the IL-2 polypeptide are synthesized on an automated peptide synthesizer.

In some embodiments, the IL-2 polypeptide is ligated from 2, 3, 4, 5, 6, 7, 8, 9, 10, or more peptide fragments. In some embodiments, the IL-2 polypeptide is ligated from 2 peptide fragments. In some embodiments, the IL-2 polypeptide is ligated from 3 peptide fragments. In some embodiments, the IL-2 polypeptide is ligated from 4 peptide fragments. In some embodiments, the IL-2 polypeptide is ligated from 2 to 10 peptide fragments.

In some embodiments, the two or more fragments of the IL-2 polypeptide are ligated together. In some embodiments, three or more fragments of the IL-2 polypeptide are ligated in a sequential fashion. In some embodiments, three or more fragments of the IL-2 polypeptide are ligated in a one-pot reaction.

In some embodiments, ligated fragments are folded. In some embodiments, folding comprises forming one or more disulfide bonds within the IL-2 polypeptide. In some embodiments, the ligated fragments are subjected to a folding process. In some embodiments, the ligated fragments are folding using methods well known in the art. In some embodiments, the ligated polypeptide or the folded polypeptide are further modified by attaching one or more polymers thereto. In some embodiments, the ligated polypeptide or the folded polypeptide are further modified by PEGylation. In some embodiments, the IL-2 polypeptide is synthetic.

### Certain Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise. In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are groups which are “attached” or “covalently attached” to different entities (e.g., amino acid residues of IL-2 polypeptides or anti-PD-1 antibodies or antigen binding fragments thereof). As used herein, “attached” or “covalently attached” means that the group is tethered to the indicated residue, and such tethering can include a spacing group (e.g., a linking group or linker as provided herein). Thus, for a group “attached” or “covalently attached” to a residue, it is expressly contemplated that chemical spacing groups are also encompassed.

“Linking group” as used herein refers to a group of one or more atoms which act as a tether between an IL-2 polypeptide and a cleavable moiety. “Linker” as used herein refers to a group of one or more atoms which act as a tether between an IL-2 polypeptide and an immune checkpoint inhibitor molecule (e.g., an anti-PD-1 antibody or antigen binding fragment thereof).

Binding affinity refers to the strength of a binding interaction between a single molecule and its ligand/binding partner. A higher binding affinity refers to a higher strength bond than a lower binding affinity. In some instances, binding affinity is measured by the dissociation constant (K_{D}) between the two relevant molecules. When comparing K_{D} values, a binding interaction with a lower value will have a higher binding affinity than a binding interaction with a higher value. For a protein-ligand interaction, K_{D} is calculated according to the following formula:

$${K}_{D} = \frac{[ L ] [ P ]}{⌈ LP ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence: 11, Extension:1, and Compositional Adjustments Conditional Compositional Score Matrix Adjustment. This alignment algorithm is also used to assess if a residue is at a “corresponding” position through an analysis of the alignment of the two sequences being compared.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA. p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer

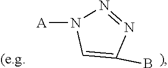

it is intended that the other regioisomer

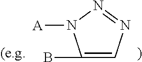

is also encompassed.

The term “subject” refers to an animal which is the object of treatment, observation, or experiment. βγ way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, the term “number average molecular weight” (Mn) means the statistical average molecular weight of all the individual units in a sample, and is defined by Formula (1):

$$Mn = \frac{\sum {N}_{i} {M}_{i}}{\sum {N}_{i}} \qquad Formula ( 1 )$$

where Mᵢ is the molecular weight of a unit and Nᵢ is the number of units of that molecular weight.

As used herein, the term “weight average molecular weight” (Mw) means the number defined by Formula (2):

$$Mw = \frac{\sum {N}_{i} {M}_{i}^{2}}{\sum {N}_{i} {M}_{i}} \qquad Formula ( 2 )$$

where Mᵢ is the molecular weight of a unit and Nᵢ is the number of units of that molecular weight.

As used herein, “peak molecular weight” (Mp) means the molecular weight of the highest peak in a given analytical method (e.g., mass spectrometry, size exclusion chromatography, dynamic light scattering, analytical centrifugation, etc.).

As used herein, “unnatural” amino acids can refer to amino acid residues in D- or L-form that are not among the 20 canonical amino acids generally incorporated into naturally occurring proteins. Non-limiting examples of unnatural amino acids include, but are not limited to, N-alpha-(9-Fluorenylmethyloxycarbonyl)-L-biphenylalanine (Fmoc-L-Bip-OH) and N-alpha-(9-Fluorenylmethyloxycarbonyl)-O-benzyl-L-tyrosine (Fmoc-L-Tyr(Bzl)-OH. Exemplary non-canonical amino acids include p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, azido-lysine (AzK), an analogue of a tyrosine amino acid; an analogue of a glutamine amino acid; an analogue of a phenylalanine amino acid; an analogue of a serine amino acid; an analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, a p-amino acid; a cyclic amino acid other than proline or histidine; an aromatic amino acid other than phenylalanine, tyrosine or tryptophan; or a combination thereof. In some embodiments, the non-canonical amino acids are selected from p-amino acids, homoamino acids, cyclic amino acids and amino acids with derivatized side chains. In some embodiments, the non-canonical amino acids comprise β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, hydroxylysine, allo-hydroxylysine, isodesmosine, allo-isoleucine, ω-methylarginine, N^{α}-methylglycine, N^{α}-methylisoleucine, N^{α}-methylvaline, γ-carboxyglutamate, ε—N,N,N-trimethyllysine, ε-N-acetyllysine, O-phosphoserine, N^{α}-acetylserine, N^{α}-formylmethionine, 3-methylhistidine, 5-hydroxylysine, and/or other similar amino acids.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

TABLE of

| | | Reaction |
|---|---|---|
| Conjugation Handle | Complementary Conjugation Handle | Product |
| Sulfhydryl | alpha-halo-carbonyl (e.g., bromoacetamide), alpha- | thioether |
| | beta unsaturated carbonyl (e.g., maleimide, | |
| | acrylamide) | |
| Azide | alkyne (e.g., terminal alkyne, substituted | triazole |
| | cyclooctyne (e.g., dibenzocycloocytne (DBCO), | |
| | difluorocyclooctyne, bicyclo[6.1.0]nonyne, etc.)) | |
| Phosphine | Azide/ester pair | amide |
| Tetrazine | trans-cyoclooctene | dihydropyrida |
| | | zine |
| Amine | Activated ester (e.g., N-hydroxysuccinimide ester, | amide |
| | pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine (e.g., O- | amide |
| trifluoroborate | carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₅ alkyl, C₃-C₈ alkyl and C₄-C₅ alkyl. Representative alkyl groups include, but are not limited to, methyl, ethyl, -propyl, 1-methyl ethyl, -butyl, -pentyl, 1,1-dimethyl ethyl, 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

The term “alkynyl” refers to a type of alkyl group in which at least one carbon-carbon triple bond is present. In one embodiment, an alkynyl group has the formula —C≡C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃, —C≡CCH₂CH, and —CH₂C≡CH.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—.

The term “heteocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7, 8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is optionally substituted with one or more additional group(s) individually and independently selected from D, halogen, —CN, —NH2, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH), —N(CH)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

As used herein, “AJICAP™ technology,” “AJICAP™ methods,” and similar terms refer to systems and methods (currently produced by Ajinomoto Bio-Pharma Services (“Ajinomoto”)) for the site specific functionalization of antibodies and related molecules using affinity peptides to deliver the desired functionalization to the desired site. General protocols for the AJICAP™ methodology are found at least in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, Yamada et al., AJICAP: Affinity Peptide Mediated Regiodivergent Functionalization of Native Antibodies. Angew. Chem., Int. Ed. 2019, 58, 5592-5597, and Fujii et al., Bioconjugate Chem. 2023, 34, 4, 728-738. In some embodiments, such methodologies site specifically incorporate the desired functionalization at lysine residues at a position selected from position 246, position 248, position 288, position 290, and position 317 of an antibody Fc region (e.g., an IgG1 Fc region) (EU numbering). In some embodiments, the desired functionalization is incorporated at residue position 248 of an antibody Fc region (EU numbering). In some embodiments, position 248 corresponds to the 18^{th} residue in a human IgG CH2 region (EU numbering).

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### Example 1: Synthesis of IL-2 Polypeptides—General Procedures

### Preparation IL-2 Linear Protein (Representative Protocols)

General strategy: An activatable IL-2 polypeptide or an IL-2 polypeptide as described herein, such as an IL-2 polypeptide having an amino acid sequence of, for example, SEQ ID NOs: 2-55, or an activatable IL-2 polypeptide otherwise described herein, can be synthesized by ligating individual peptide segments prepared by solid phase peptide synthesis (SPPS). Individual peptides were synthesized on an automated peptide synthesizer using the methods described below. Other activatable IL-2 polypeptides (e.g., those comprising other cleavable moieties provided herein, such as those provided in Table 1C or Table 1D) can be prepared using analogous methods.

Materials and solvents: Fmoc-amino acids with suitable side chain protecting groups for Fmoc-SPPS, resins polyethylene glycol derivatives used for peptide functionalization and reagents were commercially available and were used without further purification. HPLC grade CH₃CN was used for analytical and preparative RP-HPLC purification. The following Fmoc-amino acids with side-chain protecting groups were used: Fmoc-Ala-OH, Fmoc-Arg(Pbf)-OH, Fmoc-Asn(Trt)-OH, Fmoc-Asp(OtBu)-OH, Fmoc-Cit-OH, Fmoc-Cys(Acm)-OH, Fmoc-Dab(Alloc)-OH, Fmoc-Dab(Boc)-OH, Fmoc-Gln(Trt)-OH, Fmoc-Glu-OAII, Fmoc-Glu(OtBu)-OH, Fmoc-Glu(OAII)—OH, Fmoc-Gly-OH, Fmoc-His(Trt)-OH, Fmoc-Ile-OH, Fmoc-Leu-OH, Fmoc-Lys(Boc)-OH, Fmoc-Lys(Alloc)-OH, Fmoc-Lys(ivDde)-OH, Fmoc-Nle-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc or Boc-Opr-OH (Opr=5-(S)-oxaproline), Fmoc-Ygp-OH, Fmoc-Yn3-OH. Fmoc-pseudoproline dipeptides were incorporated in the synthesis if necessary.

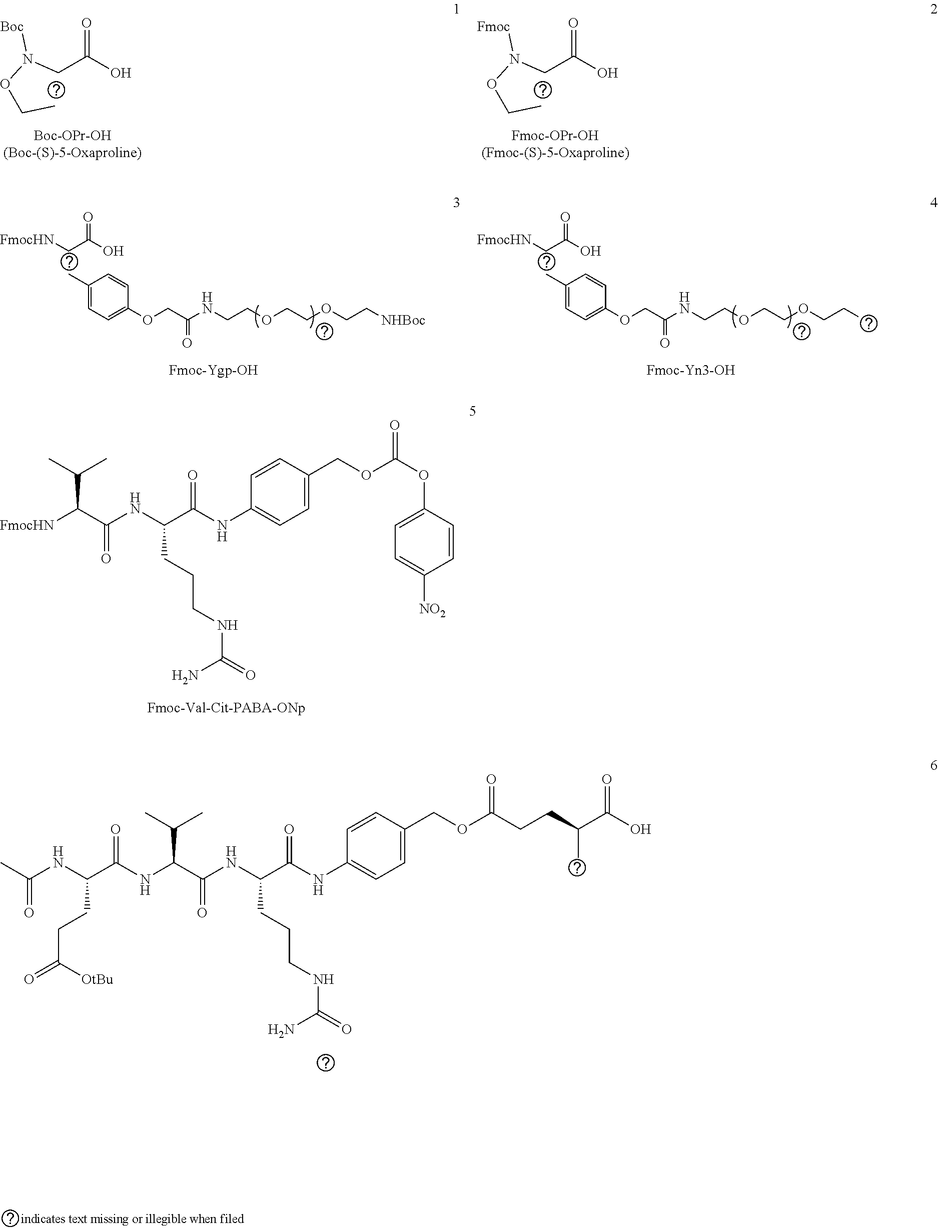

### Special Building Blocks Structures

Protocol 1—Loading of protected ketoacid derivatives (segment 1-3) on amine-based resin: 5 g of Rink-amide MBHA or Sieber Resin (1.8 mmol scale) was swollen in DMF for 30 min. Fmoc-deprotection was performed when needed by treating the resin twice with 20% piperidine in DMF (v/v) at r.t. for 10 min followed by several washes with DMF. Fmoc-AA-protected-α-ketoacid (1.8 mmol, 1.00 equiv.) was dissolved in 20 mL DMF and pre-activated with HATU (650 mg, 1.71 mmol, 0.95 equiv.) and DIPEA (396 μL, 3.6 mmol, 2.00 equiv.). The reaction mixture was added to the swollen resin. It was let to react for 6 h at r.t. under gentle agitation. The resin was rinsed thoroughly with DMF. Capping of unreacted amines on the resin was performed by addition of a solution of acetic anhydride (1.17 mL) and DIPEA (2.34 mL) in DMF (20 mL). It was let to react at r.t. for 15 min under gentle agitation. The resin was rinsed thoroughly with DCM followed by diethyl ether and dried. The loading of the resin was determined by UV quantification of dibenzofulvene to be 0.25 mmol/g.

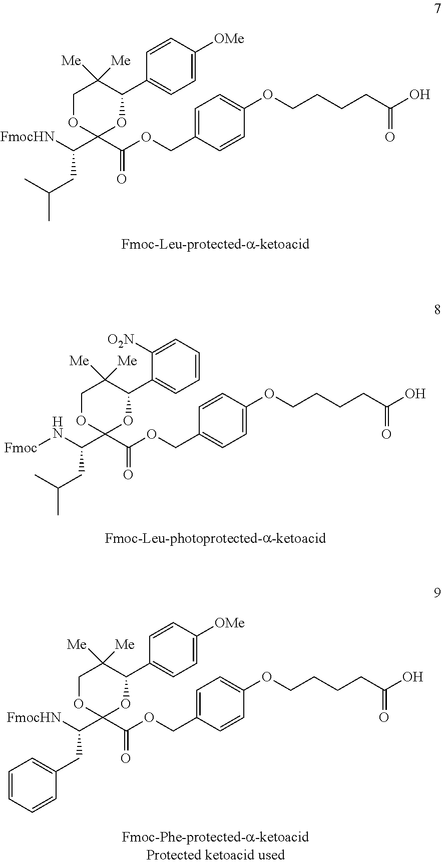

Protocol 2—Loading of Fmoc-Thr(tBu)-OH on Wang resin (segment 4): Preloading of Fmoc-Thr-OH was performed on a Wang resin. 4 g of resin (loading: 0.56 mmol/g, 2.24 mmol scale) was swollen in DMF for 15 min. The resin was treated with 20% (v/v) piperidine in DMF at r.t. for 20 min. The resin was washed several times with DMF. Fmoc-Thr(tBu)-OH (638 mg, 1.68 mmol, 0.75 equiv) and HATU (638 mg, 1.68 mmol, 0.75 equiv) were dissolved in DMF (12 mL). Pre-activation was performed at r.t. for 3 min by addition of DIPEA (585 μL, 3.36 mmol, 1.5 equiv). The reaction mixture was added to the swollen resin. It was let to react overnight at r.t. under gentle agitation. The resin was rinsed thoroughly with DMF. Capping of unreacted amines on the resin was initiated by addition of a solution of acetic anhydride (1.27 mL) and DIPEA (2.34 mL) in DMF (12 mL). It was let to react at r.t. for 15 min under gentle agitation. The resin was rinsed thoroughly with DCM and dried. The loading of the resin was measured (0.34 mmol/g).

Protocol 3—Elongation of the segment 1: The peptide segments were synthesized on an automated peptide synthesizer using Fmoc-SPPS chemistry. Couplings were performed with Fmoc-amino acid (2-16 equiv. to resin substitution), HCTU or HATU (2-16 equiv.) as coupling reagents and DIPEA or NMM (4-32 equiv.) in DMF or NMP (20-60 mL/mmol resin substitution) at r.t. or at 50° C. After pre-activation for 3 min, the solution containing the reagents was added to the resin and allowed to react for 30 min or 2 h depending on the amino acid. In some cases, double couplings were required. In some cases, the resin was treated with 20% acetic anhydride (10 equiv.) in DMF in presence of NMM (10 equiv.) for capping any unreacted free amine. Fmoc deprotections were performed with 20% piperidine or 4-Methylpiperidine in DMF (2×2 min, 40 mL/mmol resin substitution).

Protocol 4—Elongation of segment 2, 3 and 4: Fmoc-Amino acid (2-3 equiv.), DIC (3-7 equiv.), and Oxyma (2-3 equiv.) in DMF (20-60 mL/mmol resin substitution) were stirred for 5-10 min and added to the resin in one portion. The reaction mass was gently agitated under nitrogen bubbling for 2 h at 25-30° C. The solvent was drained, and the resin was washed with DMF (40 mL/mmol resin substitution, 3×5 min), DCM (40 mL/mmol resin substitution; 3×5 min). In some cases, double couplings were required. In some cases, the resin was treated with 20% acetic anhydride (10 equiv.) in DMF in presence of NMM (10 equiv.) for capping any unreacted free amine (20-60 mL/mmol resin substitution). Fmoc deprotections were performed twice 5 or 15 min with 20% piperidine in DMF (40 mL/mmol resin substitution).

Protocol 5—Alloc protection on-resin: A solution of allyl chloroformate (3 equiv.) and Cl-HOBt (6 equiv.) in DCM/DMF (1/1, v/v, 25 mL/mmol) was mixed and DIPEA (6 equiv.) was added to the mixture. After 45 min of agitation, the reaction mixture was added to the resin preswollen in DMF. The reaction was gently agitated for 1 h and then washed twice with DCM (40 mL/mmol resin substitution), twice with DMF (40 mL/mmol resin substitution) and twice with DCM (50 mL/mmol resin substitution).

Protocol 6—Fmoc deprotection of multiple PEG₂₇ derivatives: Fmoc deprotections were performed with 20% piperidine or 4-Methylpiperidine in NMP (4×10 min, 50 mL/mmol resin substitution). The resin was washed with twice with NMP (50 mL/mmol resin substitution), twice with twice with IPA (50 mL/mmol resin substitution) and twice with NMP (50 mL/mmol resin substitution).

Protocol 7—Alloc/All deprotection on resin: DMBA (20 equiv.) dissolved in DMSO (25 mL/mmol) were added to the resin preswollen in DCM followed by Pd[PPh₃]₄(0.2-0.5 equiv.) dissolved in dry DCM (10 mL/mmol resin substitution). The resin was shaken for 15-30 min and then washed twice with DCM (50 mL/mmol resin substitution), twice with DMF (50 mL/mmol resin substitution), twice with DCM (50 mL/mmol resin substitution), twice with IPA (50 mL/mmol resin substitution), and twice with Et₂O (50 mL/mmol resin substitution). The reaction and the wash were repeated once.

Protocol 8—ivDde deprotection on resin: The resin was swollen in DMF for 10 min. 10% (v/v) hydrazine monohydrate in DMF containing 0.05 M allyl alcohol (40 mL/mmol resin substitution) were added to the resin and it was let to react at room temperature for 30 min. This step was repeated three more times. The resin was then washed with DMF and DCM.

Protocol 9—Glutaric acid incorporation: dihydro-2H-pyran-2,6(3H)-dione (5 equiv.) was dissolved in DMF (37.5 mL/mmol resin substitution) and DIPEA (7 equiv.) was added. The mixture was transferred to the resin and let it shake for 2 hours at room temperature. The resin was washed twice with DMF (40 mL/mmol resin substitution), twice with DCM (10 mL/mmol resin substitution) and twice with IPA (40 mL/mmol resin substitution).

Protocol 10—2-C-Trityl glutamic acid protection: A solution of DIPEA (6 equiv.) in dry DCM (20 mL/mmol resin substitution) was added to the resin followed by a solution of 2-Cl-Trityl chloride (3 equiv.) in dry DCM (20 mL/mmol resin substitution) and shaken for 2 h. The resin was filtered, washed with DCM (40 mL/mmol resin substitution) and the protection step was repeated twice in the same conditions. The resin was washed with DCM (3×40 mL/mmol resin substitution), IPA (2×40 mL/mmol resin substitution) and NMP (3×40 mL/mmol resin substitution).

Protocol 11—2-Cl-Trityl protected glutamic acid deprotection: A solution of 40% HFIP in DCM (100 mL/mmol resin substitution) was added to the resin. The reaction mixture was shaken for 30 min. The deprotection reaction was repeated twice. The resin was washed with DCM (3×40 mL/mmol resin substitution), Et₂O (2×40 mL/mmol resin substitution), DMF (3×40 mL/mmol resin substitution).

Protocol 12—Resin cleavage (Sieber resin) of protected peptide: The resin was preswollen in DCM. A solution of 3% TFA in DCM (v/v, 40 mL/mmol resin substitution) was added on the resin and the mixture was gently agitated for 3 min. The cleavage reaction was repeated 5 times. After each reaction, the filtrates were combined and directly quenched in a 40% DIPEA in DCM solution (40 mL/mmol resin substitution). The resin was washed 3 times with DCM and the combined filtrates were added to the previous filtrates. The mother solution was concentrated under vacuum to dryness and solubilized in ACN. The solution was poured in water and the protected linear peptide as precipitate was filtered and dried.

Protocol 13—Cyclization of protected peptide in solution: The linear protected peptide was dissolved in DMF (300 mL/mmol resin substitution) with 4 equiv. of DIPEA. The peptide solution was added dropwise to a solution of HATU (2 equiv., 350 mL/mmol) in DMF over 30 min. After addition, the reaction mixture was evaporated to dryness.

Protocol 14—Resin cleavage (Rink amide resin) and/or side chain deprotection of the peptides: Once the peptide synthesis was completed, the peptides were cleaved from the resin using a cleavage cocktail (see table below) at room temperature for 2 h. The resin was filtered off, and the filtrate was concentrated and treated with cold diethyl ether, triturated and centrifuged. The ether layer was carefully decanted, the residue was suspended again in diethyl ether, triturated and centrifuged. Ether washings were repeated twice. The resulting crude peptide was dried under vacuum and stored at −20° C. An aliquot of the solid obtained was solubilized in 1:1 CH₃CN/H₂O with 0.1% TFA (v/v) and analyzed by analytical RP-HPLC using C18 column (4.6×150 mm) at 50° C. The molecular weight of the product was identified using MALDI-TOF or LC-MS.

TABLE 5
Rink amide resin cleavage cocktails

| Segment | Cleavage cocktail |
|---|---|
| Segment 1 | TFA:DODT:H₂O (95:2.5:2.5) (v/v/v) |
| Segment 2 | TFA:DODT:H₂O (95:2.5:2.5) (v/v/v) |
| Segment 3 | TFA:DODT:H₂O (95:2.5:2.5) (v/v/v) |
| Segment 4 | TFA:Phenol:TIPS:H₂O (82:8:5:5) (v/v/v/v) |

Protocol 15—Ligation of IL-2 segments 1 and 2 and photodeprotection: IL-2 Seg1 (1.2 equiv) and IL-2 Seg2 (1 equiv) were dissolved in DMSO:H₂O (9:1, v/v) containing 0.1 M oxalic acid (20 mM peptide concentration) and allowed to react at 60° C. for 22 h. The ligation vial was protected from light by wrapping it in aluminum foil. The progress of the KAHA ligation was monitored by HPLC using a C18 column (4.6×150 mm) at 60° C. with CH₃CN/H₂O containing 0.1% TFA as mobile phase, with a gradient of 5 to 95% CH₃CN in 7 min. After completion of the ligation the mixture was diluted with CH₃CN/H₂O (1:1) containing 0.1% TFA and irradiated at a wavelength of 365 nm for 1 h. The completion of photolysis reaction was confirmed by injecting a sample on HPLC using previously described method. The sample was then purified by preparative HPLC.

Protocol 16—Ligation of IL-2 segments 3 and 4 and Fmoc deprotection: IL-2-Seg3 (1.2 equiv.) and IL-2-Seg4 (1 equiv.) were dissolved in DMSO/H₂O (9.8:0.2) containing 0.1 M oxalic acid (15 mM) and allowed to react for 20 h at 60° C. The progress of the KAHA ligation was monitored by HPLC using a C18 column (4.6×150 mm) at 60° C. using ACN/H₂O containing 0.1% TFA as mobile phase, with a gradient of 30 to 70% ACN in 7 min. After completion of ligation, the reaction mixture was diluted with DMSO (6 mL) and 5% of diethylamine (300 μL) was added to the reaction mixture and shaken for 7 min at room temperature. To prepare the sample for purification, it was diluted with DMSO containing TFA. The sample was purified by preparative HPLC.

Protocol 17—Final ligation: IL-2-Seg12 (1.2 equiv.) and IL-2-Seg34 (1 equiv.) were dissolved in DMSO/H₂O (9:1) or (9.8:0.2) containing 0.1 M oxalic acid (15 mM peptide concentration) and the ligation was allowed to proceed for 24 h at 60° C. The progress of the KAHA ligation was monitored by analytical HPLC using a C18 column (4.6×250 mm) at 60° C. and ACN|/H₂O containing 0.1% TFA as mobile phase, with a gradient of 30 to 95% CH₃CN in 14 min. After completion of ligation, the reaction mixture was diluted with DMSO followed by further dilution with a mixture of (1:1, v/v) ACN:H₂O containing 0.1% TFA. The sample was purified by preparative HPLC.

Protocol 18—Acm deprotection: IL-2 linear protein with 2×Acm was dissolved in AcOH/H₂O (1:1) (0.25 mM protein concentration) and AgOAc (1% m/v) was added to the solution. The mixture was shaken for 2.5 h at 50° C. protected from light. After completion of reaction as ascertained by HPLC, the sample was diluted with ACN:H₂O (15/85, v/v) containing 0.1% TFA, and purified by preparative HPLC.

Protocol 19 Rearrangement and Folding: IL-2 linear protein (10 mg, 0.611 μmol, 15 μM) was dissolved in 6M Gu·HCl containing 0.1 M Tris pH 8.0 and 30 mM reduced glutathione. The mixture was shaken for 2 h at 45° C. to allow for rearrangement of the ester bond to yield an amide, resulting in the α-homoserine scar from the KAHA ligation. The progress of the rearrangement reaction was monitored by analytical reverse phase HPLC. After completion of rearrangement reaction as ascertained by HPLC, the sample was allowed to cool to room temperature and diluted 3-fold slowly (around 0.25 mL/min) with 0.1 M Tris and 1.5 mM oxidized glutathione, pH 8.0, while the solution was stirred. The folding was allowed to proceed for 20 h at room temperature. This resulted in dilution of the protein to a final concentration of 5 μM and oxidizing conditions that allow disulfide bonds to form. After completion of the folding reaction as ascertained by reverse phase, the sample was acidified with 10% TFA in MQ-H₂O (˜4.5% of total folding volume) until the solution reached pH 3-4. The sample was purified by preparative HPLC using a Waters Xbridge Protein BEH C4 column (20×250 mm) at room temperature (25° C.). A two-step gradient of 5 to 40 to 95% ACN with 0.1% TFA in 60 min, flow rate: 10.0 mL/min, with ACN and MQ-H₂O containing 0.1% TFA as the eluents. The fractions containing the product were pooled and lyophilized to give pure folded protein. The purity and identity of the folded protein powder was initially confirmed by high-resolution mass spectrometry. The lyophilized powder was the reconstituted in formulation buffer (10 mM sodium acetate pH 5.2, 8.4% sucrose and 0.02% PS80).

Protocol 20—Purification of the peptides and proteins: Peptide segments, ligated peptides and linear proteins were purified by RP-HPLC. Different gradients were applied for the different peptides. The mobile phase was MilliQ-H₂O with 0.1% TFA (v/v) (Buffer A) and HPLC grade ACN with 0.1% TFA (v/v) (Buffer B). Preparative HPLC was performed on a C₄ (50×250 mm) or on a C18 column (50×250 mm) at a flow rate of 40 or 55 mL/min at 40° C. or 50° C. A representative gradient used for the purification is given in the tables below. For the methods the buffers were Buffer: A: H₂O 0.1% TFA (v/v), B: ACN 0.1% TFA (v/v)

Column: C18 5 μm; 50×250 mm; Temperature: 50° C.

TABLE 6
RP-HPLC Method 1 Gradient

| Time | Flow | | |
|---|---|---|---|
| (min) | (ml/min) | A % | B % |
| 0 | 55 | 95 | 5 |
| 2 | 55 | 90 | 10 |
| 10 | 55 | 90 | 10 |
| 15.1 | 55 | 68 | 32 |
| 51 | 55 | 58 | 42 |
| 52 | 55 | 5 | 95 |
| 60 | 55 | 5 | 95 |
| 61 | 55 | 95 | 5 |
| 65 | 55 | 95 | 5 |

Characterization of the peptides: Peptide segments, ligated peptides and linear proteins were analyzed by RP-HPLC (see methods below). Peptides and proteins were characterized by high resolution Fourier-transform mass spectrometry (FTMS) using a SolariX (9.4T magnet) spectrometer (Bruker, Billerica, USA) equipped with a dual ESI/MALDI-FTICR source, using 4-hydroxy-α-cyanocinnamic acid (HCCA) as matrix.

### HPLC Methods

Buffers used: A=H₂O with 0.1% TFA (v/v), B=ACN with 0.1% TFA (v/v)

Method 1: Column: Waters XBridge C18 3.5 μm; 3×150 mm; Temperature: 50° C.

TABLE 7
RP-HPLC Method 1 Gradient

| Time | Flow | | |
|---|---|---|---|
| (min) | (mL/min) | % A | % B |
| 0 | 0.8 | 70 | 30 |
| 1.9 | 0.8 | 70 | 30 |
| 2 | 0.8 | 70 | 30 |
| 17 | 0.8 | 40 | 60 |
| 17.1 | 0.8 | 5 | 95 |
| 19 | 0.8 | 5 | 95 |
| 19.1 | 0.8 | 70 | 30 |
| 21 | 0.8 | 70 | 30 |

Method 2: Column: Waters XBridge C18 3.5 μm; 3×150 mm; Temperature: 50° C.; gradient.

TABLE 8
RP-HPLC Method 2 Gradient

| Time | Flow | | |
|---|---|---|---|
| (min) | (mL/min) | % A | % B |
| 0 | 0.8 | 80 | 20 |
| 1.9 | 0.8 | 80 | 20 |
| 2 | 0.8 | 80 | 20 |
| 17 | 0.8 | 30 | 70 |
| 17.1 | 0.8 | 5 | 95 |
| 19 | 0.8 | 5 | 95 |
| 19.1 | 0.8 | 80 | 20 |
| 21 | 0.8 | 80 | 20 |

Method 3: Column: Phenomenex Aeris 3.6 μm um Widepore XB-C18; 4.6×150 mm; Temperature: 50° C.; gradient:

TABLE 9
RP-HPLC Method 3 Gradient

| Time | Flow | | |
|---|---|---|---|
| (min) | (mL/min) | % A | % B |
| 0.02 | 1 | 70 | 30 |
| 1.5 | 1 | 70 | 30 |
| 8 | 1 | 40 | 60 |
| 8.5 | 1 | 5 | 95 |
| 10 | 1 | 5 | 95 |
| 10.5 | 1 | 95 | 5 |
| 11.95 | 1 | 95 | 5 |
| 12 | 1 | 95 | 5 |

Method 4: Column: Waters XBridge C18 3.5 μm; 3×150 mm; Temperature: 50° C.

TABLE 10
RP-HPLC Method 4 Gradient

| Time | Flow | | |
|---|---|---|---|
| (min) | (mL/min) | % A | % B |
| 0 | 0.8 | 95 | 5 |
| 2 | 0.8 | 95 | 5 |
| 17 | 0.8 | 5 | 95 |
| 17.1 | 0.8 | 5 | 95 |
| 19 | 0.8 | 5 | 95 |
| 19.1 | 0.8 | 95 | 5 |
| 21 | 0.8 | 95 | 5 |

Method 5: Column: Phenomenex Aeris 3.6 μm Widepore C4 200 Å; 4.6×150 mm; Temperature: 50° C.

TABLE 11
RP-HPLC Method 5 Gradient

| Time | Flow | | |
|---|---|---|---|
| (min) | (mL/min) | % A | % B |
| 0 | 2 | 80 | 20 |
| 1.5 | 2 | 80 | 20 |
| 5.4 | 2 | 30 | 70 |
| 5.5 | 2 | 5 | 95 |
| 6.9 | 2 | 5 | 95 |
| 7 | 2 | 80 | 20 |
| 9 | 2 | 80 | 20 |
| 9.1 | 2 | 80 | 20 |

Method 6: Column: Waters Xbridge Protein BEH; C4 300A; 2.5 μm: 3×150 mm; Temperature: room temperature

TABLE 12
RP-HPLC Method 6 Gradient

| Time | Flow | | |
|---|---|---|---|
| (min) | (mL/min) | % A | % B |
| 0 | 0.8 | 90 | 10 |
| 1.9 | 0.8 | 90 | 10 |
| 2 | 0.8 | 80 | 20 |
| 17 | 0.8 | 30 | 70 |
| 17.1 | 0.8 | 5 | 95 |
| 19 | 0.8 | 5 | 95 |
| 19.1 | 0.8 | 90 | 10 |
| 21 | 0.8 | 90 | 10 |

Method 7: Column: Waters XBridge C18 3.5 μm; 3×150 mm; Temperature: 50° C.

TABLE 13
RP-HPLC Method 7 Gradient

| Time | Flow | | |
|---|---|---|---|
| (min) | (mL/min) | % A | % B |
| 0 | 0.5 | 90 | 10 |
| 8 | 0.5 | 90 | 10 |
| 20 | 0.5 | 50 | 50 |
| 25 | 0.5 | 20 | 80 |
| 30 | 0.5 | 5 | 95 |
| 30.1 | 0.5 | 5 | 95 |
| 35 | 0.5 | 90 | 10 |

Method 8: Column: Waters XBridge C18 3.5 μm; 3×150 mm; Temperature: 50° C.

TABLE 14
RP-HPLC Method 8 Gradient

| Time | Flow | | |
|---|---|---|---|
| (min) | (mL/min) | % A | % B |
| 0 | 0.8 | 90 | 10 |
| 2 | 0.8 | 90 | 10 |
| 17 | 0.8 | 45 | 55 |
| 17.1 | 0.8 | 5 | 95 |
| 19 | 0.8 | 5 | 95 |
| 19.1 | 0.8 | 90 | 10 |
| 21 | 0.8 | 90 | 10 |

Method 9: Column: Phenomenex Aeris 3.6 μm um Widepore XB-C18; 4.6×150 mm; Temperature: 50° C.

TABLE 15
RP-HPLC Method 9 Gradient

| Time | Flow | | |
|---|---|---|---|
| (min) | (mL/min) | % A | % B |
| 1.40 | 1 | 70 | 30 |
| 1.50 | 1 | 70 | 30 |
| 7.50 | 1 | 30 | 70 |
| 7.60 | 1 | 5 | 95 |
| 10 | 1 | 5 | 95 |
| 10.50 | 1 | 95 | 5 |
| 11.80 | 1 | 95 | 5 |

Method 10: Column: Phenomenex Aeris 3.6 μm um Widepore XB-C18; 4.6×150 mm; Temperature: 50° C.

TABLE 16
RP-HPLC Method 10 Gradient

| Time | Flow | | |
|---|---|---|---|
| (min) | (mL/min) | % A | % B |
| 1.40 | 1 | 80 | 20 |
| 1.50 | 1 | 80 | 20 |
| 7.50 | 1 | 5 | 95 |
| 10.00 | 1 | 5 | 95 |
| 10.50 | 1 | 80 | 20 |
| 11.80 | 1 | 80 | 20 |

Method 11: Column: Waters XBridge C18 3.5 μm; 3×150 mm; Temperature: 50° C.

TABLE 17
RP-HPLC Method 11 Gradient

| Time | Flow | | |
|---|---|---|---|
| (min) | (mL/min) | % A | % B |
| 0 | 0.5 | 90 | 10 |
| 4 | 0.5 | 70 | 30 |
| 22 | 0.5 | 30 | 70 |
| 25 | 0.5 | 5 | 95 |
| 30 | 0.5 | 5 | 95 |
| 30.1 | 0.5 | 90 | 10 |
| 35 | 0.5 | 90 | 10 |

Loading of the 2-Chloro-Chlorotrityl Resin (2-CTC) with Fmoc-Cit-OH

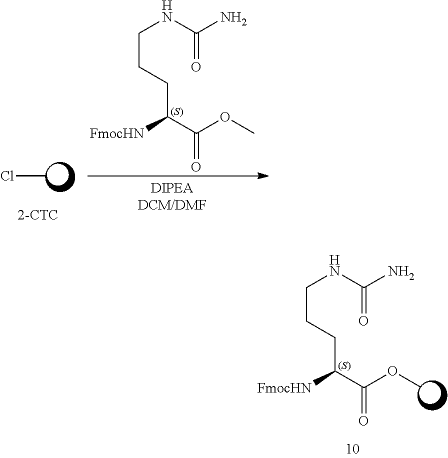

In a 500 mL-Merrifield vessel, the 2-Chlorotrityl Chloride Resin (30.32 g, 1 equiv., 48.51 mmol) was washed with DCM (300 mL) and swollen in DMF (300 mL) for 1 hour. The pre-activated solution (3 min) of Fmoc-Cit-OH (21.21 g, 1.1 Eq, 53.36 mmol), DIPEA (31.34 g, 42.2 mL, 5 equiv., 242.6 mmol) in DMF (300 mL) was added to the resin and reacted by mixing under N₂ bubbling. After filtration, the resin was washed with DMF (300 mL) and DCM (300 mL), and the unreacted resin was capped by adding of a solution of DCM/MeOH/DIPEA 17/2/1 (300 mL) and N₂ bubbling for 15 min. The resin was washed twice with DMF (300 mL), twice with DCM (300 mL), twice with DMF (300 mL), twice with IPA (300 mL) and once with DMF (300 mL).

Elongation of the Peptide and Cleavage of 12 from the Resin:

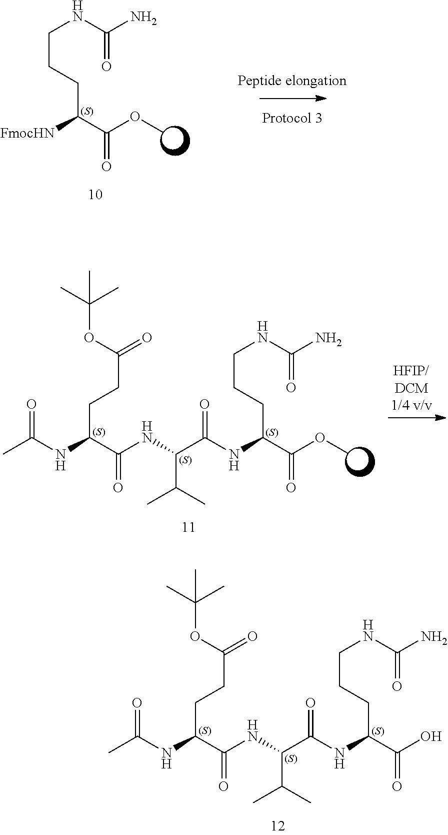

After Fmoc deprotection, elongation of the peptide was performed as described in protocol 3. Compound 12 was cleaved from the resin using the following procedure. After swelling the resin in DCM (300 mL) for 10 min, a solution of HFIP (60 mL) in DCM (240 mL) was added to the resin and shake by N₂ bubbling for 30 min. The resin was filtered and the cleavage was repeated once. The resin was then washed with DCM (300 mL and the combined filtrates were evaporated until dryness, and co-evaporated twice with DCM (100 mL). 12.8 g of pure compound 12 were obtained.

### Coupling of 4-Aminobenzyl Alcohol on Compound 12:

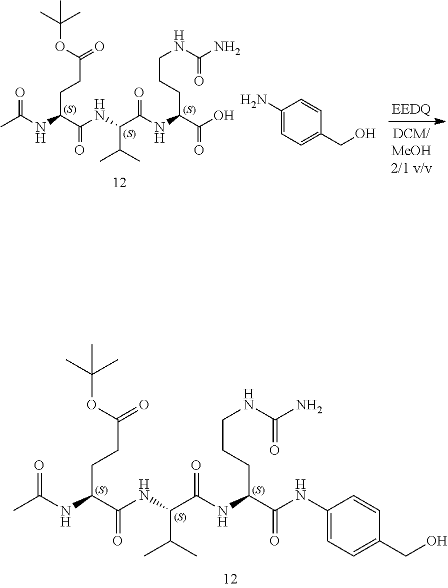

Compound 12 (12.8 g, 1 equiv., 25.5 mmol) was dissolved in a mixture of DCM (120 mL) and MeOH (60 mL). 4-aminobenzyl alcohol (3.46 g, 1.1 equiv., 28.1 mmol) was added followed by EEDQ (12.6 g, 2 equiv., 51.0 mmol). The reaction mixture was stirred, protected from light for 3 h. The reaction mixture was concentrated and suspended in Et₂O (300 mL). After filtration, the white solid was washed twice with Et₂O (100 mL). The white solid was dried under high vacuum overnight yielding 11.8 g of compound 13 (18.5 mmol, 73% yield, 95.1% purity) as a white solid.

Coupling of Fmoc-Glu-OAII on compound 13:

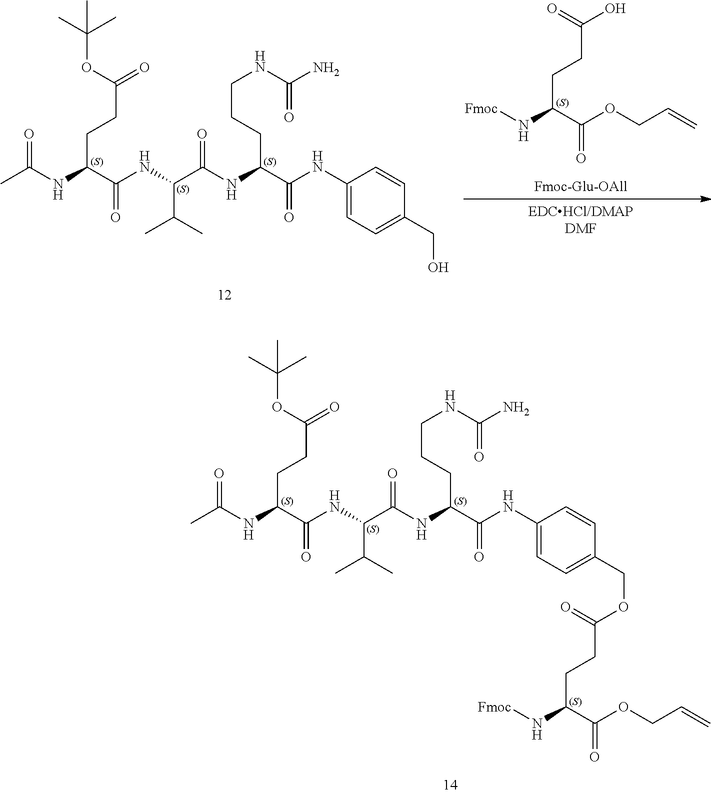

Compound 13 (11.8 g, 1 equiv., 19.4 mmol) and Fmoc-Glu-OAll (9.56 g, 1.2 equiv., 23.3 mmol) were dissolved in DMF (150 mL). EDC·HCl (7.46 g, 2 equiv., 38.9 mmol) and DMAP (0.475 g, 0.2 equiv., 3.89 mmol) were then added. The reaction was stirred overnight. The reaction mixture was then poured dropwise onto cold water (1.2 L). After 15 min stirring, the solution was filtered and the solid was washed with 3 times with water (200 mL). After 15 min drying on the frit, the solid was washed thrice with Et₂O (100 mL). After overnight drying under vacuum, compound 14 was obtained (17.6 g, 82% yield, 16 mmol, 90% purity).

### Synthesis of Compound 6:

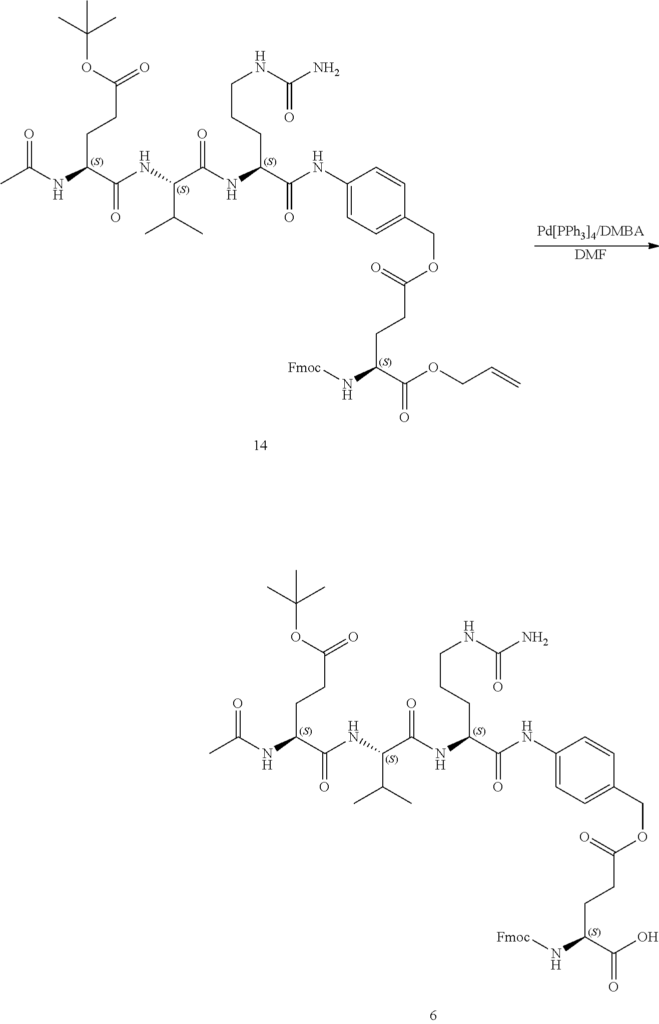

17.6 g of compound 14 (1 equiv., 17.6 mmol) and dimethylbarbituric acid (27.5 g, 10 equiv., 176 mmol) were dissolved in DMF (200 mL). Pd[PPh3]4 (408 mg, 0.02 equiv., 0.353 mmol) were added. After 45 min, L-cysteine hydrochloride (556 mg, 0.2 equiv., 3.53 mmol) were added to complex the palladium in order to remove it at the next step. After 15 min, the mixture was poured dropwise onto cold (4° C.) solution of HCl 0.001 M (pH 3) (1.8 L). After 15 min of stirring, the solution was filtered and the solid was washed with 3 times with water (300 mL). After drying under high vacuum, 13.1 g of compound 6 were obtained (12 mmol, 68% yield, 900% purity).

Described below are the synthesis of selected activatable IL-2 molecules according to the instant disclosure. The remaining activatable IL-2 polypeptides described herein are synthesized according to analogous methods.

### Example 3: Synthesis of CMP-118

Segment 1: Loading of the first KAHA monomer 7 was performed following protocol 1. monomer 7 Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain was deprotected following protocol 14. The crude peptide was purified by preparative HPLC (protocol 20). The purified segment 1 was analyzed using HPLC method 1(tR=6.58 min) and by HRMS (Formula: C₂₃₀H₃₈₉N₆₃O₆₈, found: 5123.9349 Da, theoretical: 5123.8975 Da).

Segment 2: Loading of the first KAHA monomer 8 was performed following protocol 1. Elongation of the peptide chain was performed following protocol 4 until position 41 (Boc-Opr). Segment 2 was then released from the resin and the side chain deprotected following protocol 14. The crude peptide was purified by preparative HPLC (protocol 20). The purified segment 2 was analyzed using HPLC method 9 (tR=5.39 min) and by HRMS (Formula: C₂₃₀H₃₇₆N₄₆O₇₄S, found: 5000.6732 Da, theoretical: 5000.6852 Da).

Segment 3: Loading of the first KAHA monomer 9 was performed following protocol 1. Elongation of the peptide chain was performed following protocol 4 until position 71 (Fmoc-Opr). Segment 3 was then released from the resin and the side chain deprotected following protocol 14. The crude peptide was purified by preparative HPLC (protocol 20). The purified segment 3 was analyzed using HPLC method 10 (tR=5.39 min) and by HRMS (Formula: C₁₈₄H₂₈₈N₄₇O₅₃, found: 1334.7088 Da, theoretical (M+3H)3+/3: 1334.7090 Da).

Segment 4: Loading of Fmoc-Thr(tBu)-OH was performed following protocol 2. Elongation of the peptide chain was performed following protocol 4 until position 104 (Boc-Opr). Segment 4 was then released from the resin and the side chain deprotected following protocol 14. The crude peptide was purified by preparative HPLC (protocol 20). The purified segment 3 was analyzed using HPLC method 10 (tR=7.03 min) and by HRMS (Formula: C₁₅₈H₂₄₄N₃₇O₅₂S, found (M+3H)3+/3: 1174.5748 Da, theoretical (M+3H)3+/3: 1174.5763 Da)

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.942 min) and by HRMS (Formula: C₄₄₉H₇₅₃N₁₀₇O₁₃₇S, found: 9875.5047 Da, theoretical: 9874.5059 Da).

Segment 34: Ligation of segments 3 and 4 and Fmoc-deprotection were performed as described in protocol 16. The ligation/deprotection sample was purified by preparative HPLC (protocol 20). The purified segment 34 was analyzed using HPLC method 10 (tR=6.460 min) and by HRMS (Formula: C₃₂₆H₅₁₆N₈₄O₁₀₁S, found: 7259.7696 Da, theoretical: 7259.7655 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.375 min) and by HRMS (Formula: C₇₇₄H₁₂₇₀N₁₉₂O₂₃₅S₂, found: 17091.2714 Da, theoretical: 17091.2842 Da).

Linear protein: Acm protection on cysteine residues were removed using protocol 18. The ligation sample was purified by preparative HPLC (protocol 20). The purified linear protein was analyzed using HPLC method 2 (tR=13.425 min) and by HRMS (Formula: C₇₆₇H₁₂₅₈N₁₉₀O₂₃₄S₂, found: 16949.1952 Da, theoretical: 16949.2099 Da).

Folded protein: The linear protein was rearranged, folded and purified following protocol 19. The folded protein was analyzed using HPLC method 6 (tR=13.758 min) and by HRMS (Formula: C₇₆₇H₁₂₅₆N₁₉₀O₂₃₄S₂, found: 16.947 Da, theoretical: 16.947 Da).

### Example 4: Synthesis of CMP-119

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 1 (tR=6.16 min) and by HRMS (Formula: C₂₁₅H₃₆₆N₆₀O₆₄, found: 4814.7742 Da, theoretical: 4814.7286 Da).

Segments 2, 3, 4 and ligated segment 34 were synthesized as described in Synthesis of CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.425 min) and by HRMS (Formula: C₄₃₃H₇₂₉N₁₀₅O₁₃₃S, found: 9566.3500 Da, theoretical: 9565.3370 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.225 min) and by HRMS (Formula: C₇₅₈H₁₂₄₅N₁₈₉O₂₃₂S₂, found: 16782.0630 Da, theoretical: 16782.1152 Da).

Linear protein: Acm protection on cysteine residues were removed using protocol 18. The ligation sample was purified by preparative HPLC (protocol 20). The purified linear protein was analyzed using HPLC method 2 (tR=12.875 min) and by HRMS (Formula: C₇₅₂H₁₂₃₅N₁₈₇O₂₃₀S, found: 16639.0306 Da, theoretical: 16640.3510 Da).

Folded protein: The linear protein was rearranged, folded and purified following protocol 19. The folded protein was analyzed using HPLC method 6 (tR=13.675 min) and by HRMS (Formula: C₇₅₂H₁₂₃₃N₁₈₇O₂₃₀S₂, found: 16638.0456 Da, theoretical: 16637.0227 Da).

### Example 5: Synthesis of CMP-120

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 1 (tR=6.59 min) and by HRMS (Formula: C₂₉₇H₅₀₄N₆₄O₉₆, found: 6386.6925 Da, theoretical: 6386.6611 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.808 min) and by HRMS (Formula: C₅₀₅H₈₆₇N₁₀₉O₁₆₅S, found: 11137.3008 Da, theoretical: 11136.2743 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.175 min) and by HRMS (Formula: C₈₃₀H₁₃₈₃N₁₉₃O₂₆₄S₂, found: 18354.0163 Da, theoretical: 18353.0526 Da).

Linear protein: Acm protection on cysteine residues were removed using protocol 18. The ligation sample was purified by preparative HPLC (protocol 20). The purified linear protein was analyzed using HPLC method 2 (tR=12.875 min) and by HRMS (Formula: C₈₂₄H₁₃₇₃N₁₉₁O₂₆₂S₂, found: 18211.9393 Da, theoretical: 18209.9756 Da).

Folded protein: The linear protein was rearranged, folded and purified following protocol 19. The folded protein was analyzed using HPLC method 6 (tR=13.642 min) and by HRMS (Formula: C₈₂₄H₁₃₇₁N₁₉₁O₂₆₂S₂, found: 18209.9772 Da, theoretical: 18208.9552 Da).

### Example 6: Synthesis of CMP-121

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.208 min) and by HRMS (Formula: C₂₅₂H₄₃₁N₆₃O₇₈, found: 5592.2092 Da, theoretical: 5591.1782 Da).

Segments 2, 3, 4 and ligated segment 34 were synthesized as described in Synthesis of CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.8 min) and by HRMS (Formula: C₄₇₀H₇₉₄N₁₀₈O₁₄₇S, found: 10341.7929 Da, theoretical: 10341.7866 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.34 min) and by _{HRMS} (Formula: C₇₉₄H₁₃₀₉N₁₉₃O₂₄₆S, found: 17558.5330 Da, theoretical: 17557.5621 Da).

Linear protein: Acm protection on cysteine residues were removed using protocol 18. The ligation sample was purified by preparative HPLC (protocol 20). The purified linear protein was analyzed using HPLC method 2 (tR=13.425 min) and by HRMS (Formula: C₇₈₈H₁₂₉₉N₁₉₁O₂₄₄S₂, found: 17416.4625 Da, theoretical: 17415.4879 Da).

Folded protein: The linear protein was rearranged, folded and purified following protocol 19. The folded protein was analyzed using HPLC method 6 (tR=13.725 min) and by HRMS (Formula: C₇₈₈H₁₂₉₇N₁₉₁O₂₄₄S₂, found: 17414.4875 Da, theoretical: 17414.4675 Da).

### Example 7: Synthesis of CMP-122

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.54 min) and by HRMS (Formula: C₂₃₇H₄₀₈N₆₀O₇₄, found: 5283.0474 Da, theoretical: 5282.0093 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.408 min) and by HRMS (Formula: C₄₅₅H₇₇₁N₁₀₅O₁₄₃S, found: 10033.6230 Da, theoretical: 10031.6150 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.075 min) and by HRMS (Formula: C₇₇₉H₁₂₈₆N₁₉₀O₂₄₂S₂, found: 17249.3891 Da, theoretical: 17248.3932 Da).

Linear protein: Acm protection on cysteine residues were removed using protocol 18. The ligation sample was purified by preparative HPLC (protocol 20). The purified linear protein was analyzed using HPLC method 2 (tR=13.525 min) and by HRMS (Formula: C₇₇₃H₁₂₇₆N₁₈₈O₂₄₀S₂, found: 17107.2793 Da, theoretical: 17106.3190 Da).

Folded protein: The linear protein was rearranged, folded and purified following protocol 19. The folded protein was analyzed using HPLC method 6 (tR=13.642 min) and by HRMS (Formula: C₇₇₃H₁₂₇₄N₁₈₈O₂₄₀S₂, found: 17105.3338 Da, theoretical. 17104.3033 Da).

### Example 8: Synthesis of CMP-123

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.108 min) and by HRMS (Formula: C₃₀₉H₅₄₆N₆₄O₁₀₆, found: 6853.9628 Da, theoretical: 6852.9390 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.775 min) and by HRMS (Formula: C₅₂₆H₉₀₈N₁₁₀O₁₇₅S, found: 11605.5988 Da, theoretical: 11603.5475 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.258 min) and by HRMS (Formula: C₈₀₁H₁₄₂₃N₁₉₄O₂₇₄S₂, found: 18821.3076 Da, theoretical: 18821.9570 Da).

Linear protein: Acm protection on cysteine residues were removed using protocol 18. The ligation sample was purified by preparative HPLC (protocol 20). The purified linear protein was analyzed using HPLC method 2 (tR=13.325 min) and by HRMS (Formula: C₈₄₅H₁₄₁₄N₁₉₂O₂₇₂S₂, found: 18679.2064 Da, theoretical: 18678.2515 Da).

Folded protein: The linear protein was rearranged, folded and purified following protocol 19. The folded protein was analyzed using HPLC method 6 (RT=13.608 min) and by HRMS (Formula: C₈₄₅H₁₄₁₂N₁₉₂O₂₇₂S₂, found: 18634.2064 Da, theoretical: 18633.2048 Da).

### Example 9: Synthesis of CMP-124

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 1 (tR=6.11 min) and by HRMS (Formula: C₂₅₉H₄₃₅N₇₃O₇₉S, found: 5867.2515 Da, theoretical: 5867.2068 Da).

Segment 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.408 min) and by HRMS (Formula: C₄₇₇H₇₉₈N₁₁₈O₁₄₈S₂, found: 10616.838 Da, theoretical: 10616.819 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.22 min) and by HRMS (Formula: C₈₀₂H₁₃₁₄N₂₀₂O₂₄₇S₃, found: 17833.5698 Da, theoretical: 17832.5954 Da).

Linear protein: Acm protection on cysteine residues were removed using protocol 18. The ligation sample was purified by preparative HPLC (protocol 20). The purified linear protein was analyzed using HPLC method 2 (tR=13.25 min) and by HRMS (Formula: C₇₉₆H₁₃₀₄N₂₀₀O₂₄₅S₃, found: 17691.4516 Da, theoretical. 17690.5212 Da).

Folded protein: The linear protein was rearranged, folded and purified following protocol 19. The folded protein was analyzed using HPLC method 6 (tR=13.558 min) and by HRMS (Formula: C₇₉₆H₁₃₀₂N₂₀₀O₂₄₅S₃, found: 17690.5703 Da, theoretical: 17689.5007 Da).

### Example 10: Synthesis of CMP-125

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 1 (tR=6.05 min) and by HRMS (Formula: C₂₃₃H₃₉₇N₆₅O₆₉S, found: 5244.9606 Da, theoretical: 5245.1850 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.258 min) and by HRMS (Formula: C₄₅₂H₇₆₁N₁₀₉O₁₃₈S₂, found: 9995.5548 Da, theoretical: 9994.5488 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=12.658 min) and by HRMS (Formula: C₇₇₆H₁₂₇₆N₁₉₄O₂₃₇S₃, found: 17211.2889 Da, theoretical: 17210.3243 Da).

Linear protein: Acm protection on cysteine residues were removed using protocol 18. The ligation sample was purified by preparative HPLC (protocol 20). The purified linear protein was analyzed using HPLC method 2 (tR=13.25 min) and by HRMS (Formula: C₇₇₀H₁₂₆₆N₁₉₂O₂₃₅S₃, found: 17069.1853 Da, theoretical: 17068.2500 Da).

Folded protein: The linear protein was rearranged, folded and purified following protocol 19. The folded protein was analyzed using HPLC method 6 (tR=13.558 min) and by HRMS (Formula: C₇₇₀H₁₂₆₄N₁₉₂O₂₃₅S₃, found: 17067.2894 Da, theoretical: 17066.2344 Da).

### Example 11: Synthesis of CMP-126

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.492 min) and by HRMS (Formula: C₃₁₆H₅₅₀N₇₄O₁₀₇S, found: 7129.9836 Da, theoretical: 7129.9704 Da).

Segments 2, 3, 4 and ligated segment 34 were synthesized as described in Synthesis of-CMP-118. Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.392 min) and by ESI (Formula: C534H913N119O176S, m/z found: 1081.20 Da (M+11H+)/11, m/z theoretical: 1081.08 (M+11H+)/Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.025 min) and by HRMS (Formula: C₈₅₉H₁₄₂₉N₂₀₃O₂₇₅S₃, found: 19096.4256 Da, theoretical: 19096.3542 Da).

Linear protein: Acm protection on cysteine residues were removed using protocol 18. The ligation sample was purified by preparative HPLC (protocol 20). The purified linear protein was analyzed using HPLC method 2 (tR=13.09 min) and by HRMS (Formula: C₈₅₃H₁₄₁₉N₂₀₁O₂₇₃S₃, found: 18954.2934 Da, theoretical: 18953.2848 Da).

Folded protein: The linear protein was rearranged, folded and purified following protocol 19. The folded protein was analyzed using HPLC method 6 (tR=13.308 min) and by HRMS (Formula: C₈₅₃H₁₄₁₇N₂₀₁O₂₇₃S₃, found: 18952.2909 Da, theoretical: 18952.2643 Da).

### Example 12: Synthesis of CMP-127

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 3 (tR=6.14 min) and by HRMS (Formula: C₂₈₁H₄₇₇N₇₃O₈₉S, found: 6334.5246 Da, theoretical: 6333.4848 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.358 min) and by HRMS (Formula: C₄₉₈H₈₄₁N₁₁₉O₁₅₈S₂, found: 11086.0995 Da, theoretical: 11084.0931 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=12.975 min) and by HRMS (Formula: C₈₂₃H₁₃₅₅N₂₀₃O₂₅₇S₃, found: 18301.7978 Da, theoretical: 18300.8712 Da).

Linear protein: Acm protection on cysteine residues were removed using protocol 18. The ligation sample was purified by preparative HPLC (protocol 20). The purified linear protein was analyzed using HPLC method 2 (tR=13.47 min) and by HRMS (Formula: C₈₁₇H₁₃₄₅N₂₀₁O₂₅₅S₃, found: 18157.7944 Da, theoretical: 18159.7617 Da).

Folded protein: The linear protein was rearranged, folded and purified following protocol 19. The folded protein was analyzed using HPLC method 6 (tR=13.592 min) and by HRMS (Formula: C₈₁₇H₁₃₄₃N₂₀₁O₂₅₅S₃, found: 18157.7831 Da, theoretical: 18156.7739 Da).

### Example 13: Synthesis of CMP-128

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 3 (tR=6.238 min) and by HRMS (Formula: C₂₅₃H₄₃₉N₆₅O₇₉S, found: 5711.2545 Da, theoretical: 5211.2137 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.308 min) and by HRMS (Formula: C₄₇₂H₈₀₁N₁₁₁O₁₄₈S₂, found: 10462.8460 Da, theoretical: 10461.8219 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.125 min) and by HRMS (Formula: C₇₉₇H₁₃₁₇N₁₉₅O₂₄₇S₃, found: 17678.5650 Da, theoretical: 17677.5975 Da).

Linear protein: Acm protection on cysteine residues were removed using protocol 18. The ligation sample was purified by preparative HPLC (protocol 20). The purified linear protein was analyzed using HPLC method 2 (tR=13.208 min) and by HRMS (Formula: C₇₉₁H₁₃₀₇N₁₉₃O₂₄₅S₃, found: 17536.4918 Da, theoretical: 17535.5232 Da).

Folded protein: The linear protein was rearranged, folded and purified following protocol 19. The folded protein was analyzed using HPLC method 6 (tR=13.592 min) and by HRMS (Formula: C₇₉₁H₁₃₀₅N₁₉₃O₂₄₅S₃, found: 17534.5026 Da, theoretical: 17533.5076 Da).

### Example 14: Synthesis of CMP-129

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.575 min) and by HRMS (Formula: C₃₃₇H₅₉₁N₇₅O₁₁₇S, found: 7597.2576 Da, theoretical: 7596.2484 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.342 min) and by HRMS (Formula: C₅₅₅H₉₅₄N₁₂₀O₁₈₆S₂, found: 12347.8766 Da, theoretical: 12347.8519 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.042 min) and by HRMS (Formula: C₈₈₀H₁₄₇₀N₂₀₄O₂₈₅S₃, found: 19563.6957 Da, theoretical: 19563.6275 Da).

Linear protein: Acm protection on cysteine residues were removed using protocol 18. The ligation sample was purified by preparative HPLC (protocol 20). The purified linear protein was analyzed using HPLC method 2 (RT=13.075 min) and by HRMS (Formula: C₈₇₄H₁₄₆₀N₂₀₂O₂₈₃S₃, found: 19421.6056 Da, theoretical: 19421.5532 Da).

Folded protein: The linear protein was rearranged, folded and purified following protocol 19. The folded protein was analyzed using HPLC method 6 (tR=13.258 min) and by HRMS (Formula: C₈₇₄H₁₄₅₈N₂₀₂O₂₈₃S₃, found: 19419.5512 Da, theoretical: 19419.5375 Da).

### Example 15: Synthesis of CMP-141

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.342 min) and by HRMS (Formula: C₄₀₅H₇₃₈N₆₆O₁₅₄, found: 8996.2404 Da, theoretical: 8996.2096 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.842 min) and by HRMS (Formula: C₆₂₃H₁₁₀₁N₁₁₁O₂₂₃, found: 13746.8636 Da, theoretical: 13746.8180 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.125 min) and by HRMS (Formula: C₉₄₈H₁₆₁₇N₁₉₅O₃₂₂S₂, found: 20962.5670 Da, theoretical: 20962.5935 Da).

Linear protein: Acm protection on cysteine residues were removed using protocol 18. The ligation sample was purified by preparative HPLC (protocol 20). The purified linear protein was analyzed using HPLC method 2 (tR=13.125 min) and by HRMS (Formula: C₉₄₂H₁₆₀₇N₁₉₃O₃₂₀S₂, found: 20820.4559 Da, theoretical: 20820.5193 Da).

Folded protein: The linear protein was rearranged, folded and purified following protocol 19. The folded protein was analyzed using HPLC method 6 (tR=13.325 min) and by HRMS (Formula: C₉₄₂H₁₆₀₅N₁₉₃O₃₂₀S₂, found: 20818.4898 Da, theoretical: 20818.5036 Da).

### Example 16: Synthesis of CMP-142

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.425 min) and by HRMS (Formula: C₄₆₄H₈₅₅N₆₇O₁₈₃, found: 10300.0333 Da, theoretical: 10299.9825 Da).

Segments 2, 3, 4 and ligated segment 34 were synthesized as described in Synthesis of-CMP-1/8. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=ND min) and by HRMS (Formula: C₆₈₂H₁₂₁₈N₁₁₂O₂₅₂S, found: 15050.6483 Da, theoretical: 15050.5894 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.125 min) and by HRMS (Formula: C₁₀₀₇H₁₇₃₄N₁₉₆O₃₅₁S₂, found: 22267.2959 Da, theoretical: 22267.3677 Da).

Linear protein: Acm protection on cysteine residues were removed using protocol 18. The ligation sample was purified by preparative HPLC (protocol 20). The purified linear protein was analyzed using HPLC method 2 (tR=13.142 min) and by HRMS (Formula: C₁₀₀₁H₁₇₂₄N₁₉₄O₃₄₉S₂, found: 22125.2096 Da, theoretical: 22125.2935 Da).

Folded protein: The linear protein was rearranged, folded and purified following protocol 19. The folded protein was analyzed using HPLC method 6 (tR=13.275 min) and by HRMS (Formula: C₁₀₀₁H₁₇₂₂N₁₉₄O₃₄₉S₂, found: 22123.2044 Da, theoretical: 22123.2778 Da).

### Example 17: Synthesis of CMP-143

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.708 min) and by HRMS (Formula: C₃₁₁H₅₃₈N₇₀O₁₁₀, found: 7017.9051 Da, theoretical: 7017.8773 Da).

Segments 2, 3, 4 and ligated segment 34 were synthesized as described in Synthesis of-CMP-118. For Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.525 min) and by HRMS (Formula: C₅₂₉H₉₀₁N₁₁₅O₁₇₉S, found: 11768.4916 Da, theoretical: 11767.4829 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=12.908 min) and by HRMS (Formula: C₈₅₄H₁₄₁₇N₁₉₉O₂₇₈S₂, found: 18984.3084 Da, theoretical: 18984.2611 Da).

Linear protein: Acm protection on cysteine residues were removed using protocol 18. The ligation sample was purified by preparative HPLC (protocol 20). The purified linear protein was analyzed using HPLC method 2 (tR=13.075 min) and by HRMS (Formula: C₈₅₄H₁₄₀₇N₁₉₇O₂₇₆S₂, found: 18842.1387 Da, theoretical: 18842.1869 Da).

Folded protein: The linear protein was rearranged, folded and purified following protocol 19. The folded protein was analyzed using HPLC method 6 (tR=13.375 min) and by HRMS (Formula: C₈₄₈H₁₄₀₅N₁₉₇O₂₇₆S₂, found: 18840.1711 Da, theoretical: 18840.1712 Da).

### Example 18: Synthesis of CMP-144

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.758 min) and by HRMS (Formula: C₃₅₂H₆₃₃N₆₇O₁₂₆, found: 7819.5596 Da, theoretical: 7819.5303 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.575 min) and by HRMS (Formula: C₅₇₀H₉₉₆N₁₁₂O₁₉₅S, found: 12570.1473 Da, theoretical: 12570.89 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=12.875 min) and by HRMS (Formula: C₈₉₅H₁₅₁₂N₁₉₆O₂₉₄S, found: 19785.9195 Da, theoretical: 19785.9142 Da).

Linear protein: Acm protection on cysteine residues were removed using protocol 18. The ligation sample was purified by preparative HPLC (protocol 20). The purified linear protein was analyzed using HPLC method 2 (tR=13.025 min) and by HRMS (Formula: C₈₅₉H₁₅₀₂N₁₉₄O₂₉₂S₂, found: 19644.7655 Da, theoretical: 19643.8400 Da).

Folded protein: The linear protein was rearranged, folded and purified following protocol 19. The folded protein was analyzed using HPLC method 6 (tR=13.325 min) and by HRMS (Formula: C₈₅₉H₁₅₀₀N₁₉₄O₂₉₂S₂, found: 19641.7505 Da, theoretical: 19641.8244 Da).

### Example 19: Synthesis of CMP-145

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.575 min) and by HRMS (Formula: C₄₈₂H_{891N73}O₁₈₆, found: 10684.2903 Da, theoretical: 10685.2687 Da).

Segment 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.375 min) and by HRMS (Formula; C₇₀₀H₁₂₅₄N₁₁₈O₂₅₅S, found: 15435.907 Da, theoretical: 15435.877 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.033 min) and by HRMS (Formula: C₁₀₂₅H₁₇₇₀N₂₀₂O₃₅₄S₂, found: 22652.5701 Da, theoretical: 22651.6526 Da).

Linear protein: Acm protection on cysteine residues were removed using protocol 18. The ligation sample was purified by preparative HPLC (protocol 20). The purified linear protein was analyzed using HPLC method 2 (tR=12.925 min) and by HRMS (Formula: C₁₀₁₉H₁₇₆₀N₂₀₀O₃₅₂S₂, found: 22509.4591 Da, theoretical: 22509.5784 Da).

Folded protein: The linear protein was rearranged, folded and purified following protocol 19. The folded protein was analyzed using HPLC method 6 (tR=13.175 min) and by HRMS (Formula: C₁₀₁₉H₁₇₅₈N₂₀₀O₃₅₂S₂, found: 22507.4974 Da, theoretical: 22507.5627 Da).

### Example 20: Synthesis of CMP-146

Segment 1: Loading of the first KAHA monomer 7 was performed following protocol 1. Elongation of the peptide chain was performed following protocol 3 until position 1 (Boc-Ala). Side chain Alloc deprotection of Lys (position 11) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.62 min) and by HRMS (Formula: C₂₈₈H₅₀₇N₆₃O₉₅, found: 6371.7382 Da, theoretical: 6371.6867 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.975 min) and by HRMS (Formula: C₅₀₆H₈₇₀N₁₀₈O₁₆₄S, found: 11123.2968 Da, theoretical: 11122.2951 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.025 min) and by HRMS (Formula: C831H1386N192O263S2, found: 18339.0991 Da, theoretical: 18339.0733 Da).

Linear protein: Acm protection on cysteine residues were removed using protocol 18. The ligation sample was purified by preparative HPLC (protocol 20). The purified linear protein was analyzed using HPLC method 2 (tR=13.25 min) and by HRMS (Formula: C₈₂₅H₁₃₇₆N₁₉₀O₂₆₁S₂, found: 18196.9540 Da, theoretical: 18195.9964 Da).

Folded protein: The linear protein was rearranged, folded and purified following protocol 19. The folded protein was analyzed using HPLC method 6 (tR=13.458 min) and by HRMS (Formula: C₈₂₅H₁₃₇₄N₁₉₀O₂₆₁S₂, found: 18193.9964 Da, theoretical: 18193.9807 Da).

### Example 21: Synthesis of CMP-147

Segment 1: Loading of the first KAHA monomer 7 was performed following protocol 1. Elongation of the peptide chain was performed following protocol 3 until position 1 (Boc-Ala). Side chain Alloc deprotection of Lys (position 13) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.808 min) and by HRMS (Formula: C₂₈₈H₅₀₇N₆₃O₉₅, found: 6371.7347 Da, theoretical: 6371.6867 Da).

Segments 2, 3, 4 and ligated segment 34 were synthesized as described in Synthesis of-CMP-118. For Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.592 min) and by HRMS (Formula: C₅₀₆H₈₇₀N₁₀₈O₁₆₄S, found: 11122.3235 Da, theoretical: 11122.2951 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.092 min) and by HRMS (Formula: C₈₃₁H₁₃₈₆N₁₉₂O₂₆₃S₂, found: 18339.0889 Da, theoretical: 18339.0733 Da).

Linear protein: Acm protection on cysteine residues were removed using protocol 18. The ligation sample was purified by preparative HPLC (protocol 20). The purified linear protein was analyzed using HPLC method 2 (tR=13.308 min) and by HRMS (Formula: C₈₂₅H₁₃₇₆N₁₉₀O₂₆₁S₂, found: 18196.9363 Da, theoretical: 18195.9964 Da).

Folded protein: The linear protein was rearranged, folded and purified following protocol 19. The folded protein was analyzed using HPLC method 6 (tR=13.475 min) and by HRMS (Formula: C₈₂₅H₁₃₇₄N₁₉₀O₂₆₁S₂, found: 18194.9525 Da, theoretical: 18193.9807 Da).

### Example 22: Synthesis of CMP-148

Segment 1: Loading of the first KAHA monomer 7 was performed following protocol 1. Elongation of the peptide chain was performed following protocol 3 until position 1 (Boc-Ala). Side chain Alloc deprotection of Lys (position 15) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.292 min) and by HRMS (Formula: C₂₈₈H₅₀₈N₆₄O₉₄, found: 6370.7417 Da, theoretical: 6370.7026 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.625 min) and by HRMS (Formula: C₅₀₆H₈₇₁N₁₀₉O₁₆₃S, found: 11122.3330 Da, theoretical: 11121.3110 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.142 min) and by HRMS (Formula: C₈₃₁H₁₃₈₇N₁₉₃O₂₆₂S₂, found: 18338.0174 Da, theoretical: 18338.0893 Da).

Linear protein: Acm protection on cysteine residues were removed using protocol 18. The ligation sample was purified by preparative HPLC (protocol 20). The purified linear protein was analyzed using HPLC method 2 (tR=13.225 min) and by HRMS (Formula: C₈₂₅H₁₃₇₇N₁₉₁O₂₆₀S₂, found: 18196.0136 Da, theoretical: 18195.0123 Da).

Folded protein: The linear protein was rearranged, folded and purified following protocol 19. The folded protein was analyzed using HPLC method 6 (tR=13.442 min) and by HRMS (Formula: C₈₂₅H₁₃₇₅N₁₉₁O₂₆₀S₂, found: 18193.9222 Da, theoretical: 18192.9967 Da).

### Example 23: Synthesis of CMP-149

Segment 1: Loading of the first KAHA monomer 7 was performed following protocol 1. Elongation of the peptide chain was performed following protocol 3 until position 1 (Boc-Ala). Side chain Alloc deprotection of Lys (position 19) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.975 min) and by HRMS (Formula: C₂₈₇H₅₀₄N₆₄O₉₆, found: 6386.6980 Da, theoretical: 6386.6611 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.458 min) and by HRMS (Formula: C₅₀₅H₈₆₇N₁₀₉O₁₆₅S, found: 11137.2718 Da, theoretical: 11137.2696 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.108 min) and by HRMS (Formula: C₈₃₀H₁₃₈₃N₁₉₃O₂₆₄S₂, found: 18354.0052 Da, theoretical: 18354.0478 Da).

Linear protein: Acm protection on cysteine residues were removed using protocol 18. The ligation sample was purified by preparative HPLC (protocol 20). The purified linear protein was analyzed using HPLC method 2 (tR=13.175 min) and by HRMS (Formula: C₈₂₄H₁₃₇₃N₁₉₁O₂₆₂S₂, found: 18211.9716 Da, theoretical: 18210.9709 Da).

Folded protein: The linear protein was rearranged, folded and purified following protocol 19. The folded protein was analyzed using HPLC method 6 (tR=13.408 min) and by HRMS (Formula: C₈₂₄H₁₃₇₁N₁₉₁O₂₆₂S₂, found: 18209.891 Da, theoretical: 18208.9552 Da).

### Example 24: Synthesis of CMP-150

Segment 1: Loading of the first KAHA monomer 7 was performed following protocol 1. Elongation of the peptide chain was performed following protocol 3 until position 1 (Boc-Ala). Side chain Alloc deprotection of Lys (position 22) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.508 min) and by HRMS (Formula: C₂₈₈H₅₀₇N₆₃O₉₅, found: 6371.7267 Da, theoretical: 6371.6867 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.825 min) and by HRMS (Formula: C₅₀₆H₈₇₀N₁₀₈O₁₆₄S, found: 11122.3188 Da, theoretical: 11122.2951 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.068 min) and by HRMS (Formula: C₈₃₁H₁₃₈₆N₁₉₂O₂₆₃S₂, found: 18339.0376 Da, theoretical: 18339.0733 Da).

Linear protein: Acm protection on cysteine residues were removed using protocol 18. The ligation sample was purified by preparative HPLC (protocol 20). The purified linear protein was analyzed using HPLC method 2 (tR=13.242 min) and by HRMS (Formula: C₈₂₅H₁₃₇₆N₁₉₀O₂₆₁S₂, found: 18196.9646 Da, theoretical: 18195.9964 Da).

Folded protein: The linear protein was rearranged, folded and purified following protocol 19. The folded protein was analyzed using HPLC method 6 (tR=13.425 min) and by HRMS (Formula: C₈₂₅H₁₃₇₄N₁₉₀O₂₆₁S₂, found: 18194.9580 Da, theoretical: 18193.9807 Da).

### Example 25: Synthesis of CMP-162

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using analytical method 2 (tR=10.092 min) and by HRMS (Formula: C₃₂₆H₅₆₄N₈₀O₁₁₂, found: 7396.1270 Da, theoretical: 7396.1012 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using analytical method 2 (tR=9.925 min) AND BY HRMS (FORMULA: C₅₄₄H₉₂₇N₁₂₅O₁₈₁S, FOUND: 12146.7060 DA, THEORETICAL: 12146.7095 DA).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using analytical method 2 (tR=12.592 min) and by HRMS (Formula: C₈₆₉H₁₄₄₃N₂₀₉O₂₈₀S₂, found: 19362.4650 Da, theoretical: 19362.4850 Da).

Linear protein: Acm protection on cysteine residues were removed using protocol 18. The ligation sample was purified by preparative HPLC (protocol 20). The purified linear protein was analyzed using analytical method 11 (tR=16.008 min) and by HRMS (Formula: C₈₆₃H₁₄₃₃N₂₀₇O₂₇₈S₂, found: 19220.4268 Da, theoretical: 19220.4108 Da).

Folded protein: The linear protein was rearranged, folded and purified following protocol 19. The folded protein was analyzed using ND (RT=ND min) and by HRMS (Formula: ND, found: ND Da, theoretical: ND Da).

### Example 26: Synthesis of CMP-133

Segment 1: Loading of the first KAHA monomer 7 was performed following protocol 1. Elongation of the peptide chain was performed following protocol 3 until position PEG9 from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 4 (tR=11.092 min) and by HRMS (Formula: C₂₇₅H₄₇₂N₆₄O₈₉, found: 6098.4761 Da, theoretical: 6098.4463 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.492 min) and by HRMS (Formula: C₄₉₃H₈₃₅N₁₀₉O₁₈₅S, found: 10850.0695 Da, theoretical: 10849.0547 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.625 min) and by HRMS (Formula: C₈₁₈H₁₃₅₁N₁₉₃O₂₅₇S₂, found: 18067.8077 Da, theoretical: 18064.8302 Da).

Linear protein: Acm protection on cysteine residues were removed using protocol 18. The ligation sample was purified by preparative HPLC (protocol 20). The purified linear protein was analyzed using HPLC method 7 (tR=12.158 min) and by HRMS (Formula: C₈₁₂H₁₃₄₁N₁₉₁O₂₅₅S₂, found: 17922.7039 Da, theoretical: 17922.7560 Da).

Folded protein: The linear protein was rearranged, folded and purified following protocol 19. The folded protein was analyzed using HPLC method 6 (tR=13.675 min) and by HRMS (Formula: C₈₁₂H₁₃₃₉N₁₉₁O₂₅₅S₂, found: 17920.7535 Da, theoretical: 17920.7403 Da).

### Example 27: Synthesis of CMP-134

Segment 1: Loading of the first KAHA monomer 7 was performed following protocol 1. Elongation of the peptide chain was performed following protocol 3 until position PEG4 from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 4 (tR=10.625 min) and by HRMS (Formula: C₂₈₄H₄₇₈N₇₄O₉₀S, found: 6400.5330 Da, theoretical: 6400.4906 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.842 min) and by HRMS (Formula: C₅₀₂H₈₄₁N₁₁₉O₁₅₉S₂, found: 11151.1083 Da, theoretical: 11151.0989 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 7 (tR=11.792 min) and by HRMS (Formula: C₈₂₇H₁₃₅₇N₂₀₃O₂₅₈S₃, found: 18367.8602 Da, theoretical: 18367.877 Da).

Linear protein: Acm protection on cysteine residues were removed using protocol 18. The ligation sample was purified by preparative HPLC (protocol 20). The purified linear protein was analyzed using HPLC method 2 (tR=13.358 min) and by HRMS (Formula: C₈₂₁H₁₃₄₇N₂₀₁O₂₅₆S₃, found: 18224.7887 Da, theoretical: 18225.8028 Da).

Folded protein: The linear protein was rearranged, folded and purified following protocol 19. The folded protein was analyzed using HPLC method 6 (tR=13.508 min) and by HRMS (Formula: C₈₂₁H₁₃₄₅N₂₀₁O₂₅₆S₃, found: 18223.7852 Da, theoretical: 18223.7871 Da).

### Example 28: Synthesis of CMP-135

Segment 1: Loading of the first KAHA monomer 7 was performed following protocol 1. Elongation of the peptide chain was performed following protocol 3 until position PEG16 from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 4 (tR=10.775 min) and by HRMS (Formula: C₃₃₂H₅₇₄N₇₄O₁₁₄S, found: 7458.1518 Da, theoretical: 7458.1227 Da).

Segments 2, 3, 4 and ligated segment 34 were synthesized as described in Synthesis of-CMP-118. For Segment 12: Ligation of segments 1 and 2 and photodeprotection were performed as described in protocol 15. The ligation/deprotection sample was purified by preparative HPLC (protocol 20). The purified segment 12 was analyzed using HPLC method 4 (tR=10.425 min) and by HRMS (Formula: C₅₅₀H₉₃₇N₁₁₉O₁₈₃S₂, found: 12209.7625 Da, theoretical: 12208.7310 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 7 (tR=11.792 min) and by HRMS (Formula: C₈₅₇H₁₄₅₃N₂₀₃O₂₈₂S₃, found: 19424.4226 Da, theoretical: 19424.5065 Da).

Linear protein: Acm protection on cysteine residues were removed using protocol 18. The ligation sample was purified by preparative HPLC (protocol 20). The purified linear protein was analyzed using HPLC method 2 (tR=13.208 min) and by HRMS (Formula: C₈₆₉H₁₄₄₃N₂₀₁O₂₈₀S₃, found: 19282.3817 Da, theoretical: 19282.4323 Da).

Folded protein: The linear protein was rearranged, folded and purified following protocol 19. The folded protein was analyzed using HPLC method 6 (tR=13.508 min) and by HRMS (Formula: C₈₆₉H₁₄₄₁N₂₀₁O₂₈₀S₃, found: 19280.4035 Da, theoretical: 19280.4167 Da).

### Example 29: Synthesis of CMP-140

Segment 1: Loading of the first KAHA monomer 7 was performed following protocol 1. Elongation of the peptide chain was performed following protocol 3 until position PEG9 from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 4 (tR=11.208 min) and by HRMS (Formula: C₃₀₅H₅₃₂N₆₄O₁₀₄, found: 6758.8663 Da, theoretical: 6758.8396 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.608 min) and by HRMS (Formula: C₅₂₃H₈₉₅N₁₀₉O₁₇₃S, found: 11510.4607 Da, theoretical: 11509.4481 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.558 min) and by HRMS (Formula: C₈₄₈H₁₄₁₁N₁₉₃O₂₇₂S₂, found: 18726.1753 Da, theoretical: 18726.2263 Da).

Linear protein: Acm protection on cysteine residues were removed using protocol 18. The ligation sample was purified by preparative HPLC (protocol 20). The purified linear protein was analyzed using HPLC method 2 (tR=13.642 min) and by HRMS (Formula: C₉₄₂H₁₄₀₁N₁₉₁O₂₇₀S₂, found: 18584.1364 Da, theoretical: 18584.1521 Da).

Folded protein: The linear protein was rearranged, folded and purified following protocol 19. The folded protein was analyzed using HPLC method 6 (tR=13.708 min) and by HRMS (Formula: C₄₂H₁₃₉₉N₁₉₁O₂₇₀S₂, found: 18582.1193 Da, theoretical: 18582.1364 Da).

### Example 30: Synthesis of CMP-152

Segment 1: Loading of the first KAHA monomer 7 was performed following protocol 1. Elongation of the peptide chain was performed following protocol 3 until position PEG9 from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.992 min) and by MALDI-TOF (Formula: C254H431N63O79, found: 5631.101 Da, theoretical: 5631.600 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.225 min) and by HRMS (Formula: C₄₇₂H₇₉₄N₁₀₈O₁₄₈S, found: 10380.7899 Da, theoretical: 10381.7815 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.135 min) and by MALDI-TOF (Formula: C797H1310N192O247S2, found: 17601.908 Da, theoretical: 17598.464 Da).

Linear protein: Acm protection on cysteine residues were removed using protocol 18. The ligation sample was purified by preparative HPLC (protocol 20). The purified linear protein was analyzed using HPLC method 2 (tR=13.508 min) and by HRMS (Formula: C₇₉₁H₁₃₀₀N₁₉₀O₂₄₅S₂, found: 17455.456 Da, theoretical: 17455.4828 Da).

Folded protein: The linear protein was rearranged, folded and purified following protocol 19. The folded protein was analyzed using HPLC method 6 (tR=13.825 min) and by HRMS (Formula: C₇₉₁H₁₂₉₈N₁₉₀O₂₄₅S₂, found: 17453.473 Da, theoretical: 17453.97 Da).

### Example 31: Synthesis of CMP-153

Segment 1: Loading of the first KAHA monomer 7 was performed following protocol 1. Elongation of the peptide chain was performed following protocol 3 until position Gly from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2(tR=11.908 min) and by HRMS (Formula: C₂₅₆H₄₃₄N₆₄O₈₀, found: 5688.2219 Da, theoretical: 5688.1946 Da).

Segments 2, 3, 4 and ligated segment 34 were synthesized as described in Synthesis of CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.192 min) and by HRMS (Formula. C₄₇₄H₇₉₇N₁₀₉O₁₄₆S, found: 10437.8241 Da, theoretical: 10438.803 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.136 min) and by MALDI-TOF (Formula: C799H1313N193O248S2, found: 17652.662 Da, theoretical: 17655.516 Da).

Linear protein: Acm protection on cysteine residues were removed using protocol 18. The ligation sample was purified by preparative HPLC (protocol 20). The purified linear protein was analyzed using HPLC method 2 (tR=13.542 min) and by HRMS (Formula: C₇₉₃H₁₃₀₃N₁₉₁O₂₄₆S₂, found: 17512.4840 Da, theoretical: 17512.5043 Da).

Folded protein: The linear protein was rearranged, folded and purified following protocol 19. The folded protein was analyzed using HPLC method 6 (tR=13.808 min) and by HRMS (Formula: C₇₉₃H₁₃₀₁N₁₉₁O₂₄₆S₂, found: 17510.5274 Da, theoretical: 17510.4886 Da).

### Example 32: Synthesis of CMP-136

Segment 1: Loading of the first KAHA monomer 7 was performed following protocol 1. Elongation of the peptide chain was performed following protocol 3 until position PEG9 from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.992 min) and by HRMS (Formula: C₂₇₁H₄₆₇N₆₃O₉₇, found: 5999.4505 Da, theoretical: 5999.4143 Da).

Segments 2, 3, 4 and ligated segment 34 were synthesized as described in Synthesis of CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.408 min) and by HRMS (Formula: C₄₈₉H₈₃₀N₁₀₈O₁₅₆S, found: 10751.0533 Da, theoretical: 10750.0226 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.137 min) and by MALDI-TOF (Formula: C814H1346N192O255S2, found: 17966.026 Da, theoretical: 17966.931 Da).

Linear protein: Acm protection on cysteine residues were removed using protocol 18. The ligation sample was purified by preparative HPLC (protocol 20). The purified linear protein was analyzed using HPLC method 2 (tR=13.608 min) and by HRMS (Formula: C₈₀₈H₁₃₃₆N₁₉₀O₂₅₃S₂, found: 17823.6979 Da, theoretical: 17823.7239 Da).

Folded protein: The linear protein was rearranged, folded and purified following protocol 19. The folded protein was analyzed using HPLC method 6 (tR=13.625 min) and by HRMS (Formula: C₈₀₈H₁₃₃₄N₁₉₀O₂₅₃S₂, found: 17822.7601 Da, theoretical: 17821.7083 Da).

### Example 33: Synthesis of CMP-137

Segment 1: Loading of the first KAHA monomer 7 was performed following protocol 1. Elongation of the peptide chain was performed following protocol 3 until position PEG24 from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=12.242 min) and by HRMS (Formula: C₃₃₁H₅₈₇N₆₃O₁₁₇, found: 7321.2314 Da, theoretical: 7321.2038 Da).

Segments 2, 3, 4 and ligated segment 34 were synthesized as described in Synthesis of-CMP-118. For segments 12: Ligation of segments 1 and 2 and photodeprotection were performed as described in protocol 15. The ligation/deprotection sample was purified by preparative HPLC (protocol 20). The purified segments 12 was analyzed using HPLC method 2 (tR=10.925 min) and by HRMS (Formula: C₅₄₆H₉₃₂N₁₁₈O₁₈₁S₂, found: 12109.7186 Da, theoretical: 12109.699 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.104 min) and by MALDI-TOF (Formula: C874N1466N192O285S2, found: 19288.371 Da, theoretical: 19288.521 Da).

Linear protein: Acm protection on cysteine residues were removed using protocol 18. The ligation sample was purified by preparative HPLC (protocol 20). The purified linear protein was analyzed using HPLC method 2 (tR=13.592 min) and by HRMS (Formula: C₈₆₈H₁₄₅₆N₁₉₀O₂₈₃S₂, found: 19145.488 Da, theoretical: 19145.5135 Da).

Folded protein: The linear protein was rearranged, folded and purified following protocol 19. The folded protein was analyzed using HPLC method 6 (tR=13.642 min) and by HRMS (Formula: C₈₆₈H₁₄₅₄N₁₉₀O₂₈₃S₂, found: 19143.5463 Da, theoretical: 19143.4978 Da).

### Example 34: Synthesis of CMP-138

Segment 1: Loading of the first KAHA monomer 7 was performed following protocol 1. Elongation of the peptide chain was performed following protocol 3 until position PEG4 from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=10.942 min) and by HRMS (Formula: C₂₈₀H₄₇₃N₇₃O₈₈S, found: 6301.4654 Da, theoretical: 6301.4586 Da).

Segment 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.642 min) and by HRMS (Formula: C₄₉₈H₈₃₆N₁₁₈O₁₅₇S₂, found: 11052.0573 Da, theoretical: 11052.0669 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.153 min) and by MALDI-TOF (Formula: C823H1352N202O256S3, found: 18262.385 Da, theoretical: 18269.207 Da).

Linear protein: Acm protection on cysteine residues were removed using protocol 18. The ligation sample was purified by preparative HPLC (protocol 20). The purified linear protein was analyzed using HPLC method 2 (tR=13.242 min) and by HRMS (Formula: C₈₁₇H₁₃₄₂N₂₀₀O₂₅₄S₃, found: 18126.7783 Da, theoretical: 18125.7681 Da).

Folded protein: The linear protein was rearranged, folded and purified following protocol 19. The folded protein was analyzed using HPLC method 6 (tR=13.492 min) and by HRMS (Formula: C₈₁₇H₁₃₄₀N₂₀₀O₂₅₄S₃, found: 18124.7931 Da, theoretical: 18123.7525 Da).

### Example 35: Synthesis of CMP-139

Segment 1: Loading of the first KAHA monomer 7 was performed following protocol 1. Elongation of the peptide chain was performed following protocol 3 until position PEG16 from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.358 min) and by HRMS (Formula: C₃₂₈H₅₆₉N₇₃O₁₁₂S, found: 7359.1165 Da, theoretical: 7359.0907 Da).

Segments 2, 3, 4 and ligated segment 34 were synthesized as described in Synthesis of CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.642 min) and by HRMS (Formula: C₅₄₉H₉₅₀N₁₀₈O₁₈₆S, found: 12071.8242 Da, theoretical: 12071.8122 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.111 min) and by MALDI-TOF (Formula: C871H1448N202O280S3, found: 19326.067 Da, theoretical: 19326.479 Da).

Linear protein: Acm protection on cysteine residues were removed using protocol 18. The ligation sample was purified by preparative HPLC (protocol 20). The purified linear protein was analyzed using HPLC method 2 (tR=13.308 min) and by HRMS (Formula: C₈₆₅H₁₄₃₈N₂₀₀O₂₇₈S₃, found: 19183.3659 Da, theoretical: 19183.4003 Da).

Folded protein: The linear protein was rearranged, folded and purified following protocol 19. The folded protein was analyzed using HPLC method 6 (tR=13.475 min) and by HRMS (Formula: C₈₆₅H₁₄₃₆N₂₀₀O₂₇₈S₃, found: 19181.4007 Da, theoretical: 19171.3846 Da).

### Example 36: Synthesis of CMP-154

Segment 1: Loading of the first KAHA monomer 7 was performed following protocol 1. Elongation of the peptide chain was performed following protocol 3 until position PEG9 from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=10.992 min) and by HRMS (Formula: C₂₅₀H₄₂₆N₆₂O₇₇, found: 5532.1633 Da, theoretical: 5532.1411 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 5 (tR=4.477 min) and by HRMS (Formula: C₄₆₈H₇₈₉N₁₀₇O₁₄₆S, found: 10282.7779 Da, theoretical: 10282.7495 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.094 min) and by HRMS (Formula: C₇₉₃H₁₃₀₅N₁₉₁O₂₄₅S₂, found: 17498.5038 Da, theoretical: 17498.525 Da).

Linear protein: Acm protection on cysteine residues were removed using protocol 18. The ligation sample was purified by preparative HPLC (protocol 20). The purified linear protein was analyzed using HPLC method 5 (tR=5.151 min) and by HRMS (Formula: C₇₈₇H₁₂₉₅N₁₈₉O₂₄₃S₂, found: 17356.4531 Da, theoretical: 17356.4508 Da).

Folded protein: The linear protein was rearranged, folded and purified following protocol 19. The folded protein was analyzed using HPLC method 6 (tR=13.742 min) and by HRMS (Formula: C₇₈₇H₁₂₉₃N₁₈₉O₂₄₃S₂, found: 17354.4205 Da, theoretical: 17354.4351 Da).

### Example 37: Synthesis of CMP-155

Segment 1: Loading of the first KAHA monomer 7 was performed following protocol 1. Elongation of the peptide chain was performed following protocol 3 until position Gly from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 5 (tR=4.63 min) and by HRMS (Formula: C₂₅₂H₄₂₉N₆₃O₇₈, found: 5589.2068 Da, theoretical: 5589.1626 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.525 min) and by HRMS (Formula: C₄₇₀H₇₉₂N₁₀₈O₁₄₇S, found: 10339.7723 Da, theoretical: 10339.7709 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=11.708 min) and by HRMS (Formula: C₇₉₅H₁₃₀₈N₁₉₂O₂₄₆S₂, found: 17555.512 Da, theoretical: 17555.5465 Da).

Linear protein: Acm protection on cysteine residues were removed using protocol 18. The ligation sample was purified by preparative HPLC (protocol 20). The purified linear protein was analyzed using HPLC method 2 (tR=11.725 min) and by HRMS (Formula: C₇₈₉H₁₂₉₈N₁₉₀O₂₄₄S₂, found: 17413.451 Da, theoretical: 17413.4722 Da).

Folded protein: The linear protein was rearranged, folded and purified following protocol 19. The folded protein was analyzed using HPLC method 6 (tR=13.742 min) and by HRMS (Formula: C₇₈₉H₁₂₉₆N₁₉₀O₂₄₄S₂, found: 17411.4358 Da, theoretical: 17411.4566 Da).

### Example 38: Synthesis of CMP-156

Segment 1: Loading of the first KAHA monomer 7 was performed following protocol 1. Elongation of the peptide chain was performed following protocol 3 until position Gly from the linker part. Side chain All deprotection of Glu (position 19) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 5 (tR=4.558 min) and by HRMS (Formula: C₂₄₀H₄₀₄N₆₄O₇₄, found: 5370.029 Da, theoretical: 5369.99 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 5 (tR=4.428 min) and by HRMS (Formula: C₄₅₈H₇₆₇N₁₀₉O₁₄₃S, found: 10120,6362 Da, theoretical: 10120,5986 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.077 min) and by HRMS (Formula: C₇₈₃H₁₂₈₃N₁₉₃O₂₄₂S₂ found: 17336,3652 Da, theoretical: 17336,3741 Da).

Linear protein: Acm protection on cysteine residues were removed using protocol 18. The ligation sample was purified by preparative HPLC (protocol 20). The purified linear protein was analyzed using HPLC method 2 (tR=12.642 min) and by HRMS (Formula: C₇₇₇H₁₂₇₃N₁₉₁O₂₄₀S₂, found: 17194,3931 Da, theoretical: 17194,2999 Da).

Folded protein: The linear protein was rearranged, folded and purified following protocol 19. The folded protein was analyzed using HPLC method 6 (tR=13.308 min) and by HRMS (Formula: C₇₇₇H₁₂₇₁N₁₉₁O₂₄₀S₂, found: 17192.2843 Da, theoretical: 17193.3661 Da).

### Example 39: Synthesis of CMP-157

Segment 1: Loading of the first KAHA monomer 7 was performed following protocol 1. Elongation of the peptide chain was performed following protocol 3 until position PEG4 from the linker part. Side chain All deprotection of Glu (position 19) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.725 min) and by HRMS (Formula: C₂₃₈H₄₀₁N₆₃O₇₃, found: 5313.0138 Da, theoretical: 5312.9688 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.708 min) and by HRMS (Formula: C₄₅₆H₇₆₄N₁₀₈O₁₄₂S, found: 10063.6125 Da, theoretical: 10062.5745 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.225 min) and by HRMS (Formula: C₇₈₁H₁₂₈₀N₁₉₂O₂₄₁S₂, found: 17279.3246 Da, theoretical: 17279.3527 Da).

Linear protein: Acm protection on cysteine residues were removed using protocol 18. The ligation sample was purified by preparative HPLC (protocol 20). The purified linear protein was analyzed using HPLC method 2 (tR=13.292 min) and by HRMS (Formula: C₇₇₅H₁₂₇₀N₁₉₀O₂₃₉S₂, found: 17137.2785 Da, theoretical: 13137.3669 Da).

Folded protein: The linear protein was rearranged, folded and purified following protocol 19. The folded protein was analyzed using HPLC method 6 (tR=13.342 min) and by HRMS (Formula: C₇₇₅H₁₂₆₈N₁₉₀O₂₃₉S₂, found: 17135.3369 Da, theoretical: 17135.2628 Da).

### Example 40: Synthesis of CMP-158

Segment 1: Loading of the first KAHA monomer 7 was performed following protocol 1. Elongation of the peptide chain was performed following protocol 3 until position Gly from the linker part. Side chain All deprotection of Glu (position 22) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 5 (tR=4.725 min) and by HRMS (Formula. C₂₄₁H₄₀₇N₆₁O₇₃, found: 5355,0386 Da, theoretical: 5355,0158 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 5 (tR=4.529 min) and by HRMS (Formula: C₄₇₀H₇₈₃N₁₀₉O₁₄₅S, found: 10104,6301 Da, theoretical: 10105,6241 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.105 min) and by HRMS (Formula: C₇₈₄H₁₂₈₆N₁₉₂O₂₄₁S₂, found: 17321,3931 Da, theoretical: 17321,3997 Da).

Linear protein: Acm protection on cysteine residues were removed using protocol 18. The ligation sample was purified by preparative HPLC (protocol 20). The purified linear protein was analyzed using HPLC method 2 (tR=12.792 min) and by HRMS (Formula: C₇₇₈H₁₂₇₆N₁₉₀O₂₃₉S₂, found: 17179,3934 Da, theoretical: 17179,3254 Da).

Folded protein: The linear protein was rearranged, folded and purified following protocol 19. The folded protein was analyzed using HPLC method 6 (tR=13.408 min) and by HRMS (Formula: C₇₇₈H₁₂₇₄N₁₉₀O₂₃₉S₂, found: 17178.3777 Da, theoretical: 17177.3098 Da).

### Example 41: Synthesis of CMP-159

Segment 1: Loading of the first KAHA monomer 7 was performed following protocol 1. Elongation of the peptide chain was performed following protocol 3 until position PEG4 from the linker part. Side chain All deprotection of Glu (position 22) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.858 min) and by HRMS (Formula: C₂₃₉H₄₀₄N₆₂O₇₂, found: 5297.0400 Da, theoretical: 5297.9943 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.825 min) and by HRMS (Formula: C₄₅₇H₇₆₇N₁₀₇O₁₄₁S, found: 10048.6371 Da, theoretical: 10048.6027 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.225 min) and by HRMS (Formula: C₇₈₂H₁₂₈₃N₁₉₁O₂₄₀S₂, found: 17264.3550 Da, theoretical: 17264.3782 Da).

Linear protein: Acm protection on cysteine residues were removed using protocol 18. The ligation sample was purified by preparative HPLC (protocol 20). The purified linear protein was analyzed using HPLC method 2 (tR=13.325 min) and by HRMS (Formula: C₇₇₆H₁₂₇₃N₁₈₉O₂₃₈S₂, found: 17122.3802 Da, theoretical: 17122.3040 Da).

Folded protein: The linear protein was rearranged, folded and purified following protocol 19. The folded protein was analyzed using HPLC method 6 (tR=13.342 min) and by HRMS (Formula: C₇₇₆H₁₂₇₁N₁₈₉O₂₃₈S₂, found: 17120.3456 Da, theoretical: 17120.2883 Da).

### Example 42: Synthesis of CMP-160

Segment 1: Loading of the first KAHA monomer 7 was performed following protocol 1. Elongation of the peptide chain was performed following protocol 3 until position Gly from the linker part. Side chain All deprotection of Glu (position 26) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 5 (tR=4.491 min) and by HRMS (Formula: C₂₅₂H₄₂₉N₆₃O₇₉, found: 5589.2053 Da, theoretical: 5589.1626 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 5 (tR=4.301 min) and by HRMS (Formula: C₄₇₀H₇₉₂N₁₀₈O₁₄₇S, found: 10339.8027 Da, theoretical: 10339.7709 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=12.642 min) and by HRMS (Formula: C₇₉₅H₁₃₀₈N₁₉₂O₂₄₆S₂, found: 17555.5332 Da, theoretical: 17555.5465 Da).

Linear protein: Acm protection on cysteine residues were removed using protocol 18. The ligation sample was purified by preparative HPLC (protocol 20). The purified linear protein was analyzed using HPLC method 2 (tR=12.692 min) and by HRMS (Formula: C₇₈₉H₁₂₉₈N₁₉₀O₂₄₄S₂, found: 17413.5417 Da, theoretical: 17413.4722 Da).

Folded protein: The linear protein was rearranged, folded and purified following protocol 19. The folded protein was analyzed using HPLC method 6 (tR=13.325 min) and by HRMS (Formula: C₇₈₉H₁₂₉₆N₁₉₀O₂₄₄S₂, found: 17412.5439 Da, theoretical: 17411.4566 Da).

### Example 43: Synthesis of CMP-161

Segment 1: Loading of the first KAHA monomer 7 was performed following protocol 1. Elongation of the peptide chain was performed following protocol 3 until position PEG9 from the linker part. Side chain All deprotection of Glu (position 26) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.392 min) and by HRMS (Formula: C₂₅₀H₄₂₆N₆₂O₇₇, found: 5532.1709 Da, theoretical: 5532.1411 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.875 min) and by HRMS (Formula: C₄₆₈H₇₈₉N₁₀₇O₁₄₆S, found: 10282.7801 Da, theoretical: 10282.7495 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.258 min) and by HRMS (Formula: C₇₉₃H₁₃₀₅N₁₉₁O₂₄₅S₂, found: 17498.5078 Da, theoretical: 17498.5250 Da).

Linear protein: Acm protection on cysteine residues were removed using protocol 18. The ligation sample was purified by preparative HPLC (protocol 20). The purified linear protein was analyzed using HPLC method 2 (tR=13.342 min) and by HRMS (Formula: C₇₈₇H₁₂₉₅N₁₈₉O₂₄₃S₂, found: 17356.5255 Da, theoretical: 17356.4508 Da).

Folded protein: The linear protein was rearranged, folded and purified following protocol 19. The folded protein was analyzed using HPLC method 6 (tR=13.342 min) and by HRMS (Formula: C₇₈₇H₁₂₉₃N₁₈₉O₂₄₃S₂, found: 17355.5218 Da, theoretical: 17354.4351 Da).

### Example 44: Synthesis of CMP-151

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until PEG4 from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.708 min) and by HRMS (Formula: C₂₅₄H₄₃₂N₆₄O₇₉, found: 5646.2349 Da, theoretical: 5646.184 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.158 min) and by HRMS (Formula: C₄₇₂H₇₉₅N₁₀₉O₁₄₈S, found: 10396.8346 Da, theoretical: 10396.7924 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.392 min) and by HRMS (Formula: C₇₉₇H₁₃₁₁N₁₉₃O₂₄₇S₂, found: 17612.5541 Da, theoretical: 17612.5679 Da).

Linear protein: Acm protection on cysteine residues were removed using protocol 18. The ligation sample was purified by preparative HPLC (protocol 20). The purified linear protein was analyzed using HPLC method 2 (tR=13.475 min) and by HRMS (Formula: C₇₉₁H₁₃₀₁N₁₉₁O₂₄₅S₂, found: 17470.4669 Da, theoretical: 17470.4937 Da).

Folded protein: The linear protein was rearranged, folded and purified following protocol 19. The folded protein was analyzed using HPLC method 6 (tR=13.642 min) and by HRMS (Formula: C₇₉₁H₁₂₉₉N₁₉₁O₂₄₅S₂, found: 17468.4077 Da, theoretical: 17468.478 Da).

### Example 45: Synthesis of CMP-163

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until PEG4 from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using method 2 (tR=11.408 min) and by HRMS (Formula: C₂₄₄H₄₁₂N₆₄O₇₄, found: 5426.0601 Da, theoretical: 5426.0529 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using method 2 (tR=10.792 min) and by HRMS (Formula: C₄₆₂H₇₇₅N₁₀₉O₁₄₃S, found: 10175.6731 Da, theoretical: 10176.6613 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using method 2 (tR=13.492 min) and by HRMS (Formula: C₇₈₇H₂₉₁N₁₉₃O₂₄₂S₂, found: 17392.4924 Da, theoretical: 17392.4368 Da).

Linear protein: Acm protection on cysteine residues were removed using protocol 18. The ligation sample was purified by preparative HPLC (protocol 20). The purified linear protein was analyzed using method 2 (tR=13.425 min) and by HRMS (Formula: C₇₈₁H₁₂₈₁N₁₉₁O₂₄₀S₂, found: 17250.4162 Da, theoretical: 17250.3625 Da).

Folded protein: The linear protein was rearranged, folded and purified following protocol 19. The folded protein was analyzed using ND (tR=ND min) and by HRMS (Formula: ND, found: ND Da, theoretical. ND Da).

### Example 46: Synthesis of CMP-164

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until Gly from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using method 2 (tR=11.392 min) and by HRMS (Formula: C₂₃₅H₃₉₄N₆₄O₇₀, found: 5234.9369 Da, theoretical: 5235.9323 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using method 2 (tR=10.942 min) and by HRMS (Formula: C₄₅₃H₇₅₇N₁₀₉O₁₃₉S, found: 9986.5585 Da, theoretical: 9985.538 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using method 2 (tR=13.358 min) and by HRMS (Formula: C₇₇₈H₁₂₇₃N₁₉₃O₂₃₈S₂, found: 17202.3594 Da, theoretical: 17202.3162 Da).

Linear protein: Acm protection on cysteine residues were removed using protocol 18. The ligation sample was purified by preparative HPLC (protocol 20). The purified linear protein was analyzed using method 2 (tR=13.425 min) and by HRMS (Formula: C₇₇₂H₁₂₆₃N₁₉₁O₂₃₆S₂, found: 17060.2794 Da, theoretical: 17060.242 Da).

Folded protein: The linear protein was rearranged, folded and purified following protocol 19. The folded protein was analyzed using ND (tR=ND min) and by HRMS (Formula: ND, found: ND Da, theoretical: ND Da).

### Example 47: Synthesis of CMP-165

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until Gly from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using method 2 (tR=10.525 min) and by HRMS (Formula: C₂₇₂H₄₅₁N₇₉O₈₅, found: 6187.3571 Da, theoretical: 6187.3481 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using method 2 (tR=10.492 min) and by HRMS (Formula: C₄₉₀H₈₁₄N₁₂₄O₁₅₄S, found: 10937.9718 Da, theoretical: 10937.9565 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using method 2 (tR=13.075 min) and by HRMS (Formula: C₈₁₅H₁₃₃₀N₂₀₉O₂₅₃S₂, found: 18153.7813 Da, theoretical: 18153.7320 Da).

Linear protein: Acm protection on cysteine residues were removed using protocol 18. The ligation sample was purified by preparative HPLC (protocol 20). The purified linear protein was analyzed using method 2 (tR=13.142 min) and by HRMS (Formula: C₈₀₉H₁₃₂₀N₂₀₆O₂₅₁S₂, found: 18011.6885 Da, theoretical: 18011.6578 Da).

Folded protein: The linear protein was rearranged, folded and purified following protocol 19. The folded protein was analyzed using ND (tR=ND min) and by HRMS (Formula: ND, found: ND Da, theoretical. ND Da).

### Example 48: Synthesis of CMP-166

Segment 1: Loading of the first KAHA monomer 7 was performed following protocol 1. Elongation of the peptide chain was performed following protocol 3 until position 1 (Ala). The free N-terminus Ala1 was Alloc protected using protocol 5 Side chain ivDde protection of Lys (position 23) was removed following protocol 8 and elongation was pursued on the free amine side chain following protocol 3 until PEG4 from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using analytical method 2 (RT=10.642 min) and by HRMS (Formula: C₂₈₃H₄₇₂N₈₀O₉₀, found: 6434.5081 Da, theoretical: 6434.4901 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using analytical method 2 (tR=10.525 min) and by HRMS (Formula: ND, found: ND Da, theoretical: ND Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using ND (tR=ND min) and by HRMS (Formula: ND, found: ND Da, theoretical: ND Da).

Linear protein: Acm protection on cysteine residues were removed using protocol 18. The ligation sample was purified by preparative HPLC (protocol 20). The purified linear protein was analyzed using ND (tR=ND min) and by HRMS (Formula: ND, found: ND Da, theoretical: ND Da).

Folded protein: The linear protein was rearranged, folded and purified following protocol 19. The folded protein was analyzed using ND (tR=ND min) and by HRMS (Formula: ND, found: ND Da, theoretical: ND Da).

### Example 49: Synthesis of CMP-167 (Contains Amunix Linker, to be Addressed by ES)

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until PEG9 from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using analytical method 2 (tR=ND min) and by HRMS (Formula: C₂₉₃H₄₉₂N₈₀O₉₅, found: 6654.6213 Da, theoretical: 6654.6361 Da).

Segments 2, 3, 4 and ligated segment 34 were synthesized as described in Synthesis of —C(MP-118.

Segment 12: Ligation of segments 1 and 2 and photodeprotection were performed as described in protocol 15. The ligation/deprotection sample was purified by preparative HPLC (protocol 20). The purified segment 12 was analyzed using analytical method 2 (tR=10.525 min) and by HRMS (Formula: ND, found: ND Da, theoretical: ND Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using ND (tR=ND min) and by HRMS (Formula: ND, found: ND Da, theoretical: ND Da).

Linear protein: Acm protection on cysteine residues were removed using protocol 18. The ligation sample was purified by preparative HPLC (protocol 20). The purified linear protein was analyzed using ND (tR=ND min) and by HRMS (Formula: ND, found: ND Da, theoretical: ND Da).

Folded protein: The linear protein was rearranged, folded and purified following protocol 19. The folded protein was analyzed using ND (tR=ND min) and by HRMS (Formula: ND, found: ND Da, theoretical: ND Da).

### Example 50: Synthesis of CMP-168

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side chain Alloc deprotection of Lys (position 9) was carried out using protocol 7. The linker was elongated on the Lys side chain until residue PEG4 kept Fmoc protected. 2-Cl-Trt deprotection on Glu (position 23) was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.558 min) and by HRMS (Formula: C₂₃₈H₄₀₁N₆₃O₇₃, found: 5312.9824 Da, theoretical: 5312.9688 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.008 min) and by HRMS (Formula: C₄₅₆H₇₆₄N₁₀₈O₁₄₂S, found: 10062.5919 Da, theoretical: 10062.5745 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.358 min) and by HRMS (Formula: C₇₈₁H₁₂₈₀N₁₉₂O₂₄₁S₂, found: 17280.3857 Da, theoretical: 17279.3527 Da).

Linear protein: Acm protection on cysteine residues were removed using protocol 18. The ligation sample was purified by preparative HPLC (protocol 20). The purified linear protein was analyzed using HPLC method 2 (tR=13.408 min) and by HRMS (Formula: C₇₇₅H₁₂₇₀N₁₉₀O₂₃₉S₂, found: 17137.3029 Da, theoretical: 17137.2785 Da).

Folded protein: The linear protein was rearranged, folded and purified following protocol 19. The folded protein was analyzed using ND (tR=ND min) and by HRMS (Formula: ND, found: ND Da, theoretical: ND Da).

### Example 51: Synthesis of CMP-169

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side-chain Alloc deprotection of Lys (position 9) was carried out using protocol 7. The linker was elongated on the Lys side chain until residue Gly kept Fmoc protected. 2-Cl-Trt deprotection on Glu23 was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.408 min) and by HRMS (Formula: C₂₄₀H₄₀₄N₆₄O₇₄, found: 5368.9927 Da, theoretical: 5369.9903 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.042 min) and by HRMS (Formula: C₄₅₈H₇₆₇N₁₀₉O₁₄₃S, found: 10120.6058 Da, theoretical: 10120.5986 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.292 min) and by HRMS (Formula: C₇₈₃H₁₂₈₃N₁₉₃O₂₄₂S₂, found: 17336.3845 Da, theoretical: 17336.3741 Da).

Linear protein: Acm protection on cysteine residues were removed using protocol 18. The ligation sample was purified by preparative HPLC (protocol 20). The purified linear protein was analyzed using HPLC method 2 (tR=13.375 min) and by HRMS (Formula: C₇₇₇H₁₂₇₃N₁₉₁O₂₄₀S₂, found: 17194.2984 Da, theoretical: 17194.2999 Da).

Folded protein: The linear protein was rearranged, folded and purified following protocol 19. The folded protein was analyzed using ND (tR=ND min) and by HRMS (Formula: ND, found: ND Da, theoretical: ND Da).

### Example 52: Synthesis of CMP-130

Segment 1: Loading of the first KAHA monomer 5 was performed following protocol 1. Elongation of the peptide chain was performed following protocol 3 until position 1 (Ala1). Segment 1 was then released from the resin and the side chain deprotected following protocol 14. The crude peptide was purified by preparative HPLC (protocol 20). The purified segment 1 was analyzed using HPLC method 2 (tR=11.008 min) and by HRMS (Formula: C₂₀₂H₃₄₇N₅₅O₆₁, found: 4521.6100 Da, theoretical. 4521.5799 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 8 (tR=14.792 min) and by HRMS (Formula: C₄₂₀H₇₁₀N₁₀₀O₁₃₀S, found: 9273.1906 Da, theoretical: 9272.1883 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using 13.408 (tR=13.408 min) and by HRMS (Formula: C₇₄₃H₁₂₂₆N₁₈₄O₂₂₉S₂, found: 16488.931 Da, theoretical: 16487.9638 Da).

Linear protein: Acm protection on cysteine residues were removed using protocol 18. The ligation sample was purified by preparative HPLC (protocol 20). The purified linear protein was analyzed using HPLC method 2 (tR=13.425 min) and by HRMS (Formula: C₇₃₉H₁₂₁₆N₁₈₂O₂₂₇S₂, found: 16345.8812 Da, theoretical: 16345.8896 Da). Folded protein: The linear protein was rearranged, folded and purified following protocol 19. The folded protein was analyzed using HPLC method 6 (tR=13.675 min) and by HRMS (Formula: C₇₃₉H₁₂₁₄N₁₈₂O₂₂₇S₂, found: 16344.8579 Da, theoretical: 16343.8739 Da) Example 53: Synthesis of CMP-131

Segment 1: Loading of the first KAHA monomer 5 was performed following protocol 1. Elongation of the peptide chain was performed following protocol 3 until position 1 (Boc-Ala). Side chain Alloc deprotection of Dab (position 16) was carried out following protocol 7. Building block 8 (2 equiv.) was then added to the resin in presence of DIPEA (4 equiv.) in DMF (25 mL/mmol resin substitution) for 18 h. The resin was then washed with DMF. After Fmoc deprotection, Fmoc-Glu(OtBu)-OH was coupled using protocol 3. After Fmoc deprotection, the free amine was acetylated (see procedure in protocol 3). Segment 1 was then released from the resin and the side chain deprotected following protocol 14 for only 30 min instead 2 h30. The crude peptide was purified by preparative HPLC (protocol 20). The purified segment 1 was analyzed using HPLC method 2 (tR=11.392 min) and by HRMS (Formula: C₂₂₈H₃₈₃N₆₁O₇₀, found: 5097.8669 Da, theoretical: 5097.8343 Da).

Segments 2, 3, 4 and ligated segment 34 were synthesized as described in Synthesis of-CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.975 min) and by HRMS (Formula: C₄₄H₇₄₆N₁₀₆O₁₃₉S, found: 9848.4435 Da, theoretical: 9848.4427 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using 13.375 (tR=13.375 min) and by HRMS (Formula: C₇₇₁H₁₂₆₂N₁₉₀O₂₃₈S₂, found: 17065.1877 Da, theoretical: 17065.2209 Da).

Linear protein: Acm protection on cysteine residues were removed using protocol 18. The ligation sample was purified by preparative HPLC (protocol 20). The purified linear protein was analyzed using HPLC method 2 (tR=13.725 min) and by HRMS (Formula: C₇₆₅H₁₂₅₂N₁₉₉O₂₃₆S₂, found: 16923.1382 Da, theoretical: 16923.1467 Da).

Folded protein: The linear protein was rearranged, folded and purified following protocol 19. The folded protein was analyzed using HPLC method 6 (tR=13.708 min) and by HRMS (Formula: C₇₆₅H₁₂₅₀N₁₈₈O₂₃₆S₂, found: 16921.0886 Da, theoretical: 16921.131 Da).

### Example 54A: Synthesis of CMP-132

Segment 1: Loading of the first KAHA monomer 5 was performed following protocol 1. Elongation of the peptide chain was performed following protocol 3 until position 1 using building block 9 in position 6. Segment 1 was then released from the resin and the side chain deprotected following protocol 14 with only 30 min instead 2 h30. The crude peptide was purified by preparative HPLC (protocol 20). The purified segment 1 was analyzed using HPLC method 2 (tR=11.008 min) and by HRMS (Formula: C₂₂₉H₃₈₂N₆₂O₆₈, found: 5090.8791 Da, theoretical: 5090.8397 Da).

Segments 2, 3, 4 and ligated segment 34 were synthesized as described in Synthesis of-CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.708 min) and by HRMS (Formula: C₄₄₇H₇₄₅N₁₀₇O₁₃₇S, found: 9841.4448 Da, theoretical: 9841.4481 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using 13.158 (tR=13.158 min) and by HRMS (Formula: C₇₇₂H₁₂₆₁N₁₉₁O₂₃₆S₂, found: 17058.2861 Da, theoretical: 17058.2263 Da).

Linear protein: Acm protection on cysteine residues were removed using protocol 18. The ligation sample was purified by preparative HPLC (protocol 20). The purified linear protein was analyzed using HPLC method 2 (tR=13.342 min) and by HRMS (Formula: C₇₆₆H₁₂₅₁N₁₈₉O₂₃₄S₂, found: 16916.2089 Da, theoretical: 16916.1521 Da).

Folded protein: The linear protein was rearranged, folded and purified following protocol 19. The folded protein was analyzed using HPLC method 6 (tR=13.442 min) and by HRMS (Formula: C₇66H₁₂₄₉N₁₈₉O₂₃₄S₂, found: 16914.1606 Da, theoretical: 16914.1364 Da).

### Example 54B: Synthesis CMP-313

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 13 (t_{R}=14.108 min) and by MS (ESI) (Formula: C₅₀₃H₉₃₃N₈₇O₁₉₁, found: 1408.21 Da, theoretical [M+8H]⁸⁺/8: 1408.05 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 13 (t_{R}=14.008 min) and by MS (ESI) (Formula: C₇₂₁H₁₂₉₆N₁₃₂O₂₆₀S, found: 1601.89 Da, theoretical [M+10H]¹⁰⁺/10: 1601.70 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (tR=15.525 min) and by HRMS (Formula: C₁₀₄₆H₁₈₁₂N₂₁₆O₃₅₉S₂, found: 23222.7736 Da, theoretical: 23221.9987 Da).

Linear protein: Acm protection on cysteine residues were removed using protocol 18. The ligation sample was purified by preparative HPLC (protocol 20). The purified linear protein was analyzed using HPLC method 12 (t_{R}=15.342 min) and by HRMS (Formula: C₁₀₄₀H₁₈₀₂N₂₁₄O₃₅₇S₂, found: 23079.7430 Da, theoretical: 23079.9245 Da).

Folded protein: The linear protein was rearranged, folded and purified following protocol 19. The folded protein was analyzed using HPLC method 6 (t_{R}=13.142 min) and by HRMS (Formula: C₁₀₄₀H₁₈₀₀N₂₁₄O₃₅₇S₂, found: 23078.6502 Da, theoretical: 23077.901 Da).

### Example 54C: Synthesis of CMP-314

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 13 (t_{R}=13.125 min) and by MS (ESI) (Formula: C₃₅₁H₆₂₉N₈₇O₁₁₅, found: 1131.28 Da, theoretical [M+7H]⁷⁺/7: 1130.77 Da).

Segment 2, 3, 4 and ligated segment 34 were synthesized as described in Example 3.

Segment 12: Ligation of segment 1 and 2 and photodeprotection were performed as described in protocol 15. The ligation/deprotection sample was purified by preparative HPLC (protocol 20). The purified segment 12 was analyzed using HPLC method 13 (t_{R}=13.708 min) and by MS (ESI) (Formula: C₅₆₉H₉₉₂N₁₃₂O₁₈₄S, found: 1407.88 Da, theoretical [M+9H]⁹⁺/9: 1407.55 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.542 min) and by HRMS (Formula: C₈₉₄H₁₅₀₈N₂₁₆O₂₈₃S₂, found: 19873.9230 Da, theoretical: 19874.0000 Da).

Linear protein: Acm protection on cysteine residues were removed using protocol 18. The ligation sample was purified by preparative HPLC (protocol 20). The purified linear protein was analyzed using HPLC method 12 (t_{R}=15.658 min) and by HRMS (Formula: C₈₈₈H₁₄₉₈N₂₁₄O₂₈₁S₂, found: 19731.8554 Da, theoretical: 19731.9258 Da).

Folded protein: The linear protein was rearranged, folded and purified following protocol 19. The folded protein was analyzed using HPLC method 6 (t_{R}=13.192 min) and by HRMS (Formula: C₈₈₈H₁₄₉₆N₂₁₄O₂₈₁S₂, found: 19730.7738 Da, theoretical: 19729.9101 Da).

### Example 54D: Synthesis of CMP-310

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (t_{R}=10.225 min) and by HRMS (Formula: C₄₇₃H₈₇₅N₇₃O₁₈₉, found: 10609.1397 Da, theoretical: 10609.1282 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (t_{R}=11.725 min) and by HRMS (Formula: C₆₉₁H₁₂₃₈N₁₁₈O₂₅₈S, found: 15358.7502 Da, theoretical: 15359.7366 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.342 min) and by HRMS (Formula: C₁₀₁₇H₁₇₅₆N₂₀₂O₃₅₇S₂, found: 22575.306 Da, theoretical: 22575.5121 Da).

Linear protein: Acm protection on cysteine residues were removed using protocol 18. The ligation sample was purified by preparative HPLC (protocol 20). The purified linear protein was analyzed using HPLC method 12 (t_{R}=15.675 min) and by HRMS (Formula: C₁₀₁₀H₁₇₄₄N₂₀₀O₃₅₅S₂, found: 22433.4378 Da, theoretical: 22433.3063 Da).

Folded protein: The linear protein was rearranged, folded and purified following protocol 19. The folded protein was analyzed using HPLC method 6 (t_{R}=13.408 min) and by HRMS (Formula: C₁₀₁₀H₁₇₄₂N₂₀₀O₃₅₅S₂, found: 22431.3198 Da, theoretical: 22431.4222 Da).

### Example 54E: Synthesis of CMP-311

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (t_{R}=9.958 min) and by HRMS (Formula: C₄₉₄H₉₁₄N₈₀O₁₉₁, found: 11030.4674 Da, theoretical: 11030.4447 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (t_{R}=11.792 min) and by HRMS (Formula: C₇₁₂H₁₂₇₇N₁₂₅O₂₆₀S, found: 15781.0666 Da, theoretical: 15.781.0531 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.492 min) and by HRMS (Formula: C₁₀₃₈H₁₇₉₄N₂₀₈O₃₅₉S₂, found: 17394.3791 Da, theoretical: 17393.3579 Da).

Linear protein: Acm protection on cysteine residues were removed using protocol 18. The ligation sample was purified by preparative HPLC (protocol 20). The purified linear protein was analyzed using HPLC method 12 (t_{R}=15.658 min) and by HRMS (Formula: C₁₀₃₂H₁₇₈₄N₂₀₆O₃₅₇S₂, found: 22854.6262 Da, theoretical: 22853.7591 Da).

Folded protein: The linear protein was rearranged, folded and purified following protocol 19. The folded protein was analyzed using HPLC method 6 (t_{R}=13.375 min) and by HRMS (Formula: C₁₀₃₂H₁₇₈₂N₂₀₆O₃₅₇S₂, found: 22852.5944 Da, theoretical: 22851.7435 Da).

### Example 54F—Synthesis of CMP-331

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until PEG9 from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using analytical method 2 (t_{R}=10.925 min) and by HRMS (Formula: C₂₄₅H₄₁₆N₆₄O₈₂, found: 5570.0370 Da, theoretical: 5570.0435 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (Rt=12.575 min) and by HRMS (Formula: C₄₆₃H₇₇₉N₁₀₉O₁₅₁S, found: 10320.6540 Da, theoretical: 10320.6519 Da). Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (RT=15.875 min) and by HRMS (Formula: C₇₈₉H₁₂₉₆N₁₉₂O₂₅₆S₂, found: 17536.4276 Da, theoretical: 15535.4322 Da).

Linear protein: Acm protection on cysteine residues were removed using protocol 18. The ligation sample was purified by preparative HPLC (protocol 20). The purified linear protein was analyzed using HPLC method 12 (RT=16.092 min) and by HRMS (Formula: C₇₈₃H₁₂₈₆N₁₉₀O₂₄₈S₂, found: 17394.3791 Da, theoretical: 17393.3579 Da).

Folded protein: The linear protein was rearranged, folded and purified following protocol 19. The folded protein was analyzed using HPLC method 6 (tR=13.792 min) and by HRMS (Formula: C₇₂₃H₁₂₈₄N₁₉₀O₂₄₈S₂, found: 17392.3675 Da, theoretical: 17391.3423 Da).

### Example 54G: Synthesis of CMP-326

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side-chain Alloc deprotection of Lys (position 9) was carried out using protocol 7. The linker was elongated on the Lys side chain until residue Gly kept Fmoc protected. 2-Cl-Trt deprotection on Glu23 was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (t_{R}=11.042 min) and by HRMS (Formula: C₂₂₉H₃₈₅N₆₃O₇₆, found: 5235.8248 Da, theoretical: 5235.8255 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (tR=12.442 min) and by HRMS (Formula: C₄₄₇H₇₄₈N₁₀₈O₁₄₅S, found: 9986.4494 Da, theoretical: 9986.4339 Da).

Segment 1234 (Acm protected cysteines): Ligation of segment 12 and 34 was performed as described in protocol 17. The ligation sample was purified by preparative HPLC (protocol 20).

The purified segment 1234 (Acm protected cysteines) was analyzed using HPLC method 12 (tR=15.725 min) and by HRMS (Formula: C₇₇₂H₁₂₆₄N₁₉₂O₂₄₄S₂, found: 17203.2217 Da, theoretical: 17203.2122 Da).

Linear protein: Acm protection on cysteine residues were removed using protocol 18. The ligation sample was purified by preparative HPLC (protocol 20). The purified linear protein was analyzed using HPLC method 12 (tR=16.058 min) and by HRMS (Formula: C₇₆₆H₁₂₅₄N₁₉₀O₂₄₂S₂, found: 17061.1535 Da, theoretical: 17061.1379 Da).

Folded protein: The linear protein was rearranged, folded and purified following protocol 19. The folded protein was analyzed using HPLC method 6 (tR=13.742 min) and by HRMS (Formula: C₇₆₆H₁₂₅₂N₁₉₀O₂₄₂S₂, found: 17059.1514 Da, theoretical: 17059.1223 Da).

### Example 54H-Synthesis of CMP-327

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side-chain Alloc deprotection of Lys (position 9) was carried out using protocol 7. The linker was elongated on the Lys side chain until residue Gly kept Fmoc protected. 2-Cl-Trt deprotection on Glu23 was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (t_{R}=10.842 min) and by HRMS (Formula: C₂₃₉H₄₀₃N₆₉O₇₃, found: 5410.9949 Da, theoretical: 5411.0392 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (tR=12.525 min) and by HRMS (Formula: C₄₅₇H₇₆₆N₁₁₄O₁₄₂S, found: 10161.6196 Da, theoretical: 10161.6111 Da).

Segment 1234 (Acm protected cysteines): Ligation of segment 12 and 34 was performed as described in protocol 17. The ligation sample was purified by preparative HPLC (protocol 20).

The purified segment 1234 (Acm protected cysteines) was analyzed using HPLC method 12 (tR=15.708 min) and by HRMS (Formula: C₇₈₂H₁₂₈₂N₁₉₈O₂₄₁S₂, found: 17377.3695 Da, theoretical: 17377.3867 Da).

Linear protein: Acm protection on cysteine residues were removed using protocol 18. The ligation sample was purified by preparative HPLC (protocol 20). The purified linear protein was analyzed using HPLC method 12 (tR=16.058 min) and by HRMS (Formula: C₇₇₆H₁₂₇₂N₁₉₆O₂₃₉S₂, found: 17235.2999 Da, theoretical: 17235.3124 Da).

Folded protein: The linear protein was rearranged, folded and purified following protocol 19. The folded protein was analyzed using HPLC method 6 (tR=13.742 min) and by HRMS (Formula: C₇₇₆H₁₂₇₀N₁₉₆O₂₃₉S₂, found: 17233.3213 Da, theoretical: 17233.2968 Da).

### Example 54I—CMP-300

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.392 min) and by HRMS (Formula: C₂₄₀H₄₀₄N₆₄O₇₄, found: 5370.0037 Da, theoretical: 5369.9903 Da).

Segment 2, 3, 4 and ligated segment 34 were synthesized as described elsewhere herein.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 11 (tR=12.908 min) and by HRMS (Formula: C₄₅₈H₇₆₇N₁₀₉O₁₄₃S, found: 10121.6191 Da, theoretical: 10120.5986 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 11 (t_{R}=16.525 min) and by HRMS (Formula: C₇₈₃H₁₂₈₃N₁₉₃O₂₄₂S₂, found: 17337.3684 Da, theoretical: 17336.3741 Da)

Linear protein: Acm protection on cysteine residues were removed using protocol 18. The ligation sample was purified by preparative HPLC (protocol 20). The purified linear protein was analyzed using HPLC method 11 (tR=16.592 min) and by HRMS (Formula: C₇₇₇H₁₂₇₃N₁₉₁O₂₄₀S₂, found: 17195.3045 Da, theoretical: 17194.2999 Da).

Folded protein: The linear protein was rearranged, folded and purified following protocol 19. The folded protein was analyzed using HPLC method 6 (tR=13.675 min) and by HRMS (Formula: C₇₇₇H₁₂₇₁N₁₉₁O₂₄₀S₂, found: 17192.311 Da, theoretical: 17192.2843 Da).

### Example 54J: Synthesis of CMP-318

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 11 (t_{R}=12.672 min) and by MS (ESI) (Formula: C₂₃₉H₄₀₃N₆₉O₇₃, found: 1353.81 [M+4H]⁴⁺/4 Da, theoretical: 1353.81 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=14.542 min) and by MS (ESI) (Formula: C₄₅₇H₇₆₆N₁₁₄O₁₄₂S, found: 1695.63 [M+6H]⁶⁺/6, Da, theoretical: 1694.64 Da).

Segment 1234 (Acm protected cysteines): Ligation of segments 12 and 34 was performed as described in protocol 17. The ligation sample was purified by preparative HPLC (protocol 20) The purified segment 1234 (Acm protected cysteines) was analyzed using HPLC method 12 (t_{R}=15.608 min) and by HRMS (Formula: C₇₈₂H₁₂₈₂N₁₉₈O₂₄₁S₂, found: 17377.3389 Da, theoretical: 17377.3867 Da).

Linear protein: Acm protection on cysteine residues were removed using protocol 18. The ligation sample was purified by preparative HPLC (protocol 20). The purified linear protein was analyzed using HPLC method 12 (t_{R}=15.875 min) and by HRMS (Formula: C₇₇₆H₁₂₇₂N₁₉₆O₂₃₉S₂, found: 17235.2297 Da, theoretical: 17235.3124 Da).

### Example 54K: Synthesis of CMP-319

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 13 (t_{R}=13.775 min) and by MS (ESI) (Formula: C₂₄₈H₄₂₂N₇₆O₇₃, found: 1410.36, Da, theoretical [M+4H]⁴⁺/4:1410.14 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=13.925 min) and by MS (ESI) (Formula: C₄₆₆H₇₈₅N₁₂₁O₁₄₂S, found: 1484.88, Da, theoretical [M+7]⁷⁺/7:1484.88 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.292 min) and by HRMS (Formula: C₇₉₁H₁₃₀₁N₂₀₅O₂₄₁S₂, found: 17602.5510 Da, theoretical: 17602.5568 Da).

Linear protein: Acm protection on cysteine residues were removed using protocol 18. The ligation sample was purified by preparative HPLC (protocol 20). The purified linear protein was analyzed using HPLC method 12 (t_{R}=15.408 min) and by HRMS (Formula: C₇₈₅H₁₂₉₁N₂₀₃O₂₃₉S₂, found: 17460.4515 Da, theoretical: 17460.4826 Da).

Folded protein. The linear protein was rearranged, folded and purified following protocol 19. The folded protein was analyzed using HPLC method 6 (t_{R}=13.425 min) and by HRMS (Formula: C₇₈₅H₁₂₈₉N₂₀₃O₂₃₉S₂, found: 17460.4743 Da, theoretical: 17459.2490 Da).

### Example 54L: Synthesis of CMP-320

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 13 (t_{R}=14.725 min) and by MS (ESI) (Formula: C₂₅₂H₄₂₁N₇₁O₇₇, found: 1420.76, Da, theoretical [M+4H]⁴⁺/4:1420.39 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=14.392 min) and by MS (ESI) (Formula: C₄₇₀H₇₈₄N₁₁₆O₁₄₆S, found: 1490.53 Da, theoretical [M+7H]⁷⁺/7:1490.74 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.592 min) and by HRMS (Formula: C₇₉₅H₁₃₀₀N₂₀₀O₂₄₅S₂, found: 17643.5075 Da, theoretical: 17643.5134 Da).

Linear protein: Acm protection on cysteine residues were removed using protocol 18. The ligation sample was purified by preparative HPLC (protocol 20). The purified linear protein was analyzed using HPLC method 12 (t_{R}=15.825 min) and by HRMS (Formula: C₇₈₉H₁₂₉₀N₁₉₈O₂₄₃S₂, found: 17501.4212 Da, theoretical: 17501.4391 Da).

Folded protein: The linear protein was rearranged, folded and purified following protocol 19. The folded protein was analyzed using HPLC method 6 (t_{R}=13.525 min) and by HRMS (Formula: C₇₈₉H₁₂₈₈N₁₉₈O₂₄₃S₂, found: 17500.4351 Da, theoretical: 17500.2460 Da).

### Example 54M: Synthesis of CMP-321

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 11 (t_{R}=12.425 min) and by MS (ESI) (Formula: C₂₄₄H₄₀₉N₆₉O₇₅, found: 1378.45 Da, theoretical [M+4H]⁴⁺/4:1378.34 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=14.525 min) and by MS (ESI) (Formula: C₄₆₂H₇₇₂N₁₁₄O₁₄₄S, found: 1467.25 Da, theoretical [M+7H]⁷⁺/7:1466.71 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.725 min) and by HRMS (Formula: C₇₈₇H₁₂₈₈N₁₉₈O₂₄₃S₂, found: 17475.3816 Da, theoretical: 17475.4235 Da).

Linear protein: Acm protection on cysteine residues were removed using protocol 18. The ligation sample was purified by preparative HPLC (protocol 20). The purified linear protein was analyzed using HPLC method 12 (t_{R}=15.592 min) and by HRMS (Formula: C₇₈₁H₁₂₇₈N₁₉₆O₂₄₁S₂, found: 17333.3239 Da, theoretical: 17333.3493 Da).

Folded protein: The linear protein was rearranged, folded and purified following protocol 19. The folded protein was analyzed using HPLC method 6 (t_{R}=13.525 min) and by HRMS (Formula: C₇₈₁H₁₂₇₆N₁₉₆O₂₄₁S₂, found: 17331.3375 Da, theoretical: 17332.0500 Da).

### Example 54N: Synthesis of CMP-324

Example 54N contains a modification on the side chain of the Glu23 that required a prior synthesis of a short, protected peptide. The protected peptide with structure 12 was prepared substantially as follows (SEQ ID NO: 685).

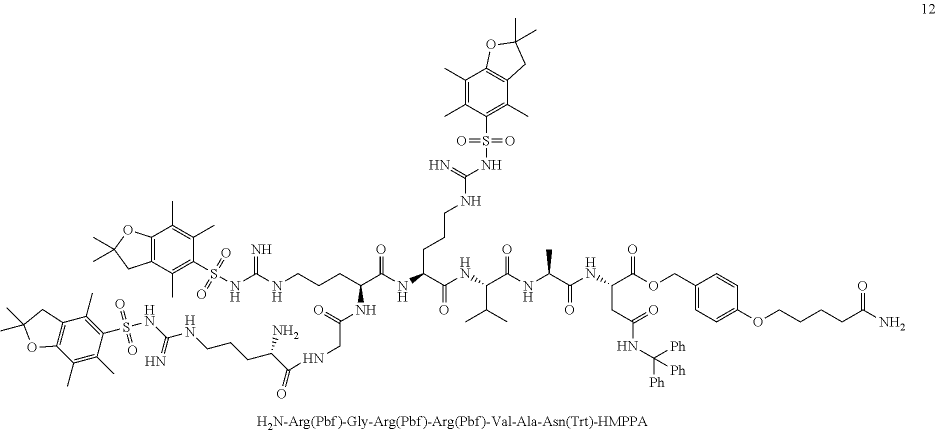 [SMILES: Cc1c(C)c(S(=O)(=O)NC(=N)NCCC[C@H](NC(=O)CNC(=O)[C@@H](N)CCCNC(=N)NS(=O)(=O)c2c(C)c(C)c3c(c2C)CC(C)(C)O3)C(=O)N[C@@H](CCCNC(=N)NS(=O)(=O)c2c(C)c(C)c3c(c2C)CC(C)(C)O3)C(=O)N[C@H](C(=O)N[C@@H](C)C(=O)N[C@@H](CC(=O)NC(c2ccccc2)(c2ccccc2)c2ccccc2)C(=O)OCc2ccc(OCCCCC(N)=O)cc2)C(C)C)c(C)c2c1OC(C)(C)C2]

Loading of 5-(4-(hydroxymethyl) phenoxy) pentanoic acid (HMPPA) on Sieber resin: 8.2 g of Fmoc-protected Sieber resin (5 mmol scale) was swollen in DMF for 30 min. Fmoc-deprotection was performed by treating the resin twice with 20% piperidine in DMF (v/v) at room temperature for 10 min followed by several washes with DMF and a final wash with DCM. 5-(4-(hydroxymethyl) phenoxy) pentanoic acid (HMPPA) (4 g, 25 mmol, 5 equiv.) was dissolved in a mixture of DCM/DMF (8:2), and DIC (7.83 mL, 50 mmol, 10 equiv.) was added to the solution. The mixture was preactivated for 45 min at room temperature before addition to the resin and it was let to react for 16 h at room temperature under gentle agitation. The resin was rinsed thoroughly with DMF and DCM. Then ester formation with Fmoc-Arg (Pbf)-OH was done by firstly preactivating the amino acid (12 mmol, 8 equiv.) dissolved in 15 mL DMF and 35 mL DCM, addition of DIC (3.73 mL, 24 mmol, 16 equiv.) and stirring the solution for 30 min at room temperature before transferring it to the resin. A solution of DMAP (36.7 mg, 0.3 mmol, 0.2 equiv.) in DMF was then added to the resin and the reaction was gently shaken for 16 h. The resin was rinsed thoroughly with DMF and DCM. After ester formation, elongation of the sequence was done according to the conditions described in protocol 3. Resin cleavage of protected peptide was carried out according to protocol 12. The crude peptide was purified by preparative HPLC (protocol 20).

Segment 1: Loading of the first KAHA monomer 5 was performed following protocol 1. Elongation of the peptide chain was performed following protocol 3 until position Leu from the linker part. Side chain Allyl deprotection of Glu (Glu23) was performed following protocol 7. Protected segment 1 was released from the Sieber resin following protocol 12 to keep the side chains protected. The coupling of the protected peptide 12 on the Seg1 was performed in solution. To do so, the cleaved Seg1 (1 equiv.) and Oxyma (4 equiv.) were dissolved in DMF. DIC (2.7 equiv.) was added to the resulting solution and the protected peptide 12 dissolved in DMF was immediately incorporated into the mixture. The pH of the reaction was adjusted to pH 8 by DIPEA addition. After 1 h at room temperature, a second portion of DIC (1.3 equiv.) was added to the reaction and the mixture was gently stirred at 40° C. for 17 h. The solution was then precipitated with H₂O, filtered off and washed twice with H₂O. The side chains on the resulting precipitate peptide were deprotected following protocol 14. The crude peptide was purified by preparative HPLC (protocol 20). The purified segment 1 was analyzed using HPLC method 13 (t_{R}=12.742 min) and by MS (ESI) (Formula: C₂₄₈H₄₂₄N₇₆O₇₄, found: 1414.80 Da, theoretical [M+4H]⁴⁺/4:1414.65 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=13.292 min) and by MS (ESI) (Formula: C₄₆₆H₇₈₇N₁₂₁O₁₄₃S, found: 1041.80 Da, theoretical [M+10H]¹⁰⁺/10:1041.52 Da).

Segment 1234 (Acm protected cysteines): Ligation of segments 12 and 34 was performed as described in protocol 17. The ligation sample was purified by preparative HPLC (protocol 20) The purified segment 1234 (Acm protected cysteines) was analyzed using HPLC method 14 (t_{R}=17.692 min) and by MS (ESI) (Formula: C₇₉₁H₁₃₀₃N₂₀₅O₂₄₂S₂, found: 1469.53 Da, theoretical [M+12H]¹²⁺/12:1469.45 Da).

Linear protein: Acm protection on cysteine residues were removed using protocol 18. The ligation sample was purified by preparative HPLC (protocol 20). The purified linear protein was analyzed using HPLC method 14 (t_{R}=17.775 min) and by HRMS (Formula: C₇₈₅H₁₂₉₃N₂₀₃O₂₄₀S₂, found: 17478.4426 Da, theoretical [M+1H]⁺: 17478.4926 Da).

Folded protein: The linear protein was rearranged, folded and purified following protocol 19. The folded protein was analyzed using HPLC method 6 (t_{R}=13.358 min) and by HRMS (Formula: C₇₈₅H₁₂₉₁N₂₀₃O₂₄₀S₂, found: 17476.4433 Da, theoretical: 17476.4775 Da).

### Example 540: Synthesis of CMP-325

Example 540 contains a modification on the side chain of the Glu23 that required a prior synthesis of a short, protected peptide. The protected peptide with structure 11 was prepared following the protocol described in example 54N.

Segment 1: Loading of the first KAHA monomer 5 was performed following protocol 1. Elongation of the peptide chain was performed following protocol 3 until position Arg from the linker part. Side chain Allyl deprotection of Glu (Glu23) was performed following protocol 7. Protected tripeptide 11 was coupled to side chain of Glu23 by adding a solution of the protected peptide 11 (1.3 equiv.), HATU (1 equiv.) and DIPEA (4 equiv.) in DMF to the resin that was previously swollen in DMF. After gently shaking overnight at room temperature, the resin was rinsed with IPA and DCM. Protected segment 1 was then released from the resin and side chains deprotected following protocol 14. The crude peptide was purified by preparative HPLC (protocol 20). The purified segment 1 was analyzed using HPLC method 13 (t_{R}=13.075 min) and by MS (ESI) (Formula: C₂₄₈H₄₂₄N₇₆O₇₄, found: 1414.74 Da, theoretical [M+4H]⁴⁺/4: 1414.64 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=13.292 min) and by MS (ESI) (Formula: C₄₆₆H₇₈₇N₁₂₁O₁₄₃S, found: 1041.70 Da, theoretical [M+10H]¹⁰⁺/10:1041.52 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=14.842 min) and by MS (ESI) (Formula: C₇₉₁H₁₃₀₃N₂₀₅O₂₄₂S₂, found: 1602.99 Da, theoretical [M+11H]¹¹⁺/11:1602.95 Da).

Linear protein: Acm protection on cysteine residues were removed using protocol 18. The ligation sample was purified by preparative HPLC (protocol 20). The purified linear protein was analyzed using HPLC method 12 (t_{R}=15.175 min) and by HRMS (Formula: C₇₈₅H₁₂₉₃N₂₀₃O₂₄₀S₂, found: 17478.4390 Da, theoretical [M+1H]: 17478.4926 Da).

Folded protein: The linear protein was rearranged, folded and purified following protocol 19. The folded protein was analyzed using HPLC method 6 (t_{R}=13.358 min) and by HRMS (Formula: C₇₈₅H₁₂₉₁N₂₀₃O₂₄₀S₂, found: 17476.4409 Da, theoretical: 17476.4775 Da).

### Example 54P: Synthesis of CMP-322

Example 54N contains a modification on the side chain of the Glu23 that required a prior synthesis of a short, protected peptide. The protected peptide with structure 10 was prepared as follows:

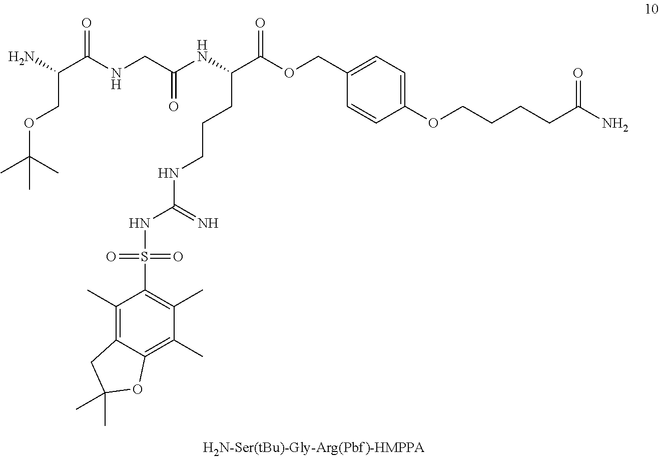 [SMILES: Cc1c(C)c(S(=O)(=O)NC(=N)NCCC[C@H](NC(=O)CNC(=O)[C@@H](N)COC(C)(C)C)C(=O)OCc2ccc(OCCCCC(N)=O)cc2)c(C)c2c1OC(C)(C)C2]

Loading of 5-(4-(hydroxymethyl) phenoxy) pentanoic acid (HMPPA) on Sieber resin: 8.2 g of Fmoc-protected Sieber resin (5 mmol scale) was swollen in DMF for 30 min. Fmoc-deprotection was performed by treating the resin twice with 20% piperidine in DMF (v/v) at room temperature for 10 min followed by several washes with DMF and a final wash with DCM. S-(4-(hydroxymethyl) phenoxy) pentanoic acid (HMPPA) (4 g, 25 mmol, 5 equiv.) was dissolved in a mixture of DCM/DMF (8:2), and DIC (7.83 mL, 50 mmol, 10 equiv.) was added to the solution. The mixture was preactivated for 45 min at room temperature before addition to the resin and it was let to react for 16 h at room temperature under gentle agitation. The resin was rinsed thoroughly with DMF and DCM. Then ester formation with Fmoc-Arg (Pbf)-OH was done by firstly preactivating the amino acid (12 mmol, 8 equiv.) dissolved in 15 mL DMF and 35 mL DCM, addition of DIC (3.73 mL, 24 mmol, 16 equiv.) and stirring the solution for 30 min at room temperature before transferring it to the resin. A solution of DMAP (36.7 mg. 0.3 mmol, 0.2 equiv.) in DMF was then added to the resin and the reaction was gently shaken for 16 h. The resin was rinsed thoroughly with DMF and DCM. After ester formation, elongation of the sequence was done according to the conditions described in protocol 3. Resin cleavage of protected peptide was carried out according to protocol 12. The crude peptide was purified by preparative HPLC (protocol 20).

Segment 1: Loading of the first KAHA monomer 5 was performed following protocol 1. Elongation of the peptide chain was performed following protocol 3 until position Leu from the linker part. Side chain Allyl deprotection of Glu (Glu23) was performed following protocol 7. Protected tripeptide 10 was coupled to side chain of Glu23 by adding a solution of the protected peptide 10 (1.3 equiv.), HATU (1 equiv.) and DIPEA (4 equiv.) in DMF to the resin that was previously swollen in DMF. After gently shaking overnight at room temperature, the resin was rinsed with IPA and DCM.

Protected segment 1 was then released from the resin and side chains deprotected following protocol 14. The crude peptide was purified by preparative HPLC (protocol 20). The purified segment 1 was analyzed using HPLC method 13 (t_{R}=14.358 min) and by MS (ESI): (Formula: C₂₂₇H₃₈₅N₆₅O₇₀, found: 1030.16 Da, theoretical [M+5H]⁵⁺/5:1030.0 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=14.625 min) and by MS (ESI) (Formula: C₄₄₅H₇₄₈N₁₁₀O₁₃₉S, found: 1650.15 Da, theoretical [M+6H]⁶⁺/6:1649.7 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.542 min) and by MS (ESI) (Formula: C₇₇₀H₁₂₆₄N₁₉₄O₂₃₈S₂, found: Da, theoretical 1070.27 [M+16H]¹⁶⁺/16:1070.5 Da).

Linear protein: Acm protection on cysteine residues were removed using protocol 18. The ligation sample was purified by preparative HPLC (protocol 20). The purified linear protein was analyzed using HPLC method 12 (t_{R}=13.558 min) and by HRMS (Formula: C₇₆₄H₁₂₅₄N₁₉₂O₂₃₆S₂, found: 16969.0801 Da, theoretical: 16969.1745 Da).

Folded protein: The linear protein was rearranged, folded and purified following protocol 19. The folded protein was analyzed using HPLC method 6 (t_{R}=13.558 min) and by HRMS (Formula: C₇₆₄H₁₂₅₂N₁₉₂O₂₃₆S₂, found: 16967.0578 Da, theoretical: 16967.1589 Da)

### Example 54Q: Synthesis of CMP-323

Example 540 contains a modification on the side chain of the Glu23 that required a prior synthesis of a short, protected peptide. The protected peptide with structure 11 was prepared following the protocol described in example 54N.

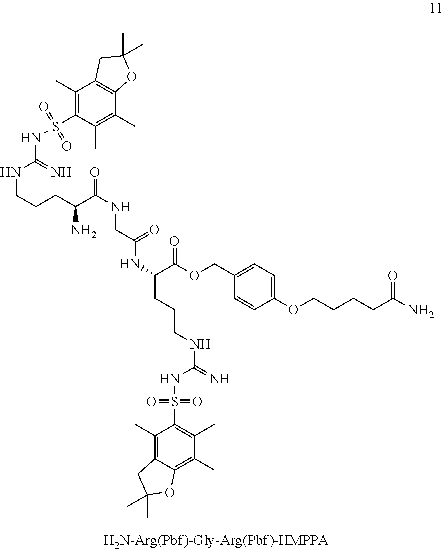 [SMILES: Cc1c(C)c(S(=O)(=O)NC(=N)NCCC[C@H](NC(=O)CNC(=O)[C@@H](N)CCCNC(=N)NS(=O)(=O)c2c(C)c(C)c3c(c2C)CC(C)(C)O3)C(=O)OCc2ccc(OCCCCC(N)=O)cc2)c(C)c2c1OC(C)(C)C2]

Segment 1: Loading of the first KAHA monomer 5 was performed following protocol 1. Elongation of the peptide chain was performed following protocol 3 until position Leu from the linker part. Side chain Allyl deprotection of Glu (Glu23) was performed following protocol 7.

Protected tripeptide 11 was coupled to side chain of Glu23 by adding a solution of the protected peptide 11 (1.3 equiv.), HATU (1 equiv.) and DIPEA (4 equiv.) in DMF to the resin that was previously swollen in DMF. After gently shaking overnight at room temperature, the resin was rinsed with IPA and DCM. Protected segment 1 was then released from the resin and side chains deprotected following protocol 14. The crude peptide was purified by preparative HPLC (protocol 20). The purified segment 1 was analyzed using HPLC method 13 (t_{R}=13.442 min) and by MS (ESI) (Formula: C₂₃₀H₃₉₂N₆₈O₆₉, found: 1304.29 Da, theoretical [M+4H]⁴⁺/4:1304.5 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=13.592 min) and by MS (ESI) (Formula: C₄₄₈H₇₅₅N₁₁₃O₁₃₈, found: 1424.91 Da, theoretical [M+7H]⁷⁺/7:1424.53 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.342 min) and by HRMS (Formula: C₇₃₃H₁₂₇₁N₁₉₇O₂₃₇S₂, found: 17180.2327 Da, theoretical: 17180.3178 Da).

Linear protein: Acm protection on cysteine residues were removed using protocol 18. The ligation sample was purified by preparative HPLC (protocol 20). The purified linear protein was analyzed using HPLC method 12 (t_{R}=15.583 min) and by HRMS (Formula: C₇₆₇H₁₂₆₁N₁₉₅O₂₃₅S₂, found: 17038.1721 Da, theoretical: 17038.2436 Da).

Folded protein: The linear protein was rearranged, folded and purified following protocol 19. The folded protein was analyzed using HPLC method 6 (t_{R}=13.508 min) and by HRMS (Formula: C₇₆₇H₁₂₅₉N₁₉₅O₂₃₅S₂, found: 17036.0969 Da, theoretical: 17036.2279 Da).

### Example 55: IL-2 Receptor βγ Reporter Assay in HEK-Blue™ Cells

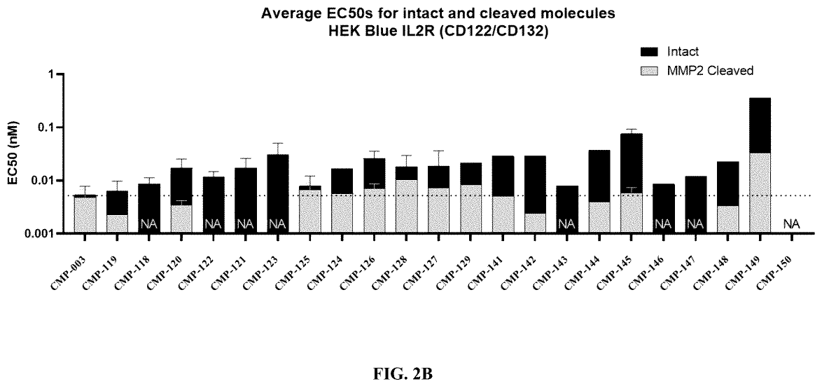

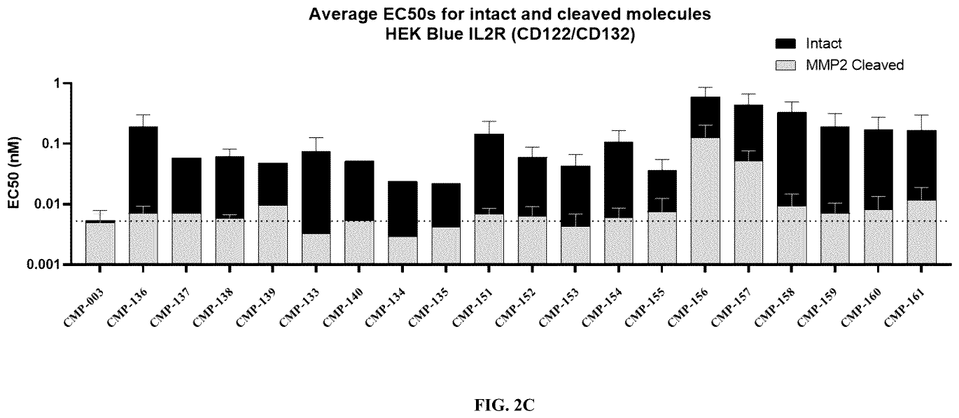

An IL-2Rβγ positive HEK-Blue reporter cell line was used to determine binding of activatable IL-2 polypeptides (and activated versions thereof) to IL-2Rβγ and subsequent downstream signaling that resulted in activation of a STAT5 transcriptional reporter. Activatable IL-2 polypeptides were either intact or MMP2 treated for activation. The general protocol was as follows: 5× 104 cells HEK-Blue™ IL-2Rβγ reporter cells (InvivoGen, ##hkb-il2bg) were seeded into each well of a 96 well plate and stimulated with 0-100 nM of IL-2 polypeptide variants at 37° C. and 5% CO2. After a 20h incubation, 20 μL of cell culture supernatant was then taken from each well and mixed with 180 μL QUANTI-Blue™ media in a 96 well plate, then incubated for 1 hour at 37° C. and 5% CO2. The absorbance signal at 620 nm was then measured on an Enspire® plate reader with 680 and 615 nm as excitation and emission wavelengths, respectively. Half Maximal Effective dose (EC50) was calculated based on a variable slope, four parameter analysis using GraphPad PRISM® software (FIGS. 2A, 2B, 2C).

TABLE 18
EC₅₀ in HEK-Blue IL-2R (CD122⁺,
CD132⁺) Reporter Assay for Selected Molecules

| | Intact molecule | | |
|---|---|---|---|
| | Average EC50 | | |
| Molecule # | (nM) | Std Deviation | n= |
| CMP-003 | 0.0054 | 0.0025 | 17 |
| CMP-130 | 1.3420 | NA | 1 |
| CMP-131 | 1.1470 | NA | 1 |
| CMP-132 | 0.0026 | NA | 1 |

TABLE 19A
EC50 in HEK-Blue IL-2R (CD122+, CD132+) Reporter
Assay for Selected Molecules

| | Intact molecule | | | MMP2 cleaved molecule | | |
|---|---|---|---|---|---|---|
| Molecule | Average | Std | | Average | Std | |
| # | EC50 (nM) | Deviation | n= | EC50 (nM) | Deviation | n= |
| CMP-003 | 0.0054 | 0.0025 | 17 | 0.0049 | 0.0020 | 10 |
| CMP-119 | 0.0063 | 0.0035 | 4 | 0.0023 | NA | 1 |
| CMP-118 | 0.0086 | 0.0027 | 3 | NA | NA | 0 |
| CMP-120 | 0.0172 | 0.0083 | 7 | 0.0034 | 0.0008 | 3 |
| CMP-122 | 0.0117 | 0.0030 | 3 | NA | NA | 0 |
| CMP-121 | 0.0174 | 0.0089 | 3 | NA | NA | 0 |
| CMP-123 | 0.0313 | 0.0196 | 3 | NA | NA | 0 |
| CMP-125 | 0.0079 | 0.0043 | 2 | 0.0067 | 0.0023 | 2 |
| CMP-124 | 0.0166 | NA | 1 | 0.0057 | NA | 1 |
| CMP-126 | 0.0261 | 0.0098 | 2 | 0.0070 | 0.0016 | 2 |
| CMP-128 | 0.0182 | 0.0114 | 4 | 0.0105 | NA | 1 |
| CMP-127 | 0.0189 | 0.0176 | 4 | 0.0073 | NA | 1 |
| CMP-129 | 0.0217 | NA | 1 | 0.0084 | NA | 1 |
| CMP-141 | 0.0288 | NA | 1 | 0.0051 | NA | 1 |
| CMP-142 | 0.0287 | NA | 1 | 0.0024 | NA | 1 |
| CMP-143 | 0.0079 | NA | 1 | NA | NA | 0 |
| CMP-144 | 0.0375 | NA | 1 | 0.0040 | NA | 1 |
| CMP-145 | 0.0759 | 0.0172 | 2 | 0.0060 | 0.0015 | 2 |
| CMP-146 | 0.0086 | NA | 1 | NA | NA | 0 |
| CMP-147 | 0.0121 | NA | 1 | NA | NA | 0 |
| CMP-148 | 0.0230 | NA | 1 | 0.0034 | NA | 1 |
| CMP-149 | 0.3603 | NA | 1 | 0.0333 | NA | 1 |
| CMP-150 | NA | NA | 0 | NA | NA | 0 |

TABLE 19B
EC50 in HEK-Blue IL-2R (CD122+, CD132+) Reporter
Assay for Macrocyclic Activatable Molecules

| | Intact molecule | | | MMP2 cleaved molecule | | |
|---|---|---|---|---|---|---|
| Molecule | Average | Std | | Average | Std | |
| # | EC50 (nM) | Deviation | n= | EC50 (nM) | Deviation | n= |
| CMP-003 | 0.0054 | 0.0025 | 17 | 0.0049 | 0.0020 | 10 |
| CMP-136 | 0.1925 | 0.1089 | 3 | 0.0071 | 0.0022 | 3 |
| CMP-137 | 0.0580 | NA | 1 | 0.0071 | NA | 1 |
| CMP-138 | 0.0614 | 0.0199 | 3 | 0.0058 | 0.0008 | 3 |
| CMP-139 | 0.0483 | NA | 1 | 0.0096 | NA | 1 |
| CMP-133 | 0.0745 | 0.0508 | 2 | 0.0032 | NA | 1 |
| CMP-140 | 0.0515 | NA | 1 | 0.0054 | NA | 1 |
| CMP-134 | 10.0237 | NA | 1 | 0.0029 | NA | 1 |
| CMP-135 | 0.0219 | NA | 1 | 0.0042 | NA | 1 |
| CMP-151 | 0.1446 | 0.0902 | 6 | 0.0068 | 0.0017 | 4 |
| CMP-152 | 0.0597 | 0.0285 | 4 | 0.0064 | 0.0027 | 4 |
| CMP-153 | 0.0432 | 0.0225 | 4 | 0.0043 | 0.0025 | 4 |
| CMP-154 | 0.1060 | 0.0588 | 4 | 0.0059 | 0.0027 | 4 |
| CMP-155 | 0.0359 | 0.0190 | 4 | 0.0075 | 0.0050 | 4 |
| CMP-156 | 0.5960 | 0.2609 | 2 | 0.1276 | 0.0755 | 2 |
| CMP-167 | 0.4441 | 0.2210 | 2 | 0.0521 | 0.0240 | 2 |
| CMP-158 | 0.3294 | 0.1610 | 2 | 0.0093 | 0.0054 | 2 |
| CMP-159 | 0.1921 | 0.1238 | 2 | 0.0071 | 0.0033 | 2 |
| CMP-160 | 0.1723 | 0.1025 | 2 | 0.0082 | 0.0053 | 2 |
| CMP-161 | 0.1683 | 0.1309 | 2 | 0.0118 | 0.0071 | 2 |

TABLE 19C
EC50 in HEK-Blue IL-2R (CD122+, CD132+) Reporter Assay for Additional Macrocyclic Activatable IL-2s

| | | | | MMP2 cleaved | | | Matriptase cleaved | | | uPa cleaved | | |
|---|---|---|---|---|---|---|---|---|---|---|---|---|
| | Intact molecule | | | molecule | | | molecule | | | molecule | | |
| | Average | | | Average | | | Average | | | Average | | |
| CMP | EC50 | Std | | EC50 | Std | | EC50 | Std | | EC50 | Std | |
| # | (nM) | Deviation | n= | (nM) | Deviation | n= | (nM) | Deviation | n= | (nM) | Deviation | n= |
| 3 | 0.0049 | 0.0022 | 31 | 0.0042 | 0.0019 | 23 | | | | | | |
| 163 | 0.1185 | 0.09178 | 2 | 0.0054 | 0.0045 | 2 | | | | | | |
| 164 | 0.1388 | 0.0664 | 2 | 0.0168 | 0.0220 | 2 | | | | | | |
| 165 | 0.3163 | 0.1071 | 7 | 0.0082 | 0.0045 | 6 | | | | | | |
| 166 | 0.3295 | 0.01301 | 2 | 0.0079 | 0.0004 | 2 | | | | | | |
| 167 | 0.1889 | 0.001626 | 2 | 0.0078 | 0.0014 | 2 | | | | | | |
| 168 | 0.8489 | 0.07022 | 2 | 0.0175 | 0.0078 | 2 | | | | | | |
| 169 | 0.1610 | 0.08704 | 2 | 0.0147 | 0.0108 | 2 | | | | | | |
| 300 | 0.711 | 0.5757 | 2 | 0.00940 | 0.008018 | 2 | | | | | | |
| 301 | 0.5906 | 0.4445 | 2 | 0.00726 | 0.001738 | 2 | | | | | | |
| 302 | 0.4361 | 0.2855 | 2 | 0.00646 | 0.003352 | 2 | | | | | | |
| 303 | 0.521 | 0.2366 | 2 | 0.02179 | 0.01902 | 2 | | | | | | |
| 304 | 0.34 | 0.2409 | 2 | 0.02098 | 0.01433 | 2 | | | | | | |
| 305 | 0.4974 | 0.439 | 2 | 0.01845 | 0.01564 | 2 | | | | | | |
| 326 | 0.6724 | 0.1154 | 2 | NA | NA | | | | | | | |
| 327 | 0.8687 | 0.0613 | 2 | 0.0481 | 0.0070 | 2 | | | | | | |
| 315 | 0.0554 | 0.0136 | 2 | 0.0418 | 0.0073 | 2 | 0.0349 | 0.0029132 | 2 | 0.0581 | 0.0043982 | 2 |
| 316 | 0.0968 | 0.0343 | 2 | 0.0963 | 0.0307 | 2 | 0.08276 | 0.0201384 | 2 | 0.08843 | 0.0276690 | 2 |
| 328 | 0.8089 | 0.1105 | 2 | 0.0964 | 0.0019 | 2 | 0.3521 | 0.0493560 | 2 | 0.57125 | 0.0102530 | 2 |
| 329 | 1.7040 | 0.2348 | 2 | 0.0702 | 0.0026 | 2 | 1.4675 | 0.2750645 | 2 | 1.4055 | 0.0035355 | 2 |
| 330 | 1.7250 | 0.3847 | 2 | 0.5580 | 0.1174 | 2 | 2.15 | 0.3379970 | 2 | 1.7325 | 0.2241528 | 2 |
| 317 | 2.2355 | 0.338704 | 2 | 0.58785 | 0.0552250 | 2 | 1.844 | 0 | 2 | | | |
| 318 | 3.3735 | 0.400929 | 2 | 0.02589 | 0.0030476 | 2 | 2.466 | 0.4935605 | 2 | | | |
| 319 | 1.05445 | 0.514137 | 2 | 0.04902 | 0.0047376 | 2 | 0.05066 | 7.071E−06 | 2 | | | |
| 320 | 0.7746 | 0.124450 | 2 | 0.03921 | 0.0029839 | 2 | 10.8007 | 0.0445477 | 2 | | | |
| 321 | 4.196 | 0.032526 | 2 | 0.1814 | 0.0117379 | 2 | 4.217 | 0.3266833 | 2 | | | |

TABLE 19D
EC50 in HEK-Blue IL-2R (CD122+, CD132+) Reporter Assay for Additional Molecules

| | | | | MMP2 cleaved | | | Matriptase cleaved | | | uPa cleaved | | |
|---|---|---|---|---|---|---|---|---|---|---|---|---|
| | Intact molecule | | | molecule | | | molecule | | | molecule | | |
| | Average | | | Average | | | EC50 | | | EC50 | | |
| CMP | EC50 | Std | | EC50 | Std | | Average | Std | | Average | Std | |
| # | (nM) | Deviation | n= | (nM) | Deviation | n= | (nM) | Deviation | n= | (nM) | Deviation | n= |
| 3 | 0.0049 | 0.0022 | 31 | 0.0042 | 0.0019 | 23 | | | | | | |
| 310 | 0.0648 | 0.0016 | 2 | 0.0532 | 0.0091 | 2 | 0.07138 | 0.00325976 | 2 | 0.07229 | 0.00014142 | 2 |
| 311 | 0.0285 | 0.0071 | 2 | 0.0024 | 0.0008 | 2 | 0.01088 | 0.00547300 | 2 | 0.00692 | 0.00277185 | 2 |
| 312 | 0.0831 | 0.0098 | 2 | 0.0081 | 0.0013 | 2 | 0.03115 | 0.00587605 | 2 | 0.02938 | 0.00478711 | 2 |
| 313 | 0.2087 | 0.0146 | 2 | 0.0130 | 0.0028 | 2 | 0.01669 | 0.00097580 | 2 | 0.16505 | 0.00841457 | 2 |
| 314 | 0.1842 | 0.0916 | 2 | 0.0097 | 0.0004 | 2 | 0.01320 | 0.00253851 | 2 | 0.11385 | 0.000212132 | 2 |
| 162 | 0.0174 | 0.0179 | 2 | 0.0056 | 0.0046 | 2 | | | | | | |

### Example 56: Biolayer Interferometry Measurement of Protein-Protein Interactions

Bilayer interferometry (BLI) assays were used to measure the binding constants between activatable IL-2 polypeptides (and activated versions thereof) to IL-2 receptor subunits or receptor complexes. BLI assays were conducted on an Octet® R8 instrument. Assay plates were 96 well, polypropylene, F-bottom (chimney well), black microplates (Greiner Bio-one, Cat. No: 655209). The biosensors were Octet® SAX2 (Sartorius, Cat. No: 18-5136). The assay buffer was 1× Kinetic Buffer (Sartorius, Cat. no: 18-5136) or Phosphate Buffered Saline (PBS) that contained 0.02% (v: v) Tween 20 (Polysorbate 20) and 0.1% (w/v) bovine serum albumin (BSA). The sensor was regenerated between measurements in 10 mM glycine pH 2.7 buffer.

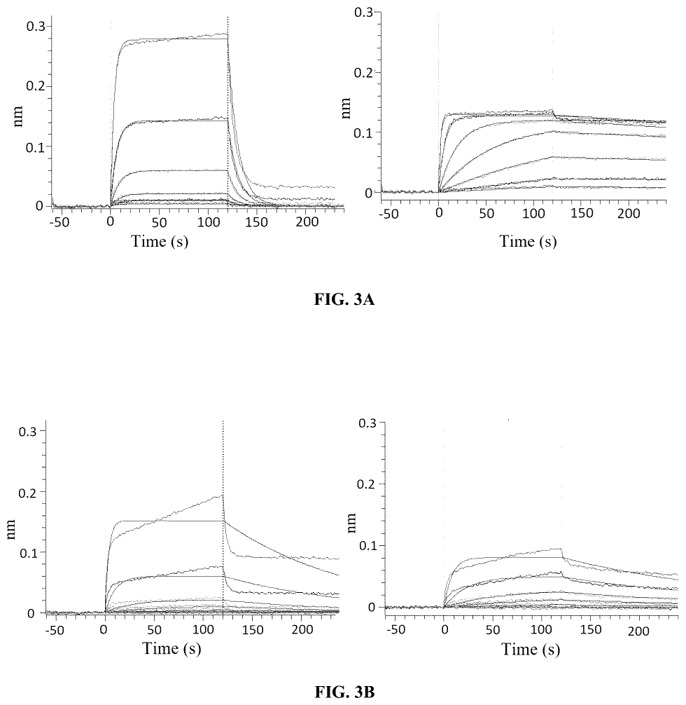

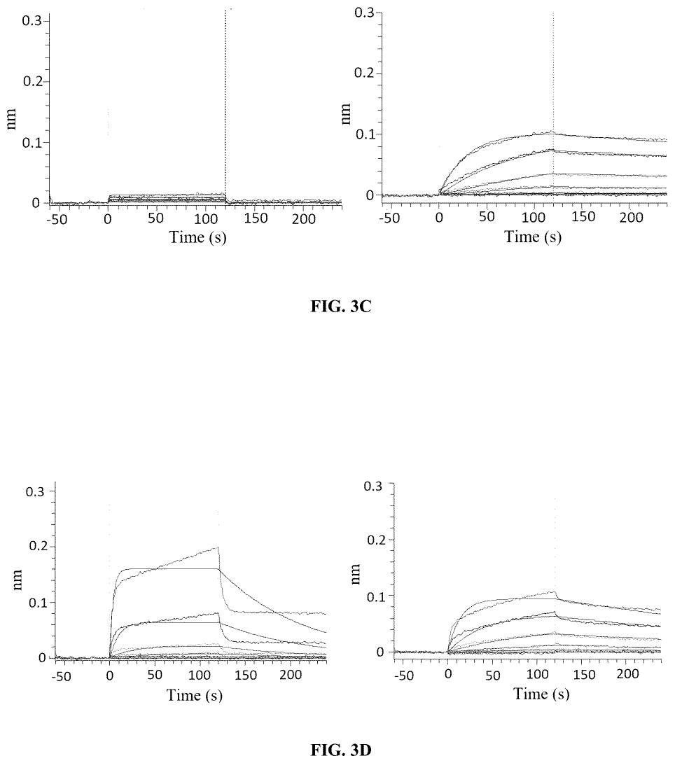

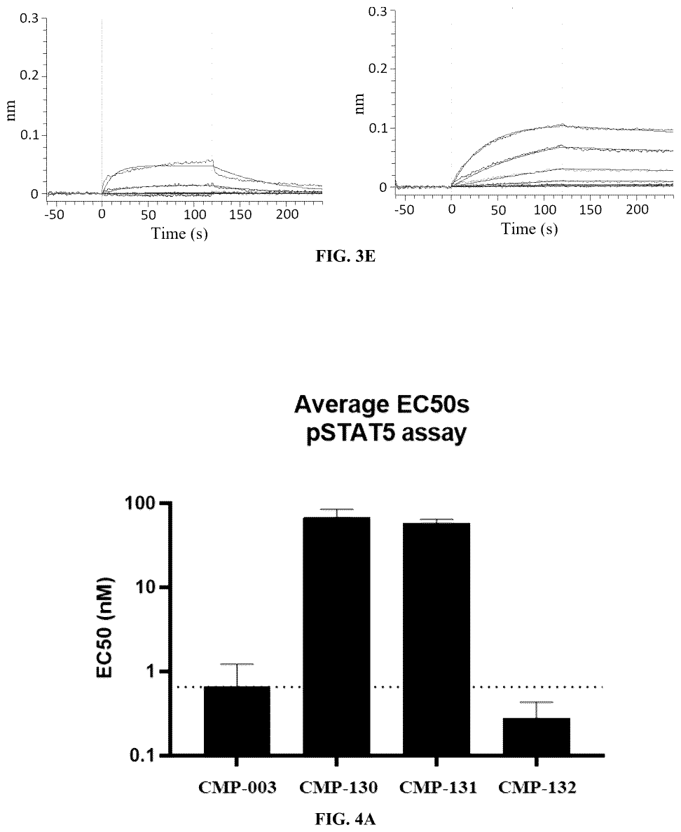

Sensor attached receptors were either recombinant human IL-2 receptor beta containing a His-tag and Avi-Tag™ (R&D Systems™ #AVI10459) or an Avi-Tag™ Fc with human IL-2R beta and IL-2R gamma attached (Acrobiosystems Cat. no: ACRB-ILG-H82F3-25UG). Receptors were reconstituted from lyophilized powder to 500 μg/ml in PBS. Analytes were diluted in Kinetic buffer to 300 nM, then serially diluted to lower concentrations. IL-2 compositions were tested for binding to CD122 or the CD122/CD132 complex: IL-2 polypeptide CMP-003 (FIG. 3A); CMP-145 (FIG. 3B); and macrocyclic activatable IL-2 polypeptides CMP-136 (FIG. 3C), CMP-138 (FIG. 3D), and CMP-151 (FIG. 3E).

TABLE 21
Binding Constants for IL-2 Compositions and IL-2 Receptor Subunit / Complex

| | CD122 Binding (IL-2R B) | | | CD122 CD132 Binding (IL-2R βγ) | | |
|---|---|---|---|---|---|---|
| IL-2 Construct | K_{D} (M) | K_{ON}(M⁻¹S⁻¹) | K_{OFF} (S⁻¹) | K_{D} (M) | K_{ON}(M⁻¹S⁻¹) | K_{OFF} (S⁻¹) |
| CMP-003 | 1.51E−07 | 6.24E+05 | 9.41E−02 | 5.39E−10 | 1.53E+06 | 8.25E−04 |
| CMP-136 | 8.44E−09 | 9.09E+05 | 7.67E−03 | 1.26E−08 | 3.96E+05 | 4.98E−03 |
| CMP-145 | 1.83E−06 | 6.26E+05 | 1.15E+00 | 1.02E−08 | 1.15E+05 | 1.17E−03 |
| CMP-138 | 1.17E−08 | 8.98E−05 | 1.05E−02 | 1.23E−08 | 2.32E+05 | 2.84E−03 |
| CMP-151 | 6.54E−08 | 2.36E+05 | 1.54E−02 | 1.25E−08 | 8.62E+04 | 1.08E−03 |
| CMP-168 | 2.507E−05 | 2.876E+03 | 7.209E−02 | 1.364E−07 | 1.617E+04 | 2.205E−03 |
| CMP-168 | 1.368E−03 | 7.888E+02 | 1.079E00 | 2.2721E−09 | 6.019E+05 | 1.638E−03 |
| MMP2 | | | | | | |
| CMP-165 | 4.42E−05 | 1.43E+04 | 6.33E−01 | 2.615E−11 | 1.202E+04 | 3.142E−07 |
| CMP-165 | 1.11E−06 | 2.13E+05 | 2.37E−01 | 1.875E−09 | 6.084E+05 | 1.141E−03 |
| MMP2 | | | | | | |
| CMP-166 | 6.14E−07 | 9.98E+05 | 6.13E−01 | 1.61E−08 | 2.56E+04 | 4.12E−04 |
| CMP-166 | 7.94E−07 | 2.83E+05 | 2.25E−01 | 1.21E−09 | 5.67E+05 | 6.84E−04 |
| MMP2 | | | | | | |
| CMP-167 | 2.15E−07 | 4.16E+06 | 9.93E−01 | 2.38E−08 | 3.64E+04 | 8.67E−04 |
| CMP-167 | 6.58E−07 | 3.45E+05 | 2.27E−01 | 1.55E−09 | 5.94E+05 | 9.23E−04 |
| MMP2 | | | | | | |
| CMP-313 | 1.632E−07 | 8.139E04 | 1.329E−02 | 3.398E−09 | 4.360E05 | 1.481E−03 |
| CMP-328 | 3.401E−11 | 4.524E07 | 1.539E−03 | 2.487E−08 | 7.783E04 | 1.936E−03 |
| CMP-319 | 5.947E−08 | 2.055E05 | 1.222E−02 | 9.193E−09 | 8.552E04 | 7.861E−04 |

### Example 57A: Assay for STAT5 Phosphorylation (pSTAT5)

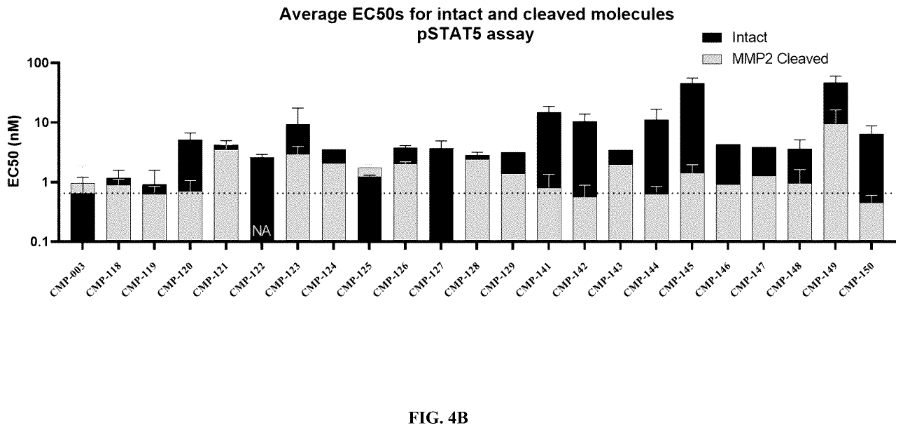

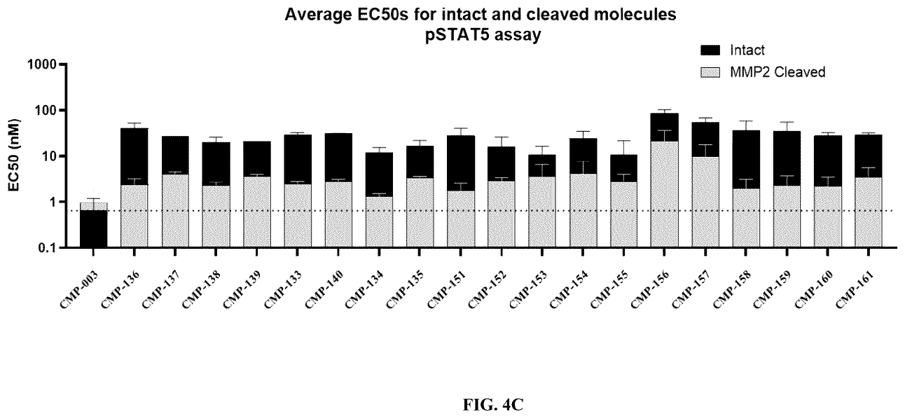

Assays were conducted to determine the effect of IL-2 compositions on STAT5 phosphorylation (pSTAT5) in T cells. Compositions of IL-2 were either intact or treated with MMP2 for activation. For pSTAT5 assays, frozen pan T cells were thawed and cultured overnight in RPMI 10% FCS, 1% Glutamine, 1% NEAA, 25 μM 2-mercaptoethanol (bMeoH), 1% NaPyrovate at 37° C., 5% CO2, 95% humidity. The next day cells were washed and resuspended in PBS. Cells were stimulated with serial dilutions of activatable IL-2 (intact) or protease cleaved IL-2 compositions for 40 minutes at 37° C., 5% CO2, 95% humidity followed by cell fixation and permeabilization with Transcription Factor Phospho Buffer Set (BD Biosciences). Then, cells were stained for 1 hour on ice with antibodies detecting human Phospho-Stat5-PE (Tyr694; 1:50, clone 47/Stat5pY694), CD25-BV421 (1:100, clone M-A251), CD45RA-BV711 (1:100, clone HI100), CD4-FITC (1:400, clone RPA-T4), CD8-APC/Cy7 (1:100, clone SK1), FOXP3-AF647 (1:50, clone 259D). Then cells were washed twice with ice cold FACS buffer. Data were acquired on a multi-color flow cytometer and data were analyzed with FlowJo software. Ec50s were determined with GraphPadPrism. Plotted results of the pSTAT5 assay results are shown in FIG. 4A, FIG. 4B, and FIG. 4C.

TABLE 22
Average EC50 For IL-2 Molecules in the pSTAT5 Assay

| | Intact Molecule | | |
|---|---|---|---|
| | Average | Std | |
| Molecule # | EC50 (nM) | Deviation | n= |
| CMP-003 | 0.65 | 0.56 | 28 |
| CMP-130 | 66.845 | 17.685 | 2 |
| CMP-131 | 56.92 | 7.28 | 2 |
| CMP-132 | 0.28 | 0.15 | 2 |

TABLE 23A
Average EC50 for Activatable IL-2
Molecules in the pSTAT5 Assay

| | Intact Molecule | | | MMP2 Cleaved Molecule | | |
|---|---|---|---|---|---|---|
| Molecule | Average | Std | | Average | Std | |
| # | EC50 (nM) | Deviation | n= | EC50 (nM) | Deviation | n= |
| CMP-003 | 0.65 | 0.56 | 28 | 0.96 | 0.90 | 19 |
| CMP-118 | 1.18 | 0.40 | 6 | 0.90 | 0.22 | 2 |
| CMP-119 | 0.91 | 0.67 | 8 | 0.63 | 0.22 | 4 |
| CMP-120 | 5.11 | 1.57 | 13 | 0.70 | 0.36 | 9 |
| CMP-122 | 4.22 | 0.75 | 6 | 3.55 | 0.68 | 2 |
| CMP-121 | 2.60 | 0.31 | 4 | NA | NA | 0 |
| CMP-123 | 9.26 | 8.26 | 6 | 2.93 | 1.05 | 2 |
| CMP-124 | 3.52 | NA | 1 | 2.07 | 0.00 | 1 |
| CMP-125 | 1.23 | 0.07 | 3 | 1.73 | 0.28 | 3 |
| CMP-126 | 3.74 | 0.37 | 3 | 2.03 | 0.17 | 3 |
| CMP-128 | 3.62 | 1.28 | 5 | 3.68 | NA | 1 |
| CMP-127 | 2.81 | 0.37 | 5 | 2.43 | NA | 1 |
| CMP-129 | 3.15 | NA | 1 | 1.40 | NA | 1 |
| CMP-141 | 14.72 | 3.95 | 2 | 0.80 | 0.55 | 2 |
| CMP-142 | 10.30 | 3.53 | 2 | 0.56 | 0.33 | 2 |
| CMP-143 | 3.41 | NA | 1 | 1.99 | NA | 1 |
| CMP-144 | 11.22 | 5.41 | 2 | 0.63 | 0.22 | 2 |
| CMP-145 | 45.34 | 10.31 | 5 | 1.431 | 0.5248 | 5 |
| CMP-146 | 4.29 | NA | 1 | 0.92 | NA | 1 |
| CMP-147 | 3.86 | NA | 1 | 1.29 | NA | 1 |
| CMP-148 | 3.57 | 1.54 | 2 | 0.95 | 0.68 | 2 |
| CMP-149 | 46.69 | 13.51 | 2 | 9.56 | 6.73 | 2 |
| CMP-150 | 6.38 | 2.39 | 2 | 0.45 | 0.15 | 2 |

TABLE 23B
Average EC50 for Macrocyclic Activatable
IL-2 Molecules in the pSTATS Assay

| | Intact molecule | | | MMP2 cleaved molecule | | |
|---|---|---|---|---|---|---|
| Molecule | Average | Std | | Average | Std | |
| # | EC50 (nM) | Deviation | n= | EC50 (nM) | Deviation | n= |
| CMP-003 | 0.6697 | 0.5436 | 34 | 0.96 | 0.9027 | 30 |
| CMP-163 | 37.06 | 18.88 | 4 | 0.76 | 0.76 | 4 |
| CMP-164 | 31.35 | 19.3 | 4 | 0.61 | 0.57 | 4 |
| CMP-165 | 57.41 | 24.29 | 4 | 1.42 | 1.00 | 4 |
| CMP-166 | 198.4 | 44.41 | 2 | 4.36 | 0.58 | 2 |
| CMP-167 | 176.5 | 34.44 | 2 | 5.31 | 0.74 | 2 |
| CMP-168 | 143.9 | 65.84 | 4 | 6.30 | 3.59 | 4 |
| CMP-169 | 53.4 | 20.4 | 4 | 5.39 | 3.30 | 4 |
| CMP-300 | 94.68 | 44.48 | 4 | 1.732 | 0.6666 | 4 |
| CMP-301 | 179.9 | 9.475 | 2 | 0.8826 | 0.05317 | 2 |
| CMP-302 | 63.41 | 20.67 | 4 | 1.427 | 0.3146 | 4 |
| CMP-303 | 97.93 | 35.85 | 4 | 4.404 | 0.8286 | 4 |
| CMP-304 | 109.5 | 0.4243 | 2 | 8.606 | 1.556 | 2 |
| CMP-305 | 128.1 | 1.838 | 2 | 3.263 | 0.4957 | 2 |

TABLE 24
Average EC50 for Macrocyclic Activatable
IL-2 Molecules in the pSTAT5 Assay

| | Intact molecule | | | MMP2 cleaved molecule | | |
|---|---|---|---|---|---|---|
| | Average | | | Average | | |
| Molecule | EC50 | Std | | EC50 | Std | |
| # | (nM) | Deviation | n= | (nM) | Deviation | n= |
| CMP-003 | 0.65 | 0.5576 | 28 | 0.96 | 0.9027 | 19 |
| CMP-136 | 40.52 | 12.09 | 5 | 2.40 | 0.84 | 5 |
| CMP-137 | 27.08 | 0.09 | 2 | 4.09 | 0.46 | 2 |
| CMP-138 | 20.06 | 6.05 | 5 | 2.32 | 0.40 | 5 |
| CMP-139 | 21.29 | NA | 1 | 3.61 | 0.43 | 2 |
| CMP-133 | 29.86 | 3.07 | 2 | 2.51 | 0.34 | 2 |
| CMP-140 | 31.56 | 0.16 | 2 | 2.86 | 0.29 | 2 |
| CMP-134 | 12.11 | 3.42 | 2 | 1.32 | 0.22 | 2 |
| CMP-135 | 16.23 | 6.10 | 2 | 3.46 | 0.18 | 2 |
| CMP-151 | 28.16 | 12.75 | 10 | 1.81 | 0.78 | 8 |
| CMP-152 | 16.07 | 10.58 | 4 | 2.95 | 0.48 | 4 |
| CMP-153 | 10.83 | 5.55 | 4 | 3.64 | 2.94 | 4 |
| CMP-154 | 24.69 | 10.27 | 4 | 4.20 | 3.52 | 4 |
| CMP-155 | 10.68 | 11.51 | 4 | 2.90 | 1.14 | 4 |
| CMP-156 | 85.97 | 18.50 | 4 | 21.29 | 15.34 | 4 |
| CMP-157 | 54.55 | 14.76 | 4 | 10.02 | 7.66 | 4 |
| CMP-158 | 36.59 | 21.92 | 4 | 1.95 | 1.21 | 4 |
| CMP-159 | 35.50 | 19.34 | 4 | 2.27 | 1.47 | 4 |
| CMP-160 | 27.76 | 5.21 | 4 | 2.21 | 1.35 | 4 |
| CMP-161 | 29.43 | 2.81 | 4 | 3.59 | 2.02 | 4 |
| CMP-162 | 2.794 | 2.435 | 2 | 0.7578 | 0.6489 | 2 |

### Example 57B: Assay for STAT5 Phosphorylation (pSTAT5) in PBMCs

PBMCs were thawed and cultured overnight in RPMI 10% FCS, 1% Glutamine, 1% NEAA, 25 μM 2BME, 1% NaPyrovate at 37° C., 5% CO2, 95% humidity. The next day cells were washed and re-suspended in PBS. Cells were stimulated with serial dilutions of activatable IL-2 for 40 minutes at 37° C., 5% CO2, 95% humidity followed by cell fixation and permeabilization with Transcription Factor Phospho Buffer Set (BD Biosciences). Then, cells were stained for 1 hour on ice with antibodies detecting human Phospho-Stat5-PE (Tyr694; 1:50, clone 47/Stat5pY694), CD25-BV421 (1:100, clone M-A251), CD45RA-BV711 (1:100, clone HI100), CD4-PE texas red (1:800, clone RPA-T4), FOXP3-AF647 (1:50, clone 259D), CD3-PE/Cy7 (1:20, cloneOkt3), CD56-AF488 (1:400, clone 5.1H11). Then cells were washed twice with ice cold FACS buffer. Data were acquired on a multi-color flow cytometer and data were analyzed with FlowJo software EC₅₀s were determined with GraphPadPrism. Results are shown in the table below (NK cell activation).

TABLE 25
Average EC50 of Selected molecules in the pSTAT5 Assay in NK cells

| | | | MMP2 cleaved | | | | Matriptase cleaved | | | uPa cleaved | | |
|---|---|---|---|---|---|---|---|---|---|---|---|---|
| | Intact molecule | | molecule | | | | molecule | | | molecule | | |
| Molecule | Average | | | Average | | | Average | | | Average | | |
| (CMP) | EC50 | Std | | EC50 | Std | | EC50 | Std | | EC50 | Std | |
| # | (nM) | Deviation | n= | (nM) | Deviation | n= | (nM) | Deviation | n= | (nM) | Deviation | n= |
| 003 | 0.092 | 0.040 | 9 | | | | | | | | | |
| 145 | 4.329 | 1.596 | 4 | 0.082 | 0.040 | 4 | | | | | | |
| 310 | 4.297 | 1.992 | 4 | 4.066 | 2.880 | 2 | 6.898 | 1.702 | 2 | 6.787 | 2.563 | 2 |
| 311 | 2.563 | 1.379 | 4 | 0.073 | 0.039 | 4 | 0.512 | 0.231 | 4 | 0.548 | 0.230 | 4 |
| 313 | 0.898 | 0.488 | 2 | 0.035 | 0.007 | 2 | 0.714 | 0.428 | 2 | 0.081 | 0.056 | 2 |
| 314 | 0.206 | 0.062 | 2 | 0.040 | 0.005 | 2 | 0.117 | 0.006 | 2 | 0.065 | 0.029 | 2 |
| 315 | 0.638 | 0.179 | 2 | 0.369 | 0.142 | 2 | 0.387 | 0.060 | 2 | 0.335 | 0.052 | 2 |
| 316 | 0.484 | 0.204 | 2 | 0.460 | 0.214 | 2 | 0.617 | 0.245 | 2 | 0.533 | 0.155 | 2 |
| 168 | 53.854 | 29.338 | 10 | 0.981 | 0.454 | 10 | | | | | | |
| 300 | 38.103 | 16.549 | 4 | 0.379 | 0.156 | 4 | | | | | | |
| 326 | 15.515 | 6.430 | 8 | 15.995 | 2.652 | 2 | 17.605 | 5.706 | 2 | 18.020 | 0.467 | 2 |
| 327 | 16.596 | 6.635 | 8 | 0.850 | 0.386 | 8 | 17.265 | 7.290 | 2 | 12.075 | 2.171 | 2 |
| 331 | 6.594 | 0.289 | 2 | 5.094 | 0.484 | 2 | | | | | | |
| 328 | 9.235 | 5.902 | 6 | 1.478 | 0.936 | 6 | 4.281 | 2.455 | 2 | 2.965 | 1.744 | 2 |
| 329 | 20.550 | 11.130 | 2 | 0.687 | 0.335 | 2 | 14.387 | 6.312 | 2 | 18.105 | 5.452 | 2 |
| 330 | 20.335 | 7.686 | 2 | 6.118 | 2.640 | 2 | 16.410 | 4.483 | 2 | 26.995 | 20.980 | 2 |
| 317 | 34.078 | 10.412 | 4 | 12.156 | 4.682 | 4 | 28.485 | 7.357 | 4 | | | |
| 318 | 37.370 | 7.284 | 4 | 0.388 | 0.193 | 4 | 33.680 | 6.177 | 4 | | | |
| 319 | 7.725 | 1.562 | 4 | 0.713 | 0.377 | 4 | 0.804 | 0.452 | 4 | | | |
| 320 | 13.247 | 4.377 | 4 | 0.571 | 0.071 | 4 | 15.668 | 3.447 | 4 | | | |
| 321 | 55.093 | 7.348 | 4 | 2.938 | 0.427 | 4 | 65.225 | 19.411 | 4 | | | |
| 136 | 2.174 | 0.693 | 2 | 0.2769 | 0.0853 | 2 | | | | | | |
| 138 | 1.8825 | 0.342 | 2 | 0.2877 | 0.0316 | 2 | | | | | | |
| 145 | 7.087 | 6.029 | 2 | 0.2459 | 0.1107 | 2 | | | | | | |
| 151 | 2.509 | 0.563 | 2 | 0.3524 | 0.0201 | 2 | | | | | | |
| 421 | 3.26 | 1.321 | 7 | | | | | | | | | |
| 422 | 15.04 | 7.932 | 2 | | | | | | | | | |
| 420 | 3.34 | 1.086 | 4 | | | | | | | | | |

cleaved controls added, CMP numbers need to be added

### Example 57C: Assay for STAT5 Phosphorylation (pSTAT5) in Mouse Splenocyte

Frozen C57BL/6 mouse splenocytes were thawed and left to recover for 2h in RPMI 10% FCS, 1% Glutamine, 1% NEAA, 25 μM 2BME, 1% NaPyrovate at 37° C., 5% CO2, 95% humidity. Afterwards, cells were washed and re-suspended in PBS. Cells were stimulated with serial dilutions of activatable IL2 for 40 minutes at 37° C., 5% CO2, 95% humidity followed by cell fixation and permeabilization with Transcription Factor Phospho Buffer Set (BD Biosciences). Then, cells were stained for 1 hour on ice with antibodies detecting mouse Phospho-Stat5-AF488 (Tyr694; 1:200, clone 47/Stat5pY694), CD25-BV421 (1:100, clone PC61), CD45R/B220-BV711 (1:100, clone 53-6.7), CD3-BV650 (1:100, clone 17A2), FOXP3-PE (1:200, clone FJK-16s), NK1.1-AF700 (1:200, clone PK136). Data were acquired on a multi-color flow cytometer and data were analyzed with FlowJo software. Ec50s were determined with GraphPadPrism. Results are shown in the table below. Efficacy plateau could not be reached for all intact and cleaved molecules.

TABLE 26
Average EC50 of molecules in the
pSTAT5 Assay in mouse splenocytes

| | Intact molecule | | | MMP2 cleaved molecule | | |
|---|---|---|---|---|---|---|
| | Average | | | Average | | |
| | EC50 | Std | | EC50 | Std | |
| CMP # | (RM) | Deviation | n= | (nM) | Deviation | n= |
| 003 | 24.035 | 8.725 | 2 | 45.725 | 26.995 | 2 |
| 136 | ND | ND | | 84.685 | 35.415 | 2 |
| 138 | ND | ND | | 75.125 | 7.185 | 2 |
| 145 | ND | ND | | 55.205 | 11.105 | 2 |
| 151 | ND | ND | | 89.22 | 21.38 | 2 |

### Example 58: Protease Cleavage and Activation Methods

In experiments where activatable IL-2 polypeptides were first subjected to treatment with MMP protease, the following protocol or one substantially analogous was used. Compositions of IL-2 were diluted to a final concentration of 30 μM in TRIS Buffer (50 mM TRIS, 10 mM CaCl2, 150 mM NaCl, 0.05% Brij35 pH 7.5). Compositions of IL-2 were further diluted to a final concentration of 15 μM with either TRIS buffer or TRIS Buffer containing pre-activated MMP at a concentration of 2 μg/ml. The final reaction volume of 40 μl was transferred in sealed tubes and incubated at 37° C. under shaking conditions for 16 hours. Cleaved/activated compositions of IL-2 and respective intact controls were used in assays or stored frozen at −80° C.

### Example 58: Protease Cleavage and Activation Methods

In experiments where activatable IL-2 polypeptides were first subjected to treatment with MMP protease, the following protocol or one substantially analogous was used. Compositions of IL-2 polypeptide (or corresponding immunocytokine) were diluted to a final concentration of 30 μM in TRIS Buffer (50 mM TRIS, 10 mM CaCl2, 150 mM NaCl, 0.05% Brij35 pH 7.5). Compositions of IL-2 (or corresponding immunocytokine) were further diluted to a final concentration of 15 pM with either TRIS buffer or TRIS Buffer containing pre-activated MMP at a concentration of 2 ng/ml. The final reaction volume of 40 μl was transferred in sealed tubes and incubated at 37° C. under shaking conditions for 16 hours. Cleaved/activated compositions of IL-2 and respective intact controls were used in assays or stored frozen at −80° C.

### Example 59: Preparation of an Anti-PD-1 Activatable Immunocytokine

A modified anti-PD-1 antibody (e.g., LZM-009 or Pembrolizumab) is prepared utilizing methods described in Examples 2-4 of US Patent Application No. US20200190165A1.

Briefly, an Ajicapped (AJICAP™ by Ajinomoto Bio-Pharma Services, which is described at least in in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1) anti-PD-1 antibody with a DBCO conjugation group is reacted with an azide containing IL-2 polypeptide (e.g., any one of SEQ ID NOs: 3-55) for 48 hours at room temperature. The crude anti-PD-1 antibody-IL-2 polypeptide activatable is separated using hydrophobic interaction chromatography, ion exchange chromatography, size exclusion chromatography, trapping by DBCO-PEG, protein A chromatography, or DBCO resin column purification.

An exemplary process for the AJICAP™ methodology is as follows: modified antibody (e.g., an anti-PD-1 antibody such as Pembrolizumab of LZM-009) comprising a DBCO conjugation handle is prepared using a protocol modified from Examples 2-4 of US Patent Publication No. US2020/0190165. Briefly, the anti-PD1 antibody with a free sulfhydryl group attached to a lysine residue side chain in the Fe region (e.g., K248, Eu numbering) is prepared by contacting the antibody with an affinity peptide configured to deliver a protected version of the sulfhydryl group (e.g., a thioester or reducible disulfide) to the lysine residue. An exemplary peptide capable of performing this reaction is shown below (SEQ ID NO: 682), as reported in Matsuda et al., Mol. Pharmaceutics 2021, 18, 4058-4066, which selectively attaches the sulfhydryl group via the NHS ester at residue K248 of the Fc region of the antibody:

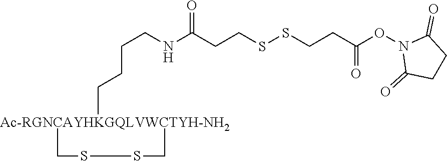

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide a sulfhydryl protecting group (e.g., the relevant portion of the affinity peptide would have a structure of

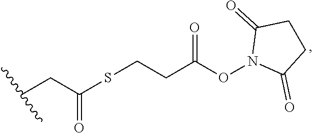 [SMILES: *CC(=O)SCCC(=O)ON1C(=O)CCC1=O]

or another of the cleavable linkers discussed herein). Such alternative affinity peptides include those described in, for example “AJICAP Second Generation: Improved Chemical Site-Specific Conjugation Technology for Antibody-Drug Conjugation Technology for Antibody-Drug Conjugate Production” (Bioconjugate (Bioconjugate Chem. 2023, 34, 4, 728-738). Exemplary affinity peptides provided therein include those shown below, wherein the left structure (SEQ ID NO: 683) targets K248 of the Fc region and the right structure (SEQ ID NO: 684) targets K288 of the Fc region (EU numbering).

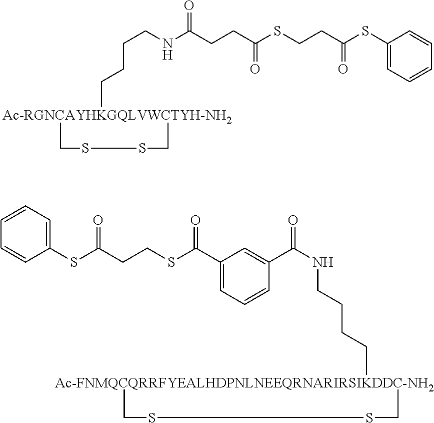

The protecting group is then removed to reveal the free sulfhydryl (e.g., by hydrolysis of thioester or reduction of a disulfide with TCEP). The free sulfhydryl is then reacted with a bifunctional reagent comprising a bromoacetamide or bromoketone group connected to the DBCO conjugation handle through a tether group (e.g., bromoacetamido-PEG-amido-DBCO, bromoacetamido-DBCO, maleimido-PEGₓ-amido-DBCO, maleimido-DBCO, p-(2-bromoacetyl)benzoyl-PEGₓ-amido-DBCO, p-(2-bromoacetyl)benzoyl-DBCO, etc.). In particular, the bifunctional reagent

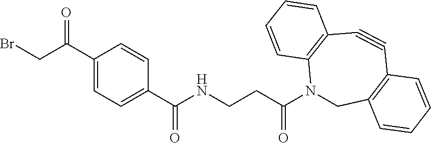 [SMILES: O=C(CBr)c1ccc(C(=O)NCCC(=O)N2Cc3ccccc3C#Cc3ccccc32)cc1]

is used to prepare the constructs provided herein. The method can be used to produce an antibody with one DBCO group present (DAR1) and/or two DBCO groups attached to the antibody (DAR2, one DBCO group linked to each Fc of the antibody). The desired azide modified activatable IL-2 polypeptide is then reacted with the DBCO modified antibody to produce the activatable immunocytokine.

In another embodiment, antibody comprising a single DBCO conjugation handle is prepared by first reacting excess anti-PD-1 antibody (e.g., pembrolizumab, LZM-009, etc) with appropriately loaded affinity peptide to introduce a single sulfhydryl after appropriate removal of protecting group (e.g., disulfide reduction or thioester cleavage). A bifunctional linking group with a sulfhydryl reactive conjugation handle and DBCO conjugation handle (e.g., bromoacetamido-PEG-amido-DBCO, bromoacetamido-DBCO, maleimido-PEGₓ-amido-DBCO, maleimido-DBCO, p-(2-bromoacetyl)benzoyl-PEGₓ-amido-DBCO, p-(2-bromoacetyl)benzoyl-DBCO, etc.) is then reacted with the single sulfhydryl to produce the single DBCO containing antibody. In the exemplified constructs provided herein, the bifunctional linking reagent used is

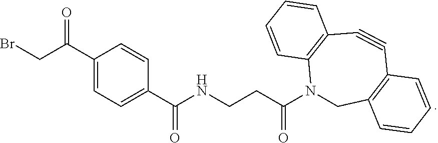 [SMILES: O=C(CBr)c1ccc(C(=O)NCCC(=O)N2Cc3ccccc3C#Cc3ccccc32)cc1]

The single DBCO containing antibody is then conjugated with a suitable azide containing activatable IL-2 to achieve an activatable immunocytokine with a DAR of 1.

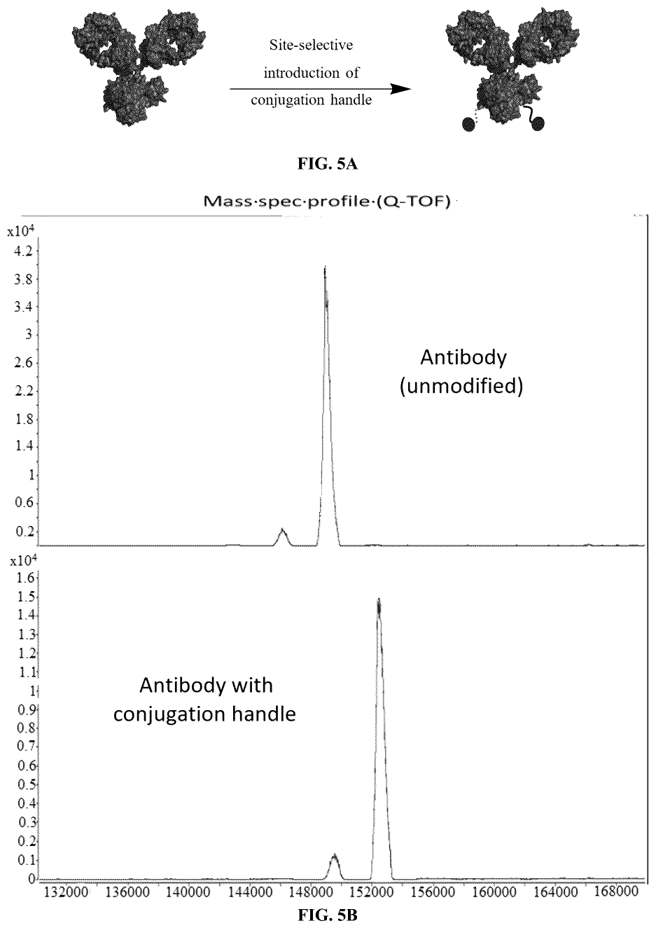

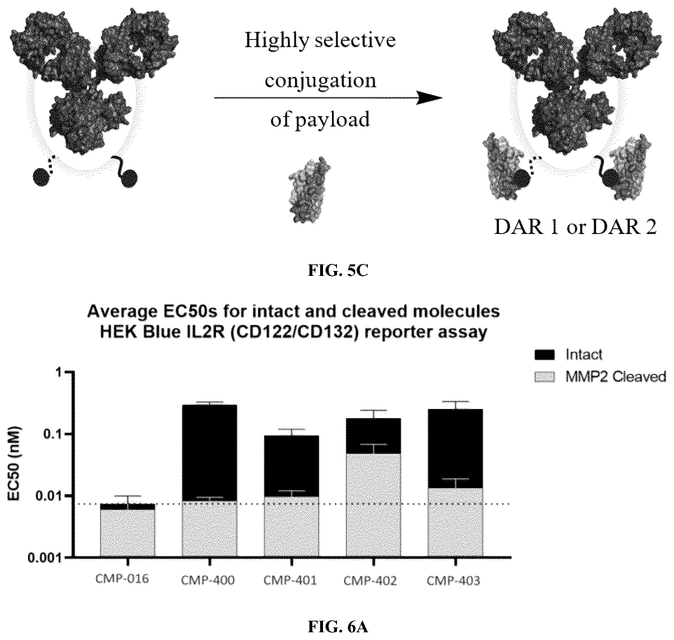

FIG. 5A shows site-selective modification of anti-PD1 antibody by chemical modification technology to introduce one or two conjugation handles. FIG. 5B shows Q-TOF mass spectra of unmodified Pembrolizumab and Pembrolizumab with conjugation to DBCO conjugation handle using AJICAP technology. Q-TOF mass spectra of LZM-009 similarly modified showed similar results. FIG. 5C shows site-selective conjugation of IL2 cytokine to generate a PD1-IL2 with DAR1 or DAR 2. Populations of PD1-IL2s with mixed DAR between 1 and 2 can also be prepared.

Representative Protocol: The following protocol was used to prepare CMP-402:
1) To LZM-009 in 50 mM NaOAc pH 5.5, AJICAP reagent dissolved in DMF was added and the resulting reaction mixture was incubated at 25° C. for 1 h. The mixture was buffer exchanged into 50 mM NaOAc pH 5.0. To isolate the desired intermediate-1 with DAR 1, a cation exchange chromatography (CIEX) was performed. The fractions containing the desired LZM-009/AJICAP peptide intermediate were concentrated by TFF and buffer exchanged to 50 mM NaOAc pH 5.5.
2) To a solution of LZM-009/AJICAP peptide intermediate from above operation, a solution of 2.0 M hydroxylamine was added, and the resulting solution was gently mixed for 1 h at 25° C. The reaction mixture was subjected to TFF to remove the cleaved AJICAP peptide and buffer exchanged to 50 mM NaOAc pH 5.5 and then to 10 mM PBSE pH 7.4 to afford sulfhydryl modified K248 LZM-009.
3) To a solution of sulfhydryl modified K248 LZM-009, bifunctional linker

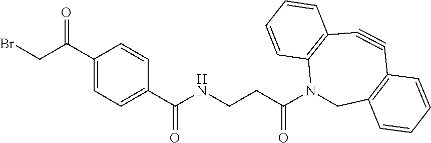 [SMILES: O=C(CBr)c1ccc(C(=O)NCCC(=O)N2Cc3ccccc3C#Cc3ccccc32)cc1]

was added and the resulting solution was gently mixed for 1.5 h at 25° C. The reaction mixture was subjected to TFF to remove excess linker and buffer exchanged to 50 mM NaOAc pH 5.0 to afford DBCO modified LZM-009.
4) DBCO modified LZM-009 was mixed with the azide containing CMP-145 payload and the resulting solution was gently mixed overnight at 25° C.
5) The resulting solution was purified by cation exchange chromatography. The fractions containing the desired product (CMP-402, DAR1) were pooled and concentrated. A polishing SEC was performed with the concentrated sample to remove any aggregates to yield high purity immunocytokine.

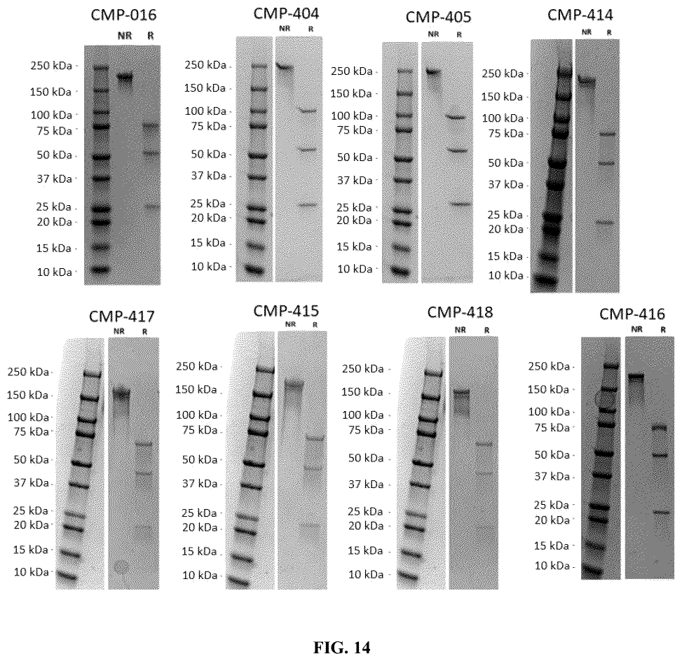

Table 27 below summarizes various immunocytokines prepared according to the methods provided herein or analogous methods described elsewhere. Representative SDS-PAGE gels of selected immunocytokines described herein are shown in FIG. 14.

TABLE 27
Characteristics of immunocytokines

| | | | | | | | RP- | SDS |
|---|---|---|---|---|---|---|---|---|
| | | | | Antibody | | Aggregates | HPLC | Result |
| | | IL-2 | | Conjugation | Endotoxin | (%) | (% | (OK = as |
| Composition | Antibody | polypeptide | DAR | site | (EU/mg) | (SEC-HPLC) | Purity) | expected) |
| CMP-400 | LZM-009 | CMP-136 | 1 | K248 | <2.9 | 1.15 | | |
| CMP-401 | LZM-009 | CMP-138 | 1 | K248 | <3.11 | 0 | | |
| CMP-402 | LZM-009 | CMP-145 | 1 | K248 | <2.75 | 3.8 | | |
| CMP-403 | LZM-009 | CMP-151 | 1 | K248 | <3.0 | 1.3 | | |
| CMP-016 (non- | LZM-009 | CMP-003 | 1 | K248 | | | 97.1 | OK |
| activatble | | | | | | | | |
| control IL-2/PD-1 | | | | | | | | |
| immunocytokine) | | | | | | | | |
| CMP-404 | LZM-009 | CMP-313 | 1 | K248 | | | 82.24 | OK |
| CMP-405 | LZM-009 | CMP-314 | 1 | K248 | | | 87.07 | OK |
| CMP-406 | LZM-009 | CMP-310 | 1 | K248 | | | | |
| CMP407 | LZM-009 | CMP-311 | 1 | K248 | | | | |
| CMP-409 | LZM-009 | CMP-331 | 1 | K248 | | | | |
| CMP-410 | LZM-009 | CMP-168 | 1 | K248 | | | | |
| CMP-411 | LZM-009 | CMP-326 | 1 | K248 | | | | |
| CMP-412 | LZM-009 | CMP-327 | 1 | K248 | | | | |
| CMP-413 | LZM-009 | CMP-300 | 1 | K248 | | | 95.64 | OK |
| CMP-414 | LZM-009 | CMP-317 | 1 | K248 | | | 96.95 | OK |
| CMP-415 | LZM-009 | CMP-318 | 1 | K248 | | | 96.04 | OK |
| CMP-416 | LZM-009 | CMP-319 | 1 | K248 | | | 97.44 | OK |
| CMP-417 | LZM-009 | CMP-320 | 1 | K248 | | | 98.33 | OK |
| CMP-418 | LZM-009 | CMP-321 | 1 | K248 | | | 97.17 | OK |
| CMP-419 | LZM-009 | CMP-324 | 1 | K248 | | 3.45 | 96.28 | OK |
| CMP-420 | LZM-009 | CMP-325 | 1 | K248 | | 2.61 | 96.48 | OK |
| CMP-421 | LZM-009 | CMP-323 | 1 | K248 | | 1.82 | | OK |

In the above table, unreported results do not imply a QC failure.

### Example 60: HEK Blue IL2 Receptor (CD122+/CD132+) Reporter Assay

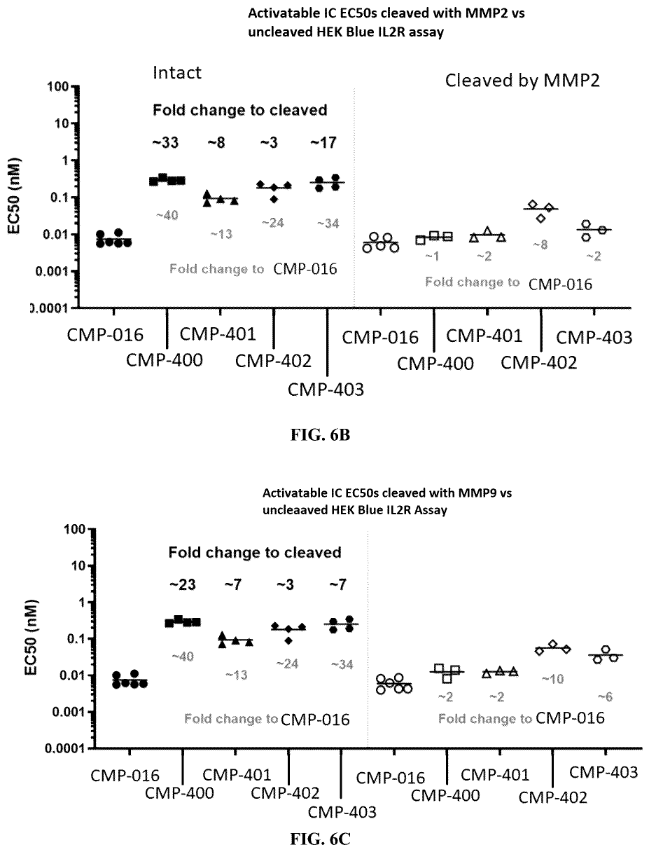

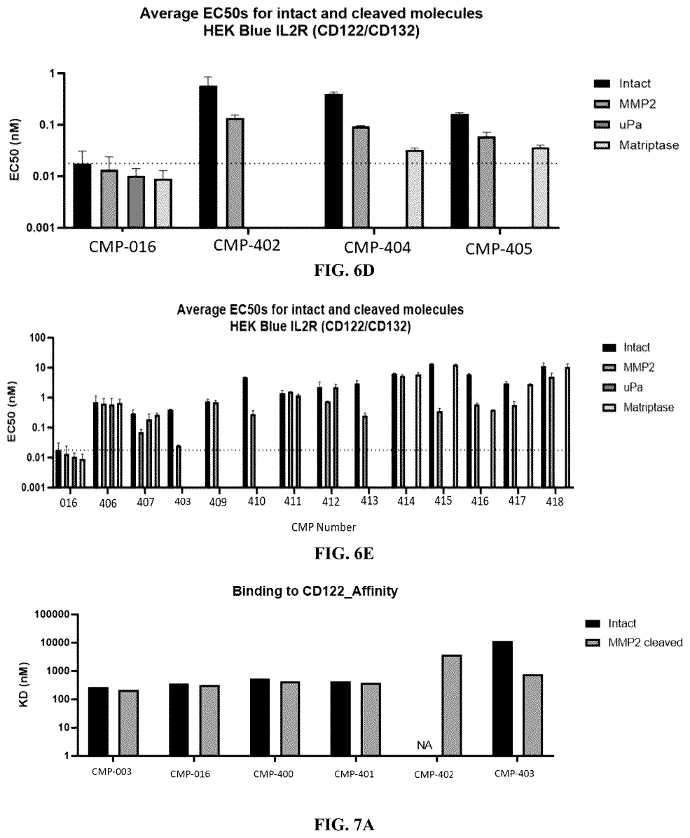

In order to assess the ability of activatable immunocytokines to signal via the IL-2 moeity, a HEK Blue reporter assay was performed in CD122+/CD132+ cells. Briefly, an IL-2R□□ positive HEK-Blue reporter cell line was used to determine binding of activatable IL-2 immunocytokines (and activated versions thereof) to IL-2R□□ and subsequent downstream signaling that resulted in activation of a STAT5 transcriptional reporter. Activatable IL-2 polypeptides were either intact or MMP2 treated for activation. The general protocol was as follows: 5×104 cells HEK-Blue™ IL-2R□□ reporter cells (InvivoGen, ##hkb-il2bg) were seeded into each well of a 96 well plate and stimulated with 0-100 nM of IL-2 immunocytokine variants at 37° C. and 5% CO2. After a 20h incubation, 20 μL of cell culture supernatant was then taken from each well and mixed with 180 μL QUANTI-Blue™ media in a 96 well plate, then incubated for 1 hour at 37° C. and 5% CO2 (with 600 rpm shaking). The absorbance signal at 630 nm was then measured on an Enspire® plate reader. Half Maximal Effective dose (EC50) was calculated based on a variable slope, four parameter analysis using GraphPad PRISM® software. Results from this experiment are shown in the table below and FIG. 6A. FIG. 6B shows this same date with the indicated fold change as compared to CMP-016 indicated. FIG. 6C shows the results of a similar experiment, except that activatable immunocytokines were treated with MMP9 rather than MMP2 for activation.

| | Intact molecule | | | MMP2 cleaved molecule | | |
|---|---|---|---|---|---|---|
| | Average | Std | | Average | Std | |
| Molecule | EC50 (nM) | Deviation | n= | EC50 (nM) | Deviation | n= |
| CMP-016 | 0.0075 | 0.0025 | 6 | 0.0061 | 0.0022 | 5 |
| CMP-400 | 0.2947 | 0.0327 | 4 | 0.0083 | 0.0012 | 3 |
| CMP-401 | 0.0942 | 0.0249 | 4 | 0.0097 | 0.0024 | 3 |
| CMP-402 | 0.1794 | 0.0626 | 4 | 0.0485 | 0.0195 | 3 |
| CMP-403 | 0.2534 | 0.0825 | 4 | 0.0134 | 0.0054 | 3 |

An analogous experiment to that described above was also performed on additional activatable immunocytokines as described herein, except that selected molecules were also assessed for activation by treatment with uPA and/or matriptase. Results for activatable IL-2 polypeptides with a cleavable peptide attached at a single point are shown in FIG. 6D and macrocyclic activatable IL-2 polypeptides are shown in FIG. 6E. Numerical results from these experiments are shown in the table below.

| | | | | MMP2 cleaved | | | uPA cleaved | | | Matriptase cleaved | | |
|---|---|---|---|---|---|---|---|---|---|---|---|---|
| | Intact molecule | | | molecule | | | molecule | | | molecule | | |
| | Average | | | Average | | | Average | | | Average | | |
| CMP | EC50 | Std | | EC50 | Std | | EC50 | Std | | EC50 | Std | |
| Number | (nM) | Deviation | n= | (nM) | Deviation | n= | (nM) | Deviation | n= | (nM) | Deviation | n= |
| CMP-016 | 0.018 | 0.013 | 2 | 0.013 | 0.011 | 2 | 0.010 | 0.004 | 2 | 0.009 | 0.004 | 2 |
| CMP-402 | 0.579 | 0.271 | 2 | 0.136 | 0.019 | 2 | | | | | | |
| CMP-404 | 0.401 | 0.033 | 2 | 0.094 | 0.003 | 2 | | | | 0.033 | 0.003 | 2 |
| CMP-405 | 0.160 | 0.013 | 2 | 0.059 | 0.012 | 2 | | | | 0.037 | 0.004 | 2 |
| CMP-016 | 0.018 | 0.013 | 2 | 0.013 | 0.011 | 2 | 0.010 | 0.004 | 2 | 0.009 | 0.004 | 2 |
| CMP-406 | 0.689 | 0.444 | 2 | 0.616 | 0.336 | 2 | 0.590 | 0.352 | 2 | 0.653 | 0.246 | 2 |
| CMP-407 | 0.291 | 0.106 | 2 | 0.072 | 0.016 | 2 | 0.194 | 0.092 | 2 | 0.267 | 0.038 | 2 |
| CMP-403 | 0.403 | 0.014 | 2 | 0.025 | 0.001 | 2 | | | | | | |
| CMP-409 | 0.762 | 0.133 | 2 | 0.697 | 0.123 | 2 | | | | | | |
| CMP-410 | 4.789 | 0.219 | 2 | 0.288 | 0.087 | 2 | | | | | | |
| CMP-411 | 1.443 | 0.315 | 2 | 1.549 | 0.047 | 2 | 1.174 | 0.159 | 2 | | | |
| CMP-412 | 2.291 | 1.077 | 2 | 0.739 | 0.027 | 2 | 2.267 | 0.538 | 2 | | | |
| CMP-413 | 2.928 | 0.787 | 4 | 0.252 | 0.053 | 4 | | | | | | |
| CMP-414 | 6.363 | 0.247 | 2 | 5.287 | 0.614 | 2 | | | | 6.023 | 0.750 | 2 |
| CMP-415 | 13.035 | 0.898 | 2 | 0.352 | 0.087 | 2 | | | | 12.370 | 0.933 | 2 |
| CMP-416 | 5.849 | 0.490 | 2 | 0.587 | 0.084 | 2 | | | | 0.390 | 0.001 | 2 |
| CMP-417 | 3.033 | 0.462 | 2 | 0.567 | 0.174 | 2 | | | | 2.773 | 0.139 | 2 |
| CMP-418 | 11.396 | 2.962 | 2 | 5.043 | 1.606 | 2 | | | | 10.635 | 2.708 | 2 |

### Example 61: CD122 and CD122/CD132 Biolayer Interferometry (BLI) of Activatable IL-2 Immunocytokines

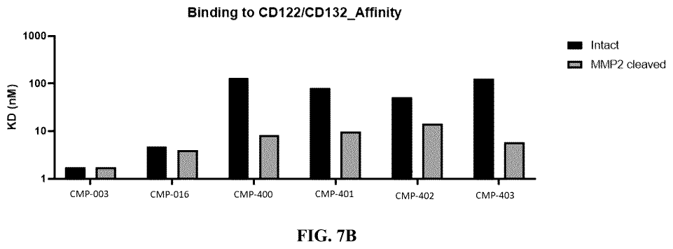

The ability of activatable immunocytokines to bind to CD122 and CD122/CD132 were assess by biolayer interferometry (see protocol in Example 56 above). The results are reported in the tables below. FIG. 7A shows the KD (nM) values of the indicated activatable immunocytokines (and corresponding controls) with CD122 before and after treatment with MMP2. FIG. 7B shows the KD (nM) values of the indicated activatable immunocytokines (and corresponding controls) with CD122/CD132 before and after treatment with MMP2.

Table of CD122 BLI

| CD122 | Intact | | | MMP2 Treated | | |
|---|---|---|---|---|---|---|
| BLI | KD | ka | kDis | KD | ka | kDis |
| Molecule | (nM) | (1/Ms) | (1/s) | (nM) | (1/Ms) | (1/s) |
| CMP-003 | 270.803 | 533419 | 0.144452 | 221.526 | 649679 | 0.143921 |
| CMP-016 | 369.93 | 305852 | 0.113144 | 317.456 | 367230 | 0.116579 |
| CMP-400 | 533.353 | 229797 | 0.122563 | 426.879 | 264785 | 0.113031 |
| CMP-401 | 441.606 | 220396 | 0.097329 | 387.52 | 258320 | 0.100104 |
| CMP-402 | NA | NA | NA | 3723.82 | 24110.5 | 0.089783 |
| CMP-403 | 11346.7 | 16670.3 | 0.189154 | 773.233 | 168690 | 0.130437 |

Table of CD122/CD132 BLI

| CD122/CD132 | Intact | | | MMP2 Treated | | |
|---|---|---|---|---|---|---|
| BLI | KD | ka | kDis | KD | ka | kDis |
| Molecule | (nM) | (1/Ms) | (1/s) | (nM) | (1/Ms) | (1/s) |
| CMP-003 | 1.77734 | 1657510 | 0.002946 | 1.73571 | 1925910 | 0.003343 |
| CMP-016 | 4.78561 | 547251 | 0.002619 | 4.06449 | 611364 | 0.002485 |
| CMP-400 | 131.046 | 179837 | 0.023567 | 8.18222 | 379810 | 0.003108 |
| CMP-401 | 82.3536 | 157700 | 0.012987 | 9.85913 | 406479 | 0.004008 |
| CMP-402 | 52.5959 | 53185.7 | 0.002797 | 14.6693 | 81730.6 | 0.001199 |
| CMP-403 | 128.974 | 33421.7 | 0.004311 | 5.85256 | 249762 | 0.001462 |

### Example 62: Antibody Characterization of Activatable IL-2 Immunocytokines

The ability of activatable IL-2 immunocytokines to perform functions associated with the antibody portion were assessed using the following assays.

PD-1 binding ELISA assay—The interaction of the activatable IL-2 immunocytokines containing anti-PD1 antibodies with PD-1 (CD279) were measured by ELISA assay. For these studies, Corning high-binding half-area plates (Fisher Scientific, Reinach, Switzerland) were coated overnight at 4° C. with 25 μl of unmodified or conjugated anti-PD1 antibodies at 2.5 pg/ml in PBS. Plates were then washed four times with 100 μl of PBS-0.2% Tween20 (wash buffer). Plate surfaces were blocked with 25 μl of PBS-1% BSA at 37° C. for 1h. Plates were then washed four times with 100 μl of wash buffer. Twenty-five microliters of recombinant biotinylated PD1/CD279 protein from Biolegend (789406, London, United Kingdom) were added in serial dilutions in PBS-T with 0.1% BSA and incubated at 37° C. for 2h. Plates were then washed four times with wash buffer. Twenty-five microliters of Streptavidin-Horseradish peroxidase (#RABHRP3, Merck, Buchs, Switzerland) diluted at 1:500 in PBS-0.02% Tween20-0.1% BSA were added to each well and incubated at Room Temperature for 30 min. Plates were then washed four times with 100 μl of wash buffer. Fifty microliters of TMB substrate reagent (#CL07, Merck, Buchs, Switzerland) were added to each well and incubated at 37° C. during 5 min. After 5 min at 37° C., Horseradish peroxidase reaction was stopped by adding 50 μl/well of 0.5M H₂SO₄ stop solution. ELISA signal was then measured at 450 nm on an EnSpire plate reader from Perkin Elmer (Schwerzenbach, Switzerland).

PD1/PD-L1 Blockade Assay—The ability of the activatable IL-2 immunocytokines to interfere with PD1/PDL1 pathway was measured using the PD-1/PD-L1 Blockade Bioassay from Promega (Cat. #J1250, Madison, WI, USA). PD-1/PD-L1 Blockade Bioassay is a bioluminescent cell-based assay based on the co-culture of effector cells with target cells mimicking an immunological synapse. Jurkat T cells expressing human PD-1 (Jurkat-Lucia-PD1) and a luciferase reporter driven by a NFAT response element (NFAT-RE) are activated by CHO-K1 cells expressing human PD-L1 (Raji-APC-L1, Cat #Rajkt-hpd1, Invivogen) and an engineered cell surface protein designed to activate Jurkat's cognate TCRs. Concurrent interaction PD-1/PD-L1 inhibits TCR signaling and represses NFAT-RE-mediated luminescence. Addition of either an anti-PD-1 or anti-PD-L1 antibody that blocks the PD-1/PD-L1 interaction releases the inhibitory signal, restoring TCR activation and resulting in a gain of signal of NFAT-RE luminescent reporter.

Briefly, PD-L1 aAPC/CHO-K1 Target cells were plated in white tissue culture −96 wells plates and cultured 24h at 37° C./5% CO2. Test molecules were measured in serial dilutions starting at and pre-incubated on target cells for 10 min before the addition of freshly thawed PD-1 Jurkat effector cells. After 24 h at 37° C./5% CO2, activity NFAT-RE luminescent reporter was evaluated by removing a 20 microliter sample to which QUANTI-Lic reagent (#rep-qlc, Invivogen) was added. Bioluminescence was then immediately read with a plate reader.

Table showing PD-1 binding and anti-PD-1 activity

| | | human PD1 | | | PD1/PD-L1 | | |
|---|---|---|---|---|---|---|---|
| | | binding | | | blockade | | |
| | | Mean | | | Mean | | |
| in vitro QC assays | | EC50 | Std. | | EC50 | Std. | |
| Molecule | Cleavage | (nM) | Dev. | n= | (nM) | Dev. | n= |
| LZM-009 | Intact | 0.05 | 0.01 | 3 | 7.34 | 1.77 | 3 |
| CMP-016 | Intact | 0.04 | 0.01 | 2 | 12.14 | / | 1 |
| CMP-016 | MMP2 | 0.04 | 0.01 | 3 | 11.63 | 1.70 | 3 |
| CMP-400 | Intact | 0.04 | / | 1 | 9.44 | / | 1 |
| CMP-400 | MMP2 | 0.04 | 0.00 | 3 | 12.66 | 2.15 | 3 |
| CMP-401 | Intact | 0.04 | / | 1 | 7.85 | / | 1 |
| CMP-401 | MMP2 | 0.04 | 0.01 | 3 | 11.73 | 1.94 | 3 |
| CMP-402 | Intact | 0.03 | / | 1 | 8.91 | / | 1 |
| CMP-402 | MMP2 | 0.04 | 0.00 | 3 | 10.24 | 1.58 | 3 |
| CMP-403 | Intact | 0.04 | / | 1 | 9.10 | / | 1 |
| CMP-403 | MMP2 | 0.05 | 0.01 | 3 | 11.47 | 4.55 | 3 |

FcRn Binding Assays—The ability of the activatable immunocytokines to bind to FcRn receptor was performed following manufacturer's instructions using the AlphaLISA FcRn Human Binding kit, Perkin Elmer, Cat #AL3095C. An analogous protocol was performed husing mouse FcRn.

FcγRI (CD64) Binding Assays—The interaction of the activatable immunocytokines with human Fc gamma receptors I (FcγRI/CD64) was measured by ELISA. Briefly, Corning high-binding half-area plates (Fisher Scientific, Reinach, Switzerland) were coated overnight at 4° C. with 25 μl of immunocytokines at 2.5 pg/ml in PBS. Plates were then washed four times with 100 μl of PBS-0.2% Tween20 (wash buffer). Plates surfaces were blocked with 25 μl of PBS1% BSA at 37° C. for 1h. Plates were then washed four times with wash buffer. Then twenty-five microliters of either recombinant Human Fc gamma RI/CD64 Protein (R&D systems, 1257-FC-050, CF) was added in serial dilutions into PBS-T-0.1% BSA and incubated at 37° C. for 2h. Plates were then washed four times with 100 μl of wash buffer. Twenty-five microliters of Streptavidin-Horseradish peroxidase (#RABHRP3, Merck, Buchs, Switzerland) diluted into PBS-T-0.1% BSA were added to each well and incubated at Room Temperature for 1h. Plates were then washed four times with wash buffer. TMB substrate reagent (#CL07, Merck, Buchs, Switzerland) were added to each well and incubated for 5 min. After 5 min, Horseradish peroxidase reaction was stopped by adding 1M H2SO4 stop solution. ELISA signal was then measured at 450 nm on a plate reader. An analogous protocol was performed with mouse CD64.

Table showing Fc receptor binding

| | | human FcRn | | | mouse FcRn | | human FcgR I | | | mouse FcgR I | | |
|---|---|---|---|---|---|---|---|---|---|---|---|---|
| | | binding | | | binding | | (CD64) binding | | | (CD64) binding | | |
| | | Mean | | | Mean | | Mean | | | Mean | | |
| in vitro QC assays | | IC50 | Std. | | EC50 | | EC50 | Std. | | EC50 | Std. | |
| Molecule | Cleavage | (nM) | Dev. | n= | (nM) | n= | (nM) | Dev. | n= | (nM) | Dev. | n= |
| LZM-009 | Intact | 5.79 | 2.84 | 3 | 0.77 | 1 | 0.32 | 0.11 | 3 | 554.40 | 417.10 | 3 |
| CMP-016 | Intact | 24.42 | / | 1 | 2.12 | 1 | 0.26 | / | 1 | 156.40 | / | 1 |
| CMP-016 | MMP2 | 22.11 | 12.94 | 3 | 2.33 | 1 | 0.43 | 0.16 | 3 | 428.80 | 275.80 | 3 |
| CMP-400 | Intact | 24.17 | / | 1 | 2.28 | 1 | 0.26 | / | 1 | 137.60 | / | 1 |
| CMP-400 | MMP2 | 20.19 | 11.78 | 3 | 1.79 | 1 | 0.42 | 0.13 | 3 | 374.00 | 202.60 | 3 |
| CMP-401 | Intact | 19.58 | / | 1 | 1.74 | 1 | 0.26 | / | 1 | 131.70 | / | 1 |
| CMP-401 | MMP2 | 15.96 | 11.75 | 3 | 1.14 | 1 | 0.40 | 0.17 | 3 | 416.20 | 286.90 | 3 |
| CMP-402 | Intact | 33.22 | / | 1 | 3.37 | 1 | 0.21 | / | 1 | 84.35 | / | 1 |
| CMP-402 | MMP2 | 20.21 | 13.60 | 3 | 2.29 | 1 | 0.32 | 0.09 | 3 | 360.20 | 245.10 | 3 |
| CMP-403 | Intact | 21.41 | / | 1 | 2.38 | 1 | 0.24 | / | 1 | 122.80 | / | 1 |
| CMP-403 | MMP2 | 13.67 | 11.69 | 3 | 2.29 | 1 | 0.48 | 0.26 | 3 | 594.30 | 446.70 | 3 |

### Example 63: pSTAT5 Activation of Pan T Cells

The ability of activatable IL-2 immunocytokines to induce STAT5 phosphorylation in pan T cells was assessed. Briefly, frozen pan T cells were thawed and cultured overnight in RPMI with 10% fetal calf serum (FCS), 1% glutamine, 1% non-essential amino acids (NEAA), 25 micromolar beta-mercaptoethanol (BME) at 37° C., 5% CO2, 95% humidity. The following day, the cells were washed and resuspended in PBS. Cells were stimulated with serial dilutions of activatable IL-2 immunocytokine for 40 minutes at 47° C., 5% CO2, 95% humidity followed by cell fixation and permeabilization with the Transcription Factor Phospho Buffer Set from BD Biosciences. Then, the cells were stained for 1 hour on ice with antibodies detecting human Phosphse-Stat5-PE (Tyr694; 1:50, clone 46/Stat5pY694), CD25-BV421 (1:100, clone M-A251), CD45RA-BV711 (1:100, clone HI100), CD4-FITC (1:400, clone RPA-T4), CD8-ApC/Cy7 (1:100, clone SK1), FOXP3-AF647 (1:50, clone 259D). Then, cells were washed twice with ice cold FACS buffer. Data were then acquired on a multi-color flow cytometer and data were analyzed with FlowJo software. EC50s were determined with GraphPadPrism. The table below shows average EC50 values for immunocytokines gated on CD8 Memory Teff cells. Representative graphs of molecules from a single donor are shown in FIG. 8. The data shows that IL-2 related activity of the activatable immunocytokines can be restored upon cleavage when there are conjugated to the IgGs of the anti-PD-1 antibodies. For some of the samples, EC50 values could not be accurately determined due to failure to reach plateau even at the highest immunocytokine tested, though clear evidence of re-activation was still observed.

Table showing average EC50s for activatable immunocytokines gated on CD8 Memory Teff cells

| | Intact molecule | | | MMP2 cleaved molecule | | |
|---|---|---|---|---|---|---|
| Compound | Average EC50 | Std | | Average EC50 | Std | |
| Number | (nM) | Deviation | n= | (nM) | Deviation | n= |
| CMP-016 | 0.7037675 | 0.921676191 | 4 | 0.2797975 | 0.256709643 | 4 |
| CMP-400 | ND | ND | | 1.695225 | 1.462645108 | 4 |
| CMP-401 | ND | ND | | 0.988175 | 0.813478268 | 4 |
| CMP-402 | ND | ND | | 29.4735 | 31.41123771 | 4 |
| CMP-403 | ND | ND | | 2.147975 | 1.811217246 | 4 |

### Example 64: pSTAT5 Activation of Peripheral Blood Mononuclear Cells (PBMCs)

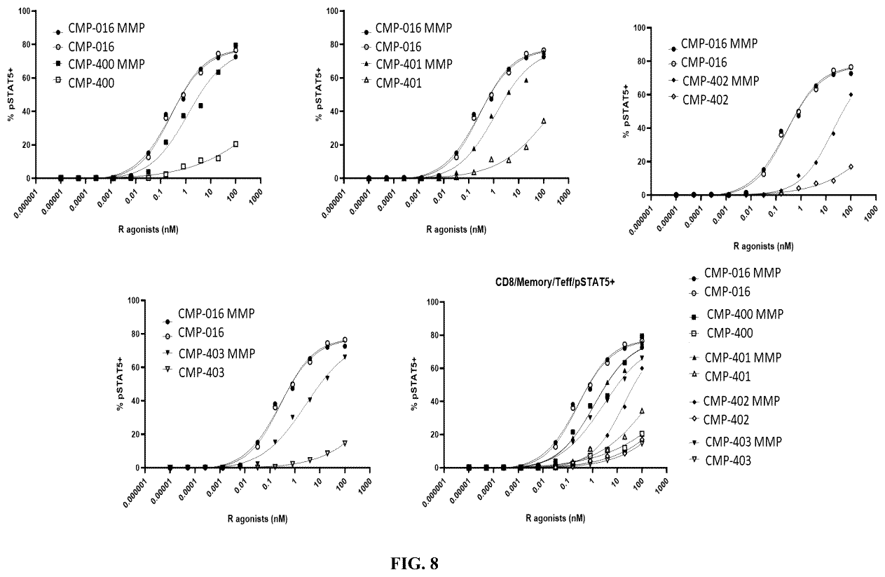

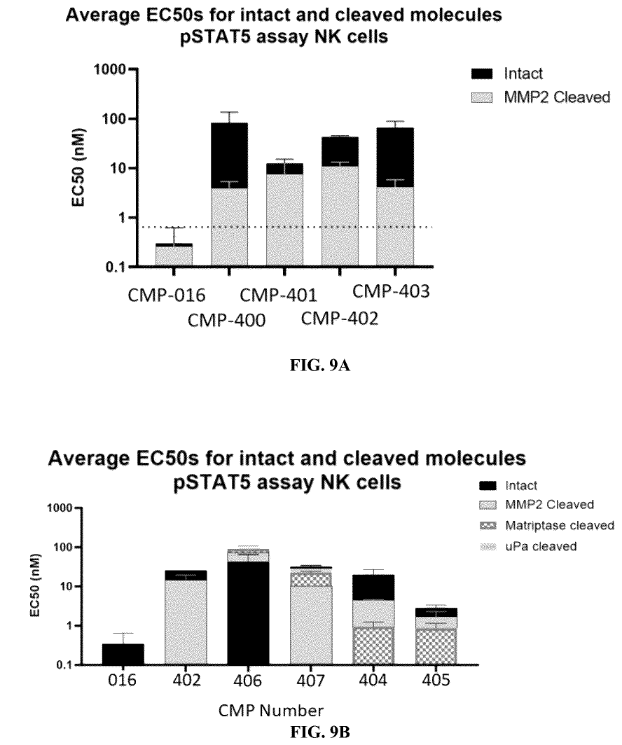

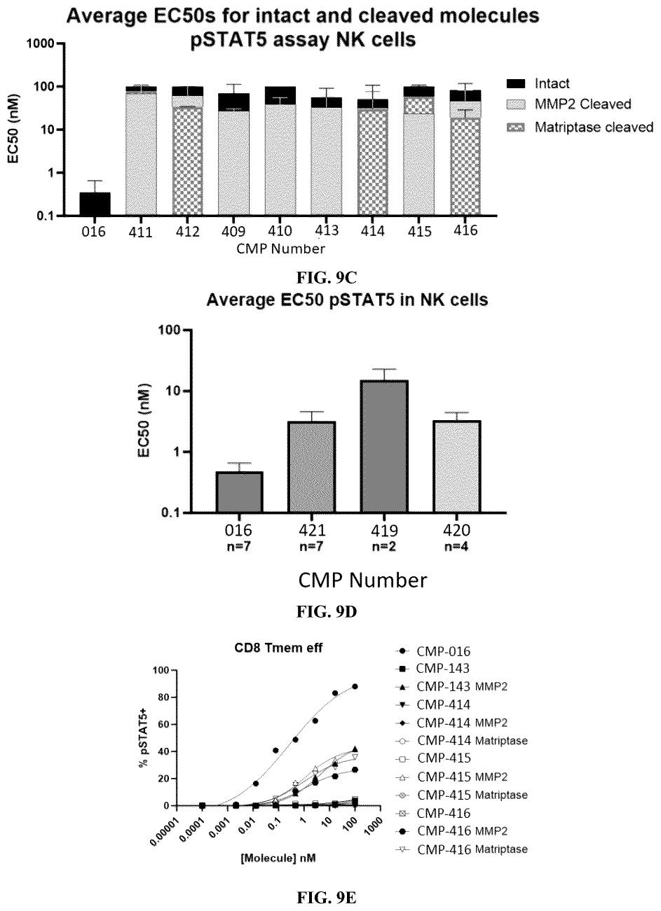

The ability of activatable IL-2 immunocytokines to induce STAT5 phosphorylation in PBMCs was assessed. Briefly, PBMCs were thawed and cultured overnight in RPMI 10% FCS, 1% Glutamine, 1% NEAA, 25 μM beta-mercapto ethanol, 1% sodium pyruvate at 37° C., 5% CO2, 95% humidity. The next day, cells were washed and resuspended in PBS. Cells were stimulated with serial dilutions of activatable immunocytokines for 40 minutes at 37° C., 5% CO2, 95% humidity followed by cell fixation and permeabilization with Transcription Factor Phospho Buffer Set (BD Biosciences). Then, cells were stained for 1 hour on ice with antibodies detecting human Phospho-Stat5-PE (Tyr694; 1:50, clone 47/Stat5pY694), CD25-BV421 (1:100, clone M-A251), CD45RA-BV711 (1:100, clone HI100), CD4-PE texas red (1:800, clone RPA-T4), FOXP3-AF647 (1:50, clone 259D), CD3-PE/Cy7 (1:20, cloneOkt3), CDS6-AF488 (1:400, clone 5.1H11). Then cells were washed twice with ice cold FACS buffer. Data were acquired on a multi-color flow cytometer and data were analyzed with FlowJo software. EC50s were determined with GraphPadPrism. Results from this experiment are shown in the table below for NK cells. FIG. 9A shows average EC50s for intact and cleaved activatable immunocytokines for NK cells as determined in this experiment.

Table showing EC50 values of activatable immunocytokines in NK cells.

| | Intact molecule | | | MMP2 cleaved molecule | | |
|---|---|---|---|---|---|---|
| | Average EC50 | Std | | Average EC50 | Std | |
| Compound | (nM) | Deviation | n= | (nM) | Deviation | n= |
| CMP-003 | 0.299481667 | 0.328717175 | 6 | 0.2619167 | 0.1595047 | 6 |
| CMP-400 | 83.23 | 53.7284791 | 4 | 3.99 | 1.3747613 | 4 |
| CMP-401 | 12.635 | 2.609224023 | 2 | 7.6 | 7.6508954 | 2 |
| CMP-402 | 43.1825 | 2.268014918 | 4 | 11.15675 | 2.1690971 | 4 |
| CMP-403 | 66.035 | 22.79490952 | 4 | 4.1685 | 1.6753062 | 4 |

An analogous experiment to that described above was also performed on additional activatable immunocytokines as described herein, except that selected molecules were also assessed for activation by treatment with uPA and/or matriptase. Results for selected molecules are shown in FIG. 9B and selected cyclic molecules are shown in FIG. 9C. Results for variants CMP-421, CMP-419, and CMP-420 (synthesized to mimic cleavage of the cleavable peptide of CMP-416 at one or more sites) is whon in FIG. 9D. Numerical results from these experiments are shown in the table below.

| | | | | MMP2 cleaved | | | Matriptase cleaved | | | uPa cleaved | | |
|---|---|---|---|---|---|---|---|---|---|---|---|---|
| | Intact molecule | | | molecule | | | molecule | | | molecule | | |
| | Average | | | Average | | | Average | | | Average | | |
| CMP | EC50 | Std | | EC50 | Std | | EC50 | Std | | EC50 | Std | |
| Number | (nM) | Deviation | n= | (nM) | Deviation | n= | (nM) | Deviation | n= | (nM) | Deviation | n= |
| CMP-016 | 0.345 | 0.307 | 22 | | | | | | | | | |
| CMP-402 | 25.260 | 0.283 | 2 | 14.765 | 4.603 | 2 | | | | | | |
| CMP-406 | 41.690 | 21.567 | 2 | 72.680 | 38.636 | 2 | 90.375 | 18.420 | 2 | 44.600 | 22.005 | 2 |
| CMP-407 | 31.600 | 1.881 | 2 | 10.167 | 0.486 | 2 | 22.320 | 1.810 | 2 | 28.720 | 6.435 | 2 |
| CMP-404 | 19.835 | 7.641 | 4 | 4.550 | 0.130 | 4 | 0.937 | 0.286 | 4 | | | |
| CMP-405 | 2.818 | 0.569 | 4 | 1.737 | 0.561 | 4 | 0.856 | 0.287 | 4 | | | |
| CMP-016 | 0.345 | 0.307 | 22 | | | | | | | | | |
| CMP-411 | 100.000 | 0.000 | 2 | 69.105 | 43.692 | 2 | 79.635 | 28.800 | 2 | | | |
| CMP-412 | 100.000 | 0.000 | 2 | 62.590 | 52.906 | 2 | 33.455 | 0.898 | 2 | | | |
| CMP-409 | 69.780 | 42.738 | 2 | 27.185 | 3.429 | 2 | | | | | | |
| CMP-410 | 100.000 | 0.000 | 2 | 38.820 | 16.659 | 2 | | | | | | |
| CMP-413 | 55.383 | 36.296 | 6 | 33.000 | 32.946 | 6 | | | | | | |
| CMP-414 | 50.767 | 56.856 | 4 | 31.631 | 47.257 | 4 | 29.911 | 47.633 | 4 | | | |
| CMP-415 | 100.000 | 0.000 | 4 | 23.418 | 14.263 | 4 | 58.743 | 49.556 | 4 | | | |
| CMP-416 | 82.135 | 35.730 | 4 | 46.443 | 35.790 | 4 | 19.025 | 9.428 | 4 | | | |

A similar experiment was performed using CD8 T memory effector cells, however accurate EC50 determination was not possible using this cell type. Representative results are shown in FIG. 9E.

### Example 64: pSTAT5 Activation of Mouse Splenocytes

The ability of activatable IL-2 immunocytokines to induce STAT5 phosphorylation in mouse splenocytes cells was assessed. Briefly, frozen C57BL/6 mouse splenocytes were thawed and left to recover for 2h in RPMI 10% FCS, 1% Glutamine, 1% non-essential amino acids, 25 M beta-mercapto ethanol, 1% sodium pyruvate at 37° C., 5% CO2, 95% humidity. Afterwards, cells were washed and resuspended in PBS. Cells were stimulated with serial dilutions of activatable immunocytokine for 40 minutes at 37° C., 5% CO2, 95% humidity followed by cell fixation and permeabilization with Transcription Factor Phospho Buffer Set (BD Biosciences). Then, cells were stained for 1 hour on ice with antibodies detecting mouse Phospho-Stat5-AF488 (Tyr694; 1:200, clone 47/Stat5pY694), CD25-BV421 (1:100, clone PC61), CD45R/B220-BV711 (1:100, clone RA3-6B2), CD4-APC (1:100, clone GK1.5), CD8a-BV510 (1:100, clone 53-6.7), CD3-BV650 (1:100, clone 17A2), FOXP3-PE (1:200, clone FJK-16s), NK1.1-AF700 (1:200, clone PK136). Then cells were washed twice with ice cold FACS buffer. Data were acquired on a multi-color flow cytometer and data were analyzed with FlowJo software. Ec50s were determined with GraphPadPrism. Results from this experiment are shown in the table below for NK cells.

Table showing EC50 values of pSTAT5 of activatable immunocytokines on mouse splenocytes.

| | Intact molecule | | | MMP2 cleaved molecule | | |
|---|---|---|---|---|---|---|
| | | Std | | | Std | |
| | Average | Devia- | | Average | Devia- | |
| Compound | EC50 (nM) | tion | n= | EC50 (nM) | tion | n= |
| CMP-016 | 61.985 | 17.115 | 2 | 65.645 | 26.365 | 2 |
| CMP-400 | ND | ND | | 230.95 | 69.75 | 2 |
| CMP-401 | ND | ND | | 167.15 | 22.15 | 2 |
| CMP-402 | ND | ND | | ND | ND | 2 |
| CMP-403 | ND | ND | | 255.55 | 8.85 | 2 |

FIG. 10 shows dose response curves for intact and MMP2 cleaved immunocytokines in CD8 cells from this experiment.

### Example 65-Plasma Stability of Activatable Immunocytokines

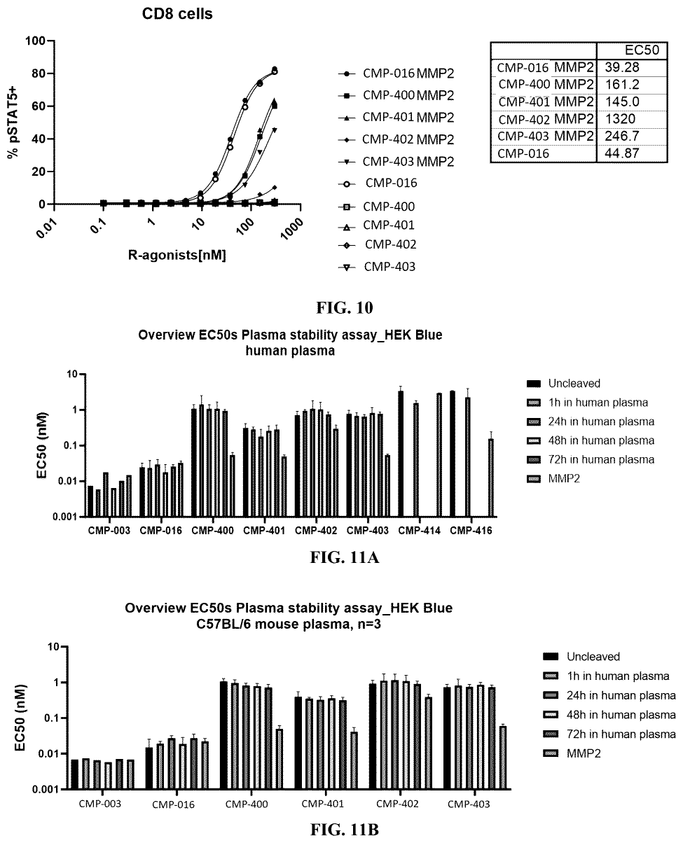

In order to assess the stability of the activatable immunocytokines (including stability of the cleavable moiety), samples of activatable immunocytokines were incubated in human and moues (C57BL/6) plasma. Briefly, activatable immunocytokines (or appropriate control molecule) were diluted to a final concentration of 1 micromolan in 90% heparinized human or C₅₇BL/6 plasma. Samples were then incubated at 37° C. under shaking (600 rpm) over a period of time with samples taken at various timepoints. At each timepoint, a 60 microliter sample was collected and frozen at −80° C. In order to assess the stability at the timepoints, a HEK Blue IL2R reporter assay was performed as described above (e.g., Example 60). The results from these assays for human plasma samples is shown in FIG. 11A and mouse plasma samples is shown in FIG. 11B. For each activatable immunocytokine sample shown in FIGS. 11A and 11B, the farthest right sample is the MMP2 treated control (i.e., fully cleaved control). These results show that the activatable immunocytokines are stable and remain substantially intact (masked) in normal, healthy donor plasma, suggesting the activatable immunocytokines would not demonstrate substantial activity in healthy (i.e., non-target) tissue.

### Example 66-In Vivo Analysis of Activatable Immunocytokines in B16F10-OVA C57BL/6 Mouse Model Transgenic for Human PD1

In order to assess the efficacy and safety of the activatable immunocytokines described herein, an in vivo experiment was performed on a mouse model of B16F10-OVA tumor cells inoculated C57BL/6 mice.

Cell Culture—The B16F10-OVA tumor cells were maintained in vitro as a monolayer culture in DMEM medium supplemented with 10% fetal bovine serum, 100 U/mL penicillin and 100 μg/mL streptomycin at 37° C. in an atmosphere of 5% CO2 in air. The tumor cells were routinely subcultured twice weekly. The cells growing in an exponential growth phase were harvested and counted for tumor inoculation.

Tumor Inoculation—Each mouse was inoculated subcutaneously at the right upper flank with B16F10-OVA tumor cells (0.05×10⁶, 1:1 with Matrigel) in 0.1 mL of PBS for tumor development. Treatments were started on day 7 after tumor inoculation when the average tumor size reached approximately 52 mm³.

Body Weight Assessment—Body weight was measured three times per week post the start of treatment; during Day 3-7 post treatment, body weights were measured daily.

Tumor Size Measurements—Tumor size was measured twice weekly in two dimensions using a caliper, and the volume was expressed in mm³ using the formula: V=0.5 a×b² where a and b are the long and short diameters of the tumor, respectively. The T/C value (in percent) is an indication of antitumor effectiveness; T and C are the mean volume of the treated and control groups, respectively, on a given day. TGI is calculated for each group using the formula: TGI (%)=[1−(Tᵢ−T₀)/(Vᵢ−V₀)]×100; Ti is the average tumor volume of a treatment group on a given day, T₀ is the average tumor volume of the treatment group on the day of treatment start, Vᵢ is the average tumor volume of the vehicle control group on the same day with Tᵢ, and V₀ is the average tumor volume of the vehicle group on the day of treatment start.

Study Groups—A study was performed on the experimental groups outlined below.

| | | | Dose | Dosing | |
|---|---|---|---|---|---|
| Group | n^{a} | Treatment | (mg/kg) | route | Schedule |
| 1 | 7 | Vehicle | — | i.v. | Single dose |
| 2 | 7 | CMP-003 | 1 | i.v. | Single dose |
| 3 | 7 | (Control IL-2) | 8 | i.v. | Single dose |
| 4 | 7 | | 10 | i.v. | Single dose |
| 5 | 7 | CMP-016 | 0.3 | i.v. | Single dose |
| 6 | 7 | (Control | 1 | i.v. | Single dose |
| 7 | 7 | Immunocytokine) | 2.5 | i.v. | Single dose |
| 8 | 7 | CMP-414 | 1 | i.v. | Single dose |
| 9 | 7 | | 8 | i.v. | Single dose |
| 10 | 7 | CMP-415 | 1 | i.v. | Single dose |
| 11 | 7 | | 8 | i.v. | Single dose |
| 12 | 7 | CMP-416 | 1 | i.v. | Single dose |
| 13 | 7 | | 8 | i.v. | Single dose |
| 14 | 7 | CMP-417 | 1 | i.v. | Single dose |
| 15 | 7 | | 8 | i.v. | Single dose |
| 16 | 7 | CMP-418 | 1 | i.v. | Single dose |
| 17 | 7 | | 8 | i.v. | Single dose |

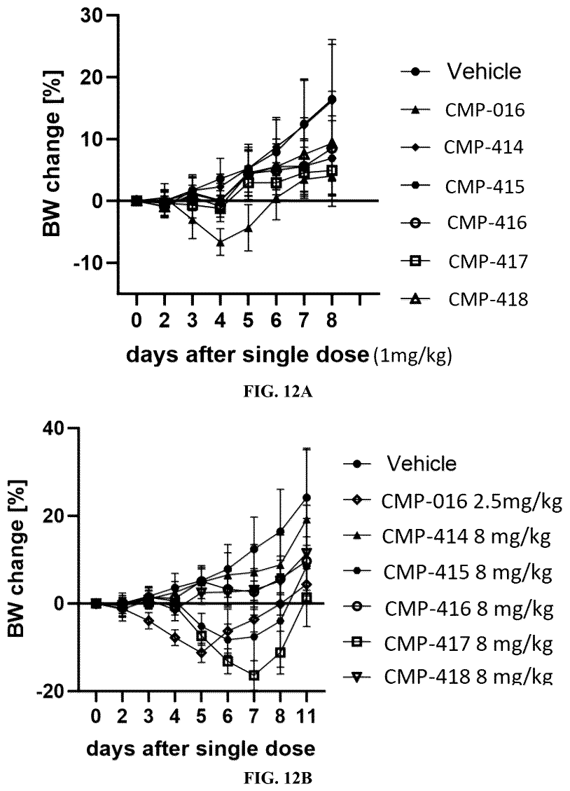

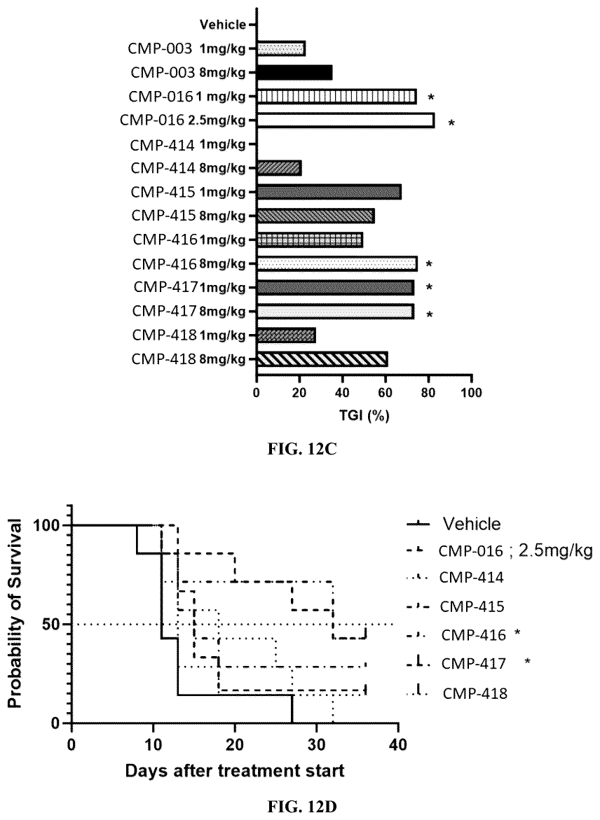

Body weight loss results of all of the 1 mg/kg dosing groups is shown in FIG. 12A. The results show that the activatable immunocytokines were better tolerated (lower or no observed weight loss following administration). Comparison of higher doses is shown in FIG. 12B, which shows the vehicle, 2.5 mg/kg CMP-016 (IL-2/PD-1 immunocytokine control), and 8 mg/kg activatable immunocytokine dosing groups. CMP-416 and CMP-418 did not show any weight loss, whereas CMP-415 and CMP-416 showed some weight loss following administration.

On day 8 of the study (all mice still alive), tumor growth inhibition was assessed. Results are shown in FIG. 12C. FIG. 12C shows that CMP-417 at both doses showed significant tumor growth inhibition, as did CMP-416 at 8 mg/kg. Control immunocytokine CMP-016 also showed significant growth inhibition. Kaplan-Meir analysis was also performed, and the results shown in FIG. 12D. CMP-416 and CMP-417 showed significant improvement in median survival.

### Example 67-Ex Vivo Assessment in NK92 PD1⁺ Cells

The efficacy of activatable immunocytokines in NK92 PD1⁺ cells was assessed using a pSTAT5 assay. Selected activatable immunocytokines were used intact or pretreated with protease (MMP2) or resected tumor homogenate supernatant (MC38, B16F10, or cervix cancer tumors). Protocols and results are provided below.

Tumor Homogenate Preparation—Resected tumors of human or murine origin were surgically resected and snap frozen. Frozen tumors were thawed at 37° C. and transferred into soft tissue homogenizing CK14 tubes prefilled with beads. 1 ml lysis buffer (150 mM NaCL, 50 mM Tris, 10 mM CaCl2, 0.05% Brji-35 at pH 7.5) was added to the tissue and shortly vortexed to mix. Tumors were lysed with a precooled tissue homogenizer (Bertin Technologies—Cryolys evolution) at 5500 rpm—4×40s. The mixture was then centrifuged at 10,000 g, 10 min, 4° C. Supernatant was collected and stored at −80° C. Protein content of the homogenate was determined by BCA. Cleavage of the activatable immunocytokines was performed by incubating the molecules at 5 micromolar with homogenate at 0.1 mg/ml at 37° C. for 18 h under shaking conditions at 600 rpm.

pSTAT5 Assay Protocol—NK92 PD1+ cells were washed and cultured overnight in 75% alpha-MEM (M8042; Sigma), 12.5% FCS, 12.5% Horse Serum, 0.1 mM B-mercaptoethanol (#31350-010 Gibco), 2 mM L-glutamine at 37° C., 5% CO2, 95% humidity. The next day cells were washed and resuspended in PBS. Cells were stimulated with serial dilutions of activatable-immunocytokines for 40 minutes at 37° C., 5% CO2, 95% humidity followed by cell fixation and permeabilization with Transcription Factor Phospho Buffer Set (BD Biosciences). Then, cells were stained for 1 hour on ice with antibodies detecting human Phospho-Stat5-AF488 (Tyr694; 1:50, clone 47/Stat5pY694), Then cells were washed twice with ice cold FACS buffer. Data were acquired on a multi-color flow cytometer and data were analyzed with FlowJo software. EC50s were determined with GraphPadPrism

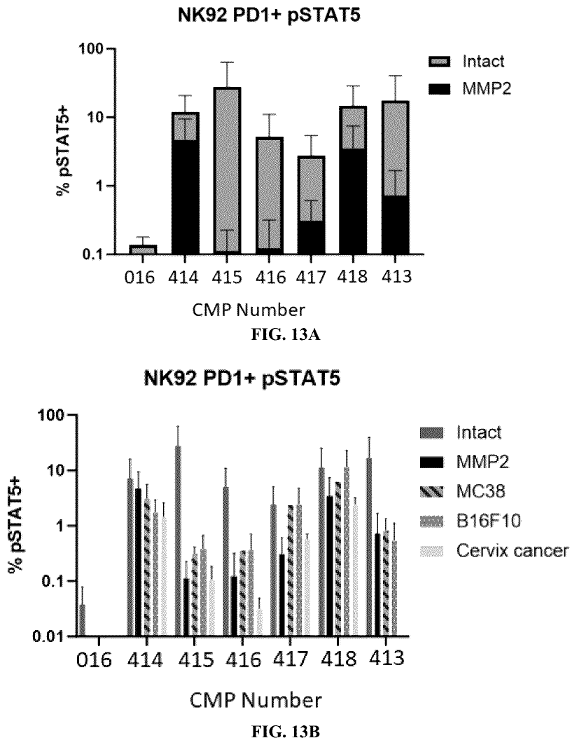

Results—FIG. 13A shows the results of the indicated molecules before and after treatment with MMP. FIG. 13B shows the results before treatment with any protease or tumor homogenate supernatante (“intact”) and after treatment. Numerical results are provided in the table below.

| | | | | | | | | | | | | | Human Cervix | | |
|---|---|---|---|---|---|---|---|---|---|---|---|---|---|---|---|
| | | | | | | | MC38 homogenate | | | B16F10 homogenate | | | cancer homogenate | | |
| | | | | MMP2 cleaved | | | cleaved molecule | | | cleaved molecule | | | cleaved molecule | | |
| | Intact molecule | | | molecule | | | 0.1 mg/ml | | | 0.1 mg/ml | | | 0.01 mg/ml | | |
| | Average | | | Average | | | Average | | | Average | | | Average | | |
| CMP | EC50 | Std | | EC50 | Std | | EC50 | Std | | EC50 | Std | | EC50 | Std | |
| Number | (nM) | Deviation | n= | (nM) | Deviation | n= | (nM) | Deviation | n= | (nM) | Deviation | n= | (nM) | Deviation | n= |
| CMP-016 | 0.038 | 0.041 | 12 | | | | | | | | | | | | |
| CMP-414 | 7.196 | 8.983 | 12 | 4.694 | 4.721 | 12 | 3.077 | 2.530 | 6 | 1.720 | 1.196 | 10 | 1.462 | 1.152 | 3 |
| CMP-415 | 27.865 | 35.608 | 9 | 0.112 | 0.114 | 9 | 0.314 | 0.100 | 2 | 0.384 | 0.286 | 7 | 0.108 | 0.076 | 3 |
| CMP-416 | 5.035 | 5.929 | 8 | 0.124 | 0.194 | 5 | 0.355 | n.a. | 1 | 0.361 | 0.344 | 6 | 0.032 | 0.017 | 2 |
| CMP-417 | 2.427 | 2.674 | 4 | 0.311 | 0.297 | 4 | 2.364 | n.a. | 1 | 2.401 | 2.380 | 3 | 0.573 | 0.144 | 2 |
| CMP-418 | 11.230 | 14.003 | 4 | 3.496 | 3.945 | 4 | 6.245 | n.a. | 1 | 11.401 | 11.565 | 3 | 2.407 | 0.783 | 2 |
| CMP-413 | 16.787 | 22.790 | 3 | 0.721 | 0.944 | 3 | 0.821 | 0.521 | 3 | 0.542 | 0.566 | 3 | | | |

1. An activatable immunocytokine, comprising:
an antibody or antigen binding fragment thereof specific for programmed cell death protein 1 (PD-1);
an interleukin-2 (IL-2) polypeptide;
a linker connecting the antibody or antigen binding fragment thereof to the IL-2 polypeptide, and
a protease cleavable peptide attached to a side chain of an amino acid residue of the IL-2 polypeptide, wherein the IL-2 polypeptide exhibits an enhanced ability to bind to at least one IL-2 receptor subunit after cleavage of the cleavable moiety compared to the ability before cleavage of the cleavable moiety.

2. (canceled)

3. (canceled)

4. The activatable immunocytokine of claim 1, wherein the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof.

5. The activatable immunocytokine of claim 1, wherein the cleavable peptide is cleavable by multiple proteases.

6. (canceled)

7. (canceled)

8. (canceled)

9. (canceled)

10. (canceled)

11. (canceled)

12. (canceled)

13. (canceled)

14. The activatable immunocytokine of claim 1, wherein the cleavable peptide is attached to residue 9, 11, 13, 15, 16 19, 22, 23, 29, or 32 of the IL-2 polypeptide, wherein residue position numbering is based on SEQ ID NO: 1 as a reference sequence.

15. The activatable immunocytokine of claim 14, wherein the cleavable moiety is attached to the IL-2 polypeptide at an additional point of attachment.

16. The activatable immunocytokine of claim 15, wherein the additional point of attachment is to the N-terminus of the IL-2 polypeptide.

17. (canceled)

18. (canceled)

19. (canceled)

20. (canceled)

21. (canceled)

22. The activatable IL-2 polypeptide of claim 1, wherein the C-terminus of the cleavable peptide is attached to the N-terminus of the IL-2 polypeptide and the N-terminus of the cleavable peptide is attached to residue 23 of the IL-2 polypeptide.

23. The activatable IL-2 polypeptide of claim 22, wherein the cleavable peptide comprises the sequence set forth in SEQ ID NO: 617 or 633.

24. (canceled)

25. (canceled)

26. The activatable IL-2 polypeptide of claim 22, wherein the cleavable moiety is directly attached residue 23 of the IL-2 polypeptide and the N-terminus of the IL-2 polypeptide.

27. (canceled)

28. (canceled)

29. (canceled)

30. (canceled)

31. (canceled)

32. The activatable immunocytokine of claim 1, wherein the IL-2 polypeptide comprises polymers covalently attached at residues 42 and 45, wherein residue position numbering is based on SEQ ID NO: 1 as a reference sequence.

33. (canceled)

34. The activatable immunocytokine of claim 1, wherein the IL-2 polypeptide comprises an amino acid sequence having at least about 80%, at least about 85%, at least about 90%, or at least about 95% sequence identity SEQ ID NO: 2 or SEQ ID NO: 3.

35. The activatable immunocytokine of claim 1, wherein the antibody or antigen binding fragment thereof is a monoclonal antibody; a humanized antibody, a grafted antibody, a chimeric antibody, a human antibody.

36. (canceled)

37. (canceled)

38. The activatable immunocytokine of claim 35, wherein the antibody or antigen binding fragment thereof comprises an IgG1 or an IgG4.

39. The activatable immunocytokine of claim 1, wherein the antibody or antigen binding fragment thereof comprises tislelizumab, baizean, sintilimab, tamrelizumab, emiplimab, cemiplimab, lambrolizumab, pembrolizumab, nivolumab, prolgolimab, forteca, penpulimab, zimberelimab, balstilimab, genolimzumab, geptanolimab, dostarlimab, serplulimab, retifanlimab, sasanlimab, spartalizumab, cetrelimab, tebotelimab, cadonilimab, pidilizumab, budigalimab, LZM-009, or a modified version thereof.

40. The activatable immunocytokine of claim 1, wherein the antibody or antigen binding fragment thereof comprises LZM-009.

41. (canceled)

42. (canceled)

43. (canceled)

44. The activatable immunocytokine of claim 1, wherein the linker comprises poly(ethylene glycol).

45. The activatable immunocytokine claim 1, wherein the linker comprises a structure 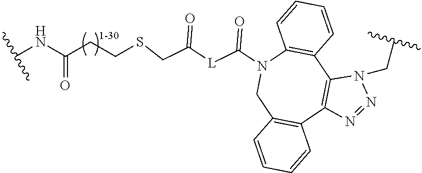
wherein 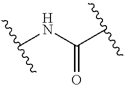 [SMILES: *NC(*)=O]
is the point of attachment to a lysine residue of the antibody or antigen binding fragment;
L is a tether group; and 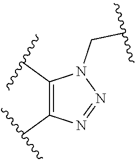 [SMILES: *Cn1nnc(*)c1*]
is a point of attachment to a tether group which connects to the IL-2 polypeptide.

46. (canceled)

47. (canceled)

48. The activatable immunocytokine of claim 47, wherein point of attachment to the antibody or antigen binding fragment thereof is at a position of a K248 amino acid residue, a K288 amino acid residue, or a K317 amino acid residue of an Fc region (EU numbering) of the antibody or antigen binding fragment thereof.

49. The activatable immunocytokine of claim 48, wherein the point of attachment to the antibody or antigen binding fragment thereof is at the K248 amino acid residue.

50. (canceled)

51. (canceled)

52. (canceled)

53. The activatable immunocytokine of claim 1, wherein the point of attachment to the IL-2 polypeptide is at amino acid residue 42 or 45, wherein amino acid residue position numbering of the modified IL-2 polypeptide is based on SEQ ID NO: 1 as a reference sequence.

54. (canceled)

55. (canceled)

56. (canceled)

57. (canceled)

58. (canceled)

59. (canceled)

60. (canceled)

61. (canceled)

62. (canceled)


SEQUENCE LISTING (745 sequences)

SEQ ID NO: 1 - protein, 133 aa, unidentified [sequence omitted]

SEQ ID NO: 2 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 3 - protein, 133 aa, synthetic construct; identical to SEQ ID NO: 2
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 4 - protein, 133 aa, synthetic construct
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQKILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 5 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 6 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 7 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 8 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 9 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 10 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 11 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 12 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 13 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 14 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 15 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 16 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 17 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 18 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 19 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 20 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 21 - protein, 133 aa, synthetic construct
  SITE 11: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTKLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 22 - protein, 133 aa, synthetic construct
  SITE 13: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLKLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 23 - protein, 133 aa, synthetic construct
  SITE 15: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLKHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 24 - protein, 133 aa, synthetic construct
  SITE 19: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLKDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 25 - protein, 133 aa, synthetic construct
  SITE 22: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLKXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 26 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 27 - protein, 133 aa, synthetic construct; identical to SEQ ID NO: 2
  SITE 1: note = Attached to another sequence via 9x polyethylene glycol
  SITE 23: note = Norleucine
  SITE 32: note = Side chain attached to another sequence via a 1,5-pentanedioic acid-9x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 28 - protein, 133 aa, synthetic construct; identical to SEQ ID NO: 2
  SITE 1: note = Attached to another sequence via 4x polyethylene glycol
  SITE 23: note = Norleucine
  SITE 32: note = Side chain attached to another sequence via a 1,5-pentanedioic acid-4x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 29 - protein, 133 aa, synthetic construct; identical to SEQ ID NO: 2
  SITE 1: note = Attached to another sequence via 16x polyethylene glycol
  SITE 23: note = Norleucine
  SITE 32: note = Side chain attached to another sequence via a 1,5-pentanedioic acid-16x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 30 - protein, 133 aa, synthetic construct; identical to SEQ ID NO: 2
  SITE 1: note = Attached to another sequence via 24x polyethylene glycol
  SITE 23: note = Norleucine
  SITE 32: note = Side chain attached to another sequence via a 1,5-pentanedioic acid-9x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 31 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..38: note = Residues linked via a 1,5-pentanedioic acid-9x polyethylene glycol linker
PLGLAGAPTSSSTKKTQLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 32 - protein, 133 aa, synthetic construct; identical to SEQ ID NO: 2
  SITE 1: note = Attached to another sequence via 9x polyethylene glycol
  SITE 23: note = Norleucine
  SITE 32: note = Side chain attached to another sequence via 1,5-pentanedioic acid
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 33 - protein, 133 aa, synthetic construct
  SITE 1: note = Attached to another sequence via 9x polyethylene glycol
  SITE 23: note = Norleucine
  SITE 29: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQXILNGIENYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 34 - protein, 133 aa, synthetic construct; identical to SEQ ID NO: 33
  SITE 1: note = Attached to another sequence via 24x polyethylene glycol
  SITE 23: note = Norleucine
  SITE 29: note = Side chain attached to another sequence via 24x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 35 - protein, 133 aa, synthetic construct; identical to SEQ ID NO: 33
  SITE 1: note = Attached to another sequence via 4x polyethylene glycol
  SITE 23: note = Norleucine
  SITE 29: note = Side chain attached to another sequence via 4x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 36 - protein, 133 aa, synthetic construct; identical to SEQ ID NO: 33
  SITE 1: note = Attached to another sequence via 16x polyethylene glycol
  SITE 23: note = Norleucine
  SITE 29: note = Side chain attached to another sequence via 16x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 37 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..35: note = Residues linked via 9x polyethylene glycol
PLGLAGAPTSSSTKKTQLQLEHLLLDLQXILNGIENYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 38 - protein, 133 aa, synthetic construct; identical to SEQ ID NO: 33
  SITE 1: note = Attached to another sequence via 9x polyethylene glycol
  SITE 23: note = Norleucine
  SITE 29: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 39 - protein, 133 aa, synthetic construct
  SITE 1: note = Attached to another sequence via 4x polyethylene glycol
  SITE 19: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLEDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 40 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..25: note = Residues linked via 4x polyethylene glycol
PLGLAGAPTSSSTKKTQLQLEHLLEDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 41 - protein, 133 aa, synthetic construct
  SITE 1: note = N-terminus attached to another sequence via 4x polyethylene glycol
  SITE 22: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLEXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 42 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..28: note = Residues linked via 4x polyethylene glycol
PLGLAGAPTSSSTKKTQLQLEHLLLDLEXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 43 - protein, 133 aa, synthetic construct
  SITE 1: note = Attached to another sequence via 9x polyethylene glycol
  SITE 23: note = Norleucine
  SITE 26: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQXILEGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 44 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..32: note = Residues linked via 9x polyethylene glycol
PLGLAGAPTSSSTKKTQLQLEHLLLDLQXILEGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 45 - protein, 133 aa, synthetic construct; identical to SEQ ID NO: 4
  SITE 1: note = Attached to another sequence via a 1,5-pentanedioic acid-9x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 46 - protein, 133 aa, synthetic construct; identical to SEQ ID NO: 4
  SITE 1: note = Attached to another sequence via a 1,5-pentanedioic acid-4x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 47 - protein, 133 aa, synthetic construct; identical to SEQ ID NO: 4
  SITE 1: note = Attached to another sequence via 1,5-pentanedioic acid
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 48 - protein, 133 aa, synthetic construct; identical to SEQ ID NO: 4
  SITE 1: note = Attached to another sequence via 1,5-pentanedioic acid
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 49 - protein, 133 aa, synthetic construct; identical to SEQ ID NO: 4
  SITE 1: note = Attached to another sequence via a 1,5-pentanedioic acid-4x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 50 - protein, 133 aa, synthetic construct; identical to SEQ ID NO: 4
  SITE 1: note = Attached to another sequence via a 1,5-pentanedioic acid-9x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 51 - protein, 133 aa, synthetic construct
  SITE 9: note = Side chain attached to another sequence
  SITE 23: note = Side chain attached to another sequence via 4x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 52 - protein, 133 aa, synthetic construct; identical to SEQ ID NO: 51
  SITE 9: note = Side chain attached to another sequence via 4x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 53 - protein, 133 aa, synthetic construct
  SITE 16: note = 2,4-diamino butyric acid
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEXLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 54 - protein, 133 aa, synthetic construct; identical to SEQ ID NO: 53
  SITE 16: note = N-(acetyl-glutyl-valinyl-citrullyl-(N-(4-aminobenzy l alcohol))-formyl)-L-lysine
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 55 - protein, 133 aa, synthetic construct; identical to SEQ ID NO: 2
  SITE 15: note = N-(acetyl-glutyl-valinyl-citrullyl-(N-(4-aminobenzy l alcohol)))ester-L-glutamate
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 56 - protein, 133 aa, synthetic construct; identical to SEQ ID NO: 51
  SITE 1: note = Attached to another sequence via 4x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 57 - protein, 133 aa, synthetic construct; identical to SEQ ID NO: 51
  SITE 1: note = Attached to another sequence via 9x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 58 - protein, 140 aa, synthetic construct
  SITE 46: note = Norleucine
  SITE 48: note = Homoserine
  SITE 49: note = O-(9x polyethylene glycol-azido)-tyrosine
  SITE 52: note = O-(8x polyethylene glycol-amino)-tyrosine
  SITE 53: note = Norleucine
  SITE 78: note = Homoserine
  SITE 111: note = Homoserine
  SITE 1..30: note = Residues linked via the side chain
GPLGLAGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 59 - protein, 139 aa, synthetic construct
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..29: note = Residues linked via 4x polyethylene glycol
PLGLAGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 60 - protein, 139 aa, synthetic construct; identical to SEQ ID NO: 59
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..29: note = Residues linked via 9x polyethylene glycol

SEQ ID NO: 61 - protein, 133 aa, synthetic construct; identical to SEQ ID NO: 4
  SITE 15: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 62 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 63 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 64 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 65 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 66 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 67 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 68 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 69 - protein, 133 aa, synthetic construct; identical to SEQ ID NO: 51
  SITE 1: note = Attached to another sequence via 4x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 70 - protein, 142 aa, synthetic construct
  SITE 48: note = Norleucine
  SITE 50: note = Homoserine
  SITE 51: note = O-(9x polyethylene glycol-azido)-tyrosine
  SITE 54: note = O-(8x polyethylene glycol-amino)-tyrosine
  SITE 55: note = Norleucine
  SITE 80: note = Homoserine
  SITE 113: note = Homoserine
  SITE 1..32: note = Residues linked via the side chain
SGRVANLVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 71 - protein, 143 aa, synthetic construct
  SITE 49: note = Norleucine
  SITE 51: note = Homoserine
  SITE 52: note = O-(9x polyethylene glycol-azido)-tyrosine
  SITE 55: note = O-(8x polyethylene glycol-amino)-tyrosine
  SITE 56: note = Norleucine
  SITE 81: note = Homoserine
  SITE 114: note = Homoserine
  SITE 1..33: note = Residues linked via the side chain
RGRRVANLVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 72 - protein, 144 aa, synthetic construct
  SITE 50: note = Norleucine
  SITE 52: note = Homoserine
  SITE 53: note = O-(9x polyethylene glycol-azido)-tyrosine
  SITE 56: note = O-(8x polyethylene glycol-amino)-tyrosine
  SITE 57: note = Norleucine
  SITE 82: note = Homoserine
  SITE 115: note = Homoserine
  SITE 1..34: note = Residues linked via the side chain
SGRVAPQPLVDAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 73 - protein, 142 aa, synthetic construct
  SITE 48: note = Norleucine
  SITE 50: note = Homoserine
  SITE 51: note = O-(9x polyethylene glycol-azido)-tyrosine
  SITE 54: note = O-(8x polyethylene glycol-amino)-tyrosine
  SITE 55: note = Norleucine
  SITE 80: note = Homoserine
  SITE 113: note = Homoserine
  SITE 1..32: note = Residues linked via the side chain
SGRVQPLVDAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 74 - protein, 136 aa, synthetic construct
  SITE 26: note = (alpha-N-seryl-glycyl-arginyl)-amido-L-glutamate
  SITE 42: note = Norleucine
  SITE 44: note = Homoserine
  SITE 45: note = O-(9x polyethylene glycol-azido)-tyrosine
  SITE 48: note = O-(8x polyethylene glycol-amino)-tyrosine
  SITE 49: note = Norleucine
  SITE 74: note = Homoserine
  SITE 107: note = Homoserine
LVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 75 - protein, 136 aa, synthetic construct; identical to SEQ ID NO: 74
  SITE 26: note = (alpha-N-arginyl-glycyl-arginyl)-amido-L-glutamate
  SITE 42: note = Norleucine
  SITE 44: note = Homoserine
  SITE 45: note = O-(9x polyethylene glycol-azido)-tyrosine
  SITE 48: note = O-(8x polyethylene glycol-amino)-tyrosine
  SITE 49: note = Norleucine
  SITE 74: note = Homoserine
  SITE 107: note = Homoserine

SEQ ID NO: 76 - protein, 136 aa, synthetic construct; identical to SEQ ID NO: 74
  SITE 26: note = Side chain attached to another sequence
  SITE 42: note = Norleucine
  SITE 44: note = Homoserine
  SITE 45: note = O-(9x polyethylene glycol-azido)-tyrosine
  SITE 48: note = O-(8x polyethylene glycol-amino)-tyrosine
  SITE 49: note = Norleucine
  SITE 74: note = Homoserine
  SITE 107: note = Homoserine

SEQ ID NO: 77 - protein, 140 aa, synthetic construct
  SITE 30: note = (alpha-N-arginyl-glycyl-arginyl)-amido-L-glutamate
  SITE 46: note = Norleucine
  SITE 48: note = Homoserine
  SITE 49: note = O-(9x polyethylene glycol-azido)-tyrosine
  SITE 52: note = O-(8x polyethylene glycol-amino)-tyrosine
  SITE 53: note = Norleucine
  SITE 78: note = Homoserine
  SITE 111: note = Homoserine
RVANLVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 78 - protein, 133 aa, synthetic construct; identical to SEQ ID NO: 51
  SITE 9: note = Side chain attached to another sequence
  SITE 23: note = Side chain attached to another sequence via 4x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 79 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 80 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 81 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 82 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 83 - protein, 133 aa, synthetic construct; identical to SEQ ID NO: 4
  SITE 1: note = N-terminus attached to another sequence via a 1,5-pentanedioic acid-9x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 84 - protein, 230 aa, synthetic construct
  VARIANT 18: note = Any naturally occurring amino acid
  VARIANT 19: note = Any naturally occurring amino acid
  VARIANT 21: note = Any naturally occurring amino acid
  VARIANT 140: note = Any naturally occurring amino acid
  VARIANT 142: note = Any naturally occurring amino acid
  VARIANT 177: note = Any naturally occurring amino acid
PKSCDKTHTCPPCPAPEXXGXPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKXTPPVLDSDGSFFLYSKLTVDKSRWQQGNVFSCSVMHEALHNHYTQKSLSLSPG

SEQ ID NO: 85 - protein, 48 aa, synthetic construct
QETNPTENLYFQQKNMQCQRRFYEALHDPNLNEEQRNARIRSIRDDDC

SEQ ID NO: 86 - protein, 61 aa, synthetic construct
QTADNQKNMQCQRRFYEALHDPNLNEEQRNARIRSIRDDCSQSANLLAEAQQLNDAQAPQA

SEQ ID NO: 87 - protein, 38 aa, synthetic construct
QETKNMQCQRRFYEALHDPNLNEEQRNARIRSIRDDDC

SEQ ID NO: 88 - protein, 38 aa, synthetic construct
QETFNKQCQRRFYEALHDPNLNEEQRNARIRSIRDDDC

SEQ ID NO: 89 - protein, 38 aa, synthetic construct
QETFNMQCQRRFYEALHDPNLNKEQRNARIRSIRDDDC

SEQ ID NO: 90 - protein, 37 aa, synthetic construct
QETFNMQCQRRFYEALHDPNLNEEQRNARIRSIKDDC

SEQ ID NO: 91 - protein, 35 aa, synthetic construct
QETMQCQRRFYEALHDPNLNEEQRNARIRSIKDDC

SEQ ID NO: 92 - protein, 34 aa, synthetic construct
QETQCQRRFYEALHDPNLNEEQRNARIRSIKDDC

SEQ ID NO: 93 - protein, 33 aa, synthetic construct
QETCQRRFYEALHDPNLNEEQRNARIRSIKDDC

SEQ ID NO: 94 - protein, 20 aa, synthetic construct
QETRGNCAYHKGQLVWCTYH

SEQ ID NO: 95 - protein, 20 aa, synthetic construct
QETRGNCAYHKGQIIWCTYH

SEQ ID NO: 96 - protein, 6 aa, synthetic construct
  VARIANT 6: note = A or P
LLQGPX

SEQ ID NO: 97 - protein, 8 aa, synthetic construct
GGLLQGPP

SEQ ID NO: 98 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 99-100 - skipped (2 entries)

SEQ ID NO: 101 - protein, 15 aa, synthetic construct
SGGPGPAGMKGLPGS

SEQ ID NO: 102 - protein, 16 aa, synthetic construct
EAGRSANHEPLGLVAT

SEQ ID NO: 103 - protein, 10 aa, synthetic construct
PQASTGRSGG

SEQ ID NO: 104 - protein, 10 aa, synthetic construct
PQGSTGRAAG

SEQ ID NO: 105 - protein, 10 aa, synthetic construct
PPASSGRAGG

SEQ ID NO: 106 - protein, 16 aa, synthetic construct
RSGVPLSLYSGSGGGK

SEQ ID NO: 107 - protein, 14 aa, synthetic construct
RSGMPYDLYHPSGK

SEQ ID NO: 108 - protein, 14 aa, synthetic construct
RGPDSGGFMLTSGK

SEQ ID NO: 109 - protein, 15 aa, synthetic construct
RGSGHEQLTVSGGSK

SEQ ID NO: 110 - protein, 14 aa, synthetic construct
RSGRAAAVKSPSGK

SEQ ID NO: 111 - protein, 20 aa, synthetic construct
RGSGISSGLLSGRSDNHSGK

SEQ ID NO: 112 - protein, 14 aa, synthetic construct
RGDLLAVVAASGGK

SEQ ID NO: 113 - protein, 15 aa, synthetic construct
RGGISSGLLSGRSGK

SEQ ID NO: 114 - protein, 15 aa, synthetic construct
SGGGKKLADEPEGGS

SEQ ID NO: 115 - protein, 13 aa, synthetic construct
GGGKFLADEPEGG

SEQ ID NO: 116 - protein, 8 aa, synthetic construct
ARLQSAAP

SEQ ID NO: 117 - protein, 14 aa, synthetic construct
ARLQSAAPAGLKGA

SEQ ID NO: 118 - protein, 17 aa, synthetic construct
GSGGPGPAGMHGLPGGS

SEQ ID NO: 119 - protein, 16 aa, synthetic construct
GGGSHTGRSAYFGGGS

SEQ ID NO: 120 - protein, 15 aa, synthetic construct
SGGPGPAGLKGAPGS

SEQ ID NO: 121 - protein, 8 aa, synthetic construct
VPLSLYSG

SEQ ID NO: 122 - protein, 11 aa, synthetic construct
SGLLSGRSDNH

SEQ ID NO: 123 - protein, 11 aa, synthetic construct
SGRSDNIGGGS

SEQ ID NO: 124 - protein, 15 aa, synthetic construct
LQESLRSKESGRSDI

SEQ ID NO: 125 - protein, 13 aa, synthetic construct
ISSGLLSGRSDNH

SEQ ID NO: 126 - protein, 8 aa, synthetic construct
LSGRSDDH

SEQ ID NO: 127 - protein, 13 aa, synthetic construct
ISSGLLSGRSDQH

SEQ ID NO: 128 - protein, 13 aa, synthetic construct
ISSGLLSGRSDNI

SEQ ID NO: 129 - protein, 6 aa, synthetic construct
PLGLAG

SEQ ID NO: 130 - protein, 8 aa, synthetic construct
GPAGMKGL

SEQ ID NO: 131 - protein, 8 aa, synthetic construct
LSGRSDQH

SEQ ID NO: 132 - protein, 8 aa, synthetic construct
LSGRSDNI

SEQ ID NO: 133 - protein, 13 aa, synthetic construct
ISSGLLSGRSDNH

SEQ ID NO: 134 - protein, 7 aa, synthetic construct
GPLGVRG

SEQ ID NO: 135 - protein, 7 aa, synthetic construct
GPLGLAR

SEQ ID NO: 136 - protein, 8 aa, synthetic construct
GPAALVGA

SEQ ID NO: 137 - protein, 8 aa, synthetic construct
GPAALIGG

SEQ ID NO: 138 - protein, 8 aa, synthetic construct
GPLNLVGR

SEQ ID NO: 139 - protein, 8 aa, synthetic construct
GPAGLVAD

SEQ ID NO: 140 - protein, 8 aa, synthetic construct
GPANLVAP

SEQ ID NO: 141 - protein, 8 aa, synthetic construct
VPLSLYSG

SEQ ID NO: 142 - protein, 10 aa, synthetic construct
SGESPAYYTA

SEQ ID NO: 143 - protein, 7 aa, synthetic construct
GGPRGLG

SEQ ID NO: 144 - protein, 6 aa, synthetic construct
HSSKLQ

SEQ ID NO: 145 - protein, 7 aa, synthetic construct
HSSKLQL

SEQ ID NO: 146 - protein, 8 aa, synthetic construct
KRALGLPG

SEQ ID NO: 147 - protein, 5 aa, synthetic construct
LEATA

SEQ ID NO: 148 - protein, 10 aa, synthetic construct
GGAANLVRGG

SEQ ID NO: 149 - protein, 10 aa, synthetic construct
SGRIGFLRTA

SEQ ID NO: 150 - protein, 5 aa, synthetic construct
PLGLA

SEQ ID NO: 151 - protein, 8 aa, synthetic construct
ESPAYYTA

SEQ ID NO: 152 - protein, 6 aa, synthetic construct
RLQLKL

SEQ ID NO: 153 - protein, 7 aa, synthetic construct
RLQLKAC

SEQ ID NO: 154 - protein, 5 aa, synthetic construct
SGRSA

SEQ ID NO: 155 - skipped

SEQ ID NO: 156 - protein, 5 aa, synthetic construct
GGGRR

SEQ ID NO: 157 - protein, 4 aa, synthetic construct
GFLG

SEQ ID NO: 158 - protein, 9 aa, synthetic construct
HSSKLQEDA

SEQ ID NO: 159 - protein, 10 aa, synthetic construct
LVLASSSFGY

SEQ ID NO: 160 - protein, 10 aa, synthetic construct
GVSQNYPIVG

SEQ ID NO: 161 - protein, 10 aa, synthetic construct
GVVQASCRLA

SEQ ID NO: 162 - protein, 6 aa, synthetic construct
DPRSFL

SEQ ID NO: 163 - protein, 6 aa, synthetic construct
PPRSFL

SEQ ID NO: 164 - protein, 4 aa, synthetic construct
DEVD

SEQ ID NO: 165 - protein, 5 aa, synthetic construct
DEVDP

SEQ ID NO: 166 - protein, 8 aa, synthetic construct
KGSGDVEG

SEQ ID NO: 167 - protein, 6 aa, synthetic construct
GWEHDG

SEQ ID NO: 168 - protein, 7 aa, synthetic construct
EDDDDKA

SEQ ID NO: 169 - protein, 9 aa, synthetic construct
KQEQNPGST

SEQ ID NO: 170 - protein, 6 aa, synthetic construct
GKAFRR

SEQ ID NO: 171 - protein, 4 aa, synthetic construct
DAFK

SEQ ID NO: 172 - protein, 4 aa, synthetic construct
DVLK

SEQ ID NO: 173 - protein, 4 aa, synthetic construct
DAFK

SEQ ID NO: 174 - protein, 7 aa, synthetic construct
ALLLALL

SEQ ID NO: 175 - protein, 10 aa, synthetic construct
SGAKPRALTA

SEQ ID NO: 176 - protein, 10 aa, synthetic construct
SGLRLAAITA

SEQ ID NO: 177 - protein, 10 aa, synthetic construct
SGESLAYYTA

SEQ ID NO: 178 - protein, 10 aa, synthetic construct
SGESPAYYTA

SEQ ID NO: 179 - protein, 10 aa, synthetic construct
SGESLRYYTA

SEQ ID NO: 180 - protein, 10 aa, synthetic construct
SGRSLSRLTA

SEQ ID NO: 181 - protein, 10 aa, synthetic construct
SGRSLRRLTA

SEQ ID NO: 182 - protein, 10 aa, synthetic construct
SGAVSWLLTA

SEQ ID NO: 183 - protein, 10 aa, synthetic construct
SGAPSWLLTA

SEQ ID NO: 184 - protein, 10 aa, synthetic construct
SGAVRWLLTA

SEQ ID NO: 185 - protein, 10 aa, synthetic construct
SGANISDLTA

SEQ ID NO: 186 - protein, 10 aa, synthetic construct
SGNRYSSLTA

SEQ ID NO: 187 - protein, 10 aa, synthetic construct
SGHMHKALTA

SEQ ID NO: 188 - protein, 10 aa, synthetic construct
SGHMHKALTA

SEQ ID NO: 189 - skipped

SEQ ID NO: 190 - protein, 12 aa, synthetic construct
GEEGEEPLGLAG

SEQ ID NO: 191 - protein, 7 aa, synthetic construct
GPLGLAG

SEQ ID NO: 192 - protein, 16 aa, synthetic construct
EAGRSANHTPAGLTGP

SEQ ID NO: 193 - protein, 17 aa, synthetic construct
GEAGRSANHTPAGLTGP

SEQ ID NO: 194-200 - skipped (7 entries)

SEQ ID NO: 201 - protein, 11 aa, synthetic construct
AGDKSPLGLAG

SEQ ID NO: 202 - protein, 11 aa, synthetic construct
AGDRSPLGLAG

SEQ ID NO: 203 - protein, 12 aa, synthetic construct
AGDRSAPLGLAG

SEQ ID NO: 204 - protein, 11 aa, synthetic construct
AWGRSPLGLAG

SEQ ID NO: 205 - protein, 12 aa, synthetic construct
AWGRSAPLGLAG

SEQ ID NO: 206 - protein, 11 aa, synthetic construct
AWGKSPLGLAG

SEQ ID NO: 207 - protein, 11 aa, synthetic construct
GAFKSPLGLAG

SEQ ID NO: 208 - protein, 11 aa, synthetic construct
SGRSAPLALAG

SEQ ID NO: 209 - protein, 10 aa, synthetic construct
SGRSPLGLAG

SEQ ID NO: 210-213 - skipped (4 entries)

SEQ ID NO: 214 - protein, 12 aa, synthetic construct
RSGRSAPLGLAG

SEQ ID NO: 215 - protein, 12 aa, synthetic construct
RSGKSAPLGLAG

SEQ ID NO: 216 - protein, 12 aa, synthetic construct
FTARSAPLGLAG

SEQ ID NO: 217 - skipped

SEQ ID NO: 218 - protein, 11 aa, synthetic construct
FTAKSPLGLAG

SEQ ID NO: 219 - protein, 12 aa, synthetic construct
RYGRSAPLGLAG

SEQ ID NO: 220 - protein, 11 aa, synthetic construct
RYGRSPLGLAG

SEQ ID NO: 221 - skipped

SEQ ID NO: 222 - protein, 12 aa, synthetic construct
KYGRSAPLGLAG

SEQ ID NO: 223 - protein, 12 aa, synthetic construct
KWGRSAPLGLAG

SEQ ID NO: 224 - skipped

SEQ ID NO: 225 - protein, 11 aa, synthetic construct
KWGRSPLGLAG

SEQ ID NO: 226-235 - skipped (10 entries)

SEQ ID NO: 236 - protein, 13 aa, synthetic construct
SSRGRRGPLGLAG

SEQ ID NO: 237 - protein, 10 aa, synthetic construct
SSRGPLGLAG

SEQ ID NO: 238 - protein, 10 aa, synthetic construct
SSRGPRGLAG

SEQ ID NO: 239-245 - skipped (7 entries)

SEQ ID NO: 246 - protein, 11 aa, synthetic construct
SGRVLPLNLSG

SEQ ID NO: 247-249 - skipped (3 entries)

SEQ ID NO: 250 - protein, 11 aa, synthetic construct
SGRVLPLGLAG

SEQ ID NO: 251 - protein, 11 aa, synthetic construct
SGRVLPMSLRM

SEQ ID NO: 252 - skipped

SEQ ID NO: 253 - protein, 11 aa, synthetic construct
SGRVLPLGLRA

SEQ ID NO: 254 - skipped

SEQ ID NO: 255 - protein, 11 aa, synthetic construct
SGRVLPLGFMG

SEQ ID NO: 256-274 - skipped (19 entries)

SEQ ID NO: 275 - protein, 20 aa, synthetic construct
  VARIANT 1..20: note = This sequence may encompass 1-10 GS repeating units
GSGSGSGSGSGSGSGSGSGS

SEQ ID NO: 276 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 277 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 278 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 279 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 280 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 281 - protein, 15 aa, synthetic construct
GGGGSGGGGSGGGGS

SEQ ID NO: 282 - protein, 20 aa, synthetic construct
GGGGSGGGGSGGGGSGGGGS

SEQ ID NO: 283-331 - skipped (49 entries)

SEQ ID NO: 332 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 333 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 447 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 335 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 336 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 337 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 338 - protein, 443 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 339 - protein, 214 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 340 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 341 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 343 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 344 - protein, 119 aa, synthetic construct
QVQLVQSGVEVKKPGASVKVSCKASGYTFTNYYMYWVRQAPGQGLEWMGGFPSNGGTNFNEKFKNRVTLTTDSSTTTAYMELKSLQFDDTAVYYCARRDYRFDMGFDYWGQGTTVTVSS

SEQ ID NO: 345 - protein, 111 aa, synthetic construct [sequence omitted]

SEQ ID NO: 346 - protein, 113 aa, synthetic construct [sequence omitted]

SEQ ID NO: 347 - protein, 107 aa, synthetic construct [sequence omitted]

SEQ ID NO: 348 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - protein, 214 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 350 - protein, 439 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 351 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 352 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 353 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 354 - protein, 443 aa, synthetic construct
QVQLVESGGGLVKPGGSLRLSCAASGFTFSNYGMSWIRQAPGKGLEWSTISGGGSNIYYADSVKGRFTISRDNAKNSLYLQMNSLRAEDTAVYYCVSYYYGIDFWGQGTSVTVSSASKYGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 355 - protein, 214 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 356 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 357 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 358 - protein, 444 aa, synthetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 359 - protein, 220 aa, synthetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 360 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 361 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - protein, 450 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 363 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 364 - protein, 496 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 365 - protein, 271 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 366 - protein, 447 aa, synthetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 367 - protein, 213 aa, synthetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 368 - protein, 113 aa, synthetic construct
QVQLVESGGGVVQPGRSLRLTCKASGLTFSSSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 369 - protein, 107 aa, synthetic construct
EIVLTQSPATLSLSPGERATLSCRASQSVSSYLAWYQQKPGQAPRLLIYTASNRATGIPARFSGSGSGTDFTLTISSLEPEDFAVYYCQQYSNWPRTFGQGTKVEIK

SEQ ID NO: 370 - protein, 120 aa, synthetic construct
EVQLQQSGPVLVKPGASVKMSCKASGYTFTSYYMYWVKQSHGKSLEWIGGVNPSNGGTNFNEKFKSKATLTVDKSSSTAYMELNSLTSEDSAVYYCARRDYRYDMGFDYWGQGTTLTVSS

SEQ ID NO: 371 - protein, 111 aa, synthetic construct
QIVLTQSPAIMSASPGEKVTMTCRASKGVSTSGYSYLHWYQQKPGSSPRLLIYLASYLESGVPVRFSGSGSGTSYSLTISRMEAEDAATYYCQHSRELPLTFGTGTRLEIK

SEQ ID NO: 372 - protein, 120 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 373 - protein, 111 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIK

SEQ ID NO: 374 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 375 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 376 - protein, 5 aa, synthetic construct
NYYMY

SEQ ID NO: 377 - protein, 17 aa, synthetic construct
GINPSNGGTNFNEKFKN

SEQ ID NO: 378 - protein, 11 aa, synthetic construct
RDYRFDMGFDY

SEQ ID NO: 379 - protein, 15 aa, synthetic construct
RASKGVSTSGYSYLH

SEQ ID NO: 380 - protein, 7 aa, synthetic construct
LASYLES

SEQ ID NO: 381 - protein, 9 aa, synthetic construct
QHSRDLPLT

SEQ ID NO: 382 - protein, 5 aa, synthetic construct
NSGMH

SEQ ID NO: 383 - protein, 17 aa, synthetic construct
VIWYDGSKRYYADSVKG

SEQ ID NO: 384 - protein, 4 aa, synthetic construct
NDDY

SEQ ID NO: 385 - protein, 11 aa, synthetic construct
RASQSVSSYLA

SEQ ID NO: 386 - protein, 7 aa, synthetic construct
DASNRAT

SEQ ID NO: 387 - protein, 9 aa, synthetic construct
QQSSNWPRT

SEQ ID NO: 388 - protein, 9 aa, synthetic construct
FTFSNYGMS

SEQ ID NO: 389 - protein, 10 aa, synthetic construct
TISGGGSNIY

SEQ ID NO: 390 - protein, 9 aa, synthetic construct
VSYYYGIDF

SEQ ID NO: 391 - protein, 11 aa, synthetic construct
KASQDVTTAVA

SEQ ID NO: 392 - protein, 7 aa, synthetic construct
WASTRHT

SEQ ID NO: 393 - protein, 9 aa, synthetic construct
QQHYTIPWT

SEQ ID NO: 394 - protein, 8 aa, synthetic construct
GLTFSSSG

SEQ ID NO: 395 - protein, 8 aa, synthetic construct
IWYDGSKR

SEQ ID NO: 396 - protein, 6 aa, synthetic construct
ATNNDY

SEQ ID NO: 397 - protein, 11 aa, synthetic construct
RASQSVSSYLA

SEQ ID NO: 398 - protein, 7 aa, synthetic construct
TASNRAT

SEQ ID NO: 399 - protein, 9 aa, synthetic construct
QQYSNWPRT

SEQ ID NO: 400 - protein, 831 aa, synthetic construct
MQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPSCPAPEFLGGPSVFLFPPKPKDTLMISRTPEVTCWVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLMQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 401 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 402 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 403 - protein, 111 aa, synthetic construct
DVVMTQSPLSLPVTLGQPASISCRASESVDTSDNSFIHWYQQRPGQSPRLLIYRSSTLESGVPDRFSGSGSGTDFTLKISRVEAEDVGVYYCQQNYDVPWTFGQGTKVEIK

SEQ ID NO: 404 - protein, 111 aa, synthetic construct
EIVLTQSPATLSLSPGERATLSCRASESVDTSDNSFIHWYQQKPGQSPRLLIYRSSTLESGIPARFSGSGSGTDFTLTISSLEPEDFAVYYCQQNYDVPWTFGQGTKVEIK

SEQ ID NO: 405 - protein, 120 aa, synthetic construct
DVQLQESGPGLVKPSQSLSLTCTVTGYSITSDYAWNWIRQFPGNKLEWMGYITYTGRTSYNPSLKSRISITRDTSKNQFFLQLNSVTTEDTATYYCAREMDYYGSTLDYWGQGTTLTVSS

SEQ ID NO: 406 - protein, 111 aa, synthetic construct
KIVLTQSPASLAVSLRQRATISCRASESVDRYGNSFIHWYQQKPGQPPKVLIYRASNLESGFPARFSGSGSRTDFTLTIDPVEADDAATYYCQQNNEDPYTFGSGTKLEIK

SEQ ID NO: 407 - protein, 118 aa, synthetic construct
QVQLQQSGPELVRPGVSVKISCKGSGYTFTDYAMHWVKQSHARTLEWIGVISTYSGDTNYNQKFKDKATMTVDKSSSTAYLELARMTSEDSAIYYCARLGITTGFAYWGQGTLVTVSA

SEQ ID NO: 408 - protein, 111 aa, synthetic construct
DIVLTQSPASLAVSLGQRATISCRASKGVSTSSYSFMHWYQQKPRQPPKLLIKYASYLESGVPARFSGSGSGTDFTLNIHPVEEEDAATYYCHHSREFPWTFGGGTKLEIK

SEQ ID NO: 409 - protein, 116 aa, synthetic construct
EVKLVESGGGLVKPGGSLKLSCAASGFTFSNYGMSWIRQTPEKGLEWVATISGGGRDTYYPDSVKGRFTISRDNVKNNLYLQMSSLRSEDTAFYYCASYYYGIDYWGQGTSVTVSS

SEQ ID NO: 410 - protein, 107 aa, synthetic construct
DIVMTQPHKEMSTSVGDRVRITCKASQDVTTAVAWYQQKPGQSPKLLIYWASTRHTGVPDRFTGSGSGTEFTLTISSVQAEDLALYYCQQHYSIPWTFGGGTKLEIK

SEQ ID NO: 411 - protein, 114 aa, synthetic construct
QVQLQQPGAELVKPGASVKMSCKASGYTFTSTWMHWVKQRPGQGLEWIGAIDPSDSYTTYNQKFKGKATLTVDTSSTTAYMQLSSLTSEDSAVYYCTRSPFDYWGQGTTLTVSS

SEQ ID NO: 412 - protein, 107 aa, synthetic construct
DIVMTQSHKEMSTSVGDRVSITCKASQDVSTAVAWYQQKPGQSPKLLIYSASYRYTGVPDRFTGSGSGTDFTFAISSVQAEDLAVYYCQQHYSHPFTFGSGTKLEIK

SEQ ID NO: 413 - protein, 120 aa, synthetic construct
DVQLQESGPGLVKPSQSLSLTCTVTGYSITSDYAWNWIRQFPGDKLEWLGYITYSGFTNYNPSLKSRISISRDTSKNQFFLQLNSVATEDTATYYCARWHGSAPWYFDYWGRGTTLTVSS

SEQ ID NO: 414 - protein, 112 aa, synthetic construct
DVLMTQTPLSLPVSLGDQASISCRSSQNIVHSDGNTYLEWYLQKPGQSPNLLIYKVSRRFSGVPDRFSGSGSGTDFTLKISRVEAEDLGVYYCFQGSHFPLTFGAGTKLELK

SEQ ID NO: 415 - protein, 117 aa, synthetic construct
DVQLQESGPDLVKPSQSLSLTCTVTGYSITSGYSWHWIRQFPGNKLEWMGFIHSSGDTNYNPSLKSRISFTRDTSKNQFFLQLSSLTDEDTATYYCATYRNWYFDVWGAGTTVTVSS

SEQ ID NO: 416 - protein, 113 aa, synthetic construct
DIVMTQSPSSLTVTAGEKVTMRCKSSQSLLNSGTQKNYLTWYQQKPGQPPKLLIYWASTRESGVPNRFTGSGSGTDFTLTISSVQAEDLSVYYCQSDYTFPLTFGGGTKLELK

SEQ ID NO: 417 - protein, 120 aa, synthetic construct [sequence omitted]

SEQ ID NO: 418 - protein, 111 aa, synthetic construct [sequence omitted]

SEQ ID NO: 419 - protein, 113 aa, synthetic construct [sequence omitted]

SEQ ID NO: 420 - protein, 107 aa, synthetic construct [sequence omitted]

SEQ ID NO: 421 - protein, 120 aa, synthetic construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSS

SEQ ID NO: 422 - protein, 111 aa, synthetic construct
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIK

SEQ ID NO: 423 - protein, 8 aa, synthetic construct
GFSFSSYT

SEQ ID NO: 424 - protein, 8 aa, synthetic construct
ISGGGRDI

SEQ ID NO: 425 - protein, 13 aa, synthetic construct
VLLTGRVYFALDS

SEQ ID NO: 426 - protein, 10 aa, synthetic construct
ESVDTSDNSF

SEQ ID NO: 427 - skipped

SEQ ID NO: 428 - protein, 6 aa, synthetic construct
NYDVPW

SEQ ID NO: 429 - protein, 8 aa, synthetic construct
GYSITSDY

SEQ ID NO: 430 - skipped

SEQ ID NO: 431 - protein, 9 aa, synthetic construct
MDYYGSTLD

SEQ ID NO: 432 - protein, 11 aa, synthetic construct
SESVDRYGNSF

SEQ ID NO: 433 - skipped

SEQ ID NO: 434 - protein, 6 aa, synthetic construct
NNEDPY

SEQ ID NO: 435 - protein, 7 aa, synthetic construct
GYTFTDY

SEQ ID NO: 436 - skipped

SEQ ID NO: 437 - protein, 7 aa, synthetic construct
GITTGFA

SEQ ID NO: 438 - protein, 11 aa, synthetic construct
SKGVSTSSYSF

SEQ ID NO: 439 - skipped

SEQ ID NO: 440 - protein, 6 aa, synthetic construct
SREFPW

SEQ ID NO: 441 - protein, 7 aa, synthetic construct
GFTFSNY

SEQ ID NO: 442 - skipped

SEQ ID NO: 443 - protein, 5 aa, synthetic construct
YYGID

SEQ ID NO: 444 - protein, 7 aa, synthetic construct
SQDVTTA

SEQ ID NO: 445 - skipped

SEQ ID NO: 446 - protein, 6 aa, synthetic construct
HYSIPW

SEQ ID NO: 447 - protein, 7 aa, synthetic construct
GYTFTST

SEQ ID NO: 448-449 - skipped (2 entries)

SEQ ID NO: 450 - protein, 7 aa, synthetic construct
SQDVSTA

SEQ ID NO: 451 - skipped

SEQ ID NO: 452 - protein, 6 aa, synthetic construct
HYSHPF

SEQ ID NO: 453 - protein, 8 aa, synthetic construct
GYSITSDY

SEQ ID NO: 454 - skipped

SEQ ID NO: 455 - protein, 8 aa, synthetic construct
GSAPWYFD

SEQ ID NO: 456 - protein, 12 aa, synthetic construct
SQNIVHSDGNTY

SEQ ID NO: 457 - skipped

SEQ ID NO: 458 - protein, 5 aa, synthetic construct
SHFPL

SEQ ID NO: 459 - protein, 8 aa, synthetic construct
GYSITSGY

SEQ ID NO: 460 - skipped

SEQ ID NO: 461 - protein, 6 aa, synthetic construct
RNWYFD

SEQ ID NO: 462 - protein, 13 aa, synthetic construct
SQSLLNSGTQKNY

SEQ ID NO: 463 - skipped

SEQ ID NO: 464 - protein, 6 aa, synthetic construct
DYTFPL

SEQ ID NO: 465 - protein, 7 aa, synthetic construct
GYFTNYY

SEQ ID NO: 466 - protein, 8 aa, synthetic construct
INPSNGGT

SEQ ID NO: 467 - protein, 13 aa, synthetic construct
ARRDYRFDMGFDY

SEQ ID NO: 468 - protein, 10 aa, synthetic construct
KGVSTSGYSY

SEQ ID NO: 469 - skipped

SEQ ID NO: 470 - protein, 9 aa, synthetic construct
QHSRDLPLT

SEQ ID NO: 471 - protein, 8 aa, synthetic construct
GITFSNSG

SEQ ID NO: 472 - protein, 8 aa, synthetic construct
IWYDGSKR

SEQ ID NO: 473 - protein, 6 aa, synthetic construct
ATNDDY

SEQ ID NO: 474 - protein, 6 aa, synthetic construct
QSVSSY

SEQ ID NO: 475 - skipped

SEQ ID NO: 476 - protein, 9 aa, synthetic construct
QQSSNWRRT

SEQ ID NO: 477 - protein, 113 aa, synthetic construct
QVQLVESGGDVVQPGGSLRLSCAASGVAFSNYGMHWVRQAPGKGLEWVAVIWYDGSNKYYADSVKGRFTISRDNSKNMLYLQMNSLRAEDTAMYYCARNDDYWGQGTLVTVSS

SEQ ID NO: 478 - protein, 107 aa, synthetic construct
EIVLTQSPATLSLSPGERATLSCRASQSVSSYLAWYQQKPGQAPRLIIYDASNRATGIPARFSGSGSGTDFTLTISSLEPEDFAVYYCQQRSNWPLTFGGGTKVEIK

SEQ ID NO: 479 - protein, 113 aa, synthetic construct
QVQLVESGGDVVQPGRSLRLSCAASGLTFTNYGFHWVRQAPGKGLEWVAVIWYDGSKKYYADSVKGRFTISRDNSKNTLYLQMNNLRAEDTAVYYCATGDDYWGQGTLVTVSS

SEQ ID NO: 480 - protein, 107 aa, synthetic construct
EIVLTQSPATLSLSPGERATLSCRASQSVSSYLAWYQQKPGQAPRLLIYDTSNRATGIPARFSGSGSGTDFTLTISSLEPEDFAVYYCQQRSNWPLTFGGGTKVEIK

SEQ ID NO: 481 - protein, 113 aa, synthetic construct
QVYLVESGGGVVQPGRSLRLSCAASGFTFSNYGMHWVRQAPGKGLEWVALIWYDGSNKYYADSVKGRFTISRDNSKNTLYLQMTSLRVEDTAVYYCASNVDHWGQGTLVTVSS

SEQ ID NO: 482 - protein, 121 aa, synthetic construct
QLQLQESGPGLVKPSETLSLTCTVSGGSLSRSSFFWGWIRQPPGKGLEWIGSIYYSGSTYYNPSLKSRVTISVDTSKNQFSLKLSSVTAADTAVYYCVRDYDILTGDEDYWGQGTLVTVSS

SEQ ID NO: 483 - protein, 113 aa, synthetic construct
QVQLVESGGGVVQPGRSLRLSCTTSGITFSSYGFHWVRQAPGKGLEWVAVIWYDGSKKYYADSVKGRFTLSRDDSKNTLYLQMNSLRAEDTAVYYCVTGDDYWGQGTLVTVSS

SEQ ID NO: 484 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 485 - protein, 121 aa, synthetic construct
QLQLQESGPGLVKPSETLSLTCSVSGGSLSRSSYFWGWIRQPPGKGLEWIASIFYSGETYFNPSLKSRVTISVDTSRNQFSLKLSSVTAADTAVYYCARDYDILTGDEDYWGQGTLVTVSS

SEQ ID NO: 486 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYYSYPRTFGQGTKVEIK

SEQ ID NO: 487 - protein, 5 aa, synthetic construct
NYGMH

SEQ ID NO: 488 - protein, 17 aa, synthetic construct
VIWYDGSNKYYADSVKG

SEQ ID NO: 489 - protein, 4 aa, synthetic construct
NDDY

SEQ ID NO: 490 - protein, 11 aa, synthetic construct
RASQSVSSYLA

SEQ ID NO: 491 - protein, 7 aa, synthetic construct
DASNRAT

SEQ ID NO: 492 - protein, 9 aa, synthetic construct
QQRSNWPLT

SEQ ID NO: 493 - protein, 5 aa, synthetic construct
NYGFH

SEQ ID NO: 494 - protein, 17 aa, synthetic construct
VIWYDGSKKYYADSVKG

SEQ ID NO: 495 - protein, 4 aa, synthetic construct
GDDY

SEQ ID NO: 496 - protein, 11 aa, synthetic construct
RASQSVSSYLA

SEQ ID NO: 497 - protein, 7 aa, synthetic construct
DTSNRAT

SEQ ID NO: 498 - protein, 9 aa, synthetic construct
QQRSNWPLT

SEQ ID NO: 499 - protein, 5 aa, synthetic construct
NYGMH

SEQ ID NO: 500 - protein, 17 aa, synthetic construct
LIWYDGSNKYYADSVKG

SEQ ID NO: 501 - protein, 4 aa, synthetic construct
NVDH

SEQ ID NO: 502 - protein, 7 aa, synthetic construct
RSSFFWG

SEQ ID NO: 503 - protein, 16 aa, synthetic construct
SIYYSGSTYYNPSLKS

SEQ ID NO: 504 - protein, 11 aa, synthetic construct
DYDILTGDEDY

SEQ ID NO: 505 - protein, 5 aa, synthetic construct
SYGFH

SEQ ID NO: 506 - protein, 17 aa, synthetic construct
VIWYDGSKKYYADSVKG

SEQ ID NO: 507 - protein, 4 aa, synthetic construct
GDDY

SEQ ID NO: 508 - protein, 11 aa, synthetic construct
RASQSVSSYLA

SEQ ID NO: 509 - protein, 7 aa, synthetic construct
DASNRAT

SEQ ID NO: 510 - protein, 9 aa, synthetic construct
QQRSNWPLT

SEQ ID NO: 511 - protein, 7 aa, synthetic construct
RSSYFWG

SEQ ID NO: 512 - protein, 16 aa, synthetic construct
SIFYSGETYFNPSLKS

SEQ ID NO: 513 - protein, 11 aa, synthetic construct
DYDILTGDEDY

SEQ ID NO: 514 - protein, 11 aa, synthetic construct
RASQGISSWLA

SEQ ID NO: 515 - protein, 7 aa, synthetic construct
AASSLQS

SEQ ID NO: 516 - protein, 9 aa, synthetic construct
QQYYSYPRT

SEQ ID NO: 517 - protein, 447 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 518 - protein, 218 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519-600 - skipped (82 entries)

SEQ ID NO: 601 - protein, 17 aa, synthetic construct
SSRGPASNRRLPLGLAG

SEQ ID NO: 602 - protein, 17 aa, synthetic construct
SSRAVFRKNLGPLGLAG

SEQ ID NO: 603 - protein, 15 aa, synthetic construct
SGRVLRKAGPQPLVD

SEQ ID NO: 604 - protein, 16 aa, synthetic construct
SGRVLTLRKAPWGLLE

SEQ ID NO: 605 - protein, 12 aa, synthetic construct
SGRVLGPAGLVG

SEQ ID NO: 606 - protein, 12 aa, synthetic construct
SGRVLGPANLVG

SEQ ID NO: 607 - protein, 11 aa, synthetic construct
SGRVLPAGLVG

SEQ ID NO: 608 - protein, 12 aa, synthetic construct
SGRVLGPLNLSG

SEQ ID NO: 609 - protein, 12 aa, synthetic construct
RQRRSAPLGLAG

SEQ ID NO: 610 - protein, 11 aa, synthetic construct
RQRRSPLGLAG

SEQ ID NO: 611 - protein, 17 aa, synthetic construct
SGRVLTLRKAGPAGLVG

SEQ ID NO: 612 - protein, 17 aa, synthetic construct
SGRVLTLRKAGPANLVG

SEQ ID NO: 613 - protein, 15 aa, synthetic construct
SGRVLRKAGPAGLVG

SEQ ID NO: 614 - protein, 15 aa, synthetic construct
SGRVLRKAGPANLVG

SEQ ID NO: 615 - protein, 11 aa, synthetic construct
SGRVLPANLVG

SEQ ID NO: 616 - protein, 9 aa, synthetic construct
SGRVAGLVG

SEQ ID NO: 617 - protein, 9 aa, synthetic construct
SGRVANLVG

SEQ ID NO: 618 - protein, 13 aa, synthetic construct
PASNRRLPLGLAG

SEQ ID NO: 619 - protein, 13 aa, synthetic construct
SSRVFRKPANLAG

SEQ ID NO: 620 - protein, 16 aa, synthetic construct
SGRVLTLRKAALPLAM

SEQ ID NO: 621 - protein, 13 aa, synthetic construct
SSRGRRGPYMLQG

SEQ ID NO: 622 - protein, 10 aa, synthetic construct
SSRGPYMLQG

SEQ ID NO: 623 - protein, 11 aa, synthetic construct
SGRVLPLGMRA

SEQ ID NO: 624 - protein, 11 aa, synthetic construct
SGRVLPYAMTA

SEQ ID NO: 625 - protein, 12 aa, synthetic construct
  SITE 6: note = Norleucine
RQRRSXPLGLAG

SEQ ID NO: 626 - protein, 12 aa, synthetic construct
KWGKSAPLGLAG

SEQ ID NO: 627 - protein, 12 aa, synthetic construct
RYGKSAPLGLAG

SEQ ID NO: 628 - protein, 13 aa, synthetic construct
RQRRSAAPLGLAG

SEQ ID NO: 629 - protein, 9 aa, synthetic construct
RQRRSVVGG

SEQ ID NO: 630 - protein, 8 aa, synthetic construct
SPLGLAGS

SEQ ID NO: 631 - protein, 10 aa, synthetic construct
RGRKVANLVG

SEQ ID NO: 632 - protein, 10 aa, synthetic construct
RQRKVANLVG

SEQ ID NO: 633 - protein, 10 aa, synthetic construct
RGRRVANLVG

SEQ ID NO: 634 - protein, 11 aa, synthetic construct
RGRKSPANLVG

SEQ ID NO: 635 - protein, 10 aa, synthetic construct
RGRKPYMLQG

SEQ ID NO: 636 - protein, 10 aa, synthetic construct
  SITE 7: note = Norleucine
RGRKPYXLQG

SEQ ID NO: 637 - protein, 11 aa, synthetic construct
RGRKSPYMLQG

SEQ ID NO: 638 - protein, 11 aa, synthetic construct
  SITE 8: note = Norleucine
RGRKSPYXLQG

SEQ ID NO: 639 - protein, 10 aa, synthetic construct
RGRKPQPLVD

SEQ ID NO: 640 - protein, 11 aa, synthetic construct
RGRKSPQPLVD

SEQ ID NO: 641 - protein, 10 aa, synthetic construct
RGRKSQPLVD

SEQ ID NO: 642 - protein, 11 aa, synthetic construct
SGRVAPYMLQG

SEQ ID NO: 643 - protein, 11 aa, synthetic construct
  SITE 8: note = Norleucine
SGRVAPYXLQG

SEQ ID NO: 644 - protein, 9 aa, synthetic construct
SGRVYMLQG

SEQ ID NO: 645 - protein, 9 aa, synthetic construct
  SITE 6: note = Norleucine
SGRVYXLQG

SEQ ID NO: 646 - protein, 11 aa, synthetic construct
SGRVAPQPLVD

SEQ ID NO: 647 - protein, 9 aa, synthetic construct
SGRVQPLVD

SEQ ID NO: 648 - protein, 6 aa, synthetic construct
RGRRGP

SEQ ID NO: 649 - protein, 10 aa, synthetic construct
RGRRPLGLAG

SEQ ID NO: 650 - protein, 15 aa, synthetic construct
RGRRVANPLGLAGSG

SEQ ID NO: 651 - protein, 13 aa, synthetic construct
RGRRPLGLAGGSG

SEQ ID NO: 652 - protein, 10 aa, synthetic construct
RGRRHSSKLQ

SEQ ID NO: 653 - protein, 12 aa, synthetic construct
SGRVANPLGGSG

SEQ ID NO: 654 - protein, 11 aa, synthetic construct
SGRVANYFGKL

SEQ ID NO: 655 - protein, 12 aa, synthetic construct
RGRRVANYFGKL

SEQ ID NO: 656 - protein, 11 aa, synthetic construct
SGRPLGYFGKL

SEQ ID NO: 657 - protein, 12 aa, synthetic construct
RGRRPLGYFGKL

SEQ ID NO: 658 - protein, 15 aa, synthetic construct
RGRRVANPLGYFGKL

SEQ ID NO: 659 - protein, 21 aa, synthetic construct
RGRRSGRAANLVRPLGYFGKL

SEQ ID NO: 660 - protein, 18 aa, synthetic construct
RGRRAANLVRPLGYFGKL

SEQ ID NO: 661 - protein, 11 aa, synthetic construct
HSSKLQYFGKL

SEQ ID NO: 662 - protein, 18 aa, synthetic construct
RGRRHSSKLQPLGYFGKL

SEQ ID NO: 663 - protein, 17 aa, synthetic construct
SGRHSSKLQPLGYFGKL

SEQ ID NO: 664 - protein, 8 aa, synthetic construct
GSGSGSGS

SEQ ID NO: 665 - protein, 8 aa, synthetic construct
SSLYSSPG

SEQ ID NO: 666 - protein, 8 aa, synthetic construct
SSLQSSPG

SEQ ID NO: 667 - protein, 8 aa, synthetic construct
SQYQSSPG

SEQ ID NO: 668 - protein, 8 aa, synthetic construct
SQLYSSPG

SEQ ID NO: 669 - protein, 8 aa, synthetic construct
SSQYSSPG

SEQ ID NO: 670 - protein, 8 aa, synthetic construct
ISQYSSAT

SEQ ID NO: 671 - protein, 7 aa, synthetic construct
KLYSSKQ

SEQ ID NO: 672 - protein, 7 aa, synthetic construct
KLFSSKQ

SEQ ID NO: 673 - protein, 7 aa, synthetic construct
RRLHYSL

SEQ ID NO: 674 - protein, 7 aa, synthetic construct
RRLNYSL

SEQ ID NO: 675 - protein, 7 aa, synthetic construct
RSSYRSL

SEQ ID NO: 676 - protein, 7 aa, synthetic construct
RSSYYSL

SEQ ID NO: 677 - protein, 7 aa, synthetic construct
KSKQHSL

SEQ ID NO: 678 - protein, 7 aa, synthetic construct
HSSKLQL

SEQ ID NO: 679 - protein, 8 aa, synthetic construct
GSSYYSGA

SEQ ID NO: 680 - protein, 8 aa, synthetic construct
GSSVYSGR

SEQ ID NO: 681 - protein, 8 aa, synthetic construct
  SITE 3: note = Norleucine
SSXYSSAG

SEQ ID NO: 682 - protein, 17 aa, synthetic construct
  SITE 1: note = Acetylated residue
  SITE 8: note = 3-((3-((2,5-dioxopyrrolidin-1-yl)oxy)-3-oxopropyl)d isulfaneyl)propanoate modified lysine
  SITE 17: note = Amidated residue
  DISULFID 4..14: note = Intrachain disulfide bond
RGNCAYHXGQLVWCTYH

SEQ ID NO: 683 - protein, 17 aa, synthetic construct
  SITE 1: note = Acetylated residue
  SITE 8: note = 4-oxo-4-((3-oxo-3-(phenylthio)propyl)thio)butanoate modified lysine
  SITE 17: note = Amidated residue
  DISULFID 4..14: note = Intrachain disulfide bond
RGNCAYHXGQLVWCTYH

SEQ ID NO: 684 - protein, 34 aa, synthetic construct
  SITE 1: note = Acetylated residue
  SITE 31: note = 3-(((3-oxo-3-(phenylthio)propyl)thio)carbonyl)benzo ate modified lysine
  SITE 34: note = Amidated residue
  DISULFID 5..34: note = Intrachain disulfide bond
FNMQCQRRFYEALHDPNLNEEQRNARIRSIXDDC

SEQ ID NO: 685 - protein, 7 aa, synthetic construct
  SITE 1: note = (2,2,4,6,7-pentamethyldihydrobenzofuran-5-sulfonyl) -L-arginine
  SITE 3: note = (2,2,4,6,7-pentamethyldihydrobenzofuran-5-sulfonyl) -L-arginine
  SITE 4: note = (2,2,4,6,7-pentamethyldihydrobenzofuran-5-sulfonyl) -L-arginine
  SITE 7: note = 5-(4-(hydroxymethyl)phenoxy)pentanoic acid modified (2S)-2-(9H-fluoren-9-ylmethoxycarbonylamino)-4-oxo-4-(trit ylamino)butanoate
RGRRVAX

SEQ ID NO: 686 - protein, 6 aa, synthetic construct
  SITE 1: note = Acetylated residue
  SITE 6: note = C-terminus attached to another sequence
PLGLAG

SEQ ID NO: 687 - protein, 6 aa, synthetic construct
  SITE 6: note = C-terminus attached to another sequence
PLGLAG

SEQ ID NO: 688 - protein, 6 aa, synthetic construct
  SITE 1: note = Acetylated residue
  SITE 6: note = C-terminus attached to another sequence via 9x polyethylene glycol
PLGLAG

SEQ ID NO: 689 - protein, 6 aa, synthetic construct
  SITE 6: note = C-terminus attached to another sequence via 9x polyethylene glycol
PLGLAG

SEQ ID NO: 690 - protein, 15 aa, synthetic construct
  SITE 1: note = Acetylated residue
  SITE 15: note = C-terminus attached to another sequence
SGGPGPAGMKGLPGS

SEQ ID NO: 691 - protein, 7 aa, synthetic construct
  SITE 7: note = C-terminus attached to another sequence
MKGLPGS

SEQ ID NO: 692 - protein, 15 aa, synthetic construct
  SITE 15: note = C-terminus attached to another sequence
SGGPGPAGMKGLPGS

SEQ ID NO: 693 - protein, 15 aa, synthetic construct
  SITE 1: note = Acetylated residue
  SITE 15: note = C-terminus attached to another sequence via 9x polyethylene glycol
SGGPGPAGMKGLPGS

SEQ ID NO: 694 - protein, 7 aa, synthetic construct
  SITE 7: note = C-terminus attached to another sequence via 9x polyethylene glycol
MKGLPGS

SEQ ID NO: 695 - protein, 15 aa, synthetic construct
  SITE 15: note = C-terminus attached to another sequence via 9x polyethylene glycol
SGGPGPAGMKGLPGS

SEQ ID NO: 696 - protein, 6 aa, synthetic construct
  SITE 6: note = C-terminus attached to another sequence
PLGLAG

SEQ ID NO: 697 - protein, 6 aa, synthetic construct
  SITE 6: note = C-terminus attached to another sequence
PLGLAG

SEQ ID NO: 698 - protein, 12 aa, synthetic construct
  SITE 12: note = C-terminus attached to another sequence
GEEGEEPLGLAG

SEQ ID NO: 699 - protein, 7 aa, synthetic construct
  SITE 1: note = Lysine attached to 27x polyethylene glycol
  SITE 7: note = C-terminus attached to another sequence
KPLGLAG

SEQ ID NO: 700 - protein, 8 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 8: note = C-terminus attached to another sequence
KKPLGLAG

SEQ ID NO: 701 - protein, 6 aa, synthetic construct
  SITE 6: note = C-terminus attached to another sequence
PLGLAG

SEQ ID NO: 702 - protein, 6 aa, synthetic construct
  SITE 6: note = C-terminus attached to another sequence
PLGLAG

SEQ ID NO: 703 - protein, 6 aa, synthetic construct
  SITE 6: note = C-terminus attached to another sequence
PLGLAG

SEQ ID NO: 704 - protein, 6 aa, synthetic construct
  SITE 6: note = C-terminus attached to another sequence
PLGLAG

SEQ ID NO: 705 - protein, 6 aa, synthetic construct
  SITE 6: note = C-terminus attached to another sequence
PLGLAG

SEQ ID NO: 706 - protein, 16 aa, synthetic construct
  SITE 16: note = C-terminus attached to another sequence
EAGRSANHTPAGLTGP

SEQ ID NO: 707 - protein, 6 aa, synthetic construct
  SITE 1: note = Attached to another sequence via a 1,5-pentanedioic acid-9x polyethylene glycol linker
  SITE 6: note = Attached to another sequence via 9x polyethylene glycol
PLGLAG

SEQ ID NO: 708 - protein, 15 aa, synthetic construct
  SITE 1: note = Attached to another sequence via a 1,5-pentanedioic acid-4x polyethylene glycol linker
  SITE 15: note = Attached to another sequence via 4x polyethylene glycol
SGGPGPAGMKGLPGS

SEQ ID NO: 709 - protein, 15 aa, synthetic construct
  SITE 1: note = Attached to another sequence via a 1,5-pentanedioic acid-16x polyethylene glycol linker
  SITE 15: note = Attached to another sequence via 16x polyethylene glycol
SGGPGPAGMKGLPGS

SEQ ID NO: 710 - protein, 6 aa, synthetic construct
  SITE 1: note = Attached to another sequence via a 1,5-pentanedioic acid-9x polyethylene glycol linker
  SITE 6: note = Attached to another sequence via 24x polyethylene glycol
PLGLAG

SEQ ID NO: 711 - protein, 7 aa, synthetic construct
  SITE 1: note = Attached to another sequence via 1,5-pentanedioic acid
  SITE 7: note = Attached to another sequence via 9x polyethylene glycol
GPLGLAG

SEQ ID NO: 712 - protein, 6 aa, synthetic construct
  SITE 1: note = Attached to another sequence via 9x polyethylene glycol
  SITE 6: note = Attached to another sequence via 9x polyethylene glycol
PLGLAG

SEQ ID NO: 713 - protein, 15 aa, synthetic construct
  SITE 1: note = Attached to another sequence via 24x polyethylene glycol
  SITE 15: note = Attached to another sequence via 24x polyethylene glycol
SGGPGPAGMKGLPGS

SEQ ID NO: 714 - protein, 15 aa, synthetic construct
  SITE 1: note = Attached to another sequence via 4x polyethylene glycol
  SITE 15: note = Attached to another sequence via 4x polyethylene glycol
SGGPGPAGMKGLPGS

SEQ ID NO: 715 - protein, 15 aa, synthetic construct
  SITE 1: note = Attached to another sequence via 16x polyethylene glycol
  SITE 15: note = Attached to another sequence via 16x polyethylene glycol
SGGPGPAGMKGLPGS

SEQ ID NO: 716 - protein, 7 aa, synthetic construct
  SITE 1: note = N-terminus attached to another sequence
  SITE 7: note = Attached to another sequence via 9x polyethylene glycol
GPLGLAG

SEQ ID NO: 717 - protein, 7 aa, synthetic construct
  SITE 1: note = N-terminus attached to another sequence
  SITE 7: note = Attached to another sequence via 4x polyethylene glycol
GPLGLAG

SEQ ID NO: 718 - protein, 7 aa, synthetic construct
  SITE 1: note = N-terminus attached to another sequence
  SITE 7: note = Attached to another sequence via 4x polyethylene glycol
GPLGLAG

SEQ ID NO: 719 - protein, 7 aa, synthetic construct
  SITE 1: note = N-terminus attached to another sequence
  SITE 7: note = Attached to another sequence via 9x polyethylene glycol
GPLGLAG

SEQ ID NO: 720 - protein, 6 aa, synthetic construct
  SITE 1: note = Attached to another sequence via a 1,5-pentanedioic acid-9x polyethylene glycol linker
  SITE 6: note = C-terminus attached to another sequence
PLGLAG

SEQ ID NO: 721 - protein, 6 aa, synthetic construct
  SITE 1: note = Attached to another sequence via a 1,5-pentanedioic acid-4x polyethylene glycol linker
  SITE 6: note = C-terminus attached to another sequence
PLGLAG

SEQ ID NO: 722 - protein, 7 aa, synthetic construct
  SITE 1: note = Attached to another sequence via 1,5-pentanedioic acid
  SITE 7: note = C-terminus attached to another sequence
GPLGLAG

SEQ ID NO: 723 - protein, 17 aa, synthetic construct
  SITE 1: note = Attached to another sequence via 1,5-pentanedioic acid
  SITE 17: note = C-terminus attached to another sequence
GEAGRSANHTPAGLTGP

SEQ ID NO: 724 - protein, 16 aa, synthetic construct
  SITE 1: note = Attached to another sequence via a 1,5-pentanedioic acid-4x polyethylene glycol linker
  SITE 16: note = C-terminus attached to another sequence
EAGRSANHTPAGLTGP

SEQ ID NO: 725 - protein, 16 aa, synthetic construct
  SITE 1: note = Attached to another sequence via a 1,5-pentanedioic acid-9x polyethylene glycol linker
  SITE 16: note = C-terminus attached to another sequence
EAGRSANHTPAGLTGP

SEQ ID NO: 726 - protein, 6 aa, synthetic construct
  SITE 1: note = N-terminus attached to another sequence
  SITE 6: note = Attached to another sequence via 4x polyethylene glycol
PLGLAG

SEQ ID NO: 727 - protein, 6 aa, synthetic construct
  SITE 1: note = Attached to another sequence via 4x polyethylene glycol
  SITE 6: note = C-terminus attached to another sequence
PLGLAG

SEQ ID NO: 728 - protein, 7 aa, synthetic construct
  SITE 1: note = N-terminus attached to another sequence
  SITE 7: note = Attached to another sequence via 4x polyethylene glycol
GPLGLAG

SEQ ID NO: 729 - protein, 7 aa, synthetic construct
  SITE 1: note = N-terminus attached to another sequence
  SITE 7: note = Attached to another sequence via 9x polyethylene glycol
GPLGLAG

SEQ ID NO: 730 - protein, 7 aa, synthetic construct
  SITE 1: note = N-terminus attached to another sequence
  SITE 7: note = C-terminus attached to another sequence
GPLGLAG

SEQ ID NO: 731 - protein, 8 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 8: note = C-terminus attached to another sequence
KKGSGSGS

SEQ ID NO: 732 - protein, 11 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 11: note = C-terminus attached to another sequence
KKSGRVANLVG

SEQ ID NO: 733 - protein, 11 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 11: note = C-terminus attached to another sequence
KKSGRVANLVG

SEQ ID NO: 734 - protein, 12 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 12: note = C-terminus attached to another sequence
KKRGRRVANLVG

SEQ ID NO: 735 - protein, 12 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 12: note = C-terminus attached to another sequence
KKRGRRVANLVG

SEQ ID NO: 736 - protein, 11 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 11: note = C-terminus attached to another sequence
KKSGRVQPLVD

SEQ ID NO: 737 - protein, 10 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 10: note = C-terminus attached to another sequence
KKGSGSGSGS

SEQ ID NO: 738 - protein, 7 aa, synthetic construct
  SITE 1: note = N-terminus attached to another sequence
  SITE 7: note = Attached to another sequence via 4x polyethylene glycol
GSGSGSG

SEQ ID NO: 739 - protein, 7 aa, synthetic construct
  SITE 1: note = N-terminus attached to another sequence
RGRRVAN

SEQ ID NO: 740 - protein, 6 aa, synthetic construct
  SITE 1: note = N-terminus attached to another sequence via 4x polyethylene glycol
  SITE 6: note = C-terminus attached to another sequence
GSGSGS

SEQ ID NO: 741 - protein, 9 aa, synthetic construct
  SITE 1: note = N-terminus attached to another sequence
  SITE 9: note = C-terminus attached to another sequence
SGRVANLVG

SEQ ID NO: 742 - protein, 10 aa, synthetic construct
  SITE 1: note = N-terminus attached to another sequence
  SITE 10: note = C-terminus attached to another sequence
RGRRVANLVG

SEQ ID NO: 743 - protein, 11 aa, synthetic construct
  SITE 1: note = N-terminus attached to another sequence
  SITE 11: note = C-terminus attached to another sequence
SGRVAPQPLVD

SEQ ID NO: 744 - protein, 9 aa, synthetic construct
  SITE 1: note = N-terminus attached to another sequence
  SITE 9: note = C-terminus attached to another sequence
SGRVQPLVD

SEQ ID NO: 745 - protein, 6 aa, synthetic construct
  SITE 1: note = Attached to another sequence via a 1,5-pentanedioic acid-9x polyethylene glycol linker
  SITE 6: note = C-terminus attached to another sequence
GSGSGS